(19) United States
(12) Patent Application Publication
(10) Pub. No.: US 2023/0136218 A1
(43) Pub. Date: May 4, 2023

(54) METHODS AND COMPOSITIONS FOR TREATING ACUTE MYELOID LEUKEMIA

(71) Applicants: President and Fellows of Harvard College, Cambridge, MA (US); The General Hospital Corporation, Boston, MA (US)

(72) Inventors: Nick Van Gastel, Cambridge, MA (US); David T. Scadden, Cambridge, MA (US)

(73) Assignees: President and Fellows of Harvard College, Cambridge, MA (US); The General Hospital Corporation, Boston, MA (US); The General Hospital Corporation, Boston, MA (US)

(21) Appl. No.: 17/762,036

(22) PCT Filed: Sep. 23, 2020
(86) PCT No.: PCT/US20/52339

§ 371 Date: Mar. 18, 2022

Related U.S. Application Data
(60) Provisional application No. 62/976,304, filed on Feb. 13, 2020.
(60) Provisional application No. 62/975,734, filed on Feb. 12, 2020.
(60) Provisional application No. 62/904,619, filed on Sep. 23, 2019.

(51) Int. Cl. A61K 31/47; A61P 35/02; A61K 31/7068; A61K 45/06; A61K 31/704

(52) U.S. Cl. CPC A61K 31/47; A61P 35/02; A61K 31/7068; A61K 45/06; A61K 31/704

(57) ABSTRACT

The disclosure relates to compositions, methods, and kits for treating leukemia, specifically acute myeloid leukemia, in a subject.

### RELATED APPLICATIONS

This application is related to and claims the benefit of U.S. Provisional Application No. 62/904,619, filed Sep. 23, 2019, U.S. Provisional Application No. 62/975,734, filed Feb. 12, 2020, and U.S. Provisional Application No. 62/976,304, filed Feb. 13, 2020. The entire teachings of the applications are incorporated herein by reference.

### BACKGROUND OF THE INVENTION

Natural selection is a fundamental concept of evolutionary biology. When a population is subjected to a stress event or “bottleneck,” only the individuals that have the ability to adapt will survive. This survival of the fittest also occurs in populations of cells (Weissman I L. Stem cells are units of natural selection for tissue formation, for germline development, and in cancer development. Proc Natl Acad Sci USA 2015; 112:8922-8928). An example of this is the clonal evolution of cancer cell populations, introduced by Peter C. Nowell in 1976 (Nowell, P C. The clonal evolution of tumor cell populations. Science 1976; 194:23-28). During cancer progression, subpopulations that possess distinct advantages outcompete other clones, which is further amplified under the selective pressure of therapy. Clonal evolution has been studied particularly well in acute myeloid leukemia (AML) (Ding L, et al. Clonal evolution in relapsed acute myeloid leukaemia revealed by whole-genome sequencing. Nature 2012; 481:506-510), a highly lethal hematopoietic cancer that despite recent advances in mutation-specific therapies is still treated predominantly by conventional chemotherapy. Genetic analysis in AML patients points to residual founder clones persisting through therapy and ultimately causing relapse (Ding L, et al. Clonal evolution in relapsed acute myeloid leukaemia revealed by whole-genome sequencing. Nature 2012; 481:506-510). However, a causal link between genetic lesions and chemotherapy resistance is often hard to established (Magee J A. Comment on: Genomics of primary chemoresistance and remission induction failure in paediatric and adult acute myeloid leukaemia. Br J Haematol 2017; 176:5-6), and even when driver mutations are found they are frequently not amenable to targeted therapies.

Besides genetic alterations, cells have other, evolutionarily ancient systems to protect them from stress, such as shifting metabolic programs (Keller M A, et al. Sulfate radicals enable a non-enzymatic Krebs cycle precursor. Nat Ecol Evol 2017; 1:83; Kültz D. Molecular and evolutionary basis of the cellular stress response. Annu Rev Physiol 2005; 67:225-257; Naviaux R K. Metabolic features of the cell danger response. Mitochondrion 2014; 16:7-17). Emerging evidence shows that AML cells have distinctive metabolic dependencies compared with their normal counterparts (Lagadinou E D, et al. BCL-2 inhibition targets oxidative phosphorylation and selectively eradicates quiescent human leukemia stem cells. Cell Stem Cell 2013; 12(3):329-341; Pei S, et al. Targeting aberrant glutathione metabolism to eradicate human acute myelogenous leukemia cells. J Biol Chem 2013; 288:33542-33558; Wang Y H, et al. Cell-state-specific metabolic dependency in hematopoiesis and leukemogenesis. Cell 2014; 158:1309-1323; Jacque N, et al. Targeting glutaminolysis has antileukemic activity in acute myeloid leukemia and synergizes with BCL-2 inhibition. Blood 2015; 126:1346-1356; Chen W L, et al. Enhanced fructose utilization mediated by SLC2A5 is a unique metabolic feature of acute myeloid leukemia with therapeutic potential. Cancer Cell 2016; 30:779-791; German N J, et al. PHD3 loss in cancer enables metabolic reliance on fatty acid oxidation via deactivation of ACC2. Mol Cell 2016; 63:1006-1020; Ni F, et al. Critical role of ASCT2-mediated amino acid metabolism in promoting leukaemia development and progression. Nat Metab 2019; 1:390-403), but whether specific pathways contribute to the development of chemoresistance in AML remains poorly understood.

### SUMMARY OF THE INVENTION

Cancer relapse begins at the moment malignant cells pass through the extreme metabolic bottleneck of stress from chemotherapy and the by-products of massive surrounding cell death. In acute myeloid leukemia (AML) complete remissions are common, but few are cured. Tracking AML cells in vivo, defining the moment of maximal response following chemotherapy, capturing persisting cells and conducting unbiased metabolic analysis revealed a metabolite profile distinct from the pre-chemo growth or post-chemo relapse phase. Persisting cells used glutamine in a distinctive manner, preferentially fueling pyrimidine synthesis and glutathione generation, but not oxidative phosphorylation. Notably, malignant cell pyrimidine synthesis also required aspartate provided by specific bone marrow stromal cells. Blunting glutamine metabolism or pyrimidine synthesis selected against residual AML cells and improved survival in AML mouse models. Timed cell-intrinsic or niche-focused metabolic disruption may exploit a transient vulnerability and induce a metabolic collapse in cancer cells thereby overcoming chemoresistance.

A novel treatment strategy for AML is described, in which inhibition of pyrimidine synthesis, in combination with one or more additional therapeutic regimens, leads to unexpected and synergistic induction of cell death. In some embodiments the invention relates to a method of treating acute myeloid leukemia in a subject, the method comprising administering an effective amount of at least one pyrimidine synthesis inhibitor (e.g., a glutamine-dependent pyrimidine synthesis inhibitor) to a subject suffering from acute myeloid leukemia who has been treated with a therapeutic, e.g., chemotherapeutic, regimen. In some embodiments the invention relates to a method of depleting acute myeloid leukemia cells, e.g., cells refractory to therapy (for example, chemotherapy) in a subject, the method comprising administering an effective amount of at least one pyrimidine synthesis inhibitor to a subject suffering from acute myeloid leukemia who has been treated with a therapeutic, e.g., chemotherapeutic, regimen.

In certain aspects, the inventions disclosed herein relate to methods of targeting and depleting (partially or completely) chemoresistant acute myeloid leukemia cells in a subject in need thereof, the method comprising administering to the subject an effective amount of an inhibitor (e.g., a pyrimidine synthesis inhibitor) and a chemotherapy treatment regimen, thereby depleting the chemoresistant acute myeloid leukemia cells in the subject. Such treatments can be administered concurrently, for example, or sequentially (for example, the subject can be treated with a chemotherapeutic regimen followed by one or more treatments with one or more inhibitors of the invention). In some embodiments the treatment cycle (chemotherapy followed by one or more treatments with one or more inhibitors of the invention) is repeated one or more times and can comprise the same or different agents. In some embodiments the chemotherapy regimen is induction chemotherapy (iCT); in some embodiments the chemotherapy regimen is acute cytoreductive chemotherapy.

In some aspects, the inventions disclosed herein relate to methods of treating acute myeloid leukemia in a subject in need thereof or methods of promoting survival of a subject suffering from acute myeloid leukemia. The methods comprise administering to the subject an effective amount of a pyrimidine synthesis inhibitor and a chemotherapy treatment regimen (e.g., acute cytoreductive chemotherapy), thereby treating acute myeloid leukemia in the subject. In some embodiments the chemotherapy treatment regimen is administered for a pre-designated period of time and the pyrimidine synthesis inhibitor is administered, e.g., in a single dose, e.g., two to four days following completion of the chemotherapy treatment regimen. In some embodiments the methods further comprise administering a second or subsequent dose of pyrimidine synthesis inhibitor; in some embodiments the second dose is administered, e.g., nine to eleven days following completion of the chemotherapy treatment regimen.

In some aspects, the inventions disclosed herein relate to methods of treating acute myeloid leukemia in a subject in need thereof or methods of promoting survival of a subject suffering from acute myeloid leukemia. The method comprises administering to the subject an effective amount of a pyrimidine synthesis inhibitor and an induction chemotherapy treatment (iCT) regimen, thereby treating acute myeloid leukemia in the subject. In some embodiments the iCT regimen comprises, e.g., administering cytarabine and doxorubicin to the subject for a period of 3-5 days, followed by administering cytarabine alone to the subject for a period of 2-4 days, and the pyrimidine synthesis inhibitor is administered for a period of time beginning 2-4 days after completion of the iCT regimen.

In some embodiments, the pyrimidine synthesis inhibitor is administered in a single dose. In some embodiments a second dose of pyrimidine synthesis inhibitor is administered, e.g., nine to eleven days after completing the iCT regimen. Second or subsequent doses of pyrimidine synthesis inhibitors can comprise the same or different inhibitory agent(s) as the previous dose(s).

In some embodiments, the pyrimidine synthesis inhibitor comprises a small molecule inhibitor. In other embodiments the inhibitor is a protein, peptide or nucleic acid molecule inhibitor. In some embodiments, the pyrimidine synthesis inhibitor comprises a dihydroorotate dehydrogenase inhibitor. In some embodiments, the pyrimidine synthesis inhibitor comprises an orotate-phosphoribosyltransferase (OPRT) inhibitor. In some embodiments, the pyrimidine synthesis inhibitor is selected from the group consisting of brequinar (BRQ), teriflunomide, leflunomide, ML390, pyrazofurin (PF), and combinations thereof. In some embodiments the pyrimidine synthesis inhibitor is a single agent; in other embodiments the pyrimidine synthesis inhibitor is a combination of two or more agents. In some embodiments the pyrimidine synthesis inhibitor is not a pan-glutamine metabolism inhibitor. In some embodiments the pyrimidine synthesis inhibitor is not DON. In some embodiments the pyrimidine synthesis inhibitor is not a glutathione synthesis inhibitor.

In some embodiments, the subject suffers from refractory or relapsed acute myeloid leukemia. In certain embodiments, the method further comprises evaluating the subject to determine if the subject has refractory or relapsed acute myeloid leukemia. In some embodiments, the subject has relapsed from complete remission of acute myeloid leukemia after chemotherapy. In certain embodiments, treating acute myeloid leukemia comprises inducing complete remission of acute myeloid leukemia in the subject or depleting, completely or partially, refractory cell populations. Treating acute myeloid leukemia may comprise inducing complete remission of acute myeloid leukemia in the subject in the absence of a relapse risk due to residual leukemic cells in the subject's bone marrow or peripheral blood.

In some embodiments, the method(s) further comprise administering an aspartate transporter inhibitor. The aspartate transporter may be, for example, SLC1A3. In some embodiments the aspartate transporter inhibitor is selected from the group consisting of DL-threo-beta-benzyloxyaspartate (TBOA), aminooxyacetic acid (AOA), hydrazinosuccinic acid, beta-methylene-DL-aspartate, and combinations thereof.

In some embodiments, the method(s) further comprise administering a glutamate-oxaloacetate transaminase 2 (GOT2) inhibitor. In some embodiments the GOT2 inhibitor is selected from the group consisting of DL-threo-beta-benzyloxyaspartate (TBOA), L-trans-Pyrrolidine-2,4-dicarboxylic acid (L-trans-2,4-PDC), 2-Amino-5,6,7,8-tetrahydro-4-(4-methoxyphenyl)-7-(naphthalen-1-yl)-5-oxo-4H-chromene-3-carbonitrile (UCPH 101), an shRNA, and combinations thereof.

In certain aspects, the inventions disclosed herein relate to methods of depleting chemoresistant acute myeloid leukemia cells in a subject, comprising administering to the subject an effective amount of one or more aspartate transporter (e.g., SLC1A3) inhibitors and a chemotherapy treatment regimen, thereby depleting (partially or fully) the chemoresistant acute myeloid leukemia cells in the subject.

In certain aspects, the inventions disclosed herein relate to methods of depleting chemoresistant acute myeloid leukemia cells in a subject, comprising administering to the subject an effective amount of one or more GOT2 inhibitors and a chemotherapy treatment regimen, thereby depleting the chemoresistant acute myeloid leukemia cells in the subject.

In certain aspects, the inventions disclosed herein relate to pharmaceutical compositions comprising an effective amount of one or more pyrimidine synthesis inhibitors, an effective amount of at least one chemotherapeutic agent to which a subject having acute myeloid leukemia may be or may become resistant or refractory, and a pharmaceutically acceptable carrier, diluent, or excipient.

In some embodiments, the chemotherapeutic regimen comprises at least one chemotherapeutic agent; in some embodiments the agent is one to which acute myeloid leukemic cells in a patient are or become resistant. In one embodiment the at least one chemotherapeutic agent comprises an antimetabolite agent (e.g., cytarabine). In some embodiments, the at least one chemotherapeutic agent comprises an anthracycline agent (e.g., doxorubicin). In certain embodiments, the at least one chemotherapeutic agent comprises an antimetabolite agent and anthracycline agent (e.g., cytarabine and doxorubicin). The chemotherapeutic regimen can comprise administration of a single agent or multiple agents from the same category or different categories/classes of agents.

In some embodiments, the pyrimidine synthesis inhibitor comprises brequinar (BRQ) or an analog thereof.

In some embodiments, the pharmaceutical composition further comprises one or more aspartate transporter inhibitors (e.g., TBOA). In some embodiments, the pharmaceutical composition further comprises one or more GOT2 inhibitors (e.g., an shRNA). In some embodiments, the pharmaceutical composition further comprises one or more aspartate transporter inhibitors and one or more GOT2 inhibitors.

In certain aspects, the inventions disclosed herein relate to pharmaceutical compositions comprising an effective amount of one or more aspartate transporter (e.g., SLC1A3) inhibitors, an effective amount of at least one chemotherapeutic agent to which a subject having acute myeloid leukemia may be or may become resistant or refractory, and a pharmaceutically acceptable carrier, diluent, or excipient.

In certain aspects, the inventions disclosed herein relate to pharmaceutical compositions comprising an effective amount of one or more GOT2 inhibitors, an effective amount of at least one chemotherapeutic agent to which a subject having acute myeloid leukemia may be or may become resistant or refractory, and a pharmaceutically acceptable carrier, diluent, or excipient.

In certain aspects, the inventions disclosed herein relate to kits comprising one or more pyrimidine synthesis inhibitors, at least one chemotherapeutic agent, and instructions for administering the pyrimidine synthesis inhibitor(s) and the at least one chemotherapeutic agent to a subject suffering from acute myeloid leukemia.

In some embodiments, the instructions further comprise directions for administering the at least one chemotherapeutic agent as part of a chemotherapy treatment regimen for the subject. In certain embodiments, the instructions further comprise directions for administering the pyrimidine synthesis inhibitor, and the at least one therapeutic agent to induce complete remission of acute myeloid leukemia in the subject (e.g., remission without risk of relapse due to complete eradication of leukemic cells in the subject).

In some embodiments, the at least one chemotherapeutic agent comprises an antimetabolite agent (e.g., cytarabine). In some embodiments, the at least one chemotherapeutic agent comprises an anthracycline agent (e.g., doxorubicin). In certain embodiments, the at least one chemotherapeutic agent comprises an antimetabolite agent and an anthracycline agent (e.g., cytarabine and doxorubicin).

In some embodiments, the pyrimidine synthesis inhibitor comprises brequinar (BRQ) or an analog thereof.

The above discussed and many other features and attendant advantages of the present invention will become better understood by reference to the following detailed description of the invention when taken in conjunction with the accompanying examples.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1D demonstrate that AML cells exhibit transient metabolic changes in response to chemotherapy. Untargeted metabolomics reveals a distinct and transient metabolic program in chemoresistant AML cells. FIG. 1A provides a schematic overview of the experimental design for untargeted metabolic profiling of freshly isolated mouse MLL-AF9 AML cells. iCT: induction chemotherapy (5 days of cytarabine 100 mg/kg+3 days of doxorubicin 3 mg/kg, I.P.). FIG. 1B provides a heatmap-based visualization of the metabolic profile of MLL-AF9 AML cells obtained from mice with vehicle treatment (vehicle group), at 3 days after induction chemotherapy (iCT group; representing the moment of maximal response) or at 10 days after iCT (relapse group). FIG. 1C provides a Metabolic Pathway Enrichment Analysis using putatively identified metabolites of the iCT versus vehicle/relapse groups showing metabolic pathways enriched in MLL-AF9 AML cells at the moment of maximal response after iCT treatment. Pathway impact is a measure for the percentage of metabolites that were measured in a given pathway, as well as the relative importance of those metabolites in that pathway. Capturing cells at the moment of maximal response showed a metabolite profile that was distinct from AML cells during the times of pre-chemo growth or post-chemo relapse. The enrichment analysis revealed hyperactivation of glutamine and glutamate metabolism, with chemoresistant cells showing high levels of glutamine, glutamate, and aspartate. FIG. 1D shows levels of glutamine, glutamate and aspartate in vehicle, iCT or relapse MLL-AF9 AML cells as measured by untargeted metabolomics. Data are represented as mean±SEM. *p<0.05, **p<0.01, ***p<0.001.

FIGS. 2A-2H demonstrate that timed inhibition of glutamine metabolism overcomes chemoresistance in AML. FIGS. 2A-2C show Kaplan-Meier survival curves of MLL-AF9 AML-bearing mice treated with iCT and/or 6-diazo-5-oxo-L-norleucine (DON; 0.3 mg/kg, I.P.) concomitantly (FIG. 2A) or sequentially (FIGS. 2B-2C). For sequential treatments, mice were either treated with two doses of DON specifically targeting the moment of maximal response (FIG. 2B), or continuously with DON every other day following iCT (FIG. 2C). T: death due to drug toxicity, \hat{ }: moment of maximal response. Inhibition of glutamine metabolism using DON during the window of maximal response reduced the number of chemoresistant cells and led to improved survival in mouse models of AML. While mice did not tolerate daily DON injections, administration of DON every other day was acceptable and further improved mouse survival compared to short term DON treatment. FIGS. 2D-2E show AML burden of MLL-AF9 AML-bearing mice treated sequentially with iCT and/or DON (regimen as in FIG. 2B), determined one day after the last dose of DON through bioluminescence imaging (FIG. 2D) or flow cytometry (FIG. 2E). FIG. 2F shows double-stranded DNA breaks in MLL-AF9 AML cells of mice treated sequentially with iCT and/or DON (regimen as in FIG. 2B), determined one day after the last dose of DON through flow cytometry for gamma-H2AX. FIG. 2G shows LC-MS-based quantification of glutamine, glutamate and aspartate in MLL-AF9 AML cells of mice treated sequentially with iCT and/or DON (regimen as in FIG. 2B), determined one day after the last dose of DON. FIG. 2H shows Kaplan-Meier survival curves of HoxA9/Meis1 AML-bearing mice treated sequentially with iCT and/or two doses of DON (0.3 mg/kg, I.P.) specifically targeting the moment of maximal response. Data are represented as mean±SEM. \hat{ }: moment of maximal response. *p<0.05, **p<0.01, ***p<0.001.

FIGS. 3A-3D demonstrate chemoresistant AML cells do not depend on glutaminase. FIG. 3A shows Kaplan-Meier survival curve of mice engrafted with MLL-AF9 AML cells transduced with scrambled short hairpin RNA (shSCR) or shRNA targeting glutaminase (shGLS), treated with or without iCT. FIG. 3B shows Kaplan-Meier survival curves of MLL-AF9 AML-bearing mice treated with iCT. FIG. 3C provides a heatmap of RNA sequencing data comparing expression of genes related to glutamine metabolism in MLL-AF9 AML cells obtained from mice treated with vehicle or chemotherapy, at the moment of maximal response. FIG. 3D provides flow cytometric analysis of SLC38A1 protein levels on the surface of MLL-AF9 AML cells and on Lin⁻cKit⁺Sca1⁺ and Lin⁻cKit⁺Sca1⁻ normal hematopoietic stem and progenitor cells, at different time during the course of iCT treatment. *p<0.05, **p<0.01, ***p<0.001.

FIGS. 4A-4H demonstrate glutamine drives AML chemoresistance by fueling pyrimidine synthesis. FIGS. 4A-4D show contribution of glutamine carbon and nitrogen to amino acids (FIG. 4A), metabolites involved in the TCA cycle (FIG. 4B), glutathione (FIG. 4C) and nucleotides (FIG. 4D) in MLL-AF9 AML cells obtained from vehicle- and iCT-treated mice at the moment of maximal response. Glu: glutamate, Pro: proline, Asp: aspartate, Asn: asparagine, aKG: alpha-ketoglutarate, Suc: succinate, Mal: malate, Cit: citrate, GSH: reduced glutathione, GSSG: oxidized glutathione, UMP: uridine 5′-monophosphate, AMP: adenosine 5′-monophosphate. Chemoresistant AML cells showed increased flux of glutamine-derived carbon to glutathione, while glutamine-derived nitrogen was used for pyrimidine synthesis. FIGS. 4E-4F show levels of GSH and GSSG (FIG. 4E) or UMP (FIG. 4F) in MLL-AF9 AML cells obtained from vehicle- and iCT-treated mice at the moment of maximal response. FIG. 4G shows Kaplan-Meier survival curves of MLL-AF9 AML-bearing mice treated sequentially with iCT and/or L-buthionine-sulfoximine (BSO, 2.23 mg/kg B.I.D., I.P.+4.45 mg/ml in drinking water; left) or brequinar (BRQ, 50 mg/kg, I.P.; right). \hat{ }: moment of maximal response. The inhibition of pyrimiding synthesis using brequinar (BRQ), but not glutathione synthesis (using BSO), during the window of maximal response extended survival, showing that chemoresistant AML cells particularly depend on glutamine-fueled pyrimidine synthesis. FIG. 4H shows LC-MS-based quantification of UMP in MLL-AF9 AML cells of mice treated sequentially with iCT and/or DON (regimen as in FIG. 2B), determined one day after the last dose of DON. *p<0.05, **p<0.01, ***p<0.001.

FIGS. 5A-5H demonstrate bone marrow stromal cell-derived aspartate supports pyrimidine synthesis in AML cells. FIG. 5A shows labeling of glutamine metabolism and TCA cycle metabolites in MLL-AF9 and HoxA9/Meis1 AML cells by ¹³C₅-glutamine in vivo and in vitro (10 minutes labeling). FIG. 5B shows LC-MS-based quantification of amino acid levels in PB and BM plasma of control mice. FC: fold change. FIG. 5C provides flow cytometric analysis of SLC1A3 protein levels on the surface of different subpopulations of bone marrow stromal cells in control mice. Stroma: total stromal cells (CD45⁻ Ter119⁻), MSC: mesenchymal stromal cells (CD45⁻ Ter119⁻CD31⁻LepR⁺), Fibro: fibroblasts (CD45⁻ Ter119⁻CD31⁻LepR⁻ Sca1⁺CD90⁺), OLC: osteolineage cells: (CD45⁻ Ter119⁻CD31⁻LepR⁻Sca1⁻CD90^{low}CD105⁺), EC: endothelial cells (CD45⁻ Ter119⁻CD31⁺CD105⁺). FIG. 5D provides flow cytometric analysis of changes in SLC1A3 protein levels on the surface of bone marrow stromal cell subpopulations obtained from vehicle- or iCT-treated MLL-AF9 bearing mice at the moment of maximal response, compared to control mice. FIG. 5C provides flow cytometric analysis of changes in GOT1 or GOT2 protein levels on the surface of MSCs obtained from vehicle- or iCT-treated MLL-AF9 bearing mice at the moment of maximal response, compared to control mice. FIG. 5F shows ¹³C₅-glutamine tracing in co-cultures of bone marrow stromal cells (BMSC) and MLL-AF9 AML cells showing transfer of glutamate and aspartate from BMSC to AML cells. FIG. 5G shows viability of MLL-AF9 AML cells in monoculture or coculture with BMSC transduced with shSCR, shGOT1 or shGOT2, in response to different doses of cytarabine (AraC) and doxorubicin (Doxo). FIG. 5H shows labeling of UMP by ¹³C₅-glutamine in MLL-AF9 AML cells obtained from vehicle- or iCT-treated mice at the moment of maximal response. *p<0.05, **p<0.01, ***p<0.001.

FIGS. 6A-6I demonstrate untargeted metabolomics reveals a distinct and transient metabolic program in chemoresistant AML cells. FIGS. 6A-6B show that a triple transgenic mouse model was used in combination with in vivo imaging to define the kinetics of leukemic growth, response to induction chemotherapy and relapse. The moment of maximal response, which is believed to be the moment of maximal selection pressure, was defined as 3-4 days after the last dose of chemotherapy. FIG. 6A provides a schematic overview of the AML mouse model used in this study. iCT: induction chemotherapy (5 days of cytarabine 100 mg/kg+3 days of doxorubicin 3 mg/kg, I.P.). FIG. 6B shows disease progression, visualized by bioluminescence imaging, in mice engrafted with MLL-AF9 AML cells at day 0, treated with vehicle (left) or iCT (right). Arrow indicates the moment of maximal response. FIG. 6C shows histological visualization of GFP-expressing MLL-AF9 AML cells in the bone marrow of vehicle treated mice (left) or mice treated with iCT (right), at the moment of maximal response. FIG. 6D provides analysis of the effect of FACS sorting on the metabolomics profile of MLL-AF9 AML cells, showing correlation of individual metabolite levels between unsorted and FACS sorted cells, obtained from in vitro culture. Metabolites of particular interest for this study, glutamine, glutamate and aspartate, are highlighted. FIG. 6E shows principal component analysis of metabolomics data of MLL-AF9 AML cells comparing vehicle, iCT and relapse groups. FIG. 6F provides levels of individual metabolites in vehicle, iCT or relapse MLL-AF9 AML cells as measured by untargeted metabolomics. FIG. 6G provides liquid chromatography-mass spectrometry (LC-MS)-based quantification of glutamine, glutamate and aspartate in vehicle, iCT or relapse MLL-AF9 AML cells. FIG. 6H provides LC-MS-based quantification of glutamine, glutamate and aspartate in vehicle- or iCT-treated MLL-AF9 AML cells isolated at the moment of maximal response and cultured in vitro for 24 hours. FIG. 6I provides LC-MS-based quantification of glutamine in in vitro cultured MLL-AF9 AML cells treated with vehicle or with 30 nM cytarabine and 10 nM doxorubicin (iCT) for 24 hours. *p<0.05, **p<0.01, ***p<0.001.

FIGS. 7A-7G demonstrate chemoresistant AML cells do not depend on glutaminase. FIG. 7A shows immunoblot detection of GLS in MLL-AF9 cells transduced with shSCR or shGLS, with β-actin as loading control. FIG. 7B shows relative number of viable MLL-AF9 AML cells obtained 72 hours after transduction of cells with shSCR or shGLS. FIG. 7C shows relative number of viable MLL-AF9 AML cells obtained 72 hours after treatment of cells with the pan-glutamine metabolism inhibitor DON or the GLS inhibitors BPTES or CB839. FIG. 7D provides gene ontology analysis for the significantly changed genes (p<0.01) in iCT- versus vehicle-treated MLL-AF9 AML cells obtained at the moment of maximal response. Top annotation clusters are shown according to their combined enrichment scores. FIGS. 7E-7F show Kaplan-Meier survival curves of AML patients grouped according to expression of SLC38A1 above or below the median. Data were obtained from the TCGA dataset (FIG. 7E) or the GSE12417 dataset (FIG. 7F). FIG. 7G shows hazard ratios (HR) and p-values for AML patient survival, bifurcated according to median expression of genes related to glutamine metabolism, in the TCGA and GSE12417 datasets. ***p<0.001.

FIGS. 8A-8D demonstrate glutamine drives AML chemoresistance by fueling pyrimidine synthesis. FIG. 8A provides time course of ¹³C labeling of glutamine metabolism and TCA cycle metabolites in peripheral blood (PB) plasma, bone marrow (BM) plasma and MLL-AF9 AML cells after a bolus tail vein injection of 10.86 mg of ¹³C₅-glutamine. FIG. 8B provides a schematic overview of expected labeling patterns from ¹³C₅-glutamine. White labels: ¹²C, red labels: ¹³C. FIG. 8C shows labeling pattern of glutamine metabolism and TCA cycle metabolites in MLL-AF9 AML cells versus liver tissue by ¹³C₅-glutamine in vivo. FIG. 8D provides a schematic overview of the first steps of pyrimidine synthesis, indicating enzymes in blue and metabolic inhibitors in red. ATP: adenosine 5′-triphosphate, BRQ: brequinar, CAD: carbamoyl-phosphate synthetase 2/aspartate transcarbamylase/dihydroorotase, DHODH: dihydroorotate dehydrogenase, GOT: glutamate-oxaloacetate transaminase, OAA: oxaloacetate, OMP: orotidine 5′-monophosphate, UMPS: uridine 5′-monophosphate synthetase.

FIGS. 9A-9H demonstrate bone marrow stromal cell-derived aspartate supports pyrimidine synthesis in AML cells. FIG. 9A shows labeling of glutamine metabolism and TCA cycle metabolites in MLL-AF9 and HoxA9/Meis1 AML cells by ¹³C₅-glutamine in vitro (24 hours labeling). FIG. 9B shows LC-MS-based quantification of glutamine metabolism and TCA cycle metabolites in MLL-AF9 and HoxA9/Meis1 AML cells obtained from mice or from in vitro cell culture. FIG. 9C shows LC-MS-based quantification of amino acid levels in PB and BM plasma of control mice, MLL-AF9 AML-bearing mice and AML-bearing mice treated with iCT, at the moment of maximal response. FIGS. 9D-9E provide a cell atlas of the mouse bone marrow stroma obtained through single cell RNA sequencing. FIG. 9D provides a t-SNE plot of 20,551 non-hematopoietic cells colored by clustering and annotated post hoc, showing 17 bone marrow stromal cell clusters. FIG. 9E provides a t-SNE plot showing expression of Gls, Got1, Got2, Glud1, Slc1a2 and Slc1a3 in the mouse bone marrow stroma. FIG. 9F shows viability of MLL-AF9 AML cells in monoculture or coculture with BMSC, in response to different doses of AraC and Doxo. FIG. 9G shows LC-MS-based quantification of glutamine in MLL-AF9 AML cells co-cultured with BMSC and treated with vehicle or with 30 nM cytarabine and 10 nM doxorubicin (iCT) for 24 hours. FIG. 9H shows immunoblot detection of GOT1 and GOT2 in BMSC transduced with shSCR, shGOT1 or shGOT2, with β-actin as loading control. *p<0.05.

FIGS. 10A-10B demonstrate cell regulatory programs present in AML cells. FIG. 10A provides a diagram demonstrating chemotherapy resistance. FIG. 10B provides a schematic showing timed metabolic collapse to overcome chemoresistance in AML. Chemoresistance is the difference between remission and cure in cancer patients, and is particularly evidence in acute myeloid leukemia (AML) where complete remissions are common, but few are cured. Genetic analysis in patients points to residual founder clones persisting through therapy and ultimately causing relapse. Most of these genetic alterations are currently not amenable to targeted therapies, and therefore the focus is on cell regulatory programs rather than genetics. Prior work has indicated that AML cells have distinctive metabolic dependencies compared with their normal counterparts. It is hypothesized that residual chemoresistant cells must pass through extreme metabolic challenges when under selection from chemotherapy and surrounded by massive cell death.

FIGS. 11A-11D demonstrate that AML cells exhibit transient metabolic changes in response to chemotherapy. Untargeted metabolomics reveals a distinct and transient metabolic program in chemoresistant AML cells. FIG. 11A provides a schematic overview of the experimental design for untargeted metabolic profiling of freshly isolated mouse MLL-AF9 AML cells. iCT: induction chemotherapy (5 days of cytarabine 100 mg/kg+3 days of doxorubicin 3 mg/kg, I.P.). FIG. 11B provides a heatmap-based visualization of the metabolic profile of MLL-AF9 AML cells obtained from mice with vehicle treatment (vehicle group), at 3 days after induction chemotherapy (iCT group; representing the moment of maximal response) or at 10 days after iCT (relapse group). FIG. 11C provides a Metabolic Pathway Enrichment Analysis using putatively identified metabolites of the iCT versus vehicle/relapse groups showing metabolic pathways enriched in MLL-AF9 AML cells at the moment of maximal response after iCT treatment. Pathway impact is a measure for the percentage of metabolites that were measured in a given pathway, as well as the relative importance of those metabolites in that pathway. Capturing cells at the moment of maximal response showed a metabolite profile that was distinct from AML cells during the times of pre-chemo growth or post-chemo relapse. The enrichment analysis revealed hyperactivation of glutamine and glutamate metabolism, with chemoresistant cells showing high levels of glutamine, glutamate, and aspartate. FIG. 11D shows levels of glutamine, glutamate and aspartate in vehicle, iCT or relapse MLL-AF9 AML cells as measured by untargeted metabolomics. Data are represented as mean±SEM. *p<0.05, **p<0.01, ***p<0.001.

FIGS. 12A-12G demonstrate timed inhibition of glutamine metabolism overcomes chemoresistance in AML. FIGS. 12A-12C provide Kaplan-Meier survival curves of MLL-AF9 AML-bearing mice treated with iCT and/or 6-diazo-5-oxo-L-norleucine (DON; 0.3 mg/kg, I.P.) concomitantly (FIG. 12A) or sequentially (FIGS. 12B-12C). For sequential treatments, mice were either treated with two doses of DON specifically targeting the moment of maximal response (FIG. 12B), or continuously with DON every other day following iCT (FIG. 12C). T: death due to drug toxicity, \hat{ }: moment of maximal response. FIGS. 12D-12E show AML burden of MLL-AF9 AML-bearing mice treated sequentially with iCT and/or DON (regimen as in FIG. 12B), determined one day after the last dose of DON through bioluminescence imaging (FIG. 12D) or flow cytometry (FIG. 12E). FIG. 12F shows double-stranded DNA breaks in MLL-AF9 AML cells of mice treated sequentially with iCT and/or DON (regimen as in FIG. 12B), determined one day after the last dose of DON through flow cytometry for gamma-H2AX. FIG. 12G provides Kaplan-Meier survival curves of HoxA9/Meis1 AML-bearing mice treated sequentially with iCT and/or two doses of DON (0.3 mg/kg, I.P.) specifically targeting the moment of maximal response. T: death due to drug toxicity. \hat{ }: moment of maximal response. FIG. 12H shows percentage of apoptotic cells in MLL-AF9 AML cells obtained from mice treated sequentially with iCT and/or DON (regimen as in FIG. 12B), determined one day after the last dose of DON through flow cytometry for active Caspase 3. The combination treatment is shown to result in more apoptotic cells. Data are represented as mean±SEM. *p<0.05, **p<0.01, ***p<0.001.

FIGS. 13A-13I demonstrate residual AML cells (e.g., chemoresistant AML cells) do not depend on glutaminase. FIG. 13A provides a Kaplan-Meier survival curve of mice engrafted with MLL-AF9 AML cells transduced with scrambled short hairpin RNA (shSCR) or shRNA targeting glutaminase (shGLS), treated with or without iCT. FIG. 13B provides Kaplan-Meier survival curves of MLL-AF9 AML-bearing mice treated sequentially with iCT. FIG. 13C provides a heatmap of RNA sequencing data comparing expression of genes related to glutamine metabolism in MLL-AF9 AML cells obtained from mice treated with vehicle or chemotherapy, at the moment of maximal response. FIG. 13D provides flow cytometric analysis of SLC38A1 protein levels on the surface of MLL-AF9 AML cells and on Lin⁻cKit⁺Sca1+ and Lin⁻cKit⁺Sca1⁻ normal hematopoietic stem and progenitor cells, at different times during the course of iCT treatment. FIG. 13E provides Kaplan-Meier survival curves of AML patients grouped according to expression of SLC38A1 above or below the median. Data were obtained from the TCGA dataset or the GSE12417 dataset. Data are represented as mean±SEM. *p<0.05, **p<0.01, ***p<0.001. FIG. 13F provides gene set enrichment analysis (Hallmark gene sets) in iCT-versus vehicle-treated MLL-AF9 AML cells obtained at the moment of maximal response. NES: normalized enrichment score. FIGS. 13G-13H show mitochondrial membrane potential as measured by tetramethylrhodamine ethyl ester (TMRE) staining (FIG. 13G) and levels of reactive oxygen species (ROS) as measured by CellROX Orange staining (FIG. 13H) in MLL-AF9 AML cells obtained from mice treated with vehicle or chemotherapy at the moment of maximal response. FIG. 13I shows flow cytometric analysis of SLC38A1 protein levels on the surface of bulk MLL-AF9 mouse AML cells (GFP⁺), leukemic granulocyte-monocyte progenitors (GMPs, also known as leukemia-initiating cells; GFP⁺Lin⁻cKit⁺Sca1⁻CD34⁺CD16/32⁺), bulk normal bone marrow cells (GFP⁻) and normal GMPs (GFP⁻Lin⁻cKit⁺Sca1⁻CD34⁺CD16/32⁺), at the moment of maximal response after iCT treatment. L-GMPs, but not normal GMPs, are shown to increase SLC38A1 levels after iCT treatment.

FIGS. 14A-14J demonstrate glutamine drives AML chemoresistance by fueling pyrimidine synthesis. FIGS. 14A-14D show contribution of glutamine carbon and nitrogen to amino acids (FIG. 14A), metabolites involved in the TCA cycle (FIG. 14B), glutathione (FIG. 14C) and nucleotides (FIG. 14D) in MLL-AF9 AML cells obtained from vehicle- and iCT-treated mice at the moment of maximal response. Glu: glutamate, Pro: proline, Asp: aspartate, Asn: asparagine, aKG: alpha-ketoglutarate, Suc: succinate, Mal: malate, Cit: citrate, GSH: reduced glutathione, GSSG: oxidized glutathione, UMP: uridine 5′-monophosphate, AMP: adenosine 5′-monophosphate. FIGS. 14E-14F show levels of GSH and GSSG (FIG. 14E) or UMP (FIG. 14F) in MLL-AF9 AML cells obtained from vehicle- and iCT-treated mice at the moment of maximal response. FIG. 14G provides Kaplan-Meier survival curves of MLL-AF9 AML-bearing mice treated sequentially with iCT and/or L-buthionine-sulfoximine (BSO, 2.23 mg/kg B.I.D., I.P.+4.45 mg/ml in drinking water; left) or brequinar (BRQ, 50 mg/kg, I.P.; right). \hat{ }: moment of maximal response. FIG. 14H shows LC-MS-based quantification of UMP in MLL-AF9 AML cells of mice treated sequentially with iCT and/or DON (regimen as in FIG. 12B), determined one day after the last dose of DON. *p<0.05, **p<0.01, ***p<0.001. FIGS. 14I-14J show flow cytometric visualization and quantification of CD34 and cKit levels (FIG. 14I) and colony-forming capacity (FIG. 14J) in AML cells obtained from mice sequentially treated with iCT and/or DON (reimen as in FIG. 2B) or BRQ (regimen as in FIG. 4G), determined four days after the last dose of iCT. Data are represented as mean±SEM. *p<0.05, **p<0.01, ***p<0.001.

FIGS. 15A-15J demonstrate bone marrow stromal cell-derived aspartate supports pyrimidine synthesis in AML cells. FIG. 15A shows labeling of glutamine metabolism and TCA cycle metabolites in MLL-AF9 and HoxA9/Meis1 AML cells by ¹³C₅-glutamine in vivo and in vitro (10 minutes labeling). FIG. 15B provides LC-MS-based quantification of amino acid levels in PB and BM plasma of control mice. FC: fold change. FIG. 15C shows flow cytometric analysis of SLC1A3 protein levels on the surface of different subpopulations of bone marrow stromal cells (BMSC) in control mice. Stroma: total stromal cells (CD45⁻Ter119⁻), MSC: mesenchymal stromal cells (CD45⁻ Ter119⁻CD31⁻LepR⁺), Fibro: fibroblasts (CD45⁻ Ter119⁻CD31⁻LepR⁻ Sca1⁺CD90⁺), OLC: osteolineage cells: (CD45⁻ Ter119⁻CD31⁻LepR⁻Sca1⁻CD90^{low}CD105⁺), EC: endothelial cells (CD45⁻ Ter119⁻CD31⁺CD105⁺). Left: SLC1A3 mean fluorescence intensity (MFI), right: percentage SLC1A3^{high} cells. FIG. 15D shows flow cytometric analysis of changes in SLC1A3 protein levels on the surface of bone marrow stromal cell subpopulations obtained from vehicle- or iCT-treated MLL-AF9 bearing mice at the moment of maximal response, compared to control mice. FIG. 15E shows flow cytometric analysis of changes in GOT1 or GOT2 protein levels on the surface of MSCs obtained from vehicle- or iCT-treated MLL-AF9 bearing mice at the moment of maximal response, compared to control mice. FIG. 15F shows ¹³C₅-glutamine tracing in co-cultures of bone marrow stromal cells (BMSC) and MLL-AF9 AML cells showing transfer of glutamate and aspartate from BMSC to AML cells. A transfer of glutamate and aspartate from BMSC to AML cells in co-cultures is shown. A portion of the aspartate may be used for UMP synthesis given the low amount of labeling in AML cells, while UMP was not detected in BMSC. FIG. 15G shows viability of MLL-AF9 AML cells in mono-culture or co-culture with BMSC transduced with shSCR, shGOT1 or shGOT2, in response to different doses of cytarabine (AraC) and doxorubicin (Doxo). FIG. 15H shows labeling of UMP by ¹³C₅-glutamine in MLL-AF9 AML cells obtained from vehicle- or iCT-treated mice at the moment of maximal response. FIGS. 15I-15J show SLC1A3 levels in AML cells. FIG. 15I provides flow cytometric analysis of SLC1A3 protein levels on the surface of stromal cells (CD45⁻ Ter119⁻), hematopoietic stem cells (HSC; Lin⁻cKit⁺Sca1+CD48⁻CD150⁺), granulocyte-monocyte progenitor cells (GMP; Lin⁻cKit⁺Sca1⁻CD34⁺CD16/32⁺) and MLL-AF9 AML cells. FIG. 15J shows expression of SLC1A3, GOT1 and GOT2 on human AML patient-derived xenograft (PDX) cells. Data obtained from Farge et al., Cancer Discov 2017 (GSE97631). AML cells express high levels of the aspartate transporter SLC1A3, which may further increase after iCT treatment. Data are represented as mean±SEM. *p<0.05, **p<0.01, ***p<0.001.

FIGS. 16A-16J demonstrate AML cells exhibit transient metabolic changes in response to chemotherapy. Untargeted metabolomics reveals a distinct and transient metabolic program in chemoresistant AML cells. FIG. 16A provides a schematic overview of the AML mouse model used in this study. iCT: induction chemotherapy (5 days of cytarabine 100 mg/kg+3 days of doxorubicin 3 mg/kg, I.P.). FIG. 16B shows disease progression, visualized by bioluminescence imaging, in mice engrafted with MLL-AF9 AML cells at day 0, treated with vehicle (left) or iCT (right). Arrow indicates the moment of maximal response. FIG. 16C shows histological visualization of GFP-expressing MLL-AF9 AML cells in the bone marrow of vehicle treated mice (left) or mice treated with iCT (right), at the moment of maximal response. FIG. 16D provides analysis of the effect of FACS sorting on the metabolic profile of MLL-AF9 AML cells, showing correlation of individual metabolite levels between unsorted and FACS-sorted cells, obtained from in vitro culture. Metabolites of particular interest for this study, glutamine, glutamate and aspartate, are highlighted. FIG. 16E shows principal component analysis of metabolomics data of MLL-AF9 AML cells comparing vehicle, iCT and relapse groups. FIG. 16F shows levels of individual metabolites in vehicle, iCT or relapse MLL-AF9 AML cells as measured by untargeted metabolomics. FIG. 16G shows liquid chromatography-mass spectrometry (LC-MS)-based quantification of glutamine, glutamate and aspartate in vehicle, iCT or relapse MLL-AF9 AML cells. FIG. 16H provides LC-MS-based quantification of glutamine, glutamate and aspartate in vehicle- or iCT-treated MLL-AF9 AML cells isolated at the moment of maximal response and cultured in vitro for 24 hours. FIG. 16I provides LC-MS-based quantification of glutamine in in vitro cultured MLL-AF9 AML cells treated with vehicle or with 30 nM cytarabine and 10 nM doxorubicin (iCT) for 24 hours. FIG. 16J provide a principal component analysis (PC2 versus PC3) of metabolomics data of MLL-AF9 AML cells comparing vehicle, iCT and relapse groups. Data are represented as mean±SEM. *p<0.05, **p<0.01, ***p<0.001.

FIG. 17 demonstrate timed inhibition of glutamine metabolism overcomes chemoresistance in AML. LC-MS-based quantification of glutamine, glutamate and aspartate in MLL-AF9 AML cells of mice treated sequentially with iCT and/or DON (regimen as in FIG. 2B), determined one day after the last dose of DON. Data are represented as mean±SEM. *p<0.05, **p<0.01, ***p<0.001.

FIGS. 18A-18H demonstrate residual AML cells (e.g., chemoresistant AML cells) do not depend on glutaminase. FIG. 18A shows immunoblot detection of GLS in MLL-AF9 cells transduced with shSCR or shGLS, with β-actin as loading control. FIG. 18B provides relative number of viable MLL-AF9 AML cells obtained 72 hours after transduction of cells with shSCR or shGLS. FIG. 18C shows relative number of viable MLL-AF9 AML cells obtained 72 hours after treatment of cells with the pan-glutamine metabolism inhibitor DON (1 μM) or the GLS inhibitors BPTES (5 μM) or CB839 (0.5 μM). FIG. 18D shows gene set enrichment analysis for the significantly changed genes (p<0.01) in iCT- versus vehicle-treated MLL-AF9 AML cells obtained at the moment of maximal response. Top annotation clusters are shown. FIG. 18E shows hazard ratios for AML patient survival, bifurcated according to median expression of genes related to glutamine metabolism, in the TCGA and GSE12417 datasets. FIG. 18F shows gene set enrichment analysis (gene ontology biological process gene sets in iCT-versus vehicle-treated MLL-AF9 AML cells obtained at themoment of maximal response. NES: normalized enrichment score. FIG. 18G shows enrichment plots for genes related to fatty acid metabolism (left) and oxidative phosphorylation (right) comparing iCT- and vehicle-treated MLL-AF9 AML cells obtained at the moment of maximal response. FIG. 18H shows expression of Gls and Gls2 in MLL-AF9 mouse AML cells as determined by RNA sequencing. Gls is shown to express at much higher levels than Gls2. Data are represented as mean±SEM (B,C) or as Hazard Ratio±95% confidence intervals (FIG. 18E). *p<0.05, **p<0.01, ***p<0.001.

FIGS. 19A-19G demonstrate glutamine drives AML chemoresistance by fueling pyrimidine synthesis. FIG. 19A shows time course of ¹³C labeling of glutamine metabolism and TCA cycle metabolites in peripheral blood (PB) plasma, bone marrow (BM) plasma and MLL-AF9 AML cells after a bolus tail vein injection of 10.86 mg of ¹³C₅-glutamine. FIG. 19B provides a schematic overview of expected labeling patterns from ¹³C₅-glutamine. White labels: ¹²C, red labels: ¹³C. FIG. 19C shows labeling pattern of glutamine metabolism and TCA cycle metabolites in MLL-AF9 AML cells versus liver tissue by ¹³C₅-glutamine in vivo. FIG. 19D provides a schematic overview of the first steps of pyrimidine synthesis, indicating enzymes in blue and metabolic inhibitors in red. ATP: adenosine 5′-triphosphate, BRQ: brequinar, CAD: carbamoyl-phosphate synthetase 2/aspartate transcarbamylase/dihydroorotase, DHODH: dihydroorotate dehydrogenase, GOT: glutamate-oxaloacetate transaminase, OAA: oxaloacetate, OMP: orotidine 5′-monophosphate, UMPS: uridine 5′-monophosphate synthetase. FIG. 19E shows Kaplan-Meier survival curves of HoxA9/Meis1 AML-bearing mice treated sequentially with iCT and/or brequinar (BRQ, 50 mg/kg, I.P.; right). \hat{ }: moment of maximal response. FIG. 19F shows flow cytometric analysis of disease burden of MLL-AF9 AML-bearing mice treated sequentially with iCT and/or BRQ (regimen as in FIG. 14G), determined four days after the last dose of iCT. FIG. 19G shows flow cytometric quantification of different subpopulations, as determined by CD34 and cKit levels, in AML cells obtained from mice sequentially treated with iCT and/or DON (regimen as in FIG. 12B) or BRQ (regimen as in FIG. 14G), determined four days after the last dose of iCT. The absolute cell counts for each of the different populations are shown. FIG. 19H shows LC-MS-based quantification of reduced glutathione (GSH) in MLL-AF9 AML cells obtained from mice treated sequentially with iCT and/or DON (regimen as in FIG. 2B), determined one day after the last dose of DON. The DON treatment is shown to be effective in suppressing the increase in GSH levels in MLL-AF9 AML cells after iCT treatment. FIG. 19I shows liquid chromatography-mass spectrometry (LC-MS)-based quantification of different metabolites related to glutamine metabolism and pyrimidine synthesis in HoxA9/Meis1 AML cells obtained from mice treated with vehicle or brequinar (BRQ) 24 prior. BRQ effectively inhibits pyrimidine synthesis. Data are represented as mean±SEM. *p<0.05, **p<0.01, ***p<0.001.

FIGS. 20A-20H demonstrate bone marrow stromal cell-derived aspartate supports pyrimidine synthesis in AML cells. FIG. 20A shows labeling of glutamine metabolism and TCA cycle metabolites in MLL-AF9 and HoxA9/Meis1 AML cells by ¹³C₅-glutamine in vitro (24 hours labeling). FIG. 20B provides LC-MS-based quantification of glutamine metabolism and TCA cycle metabolites in MLL-AF9 and HoxA9/Meis1 AML cells obtained from mice or from in vitro cell culture. FIG. 20C provides LC-MS-based quantification of amino acid levels in PB and BM plasma of control mice, MLL-AF9 AML-bearing mice and AML-bearing mice treated with iCT, at the moment of maximal response. FIGS. 20D-20E show a cell atlas of the mouse bone marrow stroma obtained through single cell RNA sequencing. (FIG. 20D) t-SNE plot of 20,551 non-hematopoietic cells colored by clustering and annotated post hoc, showing 17 bone marrow stromal cell clusters. (FIG. 20E) t-SNE plots showing expression of Gls, Got1, Got2, Glud1, Slc1a2 and Slc1a3 in the mouse bone marrow stroma. FIG. 20F shows viability of MLL-AF9 AML cells in mono-culture or co-culture with BMSC, in response to different doses of cytarabine (AraC) and doxorubicin (Doxo). FIG. 20G provides LC-MS-based quantification of glutamine in MLL-AF9 AML cells co-cultured with BMSC and treated with vehicle or with 30 nM cytarabine and 10 nM doxorubicin (iCT) for 24 hours. FIG. 20H shows immunoblot detection of GOT1 and GOT2 in BMSC transduced with shSCR, shGOT1 or shGOT2, with β-actin as loading control. Data are represented as mean±SEM. *p<0.05, ***p<0.001.

FIG. 21 provides a table listing the peak areas of 654 metabolites determined by untargeted metabolomics analysis. The first column lists putative metabolite IDs (from mzCoud, KEGG, and HMDB). The second column lists the HMDB number corresponding to the putative ID given in the first column. Column three lists the molecular weight (monoisotopic) of the metabolite. The fourth column provides the retention time of the metabolite on the HILIC column. Column five provides the mzCloud score for the metabolites that were putatively identified based on MS/MS spectra. Note: metabolites with a putative ID assigned by no mzCloud score were annotated based on their monoisotopic molecular weight using the KEGG and HMDB databases. The remaining columns provide the peak areas of different metabolites in either the vehicle (AML cells isolated from vehicle-treated mice, 3 days after the last dose), iCT (AML cells isolated from iCT-treated mice, 3 days after the last dose), or relapse (AML cells isolated from iCT-treated mice, 10 days after the last dose) cells.

FIG. 22 provides a table listing ANOVA analysis of the 340 putatively-identified metabolites, using MetaboAnalyst 4.0. Tukey's HSD was used as a posthoc test.

### DETAILED DESCRIPTION OF THE INVENTION

Chemoresistance is the difference between remission and cure in cancer patients and is particularly evident in acute myeloid leukemia (AML) where complete remissions are common, but few are cured. Prior work has indicated that AML cells have distinctive metabolic dependencies compared with their normal counterparts.

It was hypothesized that residual chemoresistant cells must pass through extreme metabolic challenges. Capturing cells at the moment of maximal response after chemotherapy showed a metabolite profile that was distinct from AML cells during pre-chemo growth or post-chemo relapse. Metabolite enrichment analysis revealed hyperactivation of glutamine metabolism, with chemoresistant cells showing high levels of glutamine, glutamate and aspartate. Pharmacological inhibition of glutamine metabolism during the window of maximal response reduced the number of chemoresistant cells and improved survival in mouse models of AML. In vivo metabolic tracer analysis revealed that chemoresistant AML cells mainly depend on glutamine nitrogen to fuel pyrimidine synthesis. Aspartate, also essential for pyrimidine synthesis, can be made from glutamine through the Krebs cycle. Interestingly, almost no glutamine carbon was detected entering the Krebs cycle even though labeled aspartate was found, indicating a different aspartate source. Single cell RNA sequencing of bone marrow stromal cells (BMSC) revealed that CXCL12-positive BMSC express high levels of aspartate synthesis genes and transporters, which further increased in the presence of AML cells. In vitro co-cultures showed that BMSC convert glutamine to aspartate, and transfer this to AML cells. Inhibition of aspartate synthesis in BMSC reduced AML cell numbers, showing the importance of this metabolic crosstalk. It is proposed that induction of a timed metabolic collapse targeting AML cells both directly and indirectly through the bone marrow niche can prevent development of chemoresistance and improve the rate of cure.

The disclosure relates to the discovery of a novel treatment strategy for leukemia (e.g., acute myeloid leukemia (AML)), in which inhibition of pyrimidine synthesis in combination with chemotherapy leads to unexpected and synergistic induction of leukemia cell death. It was found that pyrimidine synthesis inhibitors can overcome resistance to standard chemotherapy in AML. Accordingly, the disclosure contemplates the use of one or more agents (e.g., pyrimidine synthesis inhibitors) in methods, compositions, and kits for treating AML.

### Targeting Chemoresistant Cells

In some aspects, disclosed herein are methods for targeting chemoresistant leukemic cells in a population of cells. Such methods are useful for, amongst other things, treating leukemia (e.g., acute myeloid leukemia). In one embodiment, a method of targeting chemoresistant leukemic cells in a population of cells comprises contacting the population of cells with an effective amount of an inhibitor in combination with a chemotherapy treatment regimen, thereby targeting chemoresistant leukemic cells in the cell population.

It should be appreciated by those skilled in the art that the compositions and methods described herein decrease the amount or activity of leukemic cells in a population of cells. In some embodiments, the compositions and methods described herein preferably decrease the number, activity, and/or proliferation of chemoresistant leukemic cells in a population of cells. The amount or number of leukemic cells eradicated, reduced, or inhibited in any particular population of cells can be proportional to the concentration of inhibitor (e.g., pyrimidine synthesis inhibitor) to which the population of cells has been exposed. In some instances, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or as much as 100% of the leukemic cells in the population of cells are eradicated, reduced, or inhibited by exposure to or contact with an inhibitor in combination with a chemotherapy regimen. In some embodiments, at least 20% of the leukemic cells in the population of cells are eradicated, reduced, or inhibited. In some embodiments, at least 50% of the leukemic cells in the population of cells are eradicated, reduced, or inhibited. In some embodiments, at least 70% of the leukemic cells in the population of cells are eradicated, reduced, or inhibited. In some embodiments, all of the leukemic cells in the population of cells are eradicated, reduced, or inhibited. In certain embodiments, the leukemic cells are chemoresistant leukemic cells.

The present invention contemplates eradicating leukemic cells by contacting a population of cells with, or exposing the population of cells to, an inhibitor in combination with a chemotherapy regimen. In some embodiments, the leukemia cells comprise leukemia cells from an acute myeloid leukemia cell line. Exemplary acute myeloid leukemia cell lines include, but are not limited to, MLL-AF9 cells, MLL-ENL cells, Nup98-HoxA9 cells, AML1-ETO9A cells, KG-1 cells, KG-1a cells, U937 cells, THP1 cells, HL60 cells, HoxA9/Meis1 cells, and NB-4 cells. In some embodiments, the population of cells comprises primary leukocytes, such as bone marrow leukocytes and peripheral blood leukocytes. Examples of such primary leukocytes include, without limitation, stem and progenitors, mononuclear cells, myeloblasts, neutrophils, NK cells, macrophages, granulocytes, monocytes, and lineage−/cKit+/Sca1+(LKS) cells.

In some aspects an inhibitor comprises a small molecule, nucleic acid, polypeptide, peptide, drug, ion, etc. An “inhibitor” can be any chemical, entity or moiety, including without limitation synthetic and naturally-occurring proteinaceous and non-proteinaceous entities. In some embodiments, an inhibitor is nucleic acids, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, inhibitors are a small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds. In some aspects an inhibitor is a pyrimidine synthesis inhibitor, a glutamate-oxaloacetate transaminase 2 (GOT2) inhibitor, and/or an aspartate transporter inhibitor.

In some aspects a pyrimidine synthesis inhibitor is a dihydroorotate dehydrogenase inhibitor. In some aspects a pyrimidine synthesis inhibitor is an orotate-phosphoribosyltransferase (OPRT) inhibitor. In some aspects a pyrimidine synthesis inhibitor is selected from the group consisting of brequinar (BRQ), teriflunomide, leflunomide, pyrazofurin (PF), ML390, and analogs thereof. In some embodiments a pyrimidine synthesis inhibitor is brequinar (BRQ). In some embodiments a pyrimidine synthesis inhibitor is teriflunomide. In some embodiments a pyrimidine synthesis inhibitor is leflunomide. In some embodiments a pyrimidine synthesis inhibitor is pyrazofurin (PF). In some embodiments a pyrimidine synthesis inhibitor is ML390.

In some embodiments methods of targeting chemoresistant leukemic cells in a population of cells comprise inhibiting the transport of aspartate from bone marrow stromal cells (BMSCs) (e.g., LepR⁺ cells) to AML cells. In some embodiments, the methods of targeting chemoresistant leukemic cells in a population of cells comprise contacting the population of cells with an effective amount of one or more inhibitors selected from the group consisting of a pyrimidine synthesis inhibitor, an aspartate transporter inhibitor, and a glutamate-oxaloacetate transaminase 2 (GOT2) inhibitor, in combination with a chemotherapy treatment regimen. In some embodiments the methods of targeting chemoresistant leukemic cells in a population of cells comprise contacting the population of cells with an effective amount of an aspartate transporter inhibitor and/or a GOT2 inhibitor, in combination with a chemotherapy treatment regimen. In some embodiments the methods of targeting chemoresistant leukemic cells in a population of cells comprise contacting the population of cells with an effective amount of an aspartate transporter inhibitor. An aspartate transporter may be SLC1A3. In some embodiments an inhibitor of an aspartate transporter (e.g., SLC1A3) is DL-threo-beta-benzyloxyaspartate (TBOA), L-trans-Pyrrolidine-2,4-dicarboxylic acid (L-trans-2,4-PDC), 2-Amino-5,6,7,8-tetrahydro-4-(4-methoxyphenyl)-7-(naphthalen-1-yl)-5-oxo-4H-chromene-3-carbonitrile (UCPH 101), or analogs thereof. In some embodiments the methods of targeting chemoresistant leukemic cells in a population of cells further comprise contacting the population of cells with an effective amount of a glutamate-oxaloacetate transaminase 2 (GOT2) inhibitor. In some embodiments a GOT2 inhibitor is aminooxyacetic acid (AOA), hydrazinosuccinic acid, beta-methylene-DL-aspartate, or analogs thereof. In some embodiments a GOT2 inhibitor is an shRNA.

It should be appreciated that the effective amount of the agents for use in accordance with the present inventions (e.g., a pyrimidine synthesis inhibitor, an aspartate transporter inhibitor, and/or a GOT2 inhibitor) may vary, for example, depending on the inhibitor being used, the number of doses, and its location of use. In some embodiments, the effective amount of the inhibitor for in vitro or in vivo use comprises a concentration in the range of 0.01 μM to 500 μM, or alternatively within the range of 0.5 μM to 1.0 μM. In some embodiments, the effective amount of the inhibitor for in vitro or in vivo use comprises a concentration in the range of 0.10 mg/kg to 100 mg/kg, within the range of 1 mg/kg to 90 mg/kg, within the range of 10 mg/kg to 80 mg/kg, within the range of 20 mg/kg to 70 mg/kg, or within the range of 30 mg/kg to 60 mg/kg. In some embodiments, the effect amount comprises a concentration of about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, or 100 mg/kg. In some embodiments, the effective amount comprises a concentration of about 46 mg/kg, 47 mg/kg, 48 mg/kg, 49 mg/kg, 50 mg/kg, 51 mg/kg, 52 mg/kg, 53 mg/kg, 54 mg/kg or 55 mg/kg. In some embodiments, the effective amount comprises a concentration of about 45 mg/kg. In some embodiments, the effective amount comprises a concentration of about 50 mg/kg. In some embodiments, the effective amount comprises a concentration of about 55 mg/kg.

It is generally understood that synergism may occur between an inhibitor (e.g., a pyrimidine synthesis inhibitor) and the chemotherapy treatment for effectively targeting and treating chemoresistant AML cells. In some embodiments, the point of synergism between a pyrimidine synthesis inhibitor (e.g., BRQ) and chemotherapy (e.g., induction chemotherapy) may vary depending on the type of pyrimidine synthesis inhibitor used and the specific chemotherapy treatment.

In some embodiments, the contacting occurs in vitro or ex vivo. In other embodiments, the contacting occurs in vivo. In some embodiments, the in vivo contact is in a subject as described herein.

### Methods of Treatment

The disclosure contemplates various methods of treatment utilizing the compositions and kits comprising the inhibitors (e.g., a pyrimidine synthesis inhibitor, an aspartate transporter inhibitor, and/or a GOT2 inhibitor) and anti-cancer treatment regimen (e.g., chemotherapy treatments) described herein. The disclosure contemplates the treatment of any disease in which cells are chemoresistant. The inhibitors described herein can be used to treat and/or prevent such diseases.

In some aspects, the disclosure provides a method of treating acute myeloid leukemia in a subject in need thereof, the method comprising administering to the subject an effective amount of an inhibitor (e.g., a pyrimidine synthesis inhibitor, an aspartate transporter inhibitor, and/or a GOT2 inhibitor) described herein, thereby treating acute myeloid leukemia in the subject. In some embodiments, the method further comprises administering an anti-cancer treatment regimen (e.g., a chemotherapy treatment regimen) to the subject.

As used herein, “anti-cancer treatment regimen” or “anticancer agent” refers to a treatment regime that inhibits, ameliorates, reduces the symptoms of, or eliminates a proliferative disorder, for example cancer, in a subject. An “anti-cancer treatment regimen” encompasses a treatment regime that may include one or more chemical compounds (e.g., chemotherapy), one or more physical treatments such as radiation (e.g., radiotherapy), heat, or even surgery, or a combination of chemical compounds and physical treatments.

The term “chemotherapy” refers to the use of drugs to treat cancer. A “chemotherapeutic agent” is used to connote a compound or composition that is administered in the treatment of cancer. Chemotherapeutic agents disclosed herein include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these.

In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain alternative embodiments, the antimitotic agent comprises a vinca alkaloid, such as vincristine, binblastine, vinorelbine, or vindesine, or pharmaceutically acceptable salts, acids, or derivatives thereof.

The chemotherapy treatment regimen can be administered to the subject over a period of hours, days, or months. In some embodiments more than one chemotherapeutic agent is administered to the subject. The chemotherapeutic agents can be administered at the same time throughout the period, or administered at different intervals within the period.

In certain embodiments the chemotherapy treatment regimen is an acute cytoreductive chemotherapy treatment regimen.

In certain embodiments the chemotherapy treatment regimen is an induction chemotherapy treatment regimen. The induction chemotherapy treatment regimen may be any regimen that is useful for inducing complete remission of acute myeloid leukemia in a subject. In some embodiments, the induction chemotherapy comprises administering an antimetabolite agent (e.g., cytarabine) and an anthracycline agent (e.g., doxorubicin) to the subject. In some embodiments, the antimetabolite agent comprises cytarabine. In some embodiments, the anthracycline agent comprises doxorubicin. The induction chemotherapy treatment regimen can be administered to the subject over a period of hours, days, or months. The chemotherapeutic agents used in the induction chemotherapy treatment regimen can be administered at the same time throughout the period, or administered at different intervals within the period. In some embodiments, the induction chemotherapy comprises administering cytarabine and doxorubicin to the subject for a period of 5 to 7 days. In some embodiments, the induction chemotherapy comprises administering cytarabine and doxorubicin to the subject for a period of 3 to 5 days, followed by administering cytarabine alone to the subject for a period of 2 to 3 days.

The inhibitor (e.g., a pyrimidine synthesis inhibitor, an aspartate transporter inhibitor, and/or a GOT2 inhibitor) can be administered to the subject before the chemotherapy treatment regimen is administered to the subject, at the same time the chemotherapy treatment regimen is administered to the subject, after the chemotherapy treatment regimen is administered to the subject, or any combination of the above. In some embodiments the inhibitor is administered to the subject in one or more doses. In some embodiments the inhibitor is administered to the subject in one, two, three, four, five, six, seven, eight, nine, or ten doses. In some embodiments each dose is administered to the subject on a different day post-chemotherapy treatment. In some embodiments, multiple doses (e.g., two, three, or four doses) are administered to the subject on the same day.

In some embodiments, the inhibitor (e.g., a pyrimidine synthesis inhibitor, an aspartate transporter inhibitor, and/or a GOT2 inhibitor) is administered to the subject at least 1 day, 2 days, 3 days, 4 days, 5 days, 6 days, 7 days, 8 days, 9 days, 10 days, 11, days, 12 days, 13 days, 14, days or 15 days after completion of the chemotherapy treatment regimen (e.g., induction chemotherapy treatment regimen). In some embodiments the inhibitor is administered at a time following a chemotherapy treatment regimen that is identified as the moment of maximal response. In some embodiments the moment of maximal response is 3-7 days following completion of the chemotherapy treatment. In some embodiments the moment of maximal response is 3-4 days following completion of the chemotherapy treatment. In some embodiments the moment of maximal response is 5-7 days following completion of the chemotherapy treatment.

In some embodiments, the inhibitor (e.g., a pyrimidine synthesis inhibitor, an aspartate transporter inhibitor, and/or a GOT2 inhibitor) is administered to the subject in two or more doses after completion of a chemotherapy treatment regimen (e.g., an induction chemotherapy treatment regimen). In some aspects a pyrimidine synthesis inhibitor is administered in a first dose 2-4 days after completion of a chemotherapy treatment regimen and in a second dose 5-11 days after completion of the chemotherapy treatment regimen. In some aspects a pyrimidine synthesis inhibitor is administered in a first dose 2-4 days after completion of a chemotherapy treatment regimen, in a second dose 5-7 days after completion of the chemotherapy treatment regimen, and in a third dose 8-10 days after completion of the chemotherapy treatment regimen. In some aspects a pyrimidine synthesis inhibitor is administered in a first dose 2-3 days after completion of a chemotherapy treatment regimen, in a second dose 4-6 days after completion of the chemotherapy treatment regimen, in a third dose 7-9 days after completion of the chemotherapy treatment regimen, and in a fourth dose 10-12 days after completion of the chemotherapy treatment regimen. In some embodiments a first dose of pyrimidine synthesis inhibitor is administered to the subject 3 days after completion of the chemotherapy treatment regimen, a second dose of pyrimidine synthesis inhibitor is administered 6 days after completion of the chemotherapy treatment regimen, and a third dose of pyrimidine synthesis inhibitor is administered 9 days after completion of the chemotherapy treatment regimen. In some embodiments a first dose of pyrimidine synthesis inhibitor is administered to the subject 2 days after completion of the chemotherapy treatment regimen, a second dose of pyrimidine synthesis inhibitor is administered 5 days after completion of the chemotherapy treatment regimen, a third dose of pyrimidine synthesis inhibitor is administered 8 days after completion of the chemotherapy treatment regimen, and a fourth dose of pyrimidine synthesis inhibitor is administered 11 says after completion of the chemotherapy treatment regimen.

In some embodiments, a subject is administered an induction chemotherapy treatment regimen comprising 100 mg/kg cytarabine+3 mg/kg doxorubicin for 3 days, followed by chemotherapy with 100 mg/kg cytarabine in the absence of doxorubicin for 2 days. Three days after completion of the induction chemotherapy regimen brequinar (BRQ) is administered to the subject. In some embodiments, BRQ is administered to the subject in a single dose. In some embodiments 50 mg/kg BRQ is administered to the subject 2-4 days after completion of the induction chemotherapy treatment regimen. In some embodiments a second dose of BRQ is administered 9-11 days after completion of the induction chemotherapy treatment regimen. In some embodiments a first dose of 50 mg/kg BRQ is administered to the subject 3 days after completion of the induction chemotherapy treatment regimen, a second dose of 50 mg/kg BRQ is administered 6 days after completion of the induction chemotherapy treatment regimen, and a third dose of 50 mg/kg BRQ is administered 9 days after completion of the induction chemotherapy treatment regimen. In some embodiments a first dose of 50 mg/kg BRQ is administered to the subject 2 days after completion of the induction chemotherapy treatment regimen, a second dose of 50 mg/kg BRQ is administered 5 days after completion of the induction chemotherapy treatment regimen, a third dose of 50 mg/kg BRQ is administered 8 days after completion of the induction chemotherapy treatment regimen, and a fourth dose of 50 mg/kg BRQ is administered 11 says after completion of the induction chemotherapy treatment regimen. In some embodiments, administration of the pyrimidine synthesis inhibitor described herein comprises administering ascending and intermittent concentrations or doses of pyrimidine synthesis inhibitor described herein over a period of time to the subject.

As used herein, “treat,” “treatment,” “treating,” or “amelioration” when used in reference to a disease, disorder or medical condition, refers to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of, for example, acute myeloid leukemia, delay or slowing progression of acute myeloid leukemia, and an increased lifespan as compared to that expected in the absence of treatment.

In some embodiments, treating acute myeloid leukemia comprises inducing complete remission of acute myeloid leukemia in the subject. In some embodiments, treating acute myeloid leukemia comprises inducing complete remission of acute myeloid leukemia in the subject in the absence of a relapse risk due to residual leukemic cells in the subject's bone marrow or peripheral blood.

In some embodiments, the method further comprises evaluating the subject to determine if the subject has refractory or relapsed acute myeloid leukemia.

In some aspects, the disclosure provides a method of promoting survival of a subject suffering from acute myeloid leukemia, the method comprising administering to the subject an effective amount of a pyrimidine synthesis inhibitor, thereby promoting survival of the subject. The method contemplates any pyrimidine synthesis inhibitor described herein. In some embodiments, the pyrimidine synthesis inhibitor comprises brequinar (BRQ) or an analog thereof.

In some aspects, the disclosure provides a method of promoting survival of a subject suffering from acute myeloid leukemia, the method comprising administering to the subject an effective amount of an aspartate transporter inhibitor, thereby promoting survival of the subject. The method contemplates any aspartate transporter inhibitor described herein. In some embodiments, the aspartate transporter is SLC1A3. In some embodiments, the aspartate transporter inhibitor comprises DL-threo-beta-benzyloxyaspartate (TBOA), L-trans-Pyrrolidine-2,4-dicarboxylic acid (L-trans-2,4-PDC), 2-Amino-5,6,7,8-tetrahydro-4-(4-methoxyphenyl)-7-(naphthalen-1-yl)-5-oxo-4H-chromene-3-carbonitrile (UCPH 101), or analogs thereof.

In some aspects, the disclosure provides a method of promoting survival of a subject suffering from acute myeloid leukemia, the method comprising administering to the subject an effective amount of a GOT2 inhibitor, thereby promoting survival of the subject. The method contemplates any GOT2 inhibitor described herein. In some embodiments, the GOT2 inhibitor comprises aminooxyacetic acid (AOA), hydrazinosuccinic acid, beta-methylene-DL-aspartate, or analogs thereof.

In some embodiments the method further comprises administering a chemotherapy treatment regimen to the subject. In some embodiments, the method further comprises administering an induction chemotherapy treatment regimen to the subject. In some embodiments, the induction chemotherapy comprises administering an antimetabolite agent and an anthracycline agent to the subject. In some embodiments, the antimetabolite agent comprises cytarabine. In some embodiments, the anthracycline agent comprises doxorubicin. In some embodiments, the induction chemotherapy comprises administering cytarabine and doxorubicin to the patient for a period of 5-7 days. In some embodiments, the induction chemotherapy comprises administering cytarabine and doxorubicin to the patient for a period of 3-5 days, followed by administering cytarabine alone to the patient for a period of 2 days. It should be appreciated that any of the administration or dosing schedules and/or treatment regiments described herein can be used with the method.

In some embodiments, the method further comprises selecting a subject suffering from or exhibiting a terminal state of acute myeloid leukemia. In some embodiments, the subject has advanced tumor metastasis. In some embodiments, the subject has a high tumor burden.

In some embodiments, the method further comprises selecting a subject suffering from or exhibiting chemoresistant acute myeloid leukemia.

“Survival” refers to the subject remaining alive, and includes overall survival as well as progression free survival. “Overall survival” refers to the subject remaining alive for a defined period of time, such as 1 year, 2 years, 3 years, 4 years, 5 years, etc. from the time of diagnosis or treatment.

“Progression free survival” refers to the subject remaining alive, without the acute myeloid leukemia progressing or getting worse.

“Promoting survival” refers to enhancing one or more aspects of survival in a treated subject relative to an untreated subject (i.e., a subject not treated with a pyrimidine synthesis inhibitor, such as BRQ), or relative to a subject treated with an approved chemotherapeutic agent alone in the absence of administration of a pyrimidine synthesis inhibitor. In some embodiments, the pyrimidine synthesis inhibitor increases the subject's length of survival compared to the subject's length of survival in the absence of receiving the pyrimidine synthesis inhibitor. In some embodiments, the pyrimidine synthesis inhibitor increases the subject's likelihood of survival compared to the subject's likelihood of survival in the absence of receiving the pyrimidine synthesis inhibitor. In some embodiments, administration of the pyrimidine synthesis inhibitor (e.g., BRQ) to the subject increases the subject's overall survival time by at least 1%, at least 2%, at least 3%, at least 4%, at least 5%, at least 10%, at least 15%, at least 20%, at least 25%, at least 30%, at least 40%, at least 50%, at least 60%, at least 70%, at least 80%, at least 90%, or more relative to subject's overall survival time in the absence of administration of the pyrimidine synthesis inhibitor and/or compared to chemotherapy treatment alone. In some embodiments, administration of the pyrimidine synthesis inhibitor (e.g., BRQ) to the subject increases the subject's overall survival time by at least 1.1 fold, at least 1.2 fold, 1.3 fold, at least 1.4 fold, at least 1.5 fold, at least 1.6 fold, at least 1.7 fold, at least 1.8 fold, at least 1.9 fold, at least 2 fold, at least 3 fold, at least 4 fold, or, at least 5 fold or more relative to subject's overall survival time in the absence of administration of the pyrimidine synthesis inhibitor and/or compared to chemotherapy treatment alone. In some embodiments, administration of the pyrimidine synthesis inhibitor (e.g., BRQ) to the subject increases the subject's survival time by 1 day, 5 days, 10 days, 30 days, 2 months, 3 months, 4 months, 5 months, 6 months, 7 months, 8 months, 9 months, 10 months, 11 months, 1 year, 18 months, 2 years, 30 months, 3 years, 40 months, 4 years, 5 years, 6 years, 7 years, 8 years, 9 years, 10 years, 15 years, 20 years, 25 years, 30 years, 35 years, 40 years, 50 years, 55 years, 60 years, 65 years, 70 years, or 75 years or more relative to subject's overall survival time in the absence of administration of the pyrimidine synthesis inhibitor and/or compared to chemotherapy treatment alone.

In one aspect, the disclosure provides a method of inducing complete remission in a subject having relapsed or refractory acute myeloid leukemia by eradicating chemoresistant leukemic cells in the subject, the method comprising: (a) evaluating the subject to determine if the subject has relapsed or refractory acute myeloid leukemia; (b) administering to the subject an induction chemotherapy treatment regimen comprising an antimetabolite agent and an anthracycline agent for proscribed periods of time; and (c) administering to the subject a pyrimidine synthesis inhibitor, thereby inducing complete remission in the subject by eradicating chemoresistant leukemic cells in the subject.

### Subjects

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. In some embodiments, the subject suffers from acute myeloid leukemia.

In some embodiments, the subject is a patient presenting with acute myeloid leukemia. As used herein, “acute myeloid leukemia” encompasses all forms of acute myeloid leukemia and related neoplasms according to the World Health Organization (WHO) classification of myeloid neoplasms and acute leukemia, including all of the following subgroups in their relapsed or refractory state: Acute myeloid leukemia with recurrent genetic abnormalities, such as AML with t(8; 21)(q22; q22); RUNX1-RUNX1T1, AML with inv(16)(p13.1q22) or t(16; 16)(p13.1; q22); CBFB-MYH11, AML with t(9; 11)(p22; q23); MLLT3-MLL, AML with t(6; 9)(p23; q34); DEK-NUP214, AML with inv(3)(q21 q26.2) or t(3; 3)(q21; q26.2); RPN1-EVI1, AML (megakaryoblastic) with t(1; 22)(p13; q13); RBM15-MKL1, AML with mutated NPM1, AML with mutated CEBPA; AML with myelodysplasia-related changes; therapy-related myeloid neoplasms; AML, not otherwise specified, such as AML with minimal differentiation, AML without maturation, AML with maturation, acute myelomonocytic leukemia, acute monoblastic/monocytic leukemia, acute erythroid leukemia (e.g., pure erythroid leukemia, erythroleukemia, erythroid/myeloid), acute megakaryoblastic leukemia, acute basophilic leukemia, acute panmyelosis with myelofibrosis; myeloid sarcoma; myeloid proliferations related to Down syndrome, such as transient abnormal myelopoiesis or myeloid leukemia associated with Down syndrome; and blastic plasmacytoid dendritic cell neoplasm.

In some embodiments, the methods described herein further comprise selecting a subject diagnosed with acute myeloid leukemia, for example, based on the symptoms presented. Symptoms associated with acute myeloid leukemia are known to the skilled practitioner. For example, a patient can be diagnosed with acute myeloid leukemia if the subject presents with a myeloid neoplasm with 20% or more blasts in the peripheral blood or bone marrow.

In some embodiments, the methods described herein further comprise selecting a subject at risk of developing acute myeloid leukemia. For example, a subject can be selected as at risk of developing leukemia based on a family history of leukemias.

In some embodiments, a subject is selected as diagnosed with acute myeloid leukemia or at risk of developing acute myeloid leukemia based on a genetic mutation useful as a diagnostic or prognostic marker of myeloid neoplasms. Exemplary such markers include mutations of: JAK2, MPL, and KIT in MPN; NRAS, KRAS, NF1, and PTPN11 in MDS/MPN; NPM1, CEBPA, FLT3, RUNX1, KIT, WT1, and MLL in AML; and GATA1 in myeloid proliferations associated with Down syndrome (see Vardiman, et al., “The 2008 revision of the World Health Organization (WHO) classification of myeloid neoplasms and acute leukemia: rationale and important changes,” Blood 114(5), 937-951 (2009), incorporated herein by reference in its entirety).

In some embodiments, the methods described herein further comprise selecting a subject suspected of having acute myeloid leukemia. A subject suspected of having acute myeloid leukemia, for example, can be selected based on family history, diagnostic testing or based on the symptoms presented or a combination thereof.

In some embodiments, the methods described herein further comprise selecting a subject suffering from refractory or relapsed acute myeloid leukemia. As used herein, “relapsed acute myeloid leukemia” is defined as reappearance of leukemic blasts in the blood or greater than 5% blasts in the bone marrow after complete remission not attributable to any other cause. For subjects presenting with relapsed AML, more than 5% blasts on baseline bone marrow assessment is required. As used herein, “refractory acute myeloid leukemia” is defined as a failure to achieve a complete remission or complete remission with incomplete blood recovery after previous therapy. Any number of prior anti-leukemia schedules is allowed. As used herein, “complete remission” is defined as morphologically leukemia free state (i.e. bone marrow with less than 5% blasts by morphologic criteria and no Auer rods, no evidence of extramedullary leukemia) and absolute neutrophil count greater than or equal to 1,000/μL and platelets greater than 100,000/μL. As used herein, “complete remission with incomplete blood recovery” is defined as morphologically leukemia free state (i.e. bone marrow with less than 5% blasts by morphologic criteria and no Auer rods, no evidence of extramedullary leukemia) and neutrophil count less than 1,000/μL or platelets less than 100,000 μL in the blood.

In some embodiments, the methods described herein further comprise selecting a subject who relapses from complete remission of acute myeloid leukemia after receiving chemotherapy treatment regimen (e.g., an induction chemotherapy treatment regimen).

### Pharmaceutical Compositions

The disclosure contemplates compositions comprising the inhibitors (e.g., pyrimidine synthesis inhibitors, aspartate transporter inhibitors, and/or GOT2 inhibitors) described herein and at least one chemotherapeutic agent (e.g., a chemotherapeutic agent to which acute myeloid leukemia cells in a patient are or become resistant).

In some aspects, the disclosure provides a pharmaceutical composition comprising an effective amount of a pyrimidine synthesis inhibitor, and an effective amount of at least one chemotherapeutic agent as described herein.

In some embodiments, a pharmaceutical composition comprises an effective amount of a pyrimidine synthesis inhibitor, an effective amount of at least one chemotherapeutic agent, and a pharmaceutically acceptable carrier, diluent, or excipient.

In some aspects, the disclosure provides a pharmaceutical composition comprising an effective amount of a GOT2 inhibitor, and an effective amount of at least one chemotherapeutic agent as described herein.

In some embodiments, a pharmaceutical composition comprises an effective amount of a GOT2 inhibitor, an effective amount of at least one chemotherapeutic agent, and a pharmaceutically acceptable carrier, diluent, or excipient.

In some aspects, the disclosure provides a pharmaceutical composition comprising an effective amount of an aspartate transporter inhibitor, and an effective amount of at least one chemotherapeutic agent as described herein.

In some embodiments, a pharmaceutical composition comprises an effective amount of an aspartate transporter inhibitor, an effective amount of at least one chemotherapeutic agent, and a pharmaceutically acceptable carrier, diluent, or excipient.

In some aspects, the disclosure provides a pharmaceutical composition comprising an effective amount of one or more inhibitors selected from the group consisting of a pyrimidine synthesis inhibitor, a GOT2 inhibitor, and an aspartate transporter inhibitor, and an effective amount of at least one chemotherapeutic agent as described herein.

In some embodiments, a pharmaceutical composition comprises an effective amount of one or more inhibitors selected from the group consisting of a pyrimidine synthesis inhibitor, a GOT2 inhibitor, and an aspartate transporter inhibitor, an effective amount of at least one chemotherapeutic agent, and a pharmaceutically acceptable carrier, diluent, or excipient.

The compositions comprising the inhibitor(s) and the at least one chemotherapeutic agent can be used for treating acute myeloid leukemia as described herein. In some embodiments, the composition is useful for inducing complete remission of leukemia in the subject. In some embodiments, the composition is useful for inducing complete remission of acute myeloid leukemia in the subject. In some embodiments, the composition is useful for inducing complete remission of acute myeloid leukemia in the subject in the absence of a relapse risk due to residual leukemic cells in the subject's bone marrow or peripheral blood.

### Formulation and Administration

The inhibitor and/or chemotherapeutic agent described herein can be administered alone or with suitable pharmaceutical carriers, and can be in solid or liquid form such as, tablets, capsules, powders, solutions, suspensions, or emulsions. As used herein, the term “administered” refers to the placement of an inhibitor or agent described herein, into a subject by a method or route which results in at least partial localization of the inhibitor or agent at a desired site. An inhibitor and/or chemotherapeutic agent described herein can be administered by any appropriate route which results in effective treatment in the subject, i.e. administration results in delivery to a desired location in the subject where at least a portion of the composition is delivered. For a comprehensive review on drug delivery strategies see Ho et al., Curr. Opin. Mol. Ther. (1999), 1:336-3443; Groothuis et al., J. Neuro Virol. (1997), 3:387-400; and Jan, Drug Delivery Systems: Technologies and Commercial Opportunities, Decision Resources, 1998, content of all which is incorporated herein by reference. Exemplary routes of administration of the pyrimidine synthesis inhibitor (e.g., BRQ) and/or chemotherapeutic agents described herein include, without limitation, intravenous administration, e.g., as a bolus or by continuous infusion over a period of time, intramuscular, intraperitoneal, intracerobrospinal, subcutaneous, intra-articular, intrasynovial, intrathecal, oral, topical, or inhalation routes. The pyrimidine synthesis inhibitor and/or chemotherapeutic agents can be formulated in pharmaceutically acceptable compositions which comprise a therapeutically-effective amount of the inhibitor and/or agent, formulated together with one or more pharmaceutically acceptable carriers (additives) and/or diluents, or excipients. The formulations can conveniently be presented in unit dosage form and may be prepared by any of the methods well known in the art of pharmacy. Techniques, excipients and formulations generally are found in, e.g., Remington's Pharmaceutical Sciences, Mack Publishing Co., Easton, Pa. 1985, 17th edition, Nema et al., PDA J. Pharm. Sci. Tech. 1997 51:166-171.

In some embodiments, the inhibitor and/or chemotherapeutic agents described herein can be administrated encapsulated within a nanoparticle (e.g., a lipid nanoparticle). In some embodiments, inhibitors and/or chemotherapeutic agents described herein can be administered encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., endothelial cells) can also be used as pharmaceutically acceptable carriers.

The inhibitor and/or chemotherapeutic agents can be administered to a subject in combination with other pharmaceutically active agents. Exemplary pharmaceutically active agents include, but are not limited to, those found in Harrison's Principles of Internal Medicine, 13^{th} Edition, Eds. T. R. Harrison et al. McGraw-Hill N.Y., NY; Physician's Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference. In some embodiments, the pharmaceutically active agent is a conventional treatment for acute myeloid leukemia. In some embodiments, the pharmaceutically active agent is a conventional treatment for an autoimmune or inflammatory condition. The skilled artisan will be able to select the appropriate conventional pharmaceutically active agent for treating any particular disease or disease subtype using the references mentioned above based on their expertise, knowledge and experience.

The inhibitor, chemotherapeutic agent, and/or the other pharmaceutically active agent can be administered to the subject in the same pharmaceutical composition or in different pharmaceutical compositions (at the same time or at different times). For example, an inhibitor and at least one chemotherapeutic agent can be formulated in the same composition or in different compositions.

The pharmaceutical compositions can be included in a container, pack, or dispenser together with instructions for administration.

As used herein, “effective amount”, “effective amounts”, or “therapeutically effective amounts” means an amount of the agent (e.g., pyrimidine synthesis inhibitor) which is effective to eradicate a majority or all of the leukemic cells (e.g., stem or progenitor cells) in a population of cells or a subject. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in the acute myeloid leukemia or autoimmune or inflammatory disorder.

### Kits

One or more inhibitors and/or chemotherapeutic agents described herein can be provided in a kit. The kit includes (a) the inhibitor, e.g., a composition that includes the inhibitor (e.g., a pyrimidine synthesis inhibitor), (b) the at least one chemotherapeutic agent, and (c) informational material. In some aspects the kit includes one, two, or three inhibitors (e.g., a pyrimidine synthesis inhibitor, a GOT2 inhibitor, and/or an aspartate transporter inhibitor). The informational material can be descriptive, instructional, marketing or other material that relates to the methods described herein and/or the use of the inhibitors and agents for the methods described herein. For example, the informational material describes methods for administering the inhibitors and chemotherapeutic agents to a subject for treating acute myeloid leukemia.

The informational material can include instructions to administer the inhibitors and chemotherapeutic agents described herein in a suitable manner, e.g., in a suitable dose, dosage form, or mode of administration. In some embodiments, the instructions recommend administering an effective amount of a pyrimidine synthesis inhibitor (e.g., BRQ). In some embodiments, the instructions recommend administering a pyrimidine synthesis inhibitor in an amount of 50 mg/kg once at two to four days post-completion of an induction chemotherapy treatment regimen and once at nine to eleven days post-completion of an induction chemotherapy treatment regimen. The informational material can include instructions for selecting a suitable subject, e.g., a human, e.g., a human suffering from relapsed or refractory acute myeloid leukemia. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the inhibitor and/or its use in the methods described herein. Of course, the informational material can also be provided in any combination of formats.

In addition to the inhibitor (e.g., pyrimidine synthesis inhibitor) and the at least one chemotherapeutic agent, the kit can include other ingredients, such as a solvent or buffer, a stabilizer or a preservative, and/or an agent for treating a condition or disorder described herein, e.g. acute myeloid leukemia. Alternatively, the other ingredients can be included in the kit, but in different compositions or containers than the inhibitor and the chemotherapeutic agent. In such embodiments, the kit can include instructions for admixing the inhibitor, the chemotherapeutic agent, and the other ingredients, or for using the inhibitor and the chemotherapeutic agent together with the other ingredients.

The inhibitor (e.g., pyrimidine synthesis inhibitor) described herein can be provided in any form, e.g., liquid, dried or lyophilized form. It is preferred that the inhibitor be substantially pure and/or sterile. When the inhibitor is provided in a liquid solution, the liquid solution preferably is an aqueous solution, with a sterile aqueous solution being preferred. When the inhibitor is provided as a dried form, reconstitution generally is by the addition of a suitable solvent. The solvent, e.g., sterile water or buffer, can optionally be provided in the kit.

The kit can include one or more containers for the composition containing the inhibitor(s) and the chemotherapeutic agent(s). In some embodiments, the kit contains separate containers, dividers or compartments for the inhibitor (e.g., in a composition), the chemotherapeutic agent, and informational material. For example, the inhibitor and the chemotherapeutic agent can each be contained in a bottle, vial, or syringe, and the informational material can be contained in a plastic sleeve or packet. In other embodiments, the separate elements of the kit are contained within a single, undivided container. For example, the inhibitor (e.g., in a composition) and the chemotherapeutic agent are contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the inhibitor (e.g., in a composition) and the chemotherapeutic agent. For example, the kit includes a plurality of syringes, ampules, foil packets, or blister packs, each containing a single unit dose of the agent. The containers of the kits can be air tight and/or waterproof.

In some aspects, a kit comprises: a pyrimidine synthesis inhibitor, at least one chemotherapeutic agent, and instructions for administering the pyrimidine synthesis inhibitor and the at least one chemotherapeutic agent to a subject suffering from acute myeloid leukemia.

In some embodiments, the instructions further comprise directions for administering the at least one chemotherapeutic agent as part of an induction chemotherapy treatment regimen for the subject.

In some embodiments, the instructions further comprise directions for administering the pyrimidine synthesis inhibitor, and the at least one therapeutic agent to induce complete remission of acute myeloid leukemia in the subject.

In some embodiments, the instructions further comprise directions for administering the pyrimidine synthesis inhibitor, and the at least one therapeutic agent to induce complete remission of acute myeloid leukemia in the subject, without risk of relapse by completely eradicating leukemic cells in the subject.

### Agents

Without wishing to be bound by any theory, the agents (e.g., pyrimidine synthesis inhibitors) disclosed herein inhibit pyrimidine synthesis. Accordingly, while certain aspects of the invention relate to the use of certain pyrimidine synthesis inhibitors (e.g., BRQ and analogs thereof), it should be understood that the present inventions are not limited to such pyrimidine synthesis inhibitors. Rather, contemplated herein are any means of interfering with pyrimidine synthesis and thereby eradicating leukemic cells (e.g., chemoresistant leukemic cells).

For example, in certain aspects, the methods, kits and compositions disclosed herein may comprise any agents or compositions that are capable of or useful for inhibiting pyrimidine synthesis. In some aspects, inhibiting the transport of aspartate from bone marrow stromal cells to AML cells inhibits pyrimidine synthesis. In some embodiments, a GOT2 inhibitor and/or an aspartate transporter inhibitor act as a pyrimidine synthesis inhibitor (e.g., by limiting aspartate availability). Exemplary types of agents that can be used as pyrimidine synthesis inhibitors include small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof. In some aspects, the pyrimidine synthesis inhibitor is brequinar (BRQ) or analogs thereof.

The disclosure contemplates the use of an agent in combination with at least one additional chemotherapeutic agent, such as a chemotherapeutic agent, in the methods, compositions, and kits described herein. The disclosure contemplates the use of any chemotherapeutic agent that is useful for treating cancer (e.g., leukemia). Exemplary chemotherapeutic agents that can be administered in combination with the pyrimidine synthesis inhibitor of the present invention include alkylating agents (e.g. cisplatin, carboplatin, oxaloplatin, mechlorethamine, cyclophosphamide, chorambucil, nitrosureas); anti-metabolites (e.g. methotrexate, pemetrexed, 6-mercaptopurine, dacarbazine, fludarabine, 5-fluorouracil, arabinosycytosine, capecitabine, gemcitabine, decitabine); plant alkaloids and terpenoids including vinca alkaloids (e.g. vincristine, vinblastine, vinorelbine), podophyllotoxin (e.g. etoposide, teniposide), taxanes (e.g. paclitaxel, docetaxel); topoisomerase inhibitors (e.g. notecan, topotecan, amasacrine, etoposide phosphate); antitumor antibiotics (dactinomycin, doxorubicin, epirubicin, and bleomycin); ribonucleotides reductase inhibitors; antimicrotubules agents; and retinoids. (See, e.g., Cancer: Principles and Practice of Oncology (V. T. DeVita, et al., eds., J.B. Lippincott Company, 9^{th} ed., 2011; Brunton, L., et al. (eds.) Goodman and Gilman's The Pharmacological Basis of Therapeutics, 12^{th} Ed., McGraw Hill, 2010).

The compositions, methods, and kits described herein contemplate the use of at least one chemotherapeutic agent, particularly one to which AML cells in a patient are or become resistant (e.g., by any resistance mechanism). In some embodiments, the at least one chemotherapeutic agent comprises an antimetabolite agent. In some embodiments, the at least one chemotherapeutic agent comprises cytarabine. In some embodiments, the at least one chemotherapeutic agent comprises an anthracycline agent. In some embodiments, the at least one chemotherapeutic agent comprises doxorubicin. In some embodiments, the at least one chemotherapeutic agent comprises an antimetabolite agent and an anthracycline agent. In some embodiments, the at least one chemotherapeutic agent comprises cytarabine and the anthracycline agent comprises doxorubicin. It should be appreciated that administration of a pyrimidine synthesis inhibitor described herein (e.g., BRQ) selectively targets leukemic cells by, in part, overcoming chemoresistance exhibited by leukemic cells, such as pyrimidine synthesis-mediated chemoresistance.

### Some Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. As used herein the term “comprising” or “comprises” is used in reference to compositions, methods, kits and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, kits and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following example illustrates some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1: Induction of a Timed Metabolic Collapse Overcomes Chemoresistance in Acute Myeloid Leukemia

In this study, it is shown that chemoresistant AML cells exhibit transient metabolic adaptations, revealed by a unique metabolite profile but not evident at the gene expression level. These dynamic metabolic changes can be targeted to improve AML cell elimination and delay relapse, and are supported by metabolic crosstalk with local bone marrow niche cells.

### Results

AML Cells Exhibit Transient Metabolic Changes in Response to Chemotherapy

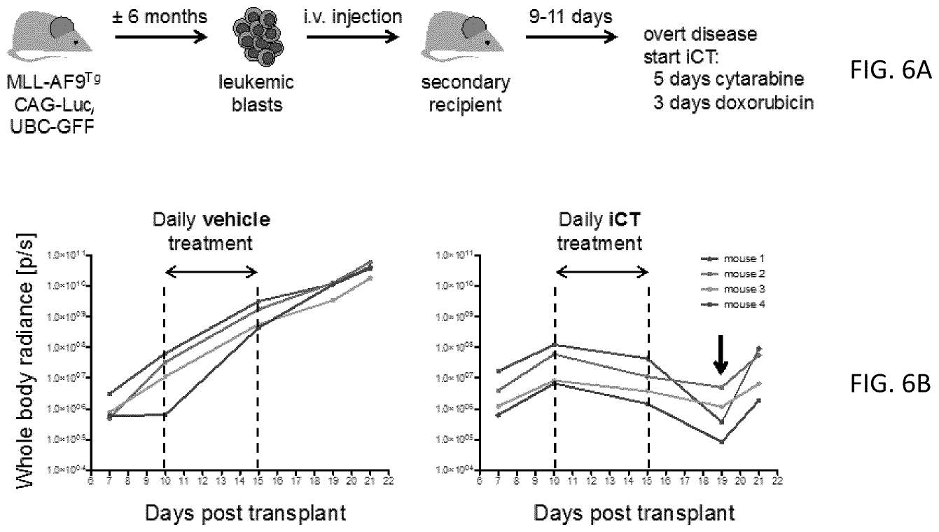

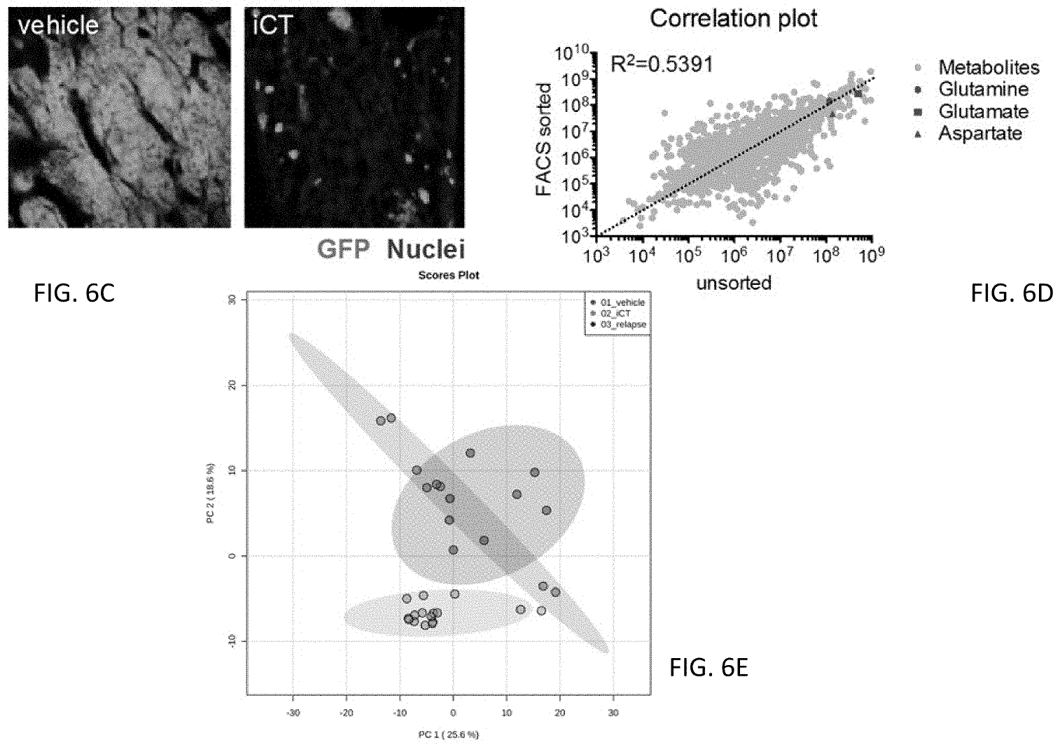

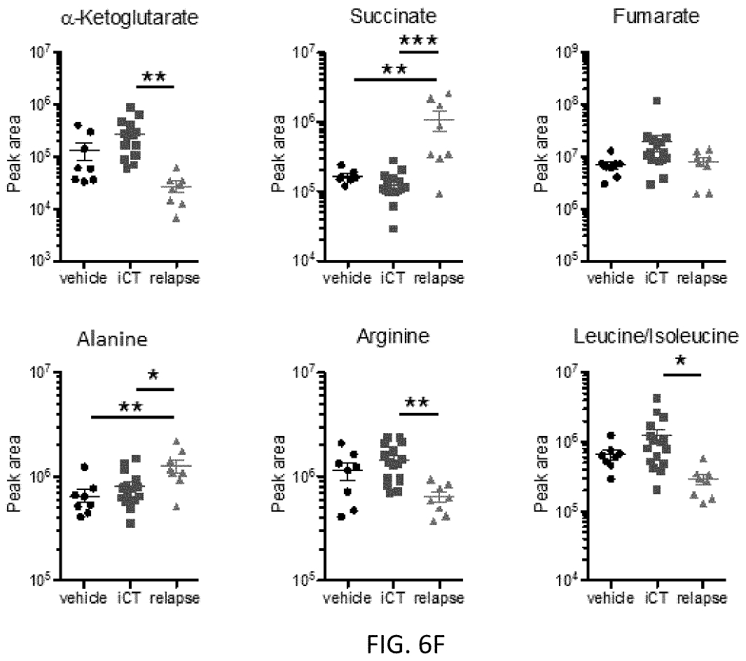

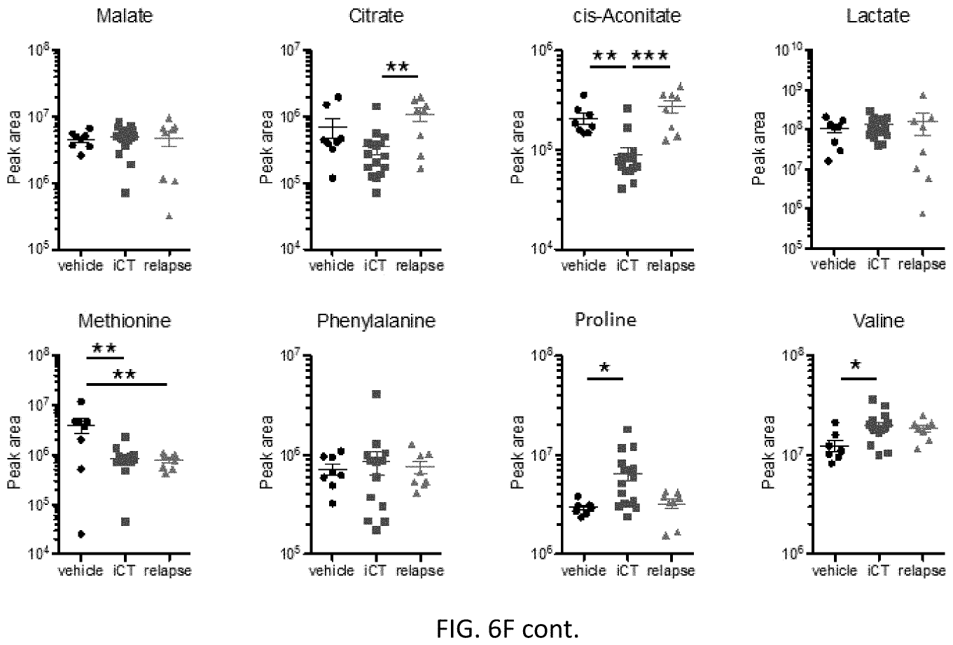

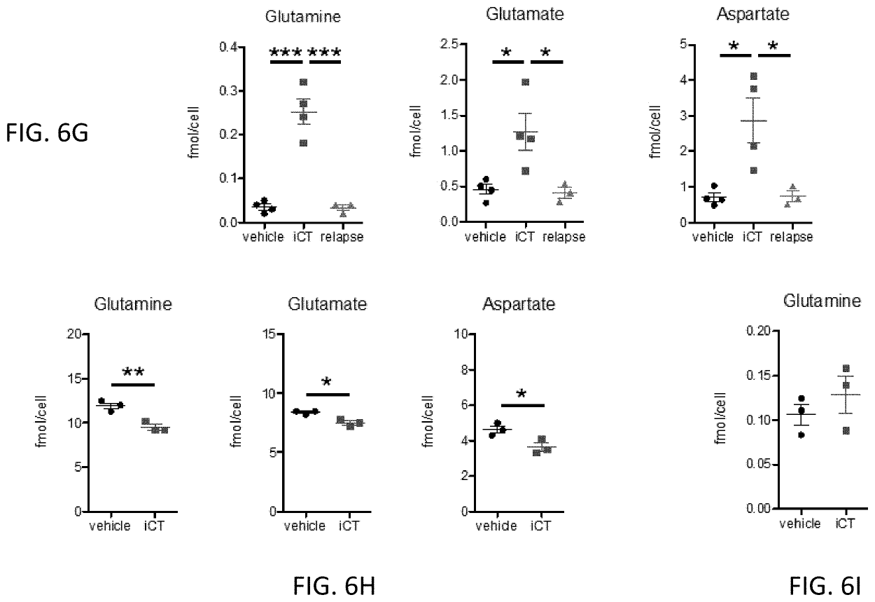

It remains unclear how certain AML cells manage to survive the extreme stress of chemotherapy, characterized not only by the presence of the chemotherapy drugs, but also by massive neighboring cell death and local niche alterations. It was hypothesized that cancer cells must pass through extreme metabolic challenges when under selection from chemotherapy and surrounded by massive cell death in order to become the source of relapse. The conditions of relapse are not evident at the time resistance is clinically evident and generally studied. Rather, relapse is declared at the point of maximal metabolic stress from toxic chemotherapy and a hostile microenvironment laden with byproducts of massive neighboring cell death. Functionally, this can be identified as the “point of maximal response” after chemotherapy since thereafter resistant cells have begun the regrowth that characterizes relapse. To determine this moment of maximal response, a triple transgenic mouse model was used in which cells express MLL-AF9, driving leukemia development, as well as luciferase and GFP (FIG. 6A). Culture-expanded bone marrow cells derived from terminally ill primary mice were intravenously transplanted into secondary wildtype recipients, leading to the establishment of a fast-developing disease that can be monitored through bioluminescence imaging (FIG. 6B). Mice were treated with an induction chemotherapy (iCT) regimen that closely mimics the one used in the clinic (cytarabine for 5 days+doxorubicin for the first 3 days) or with vehicle, followed disease progression, and the moment of maximal response was discovered to occur around three days after the last dose of chemotherapy (FIGS. 6B-6C).

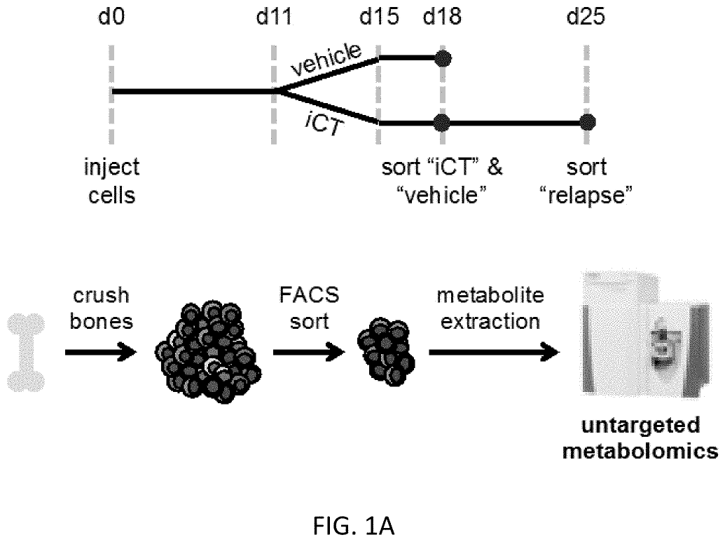

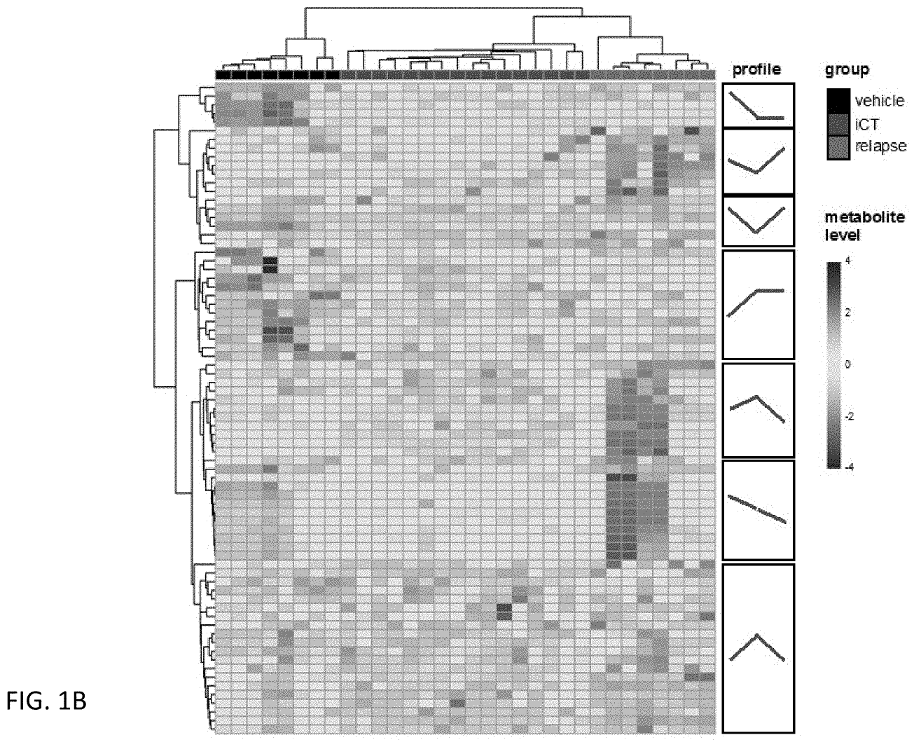

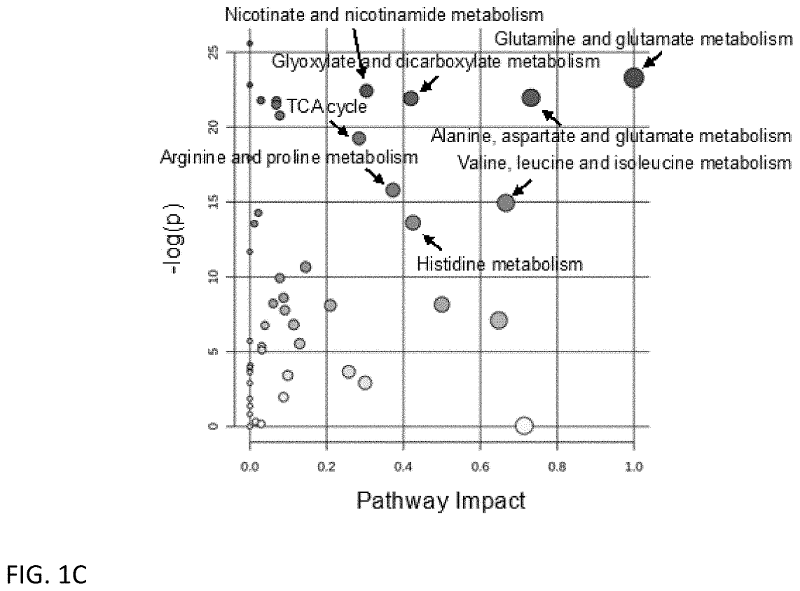

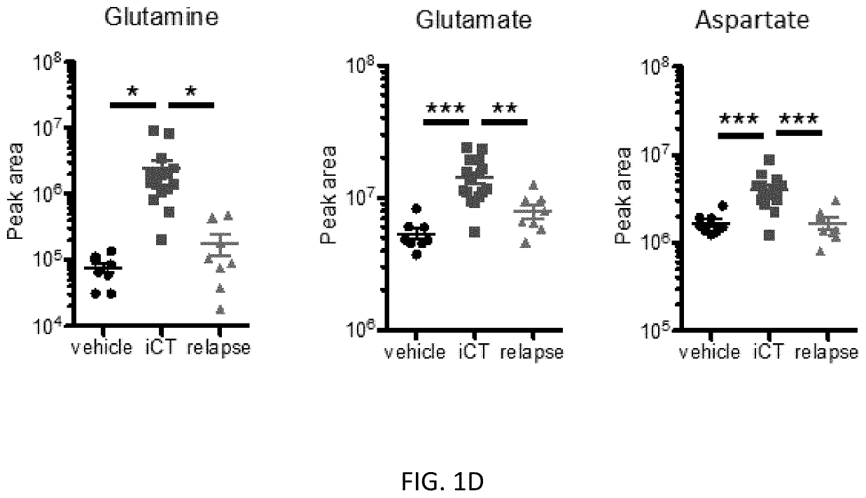

A methodology to investigate the metabolic profile of freshly isolated AML cells was then developed. Bone marrow cells of mice subjected to vehicle treatment were isolated, at the time of maximal response after iCT, or after relapse, sorted GFP AML cells using fluorescence-activated cell sorting (FACS) and extracted polar metabolites (FIG. 1A). Samples were kept at 4° C. in phosphate-buffered saline at all times to minimize metabolic disturbance [15], and pilot experiments were performed to confirm that subjecting the cells to FACS minimally impacts their metabolite profile (FIG. 6D). The levels of intracellular metabolites in 25,000 sorted cells were then determined by untargeted metabolomics analysis using liquid chromatography-coupled tandem mass spectrometry (LC-MS/MS). 654 metabolites were measured, or which 129 were identified based on their MS/MS spectra using the mzCloud database, and another 211 could be putatively identified based on their molecular weight by searching the Human Metabolome database and KEGG Compound database. The 340 putatively identified metabolites were then further analyzed using the MetaboAnalyst software. Statistical analysis revealed that 125 metabolites differed significantly between the groups (ANOVA, FDR<0.01), and unsupervised clustering of samples based on the 75 most significantly different metabolites revealed the specific metabolic signature of each group (FIG. 1B). Principal component analysis showed that while groups clustered closely together, the iCT group separated from the vehicle and relapse groups (FIG. 6E).

Metabolic pathway enrichment analysis using the (putatively) identified metabolites revealed the related “glutamine and glutamate metabolism” and “alanine, aspartate and glutamate metabolism” as the top pathways distinguishing iCT-treated cells from vehicle- and relapse-treated cells (FIG. 1C). Glutamine, glutamate and aspartate, central metabolites in these pathways, are low in vehicle-treated cells, high in the residual cells at the moment of maximal response to iCT, and low again after relapse (FIG. 1D), suggesting a dynamic role in the immediate stress response to chemotherapy. Related metabolites, including tricarboxylic acid (TCA) cycle metabolites (alpha-ketoglutarate, succinate, fumarate, malate, citrate, cis-aconitate), lactate, and most other amino acids (alanine, arginine, leucine/isoleucine, methionine, phenylalanine, valine) did not show these transient changes (FIG. 6F). the changes in glutamine, glutamate and aspartate were confirmed in an independent, targeted LC-MS experiment (FIG. 6G). Interestingly, when AML cells from vehicle or iCT-treated mice were isolated at the moment of maximal response and cultured in vitro for 24 hours, the differences in glutamine, glutamate and aspartate reversed (FIG. 6H). A difference in glutamine levels was not found when cells were treated with iCT in culture (FIG. 6I), showing that the metabolic profile of AML cells is highly dependent on the local microenvironment.

Timed Inhibition of Glutamine Metabolism Overcomes Chemoresistance in AML

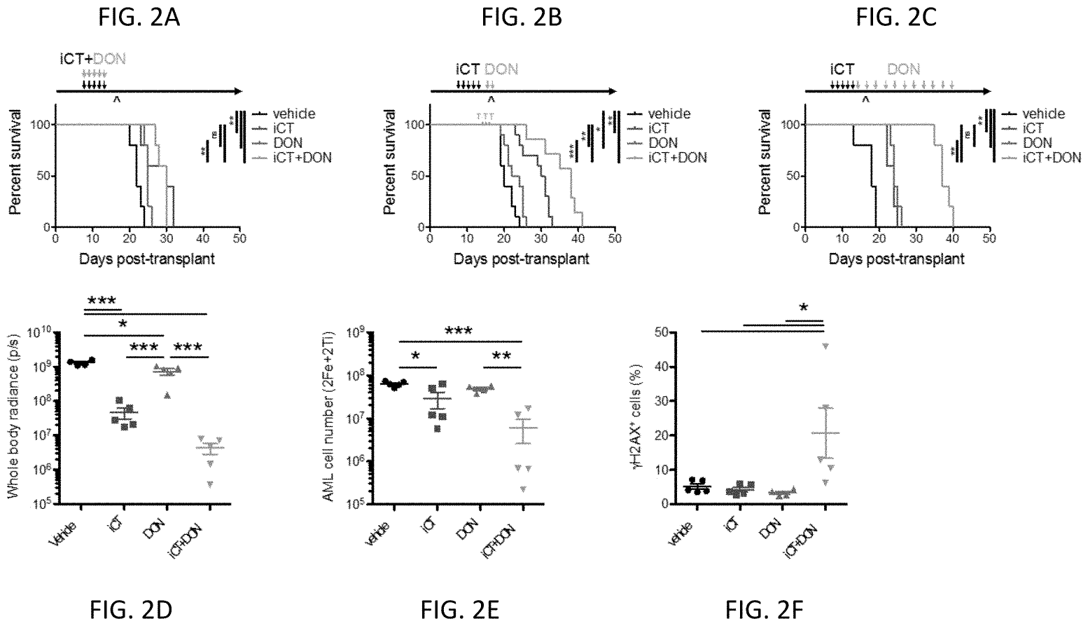

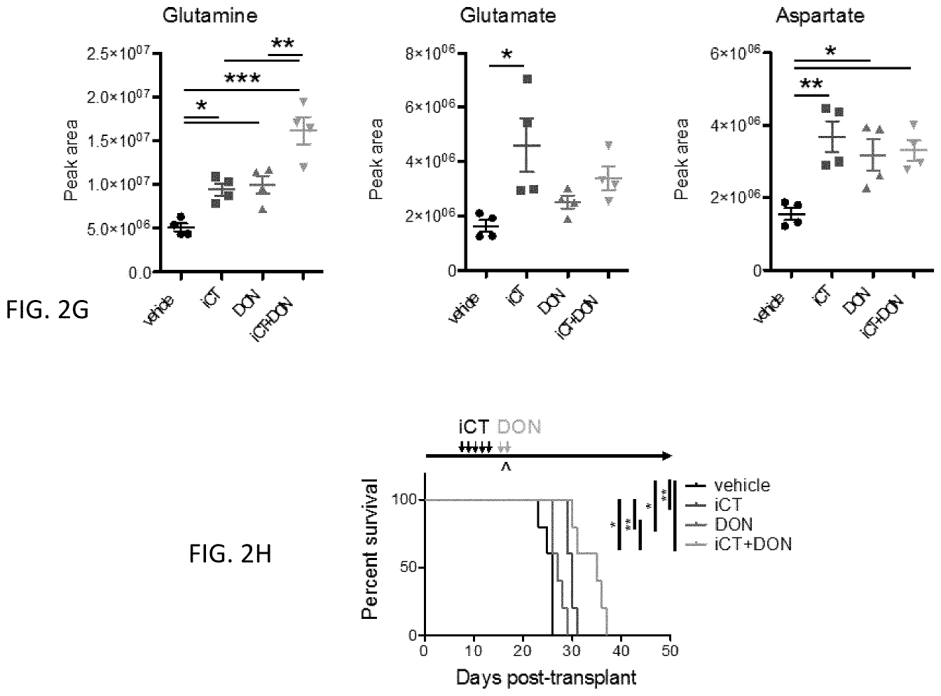

To determine whether the observed increase in glutamine metabolism-associated metabolites causes chemoresistance, whether residual AML cells would be more susceptible to broad inhibition of glutamine metabolism using 6-diazo-5-oxo-L-norleucine (DON), a glutamine analog and antagonist that inhibits all glutamine-dependent enzymes was assessed [16]. When DON was given for 5 days at the same time as iCT, no difference in survival was seen (FIG. 2A). However, when DON treatment was started after completion of the iCT regimen and covered the moment of maximal response, either daily for a short time (FIG. 2B) or continuously every other day (FIG. 2C), a clear survival benefit was observed. Short term treatment with DON after iCT improved elimination of AML cells (FIGS. 2D-2E), delayed relapse (FIG. 2D) and increased double stranded DNA breaks in residual cells (FIG. 2F). Treatment of mice with DON lead to accumulation of glutamine in AML cells while normalizing the increase in glutamate induced by iCT treatment, showing that DON effectively inhibits glutamine metabolism in AML cells (FIG. 2G). Interestingly, aspartate levels did not follow a similar profile as glutamate, and remained high in mice treated with iCT followed by DON (FIG. 2F). The effect of DON on chemoresistant cells was confirmed in a different mouse model of AML, driven by lentiviral HoxA9/Meis1 overexpression, which also showed enhanced AML cell elimination and improved mouse survival when treated with DON after completion of the iCT regimen (FIG. 2H). These data show that activation of glutamine metabolism protects AML cells at the moment of maximal stress, and reveal the potential of timed inhibition of glutamine metabolism to eliminate chemoresistant AML cells.

Chemoresistant AML Cells do not Depend on Glutaminase

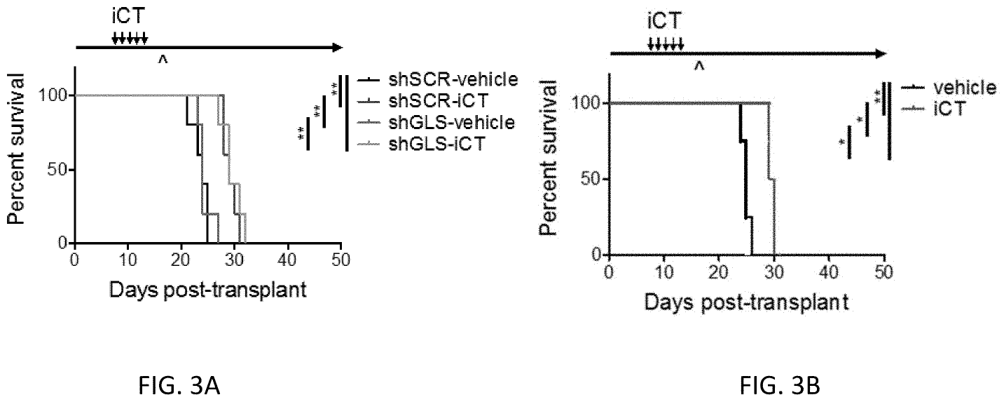

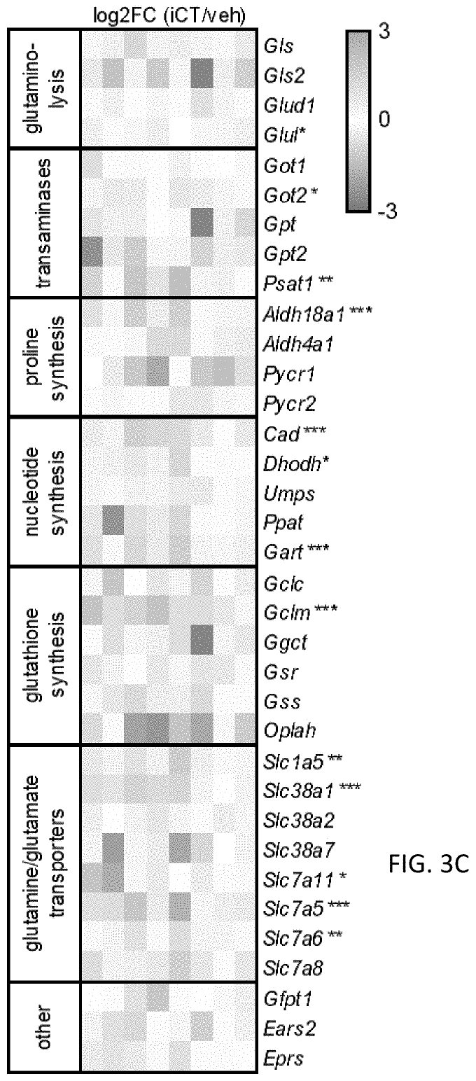

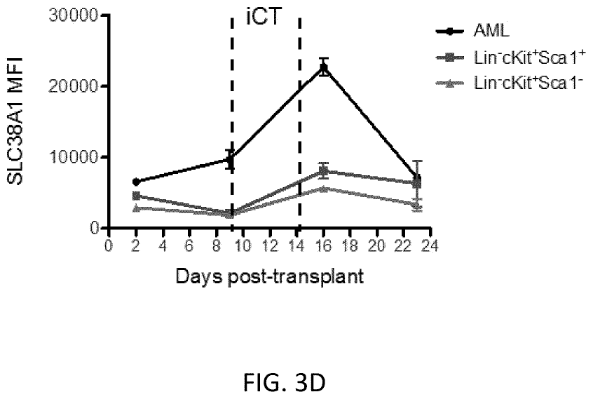

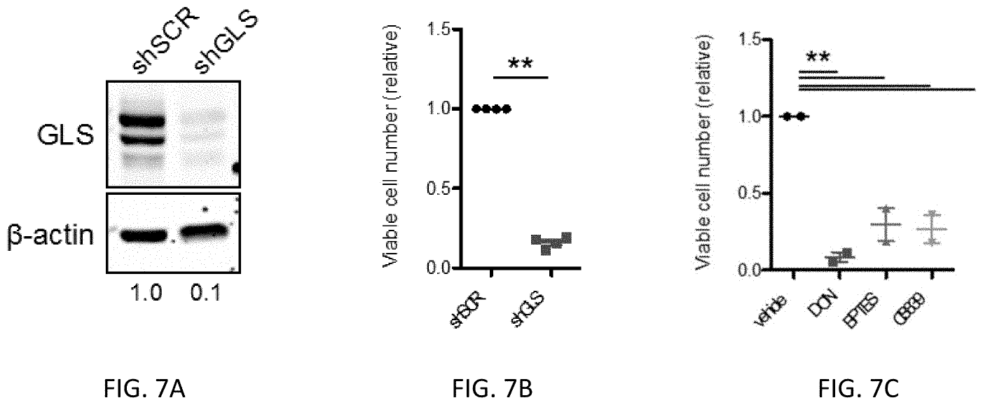

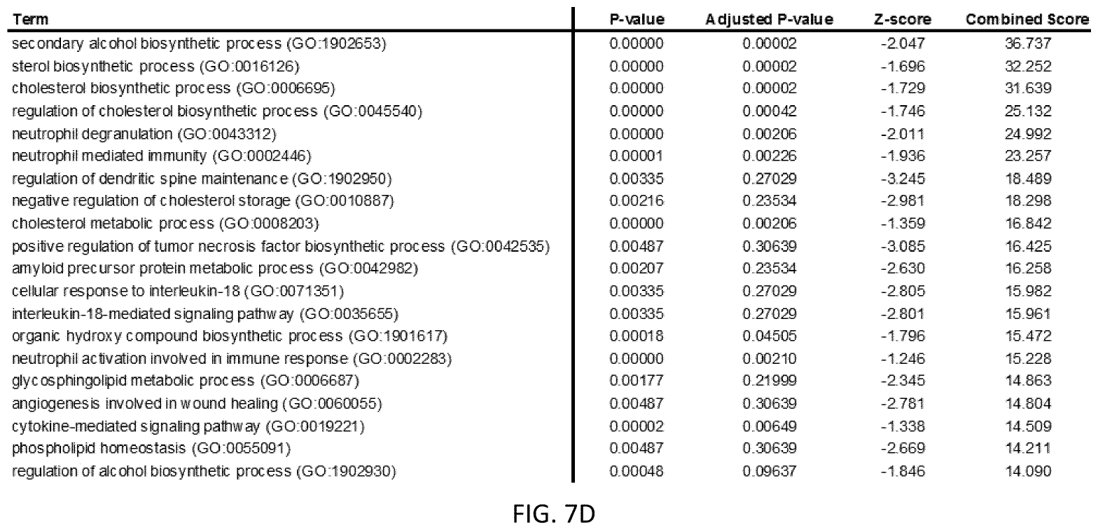

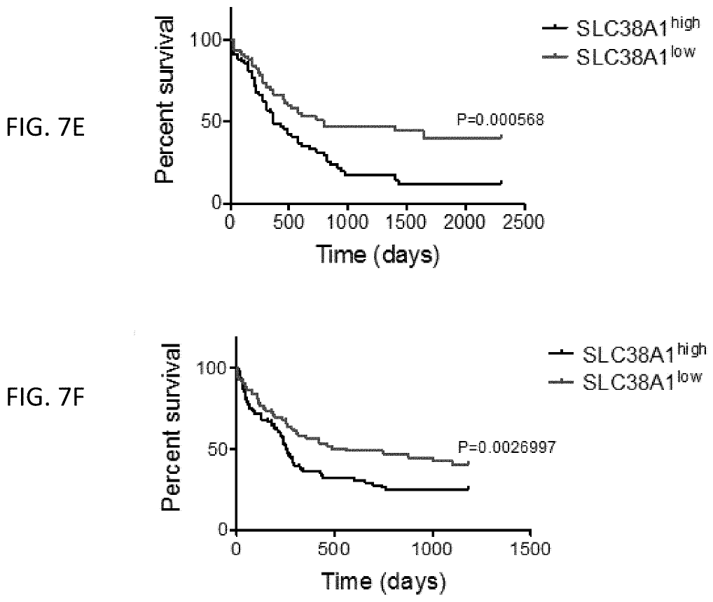

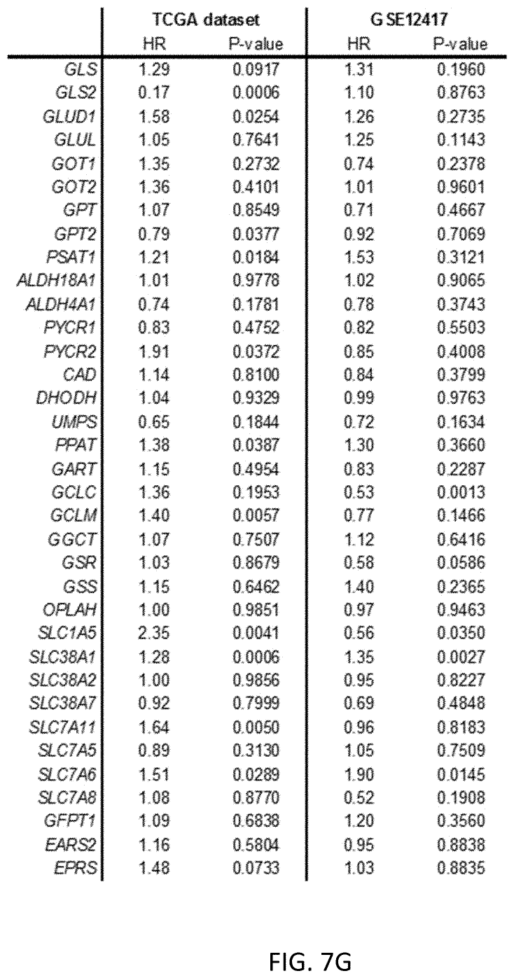

While DON has been tested in Phase I and II clinical trials for both solid tumors and leukemia in the 1980s, it was abandoned due to limited efficacy as a single agent and the occurrence of dose-dependent side effects including nausea, vomiting and mucositis [16]. Due to these toxicity issues, which were observed in the described mouse model (FIG. 2B), an alternative way to inhibit glutamine metabolism in chemoresistant AML cells was sought. The enzyme glutaminase (GLS) has been shown to be a metabolic vulnerability in AML [11,17,18], but it is unknown whether it also plays a role in chemoresistance. Initially the genetic or pharmacological inhibition of GLS reduced AML cell viability in vitro was confirmed (FIGS. 7A-7C), in line with previous studies. Unexpectedly, GLS knockdown had no effect on disease progression or response to iCT in vivo (FIG. 3A). In addition, treatment of mice with a GLS inhibitor during a short window at the moment of maximal response after iCT did not enhance AML cell elimination or mouse survival. These results again highlight that cellular metabolism and metabolic dependencies are microenvironment dependent, and further show that chemoresistant cells, while critically depending on glutamine metabolism, do not require the enzyme glutaminase for their survival.

To better understand the metabolic fate of glutamine in chemoresistant AML cells, the transcriptomic profile of AML cells sorted from vehicle- and iCT-treated mice at the moment of maximal response were analyzed. The expression levels of most enzymes that metabolize glutamine or glutamate were unchanged in chemoresistant cells (FIG. 3C). While some individual enzymes showed increased expression post chemotherapy, no single pathway stood out as particularly altered in chemoresistant AML cells (FIG. 3C). In accordance, enrichment analysis comparing vehicle- and iCT-treated cells did not return any glutamine-related pathways amongst the top hits (FIG. 7D). Increased expression of several glutamine transporters was observed, including Slc1a5, Slc38a1, Slc7a5 and Slc7a6, in chemoresistant AML cells (FIG. 3C). Flow cytometric analysis confirmed a transient increase in SLC38A1 protein levels after chemotherapy, which was not seen in normal hematopoietic stem and progenitor cell populations (FIG. 3D), suggesting that the changes in glutamine metabolism in response to chemotherapy are specific for AML cells. Two publicly available human AML survival datasets were also analyzed, and it was found that high expression of SLC38A1 strongly associates with poor overall survival (FIGS. 7E-7F), which was not found for any other glutamine metabolism-related gene (FIG. 7G). Together, these data show that the transient increase in glutamine metabolism in chemoresistant AML cells seen at the metabolite level is not reflected at the mRNA level, with the exception of high expression of glutamine transporters that may also be linked to poor outcomes in AML patients.

Glutamine Metabolism Drives AML Chemoresistance by Fueling Pyrimidine Synthesis

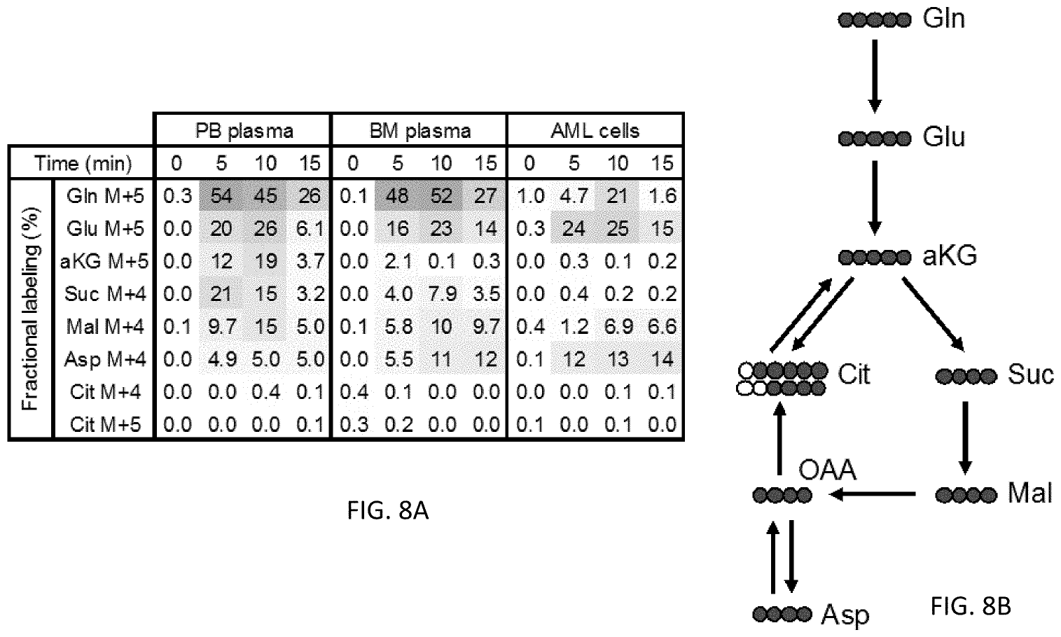

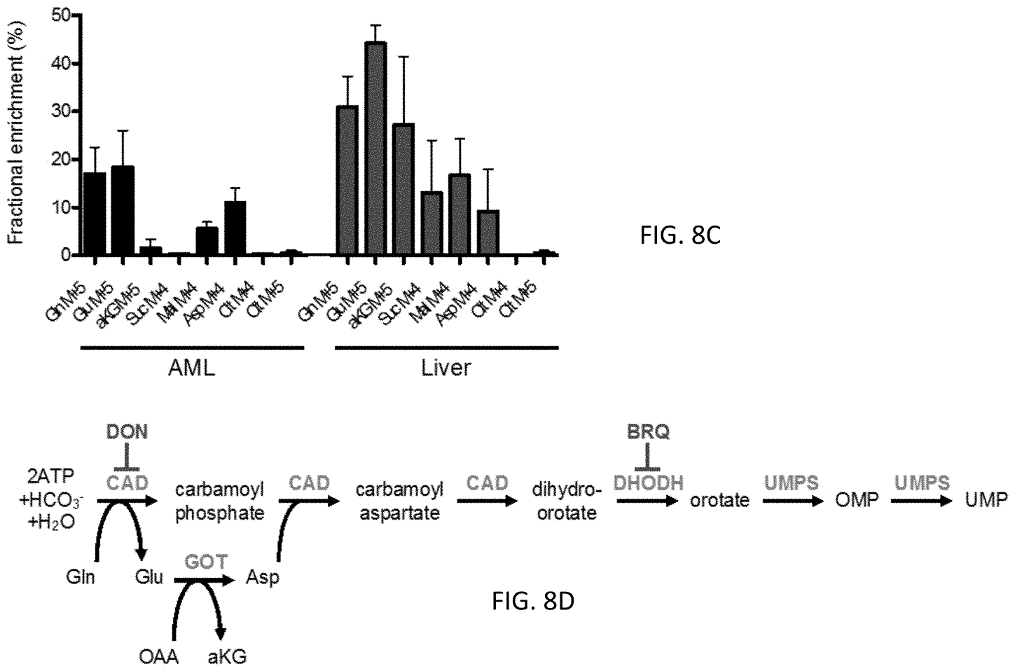

Since transcriptomic analyses did not provide a clear answer, in vivo stable isotope tracing was performed to unravel the metabolic fate of glutamine in chemoresistant AML cells. Intravascular injection of a bulk dose of ¹³C₅-glutamine lead to rapid labeling of glutamine pools in the peripheral blood and bone marrow plasma of mice, which was maintained at 45-55% labeling for 10 minutes, after which it declined (FIG. 8A). Labeling of glutamine in AML cells occurred slower, with only 21% of the pool labeled at 10 minutes, and a rapid decline thereafter. Based on these results, the 10 minutes time point was chosen as the preferred moment for subsequent analyses. Continuous infusion of glutamine tracer to improve the labeling rate in AML cells was not performed due to the fact that other metabolites downstream of glutamine start to get labeled in the peripheral blood after ¹³C₅-glutamine injection as well (FIGS. 8A-8B), which could seriously confound results during longer labeling periods. One interesting finding of these pilot experiments was that while AML cells show labeling of glutamine and glutamate pools after ¹³C₅-glutamine injection, there is almost no labeling of the TCA cycle intermediates alpha-ketoglutarate and succinate. To investigate whether this was biologically relevant or due to technical limitations of the approach, the labeling pattern from ¹³C₅-glutamine was compared in AML cells versus the liver, a tissue known to metabolize glutamine [19]. In contrast to AML cells, the liver showed extensive conversion of glutamine into TCA cycle metabolites with a gradual decrease going downstream, in line with the expected pattern for cells using glutamine to fuel the TCA cycle (FIG. 8C). This shows that in contrast to previous reports describing glutamine contribution to the TCA cycle in vitro [17,18], under steady-state conditions in vivo this does not seem to be a predominant fate of glutamine in AML cells.

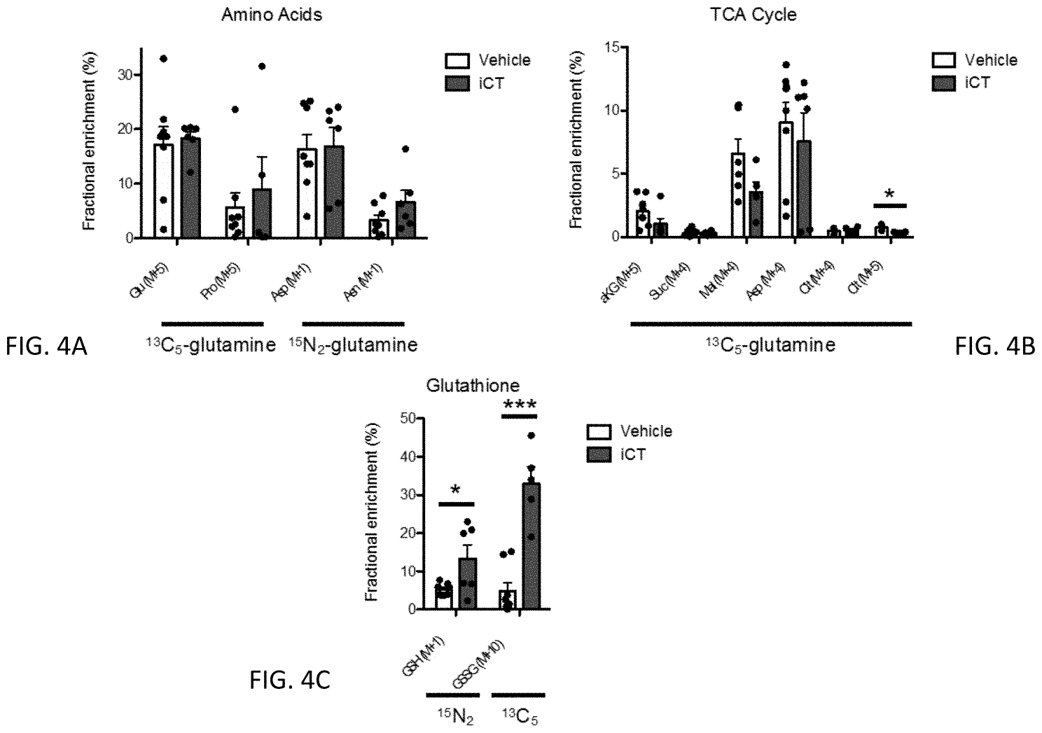

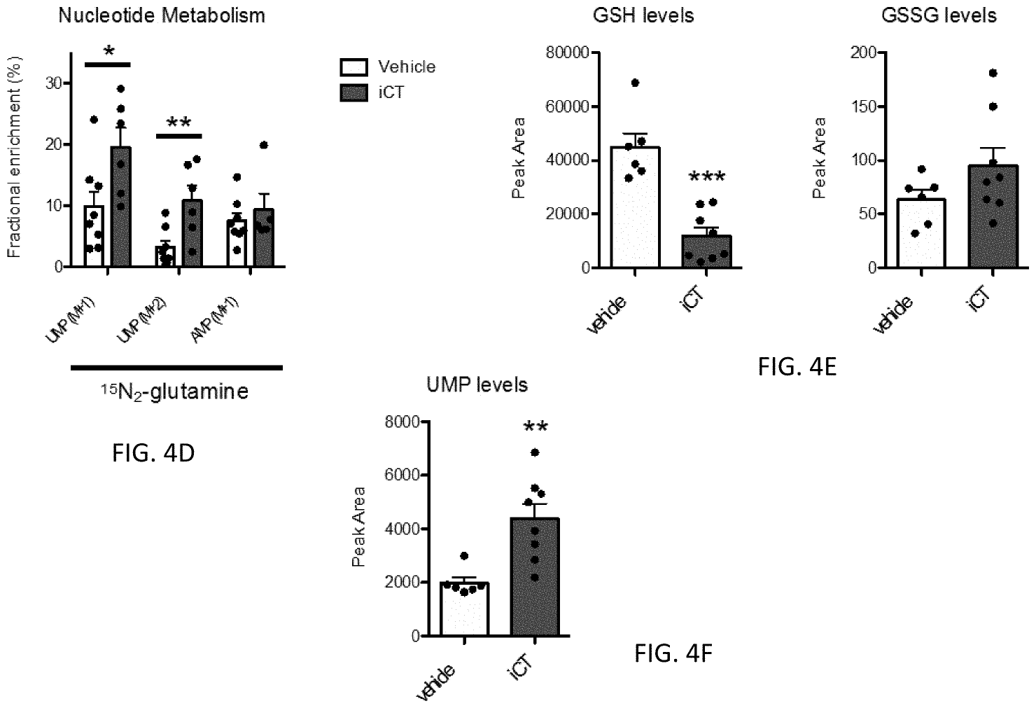

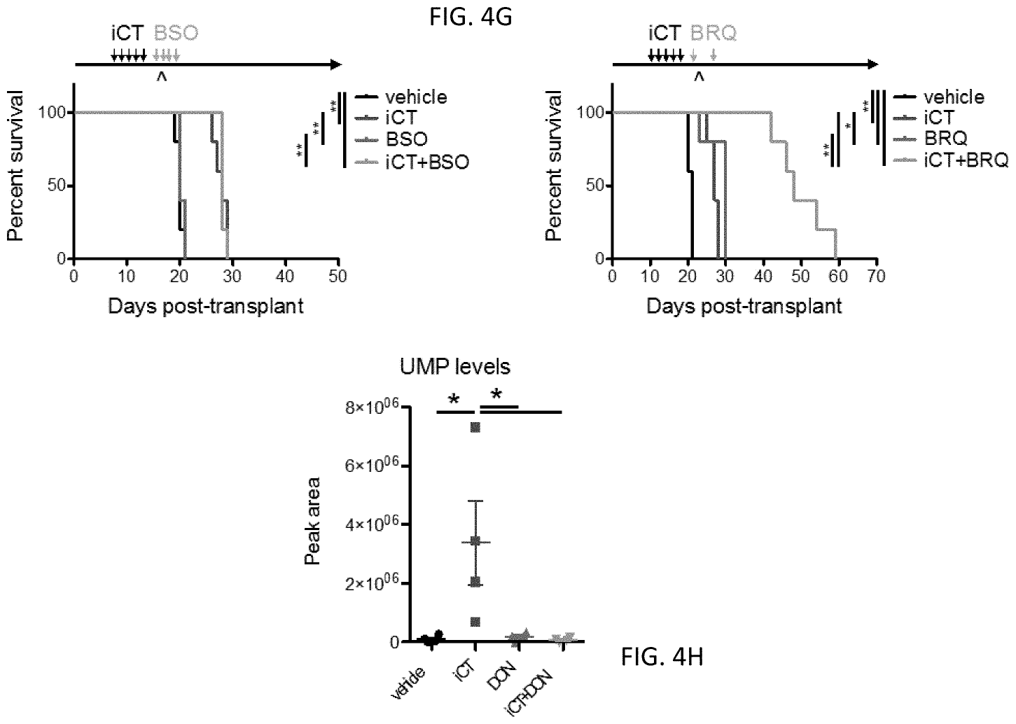

To understand how chemoresistant AML cells utilize glutamine, ¹³C₅-glutamine or ¹⁵N₂-glutamine was injected intravenously into vehicle- and iCT-treated mice at the moment of maximal response. Analysis of labeling patterns in AML cells showed no changes in the contribution of glutamine-derived carbon or nitrogen to amino acids or TCA cycle metabolites (FIGS. 4A-4B). A substantial increase of glutamine carbon and nitrogen incorporation into glutathione in chemoresistant AML cells was found (FIG. 4C). In addition, increased glutamine nitrogen incorporation in nucleotides was observed (FIG. 4D), especially into the pyrimidine uridine 5′-monophosphate (UMP). UMP gets one nitrogen from glutamine and one from aspartate, which in turn can be synthetized from glutamine-derived glutamate through transaminase activity (FIG. 8D). Increased fractions of UMP both containing one or two labeled nitrogen atoms was observed, showing that chemoresistant AML cells fuel pyrimidine synthesis with glutamine nitrogen both directly and indirectly through aspartate. Investigation of metabolite pool sizes revealed decreased levels of reduced glutathione (GSH) and unchanged levels of oxidized glutathione (GSSG) in chemoresistant cells (FIG. 4E). Together with the tracing results (FIG. 4C), this suggest that chemoresistant AML cells ramp up glutathione synthesis to replenish depleted GSH pools. Absolute levels of UMP in contrast were increased in chemoresistant cells (FIG. 4F), in line with increased pyrimidine synthesis fueled by glutamine. Chemoresistant AML cells thus require glutamine-derived carbon and nitrogen to fuel glutathione synthesis and glutamine nitrogen to generate high levels of pyrimidine nucleotides.

Next, the potential of targeting glutathione synthesis and nucleotide synthesis individually to eliminate chemoresistant AML cells was investigated. To inhibit glutathione biosynthesis the compound L-buthionine-sulfoximine (BSO) was used, which inhibits glutamate-cysteine ligase (GCL). Treatment of AML-bearing mice with BSO for 4 days following iCT showed no difference in survival compared to iCT alone (FIG. 4G). Then pyrimidine synthesis was blocked using brequinar (BRQ), which inhibits the enzyme dihydroorotate dehydrogenase (DHODH) (FIG. 8D). Administration of only two doses of BRQ following iCT was sufficient to significantly extend survival of AML-bearing mice compared to iCT treatment alone (FIG. 4G). These data show that chemoresistant AML cells require glutamine-dependent pyrimidine synthesis for their survival, while glutathione synthesis is not essential. In line with these findings, it was found that the pan-glutamine metabolism inhibitor DON prevented the increase of UMP levels observed in chemoresistant AML cells (FIG. 4H), confirming that these cells require glutamine to synthesize pyrimidines.

Bone Marrow Stromal Cell-Derived Aspartate Supports Pyrimidine Synthesis in AML Cells

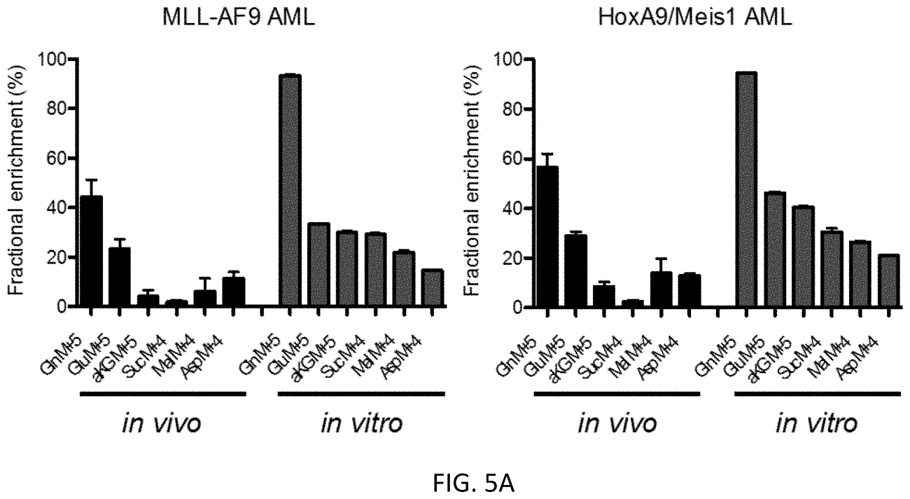

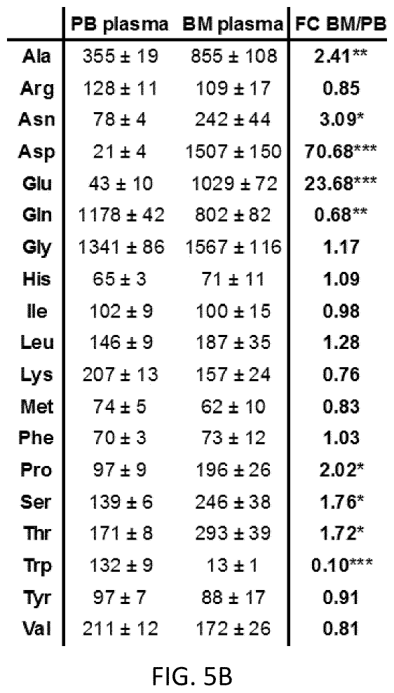

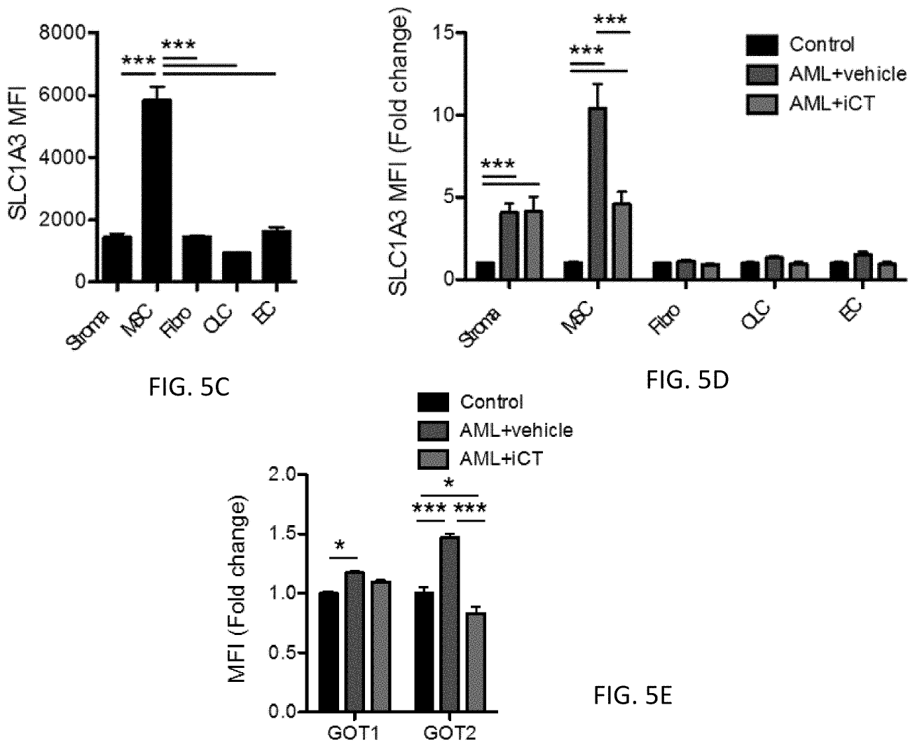

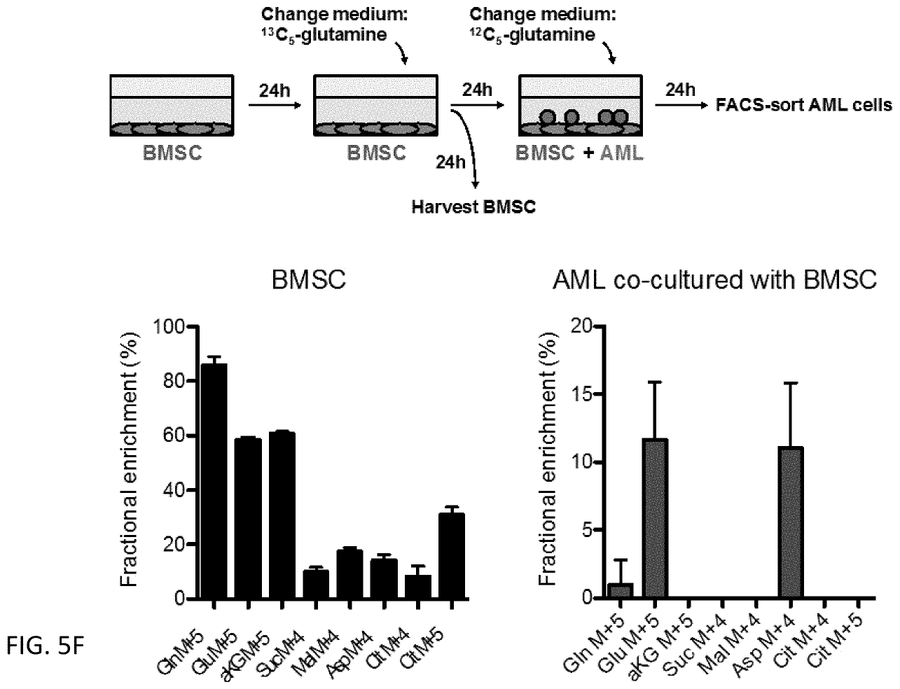

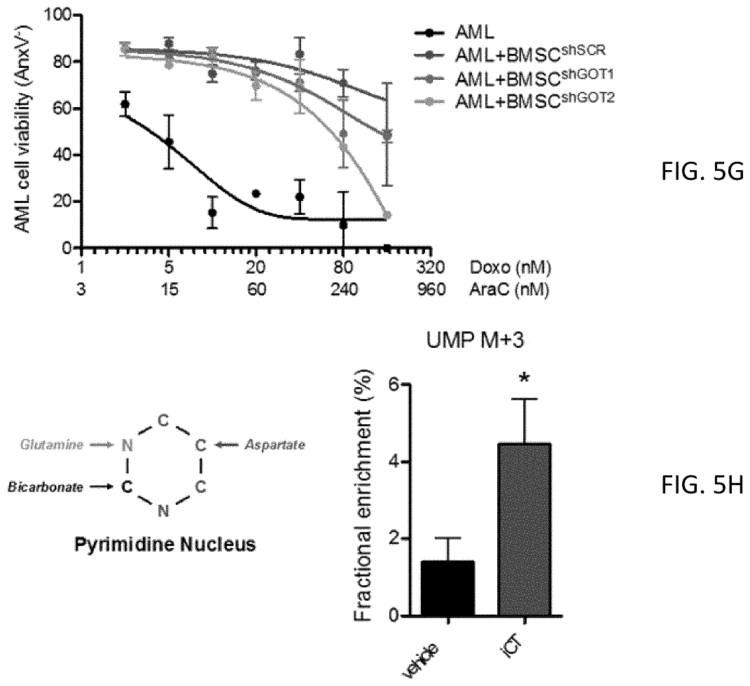

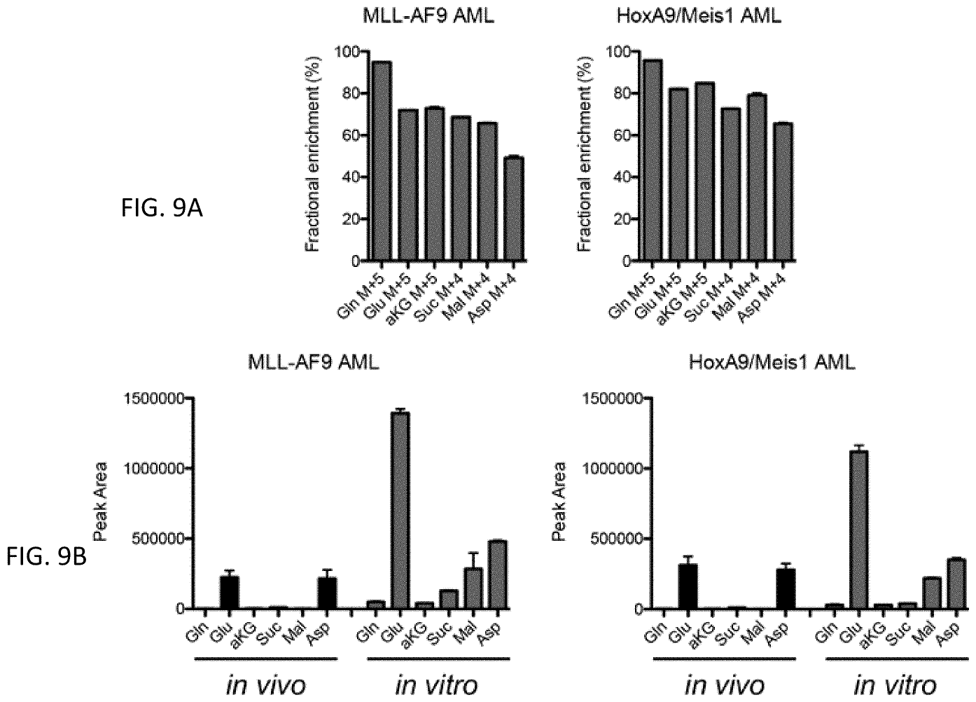

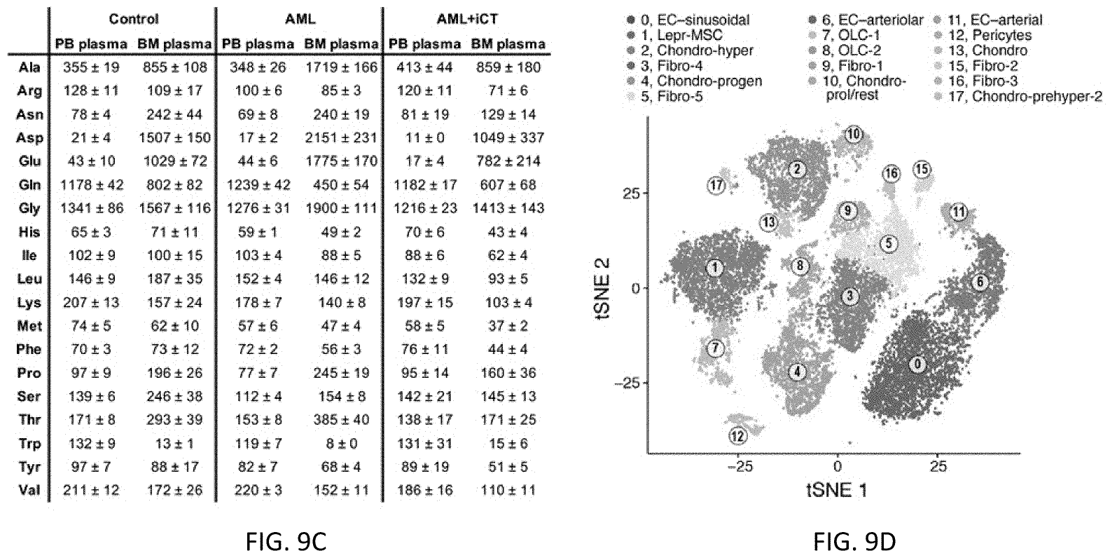

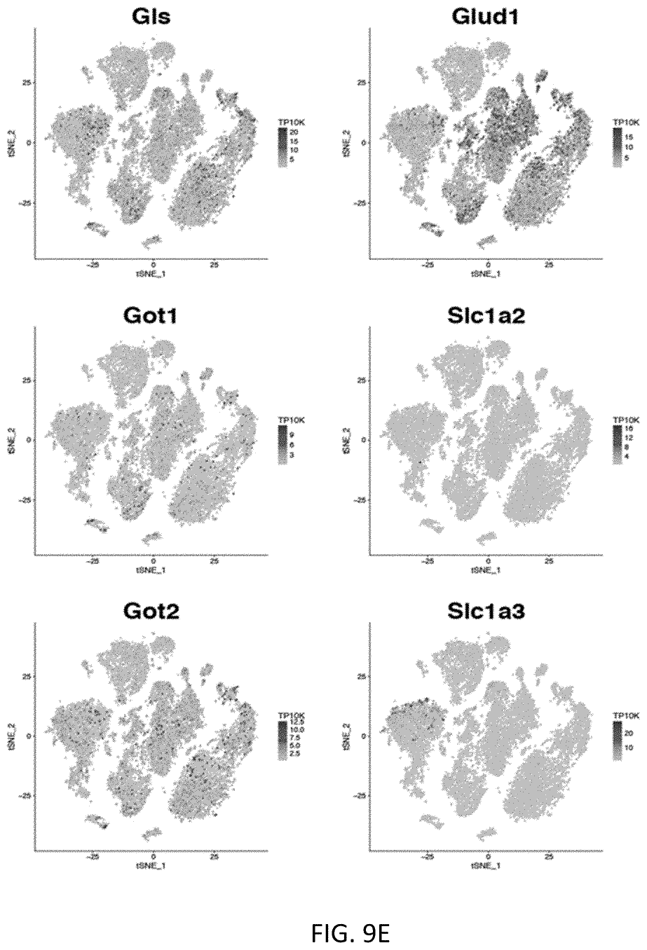

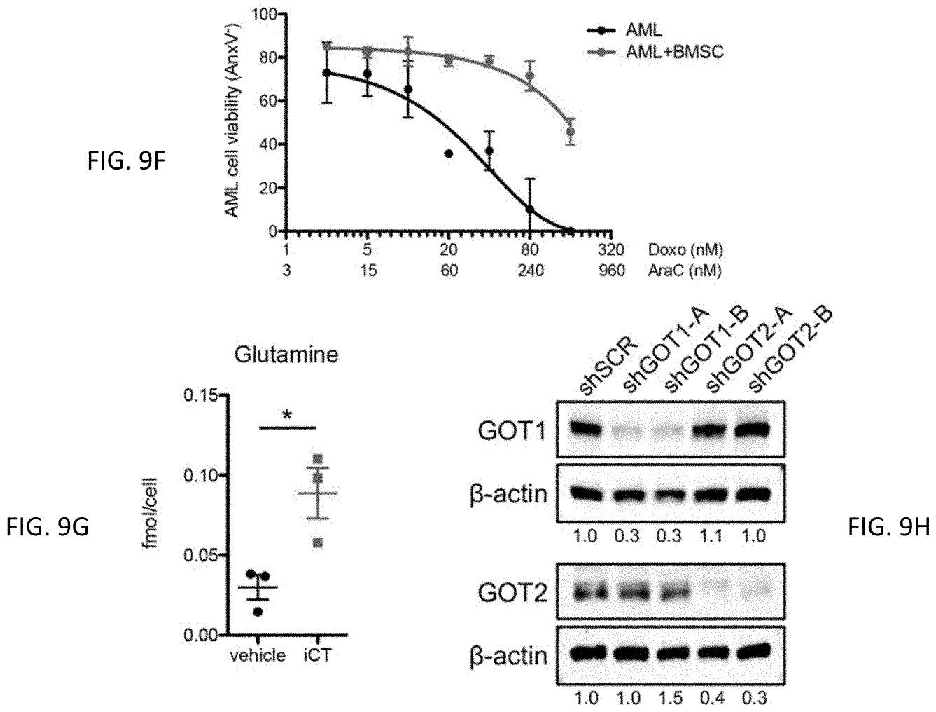

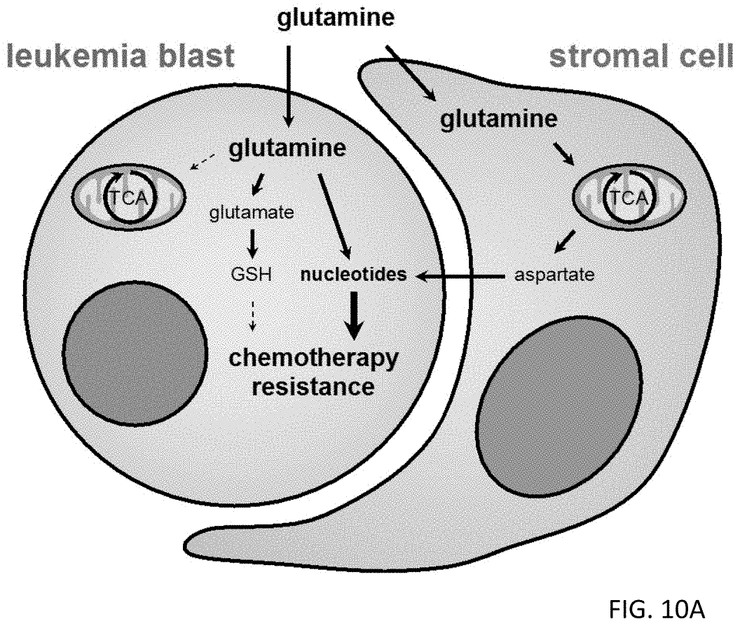

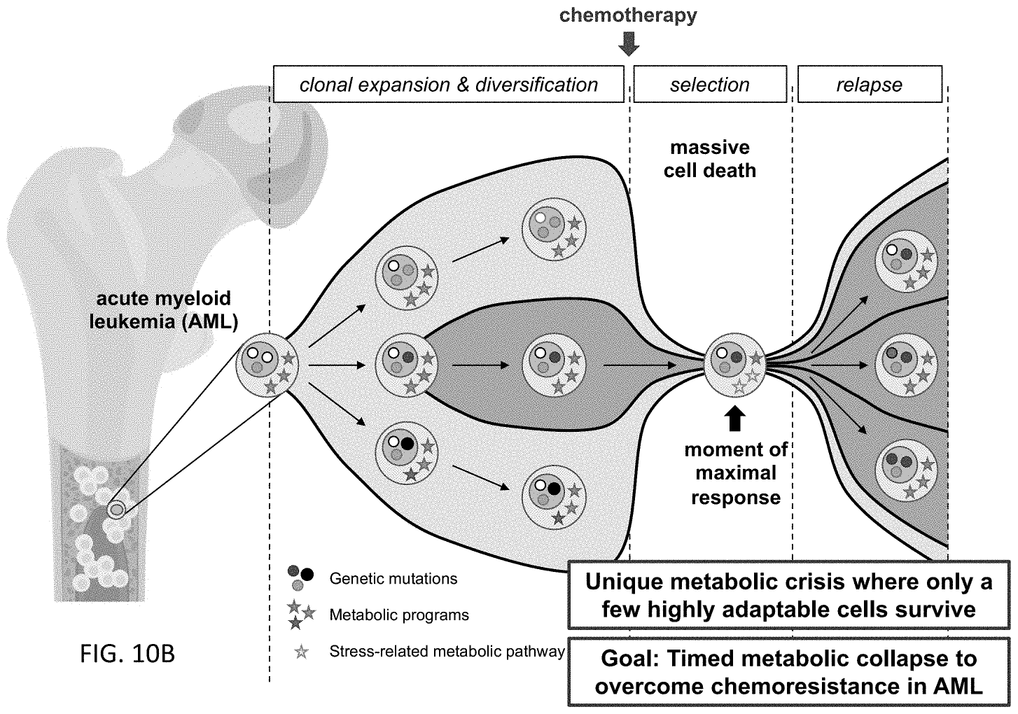

In vivo ¹³C₅-glutamine tracing experiments showed that AML cells have substantially different labeling patterns compared to the liver (FIG. 8C). While TCA metabolites in liver show gradually decreasing ¹³C enrichment (glutamine/glutamate>alpha-ketoglutarate>succinate/malate>aspartate>citrate), AML cells show hardly any labeling of alpha-ketoglutarate, succinate and citrate, but substantial labeling of the malate and especially aspartate pools. Since aspartate is another metabolite essential for pyrimidine synthesis, the origins of this particular labeling pattern were further explored. Metabolic tracing using ¹³C₅-glutamine was performed in MLL-AF9- and HoxA9/Meis1-driven AML lines, both in vivo and in vitro, with the same 10 minutes labeling duration. Interestingly, both AML models showed the unique labeling patterns only in vivo, while in vitro they exhibited the gradually decreasing labeling patterns similar to that seen in the liver samples in vivo (FIG. 5A). Longer in vitro labeling increased overall tracer enrichment, but did not change the labeling patterns (FIG. 9A). These results show that the ¹³C₅-glutamine labeling pattern seen in AML cells in vivo is not due to technical constraints of the approach such as labeling time or the cell model used, but reflects a unique metabolic program of AML cells in vivo.

Glutamine carbon cannot end up in aspartate without going through alpha-ketoglutarate and succinate in the TCA cycle (FIG. 9B). One possibility is that the interconversion of glutamate to aspartate occurs so rapid that labeling in the intermediates is not seen. However, further analysis of the in vivo ¹³C₅-glutamine tracing experiments revealed that labeling of glutamate and aspartate pools reached its peak in AML cells before labeling of glutamine pools (FIG. 9A). It was also found that AML cells in vivo have very small pools of alpha-ketoglutarate, succinate and malate compared to glutamate and aspartate (FIG. 9B), making it unlikely to not see substantial labeling of these metabolites even if the interconversion rates are high. The data thus suggest that AML cells in vivo obtain (glutamine-derived) aspartate from another source. Analysis of amino acid levels in peripheral blood revealed that aspartate is the lowest of all amino acids in the circulation (FIG. 5B), in accordance with previous reports [20,21]. However, when amino acid levels in the bone marrow plasma were compared to those in the peripheral blood plasma it was found that while most amino acids do not differ substantially, aspartate levels are 70-fold higher, making it the second most abundant amino acid in bone marrow plasma (FIG. 5B). Glutamate, also low abundance in peripheral blood, was 23-fold higher in bone marrow, while glutamine levels were 30 percent lower. Interestingly, in mice engrafted with AML cells aspartate levels were further increased in the bone marrow plasma, while after iCT treatment aspartate levels were significantly lower (FIG. 9C). Glutamate levels followed a similar trend, while glutamine concentrations showed the opposite profile. These data suggest that AML cells may obtain aspartate from a local bone marrow source, which may be of particular importance after iCT given the increased levels of aspartate in chemoresistant AML cells (FIG. 1E) and the importance of this metabolite for pyrimidine synthesis.

To identify the bone marrow cells responsible for local aspartate secretion, expression of genes encoding for enzymes involved in glutamine-to-aspartate conversion and for aspartate transporters in a single cell RNA sequencing dataset of the mouse bone marrow stroma were analyzed (FIG. 9D) [22]. Most enzymes involved in aspartate synthesis from glutamine, including Gls, glutamate-oxaloacetate transaminase 1 (Got1), Got2 and glutamate dehydrogenase (Glud1), were expressed throughout the bone marrow stroma, including in Leptin Receptor (LepR)⁺CXCL12⁺ mesenchymal stromal cells (cluster 1), S100A4⁺ fibroblasts (clusters 3, 5, 9, 15, 16), CD31⁺ endothelial cells (clusters 0, 6, 11), osteocalcin osteolineage cells (clusters 7, 8), aggrecan⁺ chondrocytes (2, 4, 10, 13, 17) and Nestin⁺NG2⁺ pericytes (cluster 12) (FIG. 9E). In contrast, the aspartate/glutamate transporter Slc1a3 was expressed almost exclusively in LepR⁺ stromal cells (FIG. 9E), which were confirmed at the protein level (FIG. 5C). The presence of AML cells increased SLC1A3 levels in stromal cells and particularly in LepR⁺ cells, while iCT treatment again reduced SLC1A3 levels in LepR⁺ cells, although they remained significantly higher compared to control mice without AML (FIG. 5D). The presence of AML cells also increased the levels of the aspartate transaminases GOT1 and particularly GOT2 in LepR⁺ cells (FIG. 5E), thus indicating that AML cells signal to BMSCs and specifically alter their metabolism to increase aspartate production and secretion. To further explore a possible exchange of aspartate between bone marrow stromal cells (BMSCs) and AML cells, in vitro cocultures were set up. In line with previous studies, it was found that the presence of BMSCs protected AML cells from iCT treatment. Of note, an increase in glutamine levels in AML cells exposed to iCT in cocultures was observed (FIG. 9G), which was not seen in AML monocultures (FIG. 6I), but which does not mirror the in vivo response of AML cells to iCT (FIG. 1D). This shows that the presence of stromal cells improves mimicking of the in vivo metabolic microenvironment of AML cells. ¹³C₅-glutamine tracing experiments were performed in which the stromal cells were labeled for 24 hours, before washing away the tracer and adding AML cells in medium containing unlabeled glutamine. Again 24 hours later AML cells were sorted and stable isotope labeling patterns analyzed. BMSCs metabolized glutamine in the TCA cycle, and synthetized aspartate using glutamine carbons, with about 15% of the aspartate pool fully labeled (FIG. 5F). At least part of this aspartate was then transferred to the AML cells, which showed 11% of their aspartate pool labeled, confirming the existence of metabolic crosstalk between BMSCs and AML cells. Interestingly, AML cells also showed uptake of labeled glutamate produced by BMSCs, the only other amino acid enriched in the bone marrow plasma (FIG. 5B, FIG. 9C), highly abundant in AML cells in vivo (FIG. 9B) and increased after iCT treatment (FIG. 1D). The importance of this metabolic crosstalk was analyzed by knocking down GOT1 or GOT2 in BMSCs (FIG. 9H). Loss of GOT2 in BMSCs in particular partially sensitized AML cells to iCT treatment, confirming that aspartate exchange contributes to the chemoprotective effect BMSCs on AML cells (FIG. 5G). Together, these data show that BMSCs convert glutamine to aspartate, which is then transferred to AML cells and helps them survive iCT exposure.

Since aspartate is an essential metabolite for pyrimidine synthesis, evidence as sought in the in vivo tracing data that glutamine-derived aspartate is used by chemoresistant AML cells to fuel UMP synthesis. The ¹⁵N₂-glutamine tracing data showed that both single and double ¹⁵N-labeled UMP was increased in chemoresistant AML cells (FIG. 4D). Since the pyrimidine nucleus gets one nitrogen from glutamine and one from aspartate, this suggest that the glutamine-derived aspartate provided by the BMSCs is indeed used to support increased pyrimidine synthesis in chemoresistant AML cells. To further confirm this UMP containing three ¹³C atoms in the ¹³C₅-glutamine in vivo tracing data was looked for, since aspartate, but not glutamine, contributes three carbons to UMP. Increased pools of ¹³C₃-UMP were found in chemoresistant AML cells (FIG. 5H), further supporting the concept that glutamine fuels pyrimidine synthesis in chemoresistant AML cells both directly and indirectly via stromal cell-derived aspartate.

### Discussion

Despite the clear clinical need to prevent or delay cancer relapse, it remains poorly understood how certain tumor cells manage to survive the extreme stress of chemotherapy. In the current study, the metabolic profile of AML cells is defined as they pass through that time of intense selection pressure. It was found that residual AML cells exhibit transient metabolic adaptations driving their resistance to chemotherapy, and it was shown in mouse models of AML that timed manipulation of specific metabolic pathways, such as glutamine metabolism and pyrimidine synthesis, holds therapeutic potential.

The moment of maximal response immediately following chemotherapy is a time when the metabolic milieu of the bone marrow microenvironment is maximally hostile, representing a distinctive selection moment that the leukemia cells ultimately causing relapse must tolerate and pass through. To investigate the metabolic profile of AML cells at this specific time point, a pipeline was developed for untargeted metabolomics of freshly-isolated AML cells from mice. While this approach provides a broad snapshot of cellular metabolism in an unbiased manner, it also brings several challenges. First, cell isolation, sorting and sample processing must be performed in a fast but highly standardized manner, limiting the time between animal euthanasia and metabolite extraction to minimize metabolic changes. It was found that performing all steps at 4° C. greatly reduces procedural artefacts. Second, as with all metabolomics analyses, batch effects occur between individual experiments, but these seem to be even stronger when using low cell numbers. Including a reference sample that is taken in each experiment helps significantly in correcting for this. Thirdly, and most importantly, data processing and analysis for untargeted metabolomics is highly labor-intensive. Inefficient peak alignment and limited MS/MS data, both more prominent due to the use of low cell numbers, requires substantial manual data processing and brings more uncertainty in metabolite identification. Again, the use of a reference sample consisting of larger cell numbers can help, especially with improving automatic metabolite annotation from cloud-based spectral databases as the larger cell numbers give better MS/MS data used for compound identification. Despite these challenges, the described approach proved to be highly valuable and brought a unique look into the metabolic program of AML cells in their native environment, which would not have been obtained through transcriptomic analysis alone. In addition, a key metabolic feature of chemoresistant cells, high glutamine levels, was lost once cells were isolated and cultured, and was also not observed when AML cells were treated with iCT in vitro. These results indicate that the dynamic metabolic adaptations of AML cells in response to chemotherapy are regulated at the pathway activity level, and are highly dependent on the local microenvironment. This notion is further underscored by a metabolic tracer analysis, which revealed striking differences in glutamine metabolism in AML cells in vitro versus in vivo. The low contribution of glutamine to the TCA cycle in AML cells in vivo may explain the poor efficacy of GLS inhibition, even though this appears to be a promising target based on in vitro assays, and the peculiar nutritional composition of the bone marrow microenvironment may even further contribute to this lack of effect. The importance of examining the metabolism of cells in their native environment is supported by other recent studies that have shown that artificial in vitro conditions can create metabolic dependencies that do not exist in vivo [20,23].

Many studies have investigated the role of the bone marrow niche in AML progression and chemoresistance [24]. However, most of these studies have focused on the role of adhesion molecules and cytokines. Very few studies have investigated the nutritional aspects of niche support in leukemia, with one report highlighting a role for bone marrow stromal cell-derived cysteine in chronic lymphocytic leukemia [25], and a second study showing the importance of lipids provided by adipocytes for AML stem cells residing in adipose tissue [26]. It is now shown that aspartate provided by LepR⁺ stromal cells in the bone marrow can fuel nucleotide synthesis in AML cells, which becomes of particular importance in the setting of chemotherapy treatment. These findings again highlight the question of how much of chemoresistance development is determined by genetic alterations versus microenvironmental signals. Are there hotspots of aspartate production in the bone marrow that constitute a protective niche, and is there competition between AML subclones for occupancy of these aspartate-rich locations? The transgenic transplant-based mouse models used in the current study most likely do not possess the clonal complexity to answer this question. However, the documentation of the SLC1A3^{high} subset of LepR⁺ stromal cells may provide the means to identify this putative chemoprotective niche in AML models that capture clonal evolution, such as transgenic mouse models with more mutational complexity or patient-derived xenografts, or even directly in patient samples. In addition, it will be of interest to examine whether this metabolic crosstalk with stromal cell subsets also plays a role in normal hematopoiesis, given that the bone marrow is unexpectedly rich in aspartate compared to peripheral blood, also in control mice,

### REFERENCES

1. Weissman I L. Stem cells are units of natural selection for tissue formation, for germline development, and in cancer development. Proc Natl Acad Sci USA 2015; 112:8922-8928.
2. Nowell, P C. The clonal evolution of tumor cell populations. Science 1976; 194:23-28.
3. Ding L, Ley T J, Larson D E, Miller C A, Koboldt D C, Welch J S, Ritchey J K, Young M A, Lamprecht T, McLellan M D, McMichael J F, Wallis J W, Lu C, Shen D, Harris C C, Dooling D J, Fulton R S, Fulton L L, Chen K, Schmidt H, Kalicki-Veizer J, Magrini V J, Cook L, McGrath S D, Vickery T L, Wendl M C, Heath S, Watson M A, Link D C, Tomas son M H, Shannon W D, Payton J E, Kulkarni S, Westervelt P, Walter M J, Graubert T A, Mardis E R, Wilson R K, DiPersio J F. Clonal evolution in relapsed acute myeloid leukaemia revealed by whole-genome sequencing. Nature 2012; 481:506-510.
4. Magee J A. Comment on: Genomics of primary chemoresistance and remission induction failure in paediatric and adult acute myeloid leukaemia. Br J Haematol 2017; 176:5-6.
5. Keller M A, Kampjut D, Harrison S A, Ralser M. Sulfate radicals enable a non-enzymatic Krebs cycle precursor. Nat Ecol Evol 2017; 1:83.
6. Kültz D. Molecular and evolutionary basis of the cellular stress response. Annu Rev Physiol 2005; 67:225-257.
7. Naviaux R K. Metabolic features of the cell danger response. Mitochondrion
8. Lagadinou E D, Sach A, Callahan K, Rossi R M, Neering S J, Minhajuddin M, Ashton J M, Pei S, Grose V, O'Dwyer K M, Liesveld J L, Brookes P S, Becker M W, Jordan C T. BCL-2 inhibition targets oxidative phosphorylation and selectively eradicates quiescent human leukemia stem cells. Cell Stem Cell 2013; 12(3):329-341.
9. Pei S, Minhajuddin M, Callahan K P, Balys M, Ashton J M, Neering S J, Lagadinou E D, Corbett C, Ye H, Liesveld J L, O'Dwyer K M, Li Z, Shi L, Greninger P, Settleman J, Benes C, Hagen F K, Munger J, Crooks P A, Becker M W, Jordan C T. Targeting aberrant glutathione metabolism to eradicate human acute myelogenous leukemia cells. J Biol Chem 2013; 288:33542-33558.
10. Wang Y H, Israelsen W J, Lee D, Yu V W C, Jeanson N T, Clish C B, Cantley L C, Vander Heiden M G, Scadden D T. Cell-state-specific metabolic dependency in hematopoiesis and leukemogenesis. Cell 2014; 158:1309-1323.
11. Jacque N, Ronchetti A M, Larrue C, Meunier G, Birsen R, Willems L, Saland E, Decroocq J, Maciel T T, Lambert M, Poulain L, Hospital M A, Sujobert P, Joseph L, Chapuis N, Lacombe C, Moura I C, Demo S, Sarry J E, Recher C, Mayeux P, Tamburini J, Bouscary D. Targeting glutaminolysis has antileukemic activity in acute myeloid leukemia and synergizes with BCL-2 inhibition. Blood 2015; 126:1346-1356.
12. Chen W L, Wang Y Y, Zhao A, Xia L, Xie G, Su M, Zhao L, Liu J, Qu C, Wei R, Rajani C, Ni Y, Cheng Z, Chen Z, Chen S J, Jia W. Enhanced fructose utilization mediated by SLC2A5 is a unique metabolic feature of acute myeloid leukemia with therapeutic potential. Cancer Cell 2016; 30:779-791.
13. German N J, Yoon H, Yusuf R Z, Murphy J P, Finley L W, Laurent G, Haas W, Satterstrom F K, Guarnerio J, Zaganjor E, Santos D, Pandolfi P P, Beck A H, Gygi S P, Scadden D T, Kaelin W G Jr, Haigis M C. PHD3 loss in cancer enables metabolic reliance on fatty acid oxidation via deactivation of ACC2. Mol Cell 2016; 63:1006-1020.
14. Ni F, Yu W M, Li Z, Graham D K, Jin L, Kang S, Rossi M R, Li S, Broxmeyer H E, Qu C K. Critical role of ASCT2-mediated amino acid metabolism in promoting leukaemia development and progression. Nat Metab 2019; 1:390-403.
15. Dietmair S, Timmins N E, Gray P P, Nielsen L K, Kromer J O. Towards quantitative metabolomics of mammalian cells: development of a metabolite extraction protocol. Anal Biochem 2010; 404:155-164.
16. Lemberg K M, Vornov J J, Rais R, Slusher B S. We're Not “DON” Yet: Optimal Dosing and Prodrug Delivery of 6-Diazo-5-oxo-L-norleucine. Mol Cancer Ther 2018; 17:1824-1832.
17. Matre P, Velez J, Jacamo R, Qi Y, Su X, Cai T, Chan S M, Lodi A, Sweeney S R, Ma H, Davis R E, Baran N, Haferlach T, Su X, Flores E R, Gonzalez D, Konoplev S, Samudio I, DiNardo C, Majeti R, Schimmer A D, Li W, Wang T, Tiziani S, Konopleva M. Inhibiting glutaminase in acute myeloid leukemia: metabolic dependency of selected AML subtypes. Oncotarget 2016; 7:79722-79735.
18. Gregory M A, Nemkov T, Park H J, Zaberezhnyy V, Gehrke S, Adane B, Jordan C T, Hansen K C, D'Alessandro A, DeGregori J. Targeting Glutamine Metabolism and Redox State for Leukemia Therapy. Clin Cancer Res 2019; 25:4079-4090.
19. Haussinger D, Schliess F. Glutamine metabolism and signaling in the liver. Front Biosci 2007; 12:371-391.
20. Cantor J R, Abu-Remaileh M, Kanarek N, Freinkman E, Gao X, Louissaint A Jr, Lewis C A, Sabatini D M. Physiologic medium rewires cellular metabolism and reveals uric acid as an endogenous inhibitor of UMP synthase. Cell 2017; 169:258-272.
21. Tardito S, Oudin A, Ahmed S U, Fack F, Keunen O, Zheng L, Miletic H, Sakariassen P Ø, Weinstock A, Wagner A, Lindsay S L, Hock A K, Barnett S C, Ruppin E, Mørkve S H, Lund-Johansen M, Chalmers A J, Bjerkvig R, Niclou S P, Gottlieb E. Glutamine synthetase activity fuels nucleotide biosynthesis and supports growth of glutamine-restricted glioblastoma. Nat Cell Biol 2015; 17:1556-1568.
22. Baryawno N, Przybylski D, Kowalczyk M S, Kfoury Y, Severe N, Gustafsson K, Kokkaliaris K D, Mercier F, Tabaka M, Hofree M, Dionne D, Papazian A, Lee D, Ashenberg O, Subramanian A, Vaishnav E D, Rozenblatt-Rosen O, Regev A, Scadden D T. A cellular taxonomy of the bone marrow stroma in homeostasis and leukemia. Cell 2019; 177:1915-1932.
23. Muir A, Danai L V, Gui D Y, Waingarten C Y, Lewis C A, Vander Heiden M G. Environmental cystine drives glutamine anaplerosis and sensitizes cancer cells to glutaminase inhibition. Elife 2017; 6:e27713.
24. Behrmann L, Wellbrock J, Fiedler W. Acute myeloid leukemia and the bone marrow niche—take a closer look. Front Oncol 2018; 8:444.
25. Zhang W, Trachootham D, Liu J, Chen G, Pelicano H, Garcia-Prieto C, Lu W, Burger J A, Croce C M, Plunkett W, Keating M J, Huang P. Stromal control of cystine metabolism promotes cancer cell survival in chronic lymphocytic leukaemia. Nat Cell Biol 2012; 14:276-286.
26. Ye H, Adane B, Khan N, Sullivan T, Minhajuddin M, Gasparetto M, Stevens B, Pei S, Balys M, Ashton J M, Klemm D J, Woolthuis C M, Stranahan A W, Park C Y, Jordan C T. Leukemic stem cells evade chemotherapy by metabolic adaptation to an adipose tissue niche. Cell Stem Cell 2016; 19:23-37.
27. Corral J, Lavenir I, Impey H, Warren A J, Forster A, Larson T A, Bell S, McKenzie A N, King G, Rabbitts T H. An Mll-AF9 fusion gene made by homologous recombination causes acute leukemia in chimeric mice: a method to create fusion oncogenes. Cell 1996; 85:853-861.

### Example 2: Induction of a Timed Metabolic Collapse to Overcome Cancer Chemoresistance

This Example both re-presents certain data from Example 1 and provides additional data.

Per evolutionary biology, when a population is subjected to a stress event or “bottleneck”, only the individuals that have the ability to adapt will survive. Populations of cells behave similarly (Weissman, 2015) as Peter C. Nowell proposed in 1976 to explain the clonal evolution of cancer cell populations (Nowell, 1976). Clonal evolution has been studied particularly well in acute myeloid leukemia (AML) (Ding et al., 2012a), a highly lethal hematopoietic cancer where residual founder clones persist through therapy and ultimately cause relapse. Yet, genetic analysis has provided only limited insight into chemotherapy resistance commonly observed in AML, and driver mutations are frequently not amenable to targeted therapies (Magee, 2017). It has been shown that AML cells have distinctive metabolic dependencies compared with their normal counterparts, often irrespective of the driving mutations (Chen et al., 2016, German et al., 2016, Jacque et al., 2015, Lagadinou et al., 2013, Ni et al., 2019, Pei et al., 2013, Sykes et al., 2016, Wang et al., 2014). Metabolic networks also provide cells with evolutionary-ancient stress protection systems that are independent of transcriptional changes, providing a first line defense mechanism to cope with exogenous pressure (Keller et al., 2017, Kültz, 2005, Naviaux, 2014).

It was hypothesized that the stress of chemotherapy imposes an extreme metabolic bottleneck through which resistant cells must pass to cause relapse. That stress state is transient, represented by the challenges of chemotherapeutic agents and cell degradation products from massive cell death in the local milieu. It will not be reflected in relapse cells at the time cancer reemergence is clinically evident, since much of the local environment and intracellular imbalances will have re-equilibrated. Rather, it can only be observed in cells extracted from the in vivo context where maximal cell death has occurred and chemotherapy by-products are being cleared. This transient moment of stress that drives initial leukemia regeneration has been described in patients as well as patient-derived xenografts, and is characterized by the emergence of leukemia-regenerating cells which give rise to relapse (Boyd et al., 2018). Defining metabolic dependencies of these residual leukemia-regenerating cells may reveal vulnerabilities that can be therapeutically exploited. It was sought to determine whether timed, targeted metabolic perturbations can induce metabolic collapse of residual malignant cells to overcome chemoresistance.

Methods were developed to track real-time changes to AML in live animals, capture cells at particular times, acquire unbiased metabolic profiles of the cells and test inferred dependencies in vitro and in vivo. Specific alterations were developed in cell metabolism that allow AML cells to persist through the selection stress of chemotherapy in part by co-opting metabolic activity of neighboring stromal cells and it was shown that these can be manipulated in a timed manner to improve survival from the disease.

### Results

AML Cells Exhibit Transient Metabolic Changes in Response to Chemotherapy

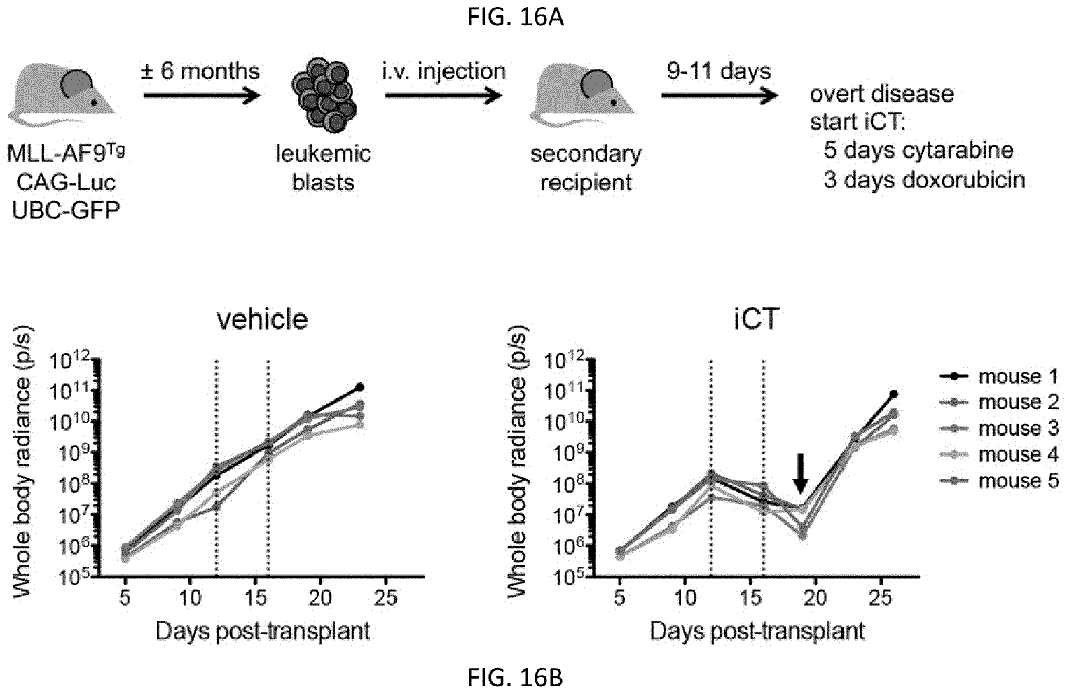

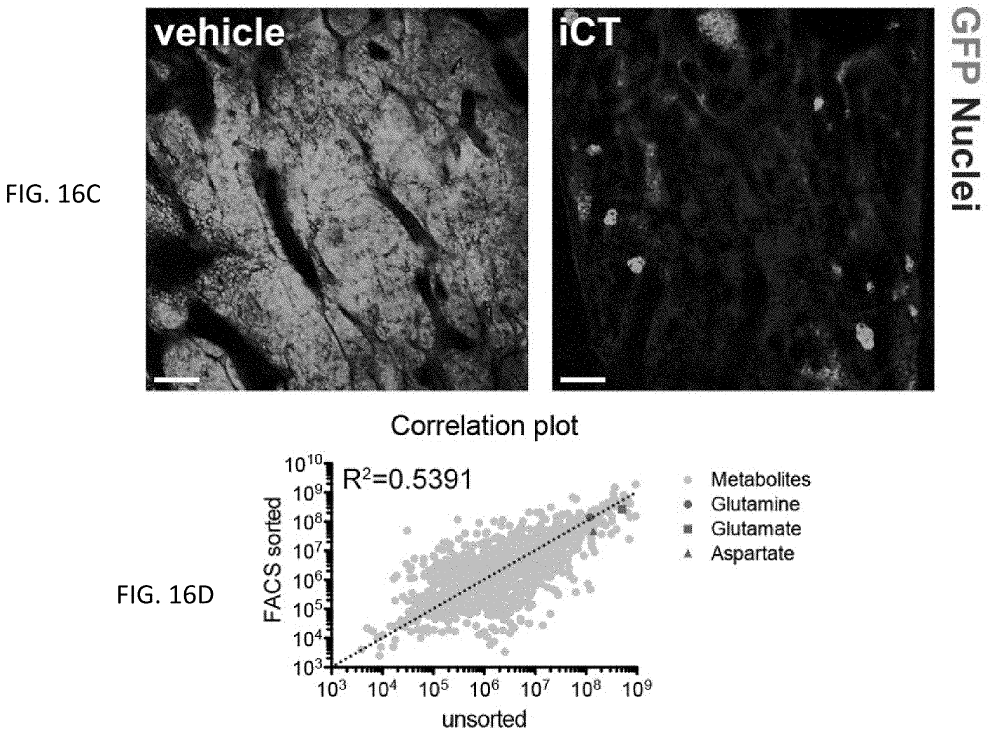

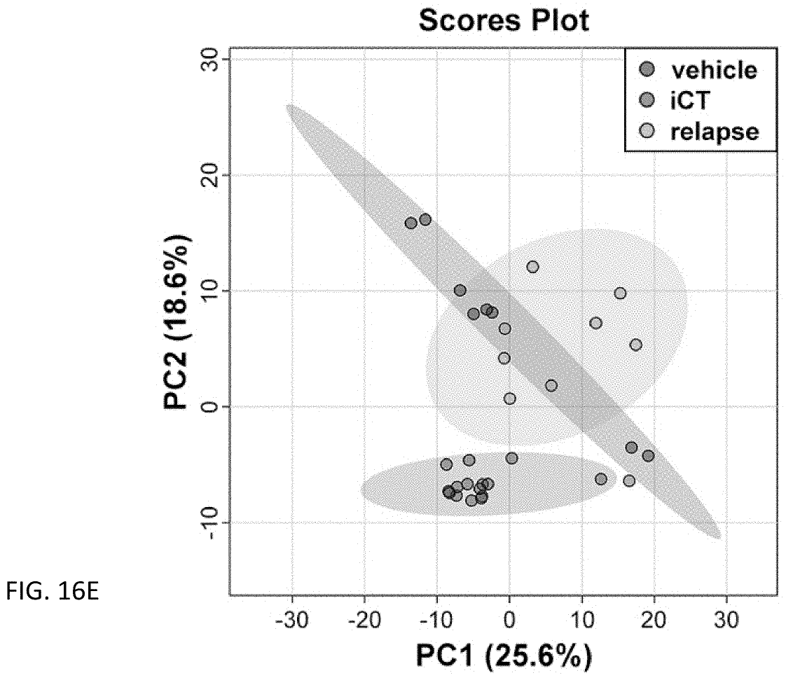

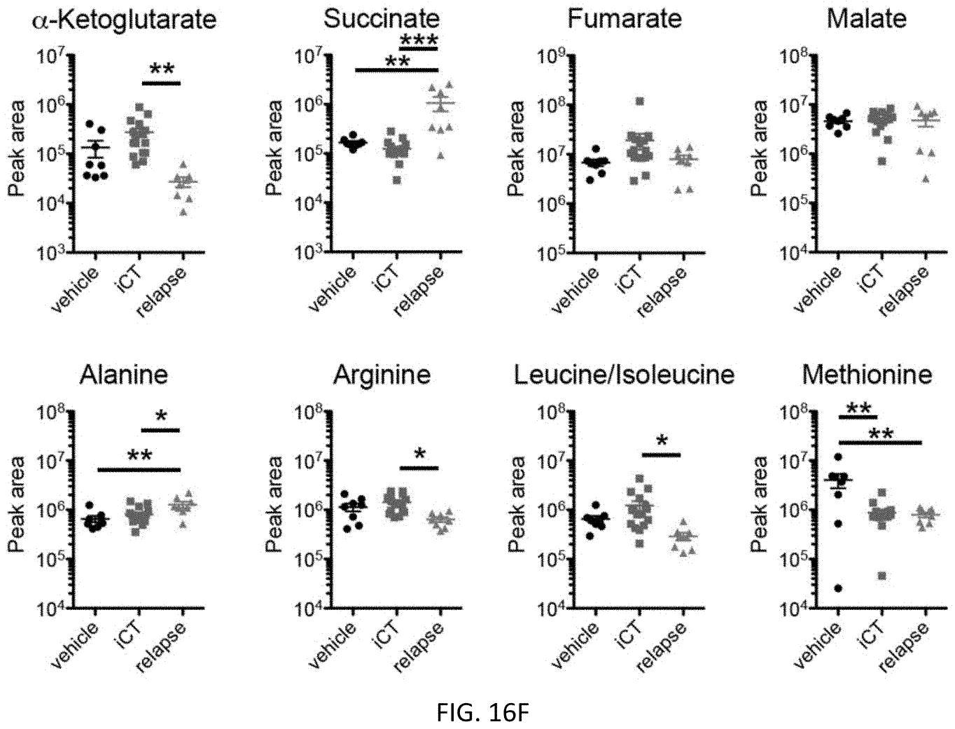

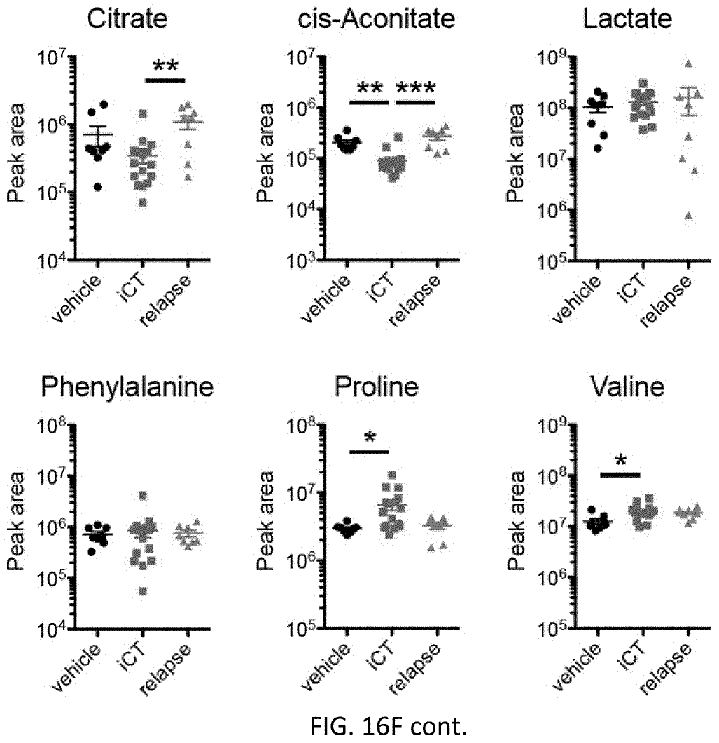

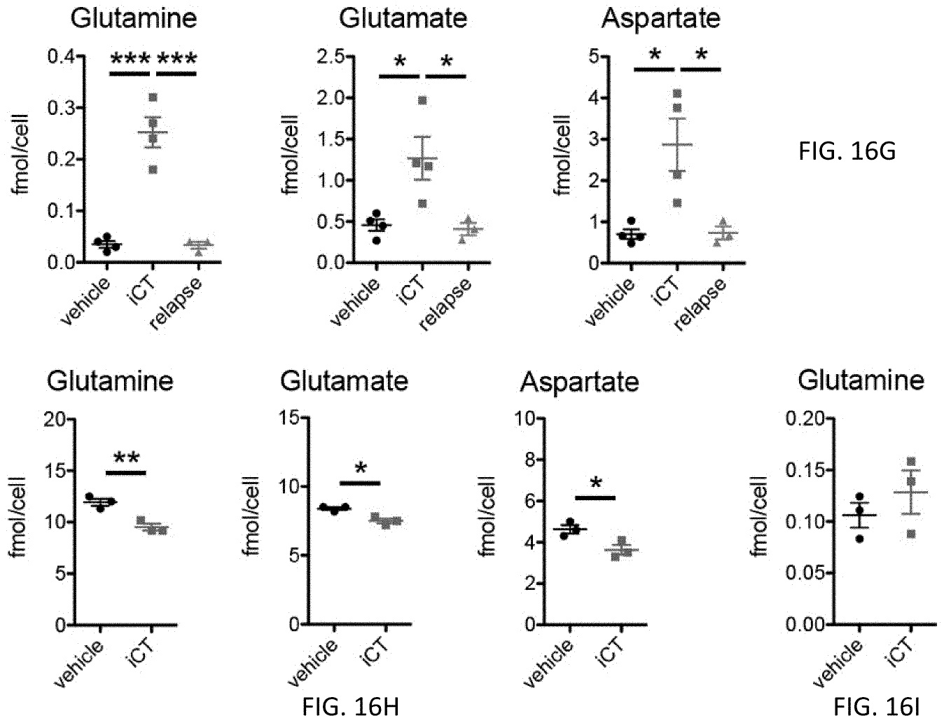

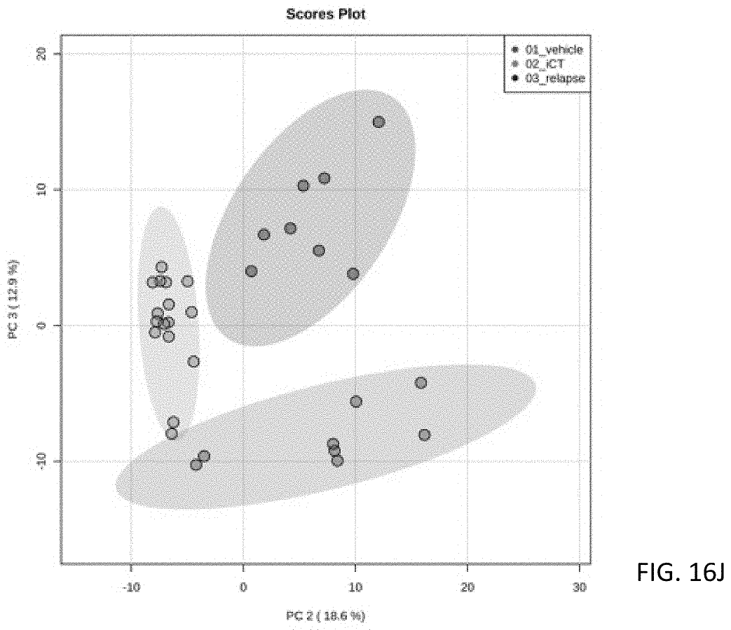

Relapse is declared at the point of maximal metabolic stress from toxic chemotherapy and a hostile microenvironment laden with byproducts of massive neighboring cell death. Functionally, this can be identified as the “point of maximal response” after chemotherapy since thereafter resistant cells have begun the regrowth that characterizes relapse. To determine this moment, a triple transgenic mouse model in which cells express MLL-AF9 was used, driving leukemia development, as well as luciferase and GFP (FIG. 16A). Culture-expanded bone marrow cells derived from terminally ill primary mice were intravenously transplanted into secondary wildtype recipients, leading to the establishment of a fast-developing disease that can be monitored through bioluminescence imaging (FIG. 16B). Mice were treated with an induction chemotherapy (iCT) regimen that closely mimics the one used in the clinical care of people (cytarabine for 5 days+doxorubicin for the first 3 days) or with vehicle, disease progression was followed, and it was discovered that the moment of maximal response occurs approximately three days after the last dose of chemotherapy (FIGS. 16B-16C).

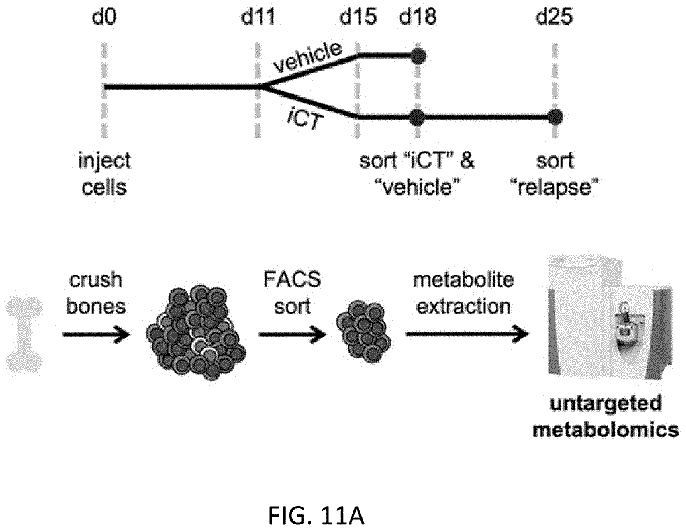

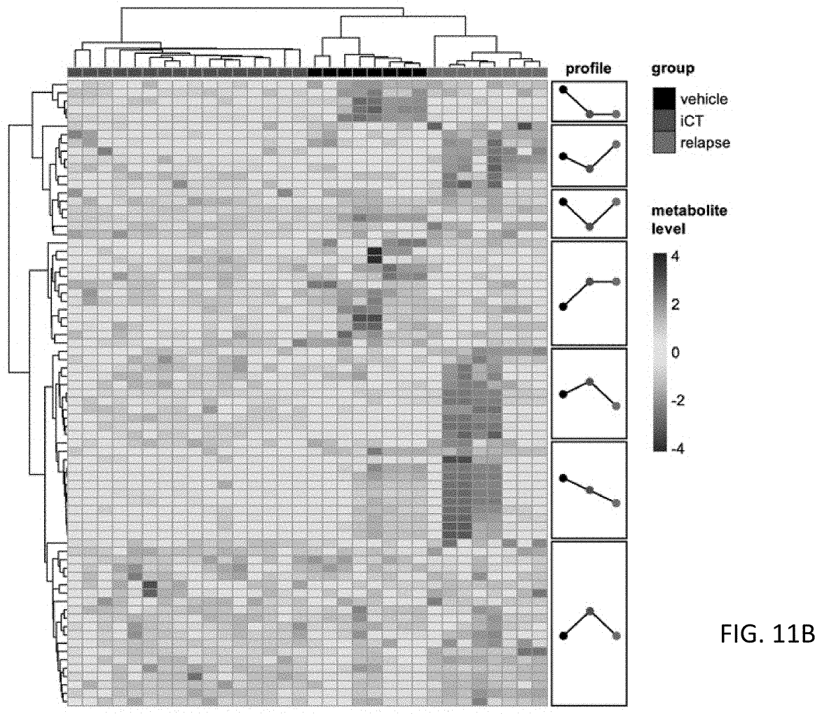

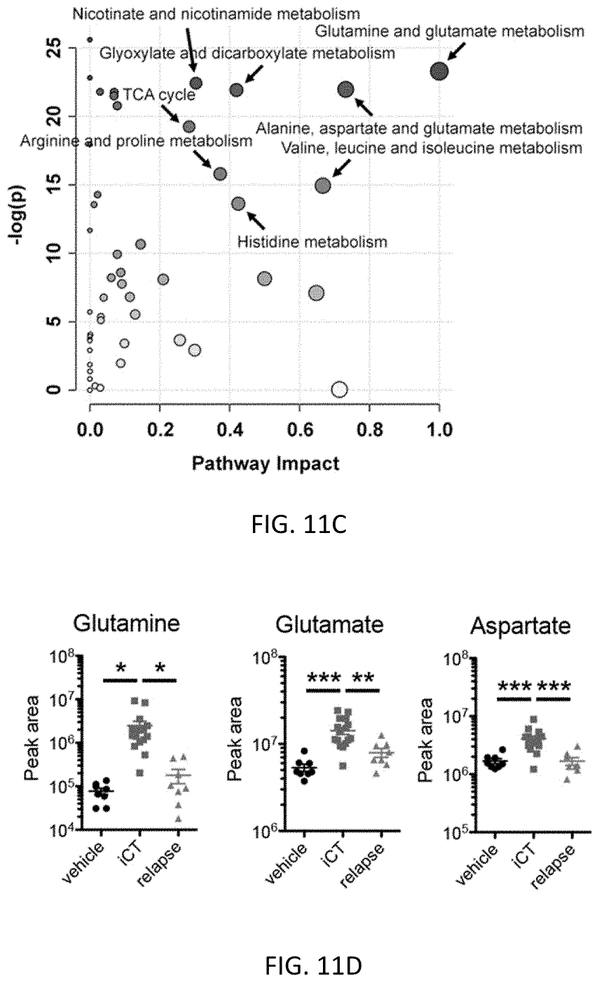

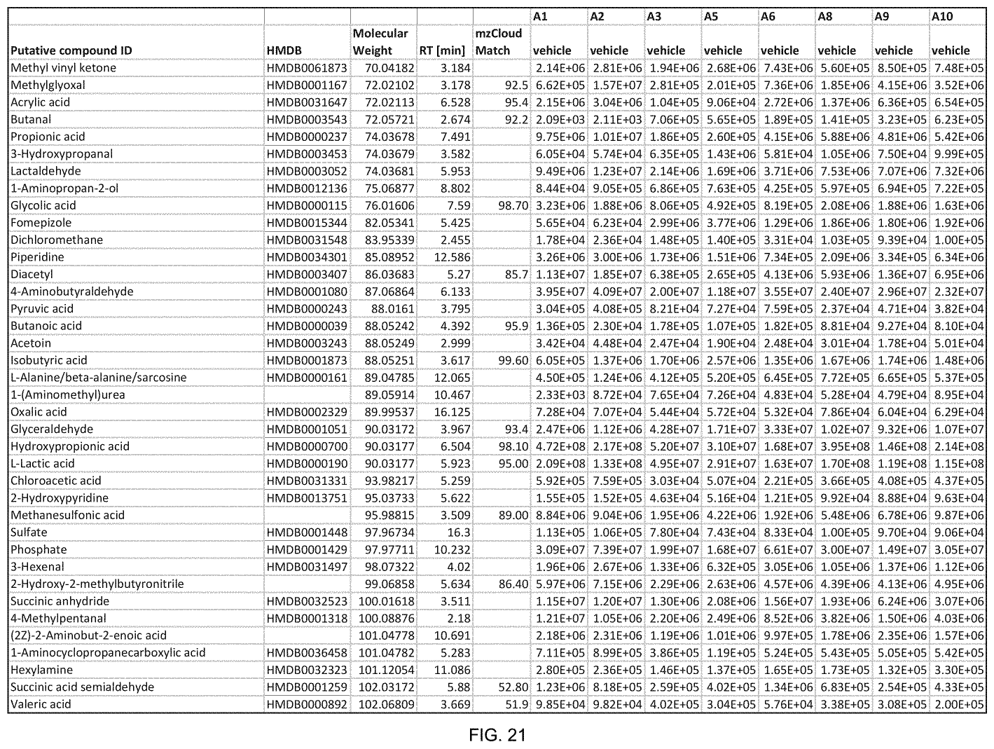

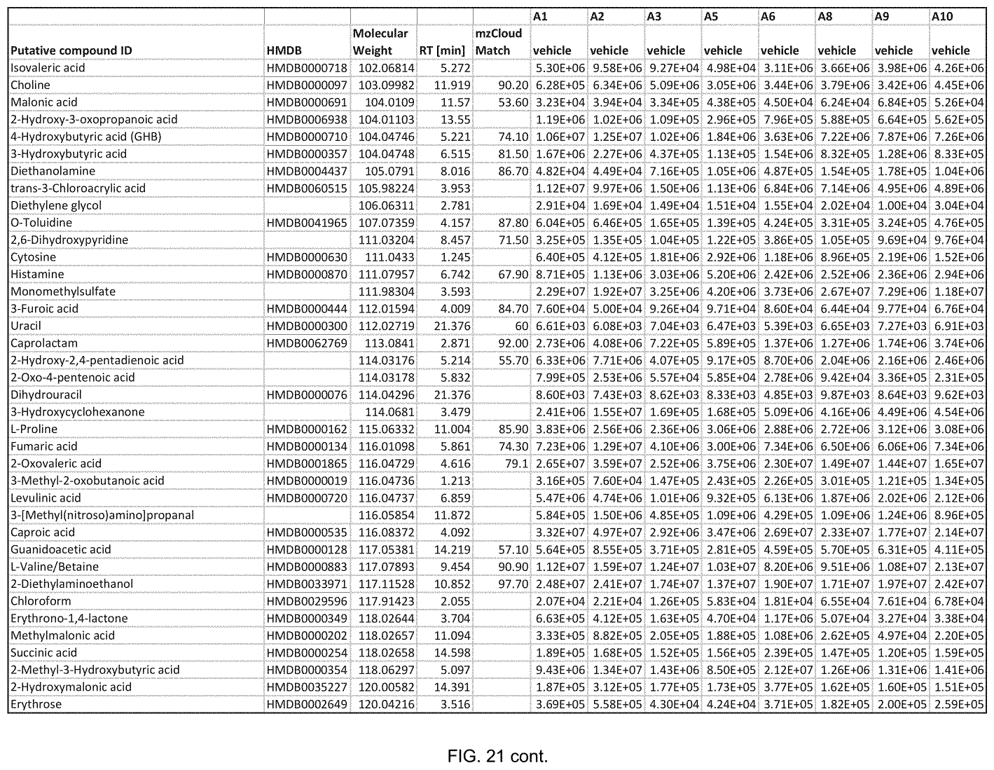

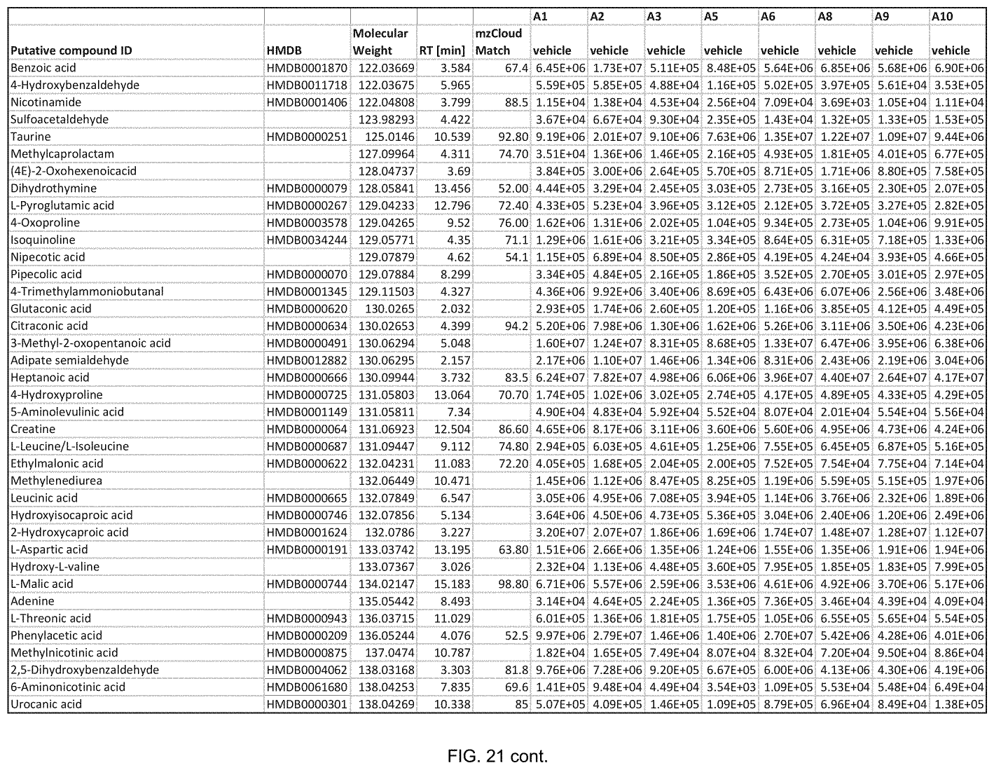

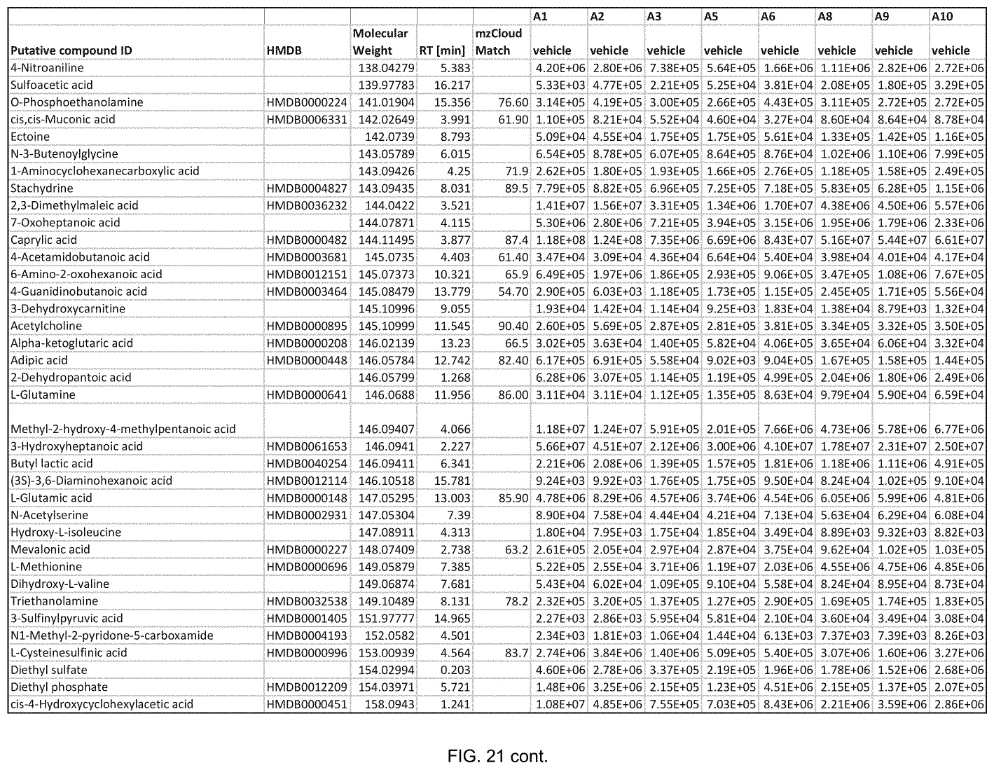

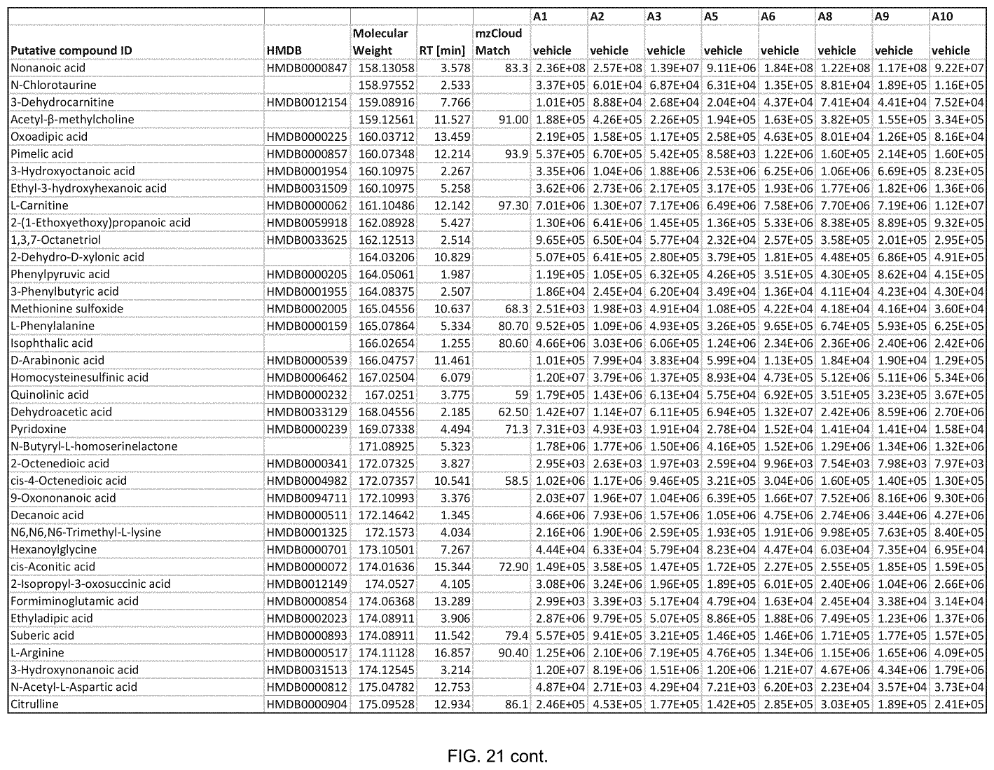

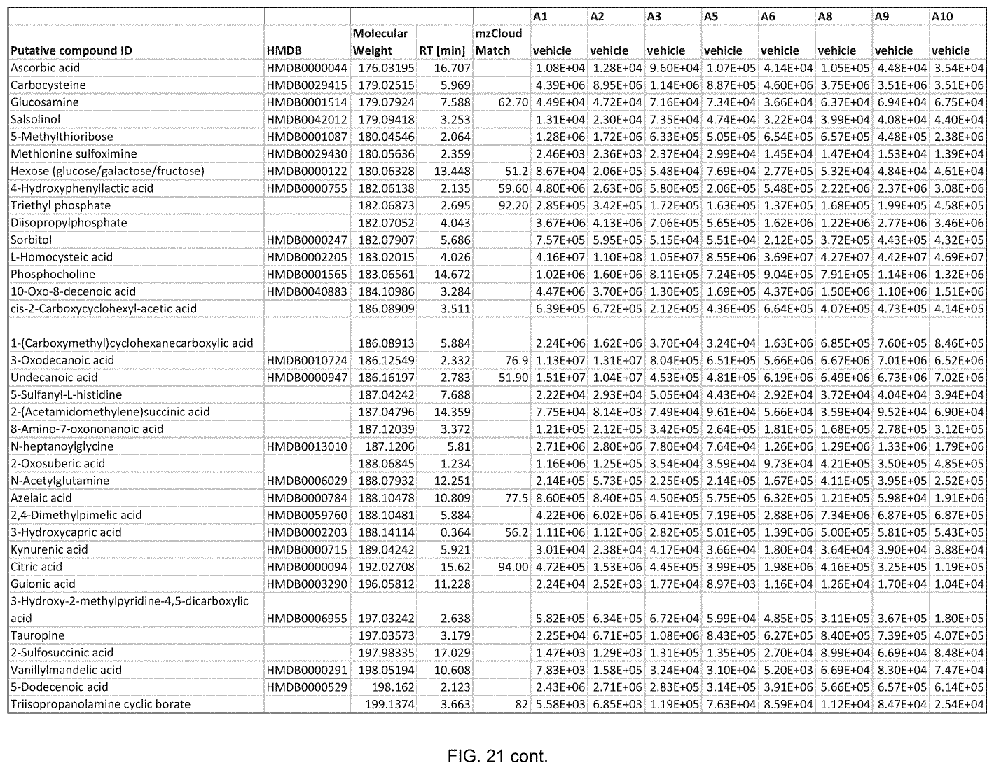

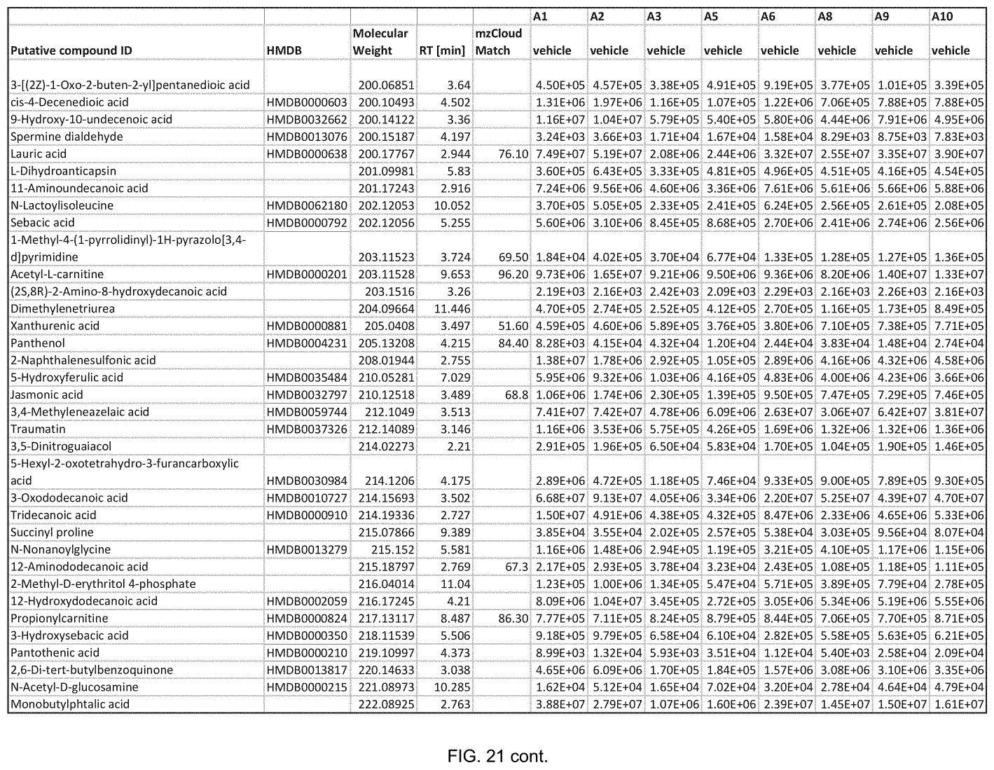

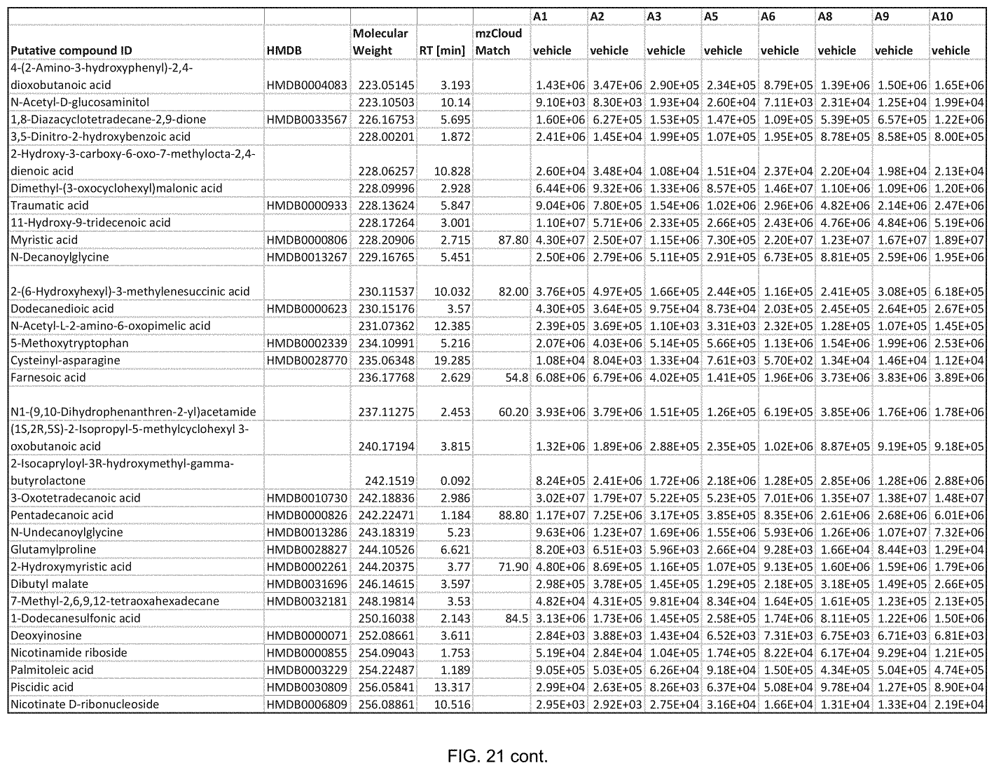

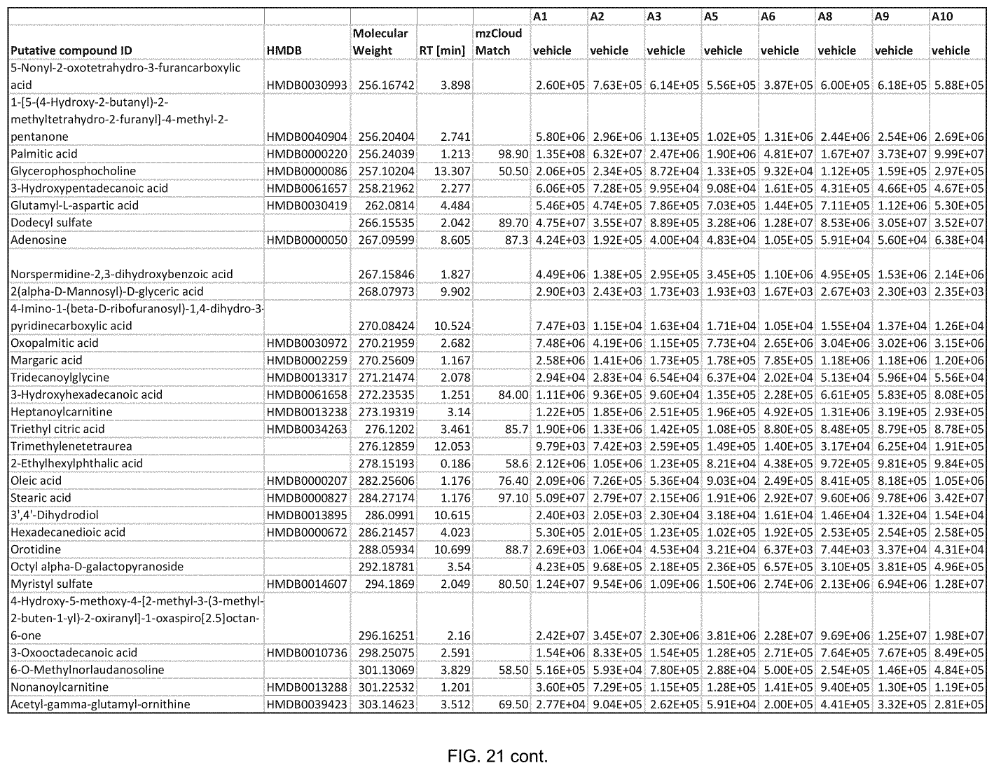

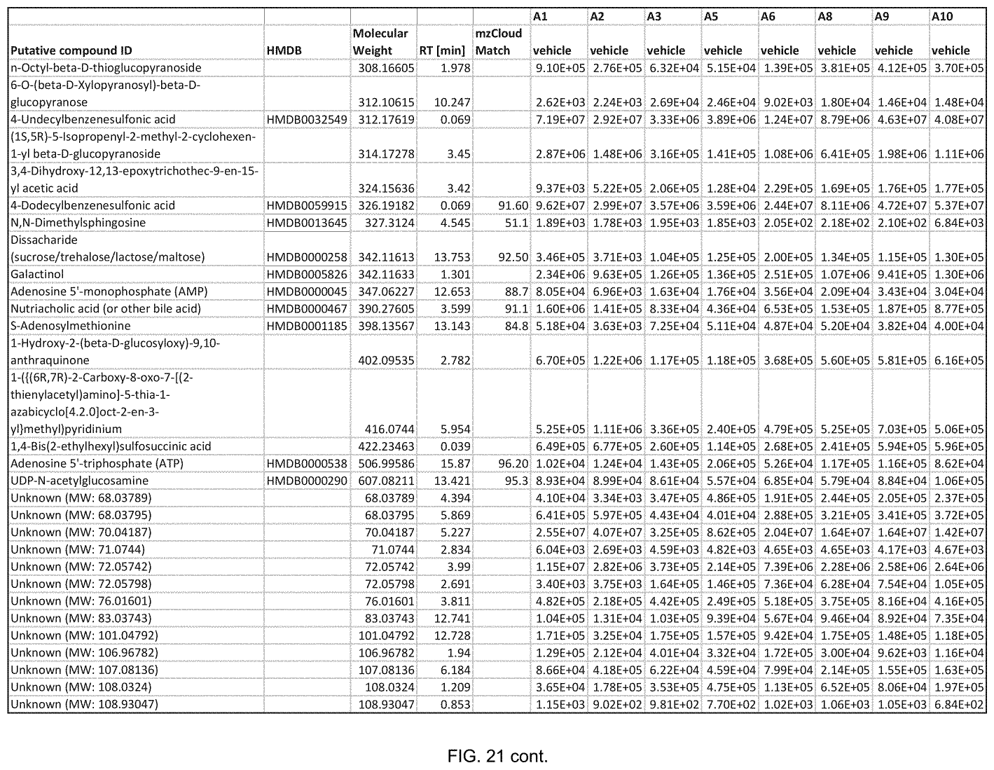

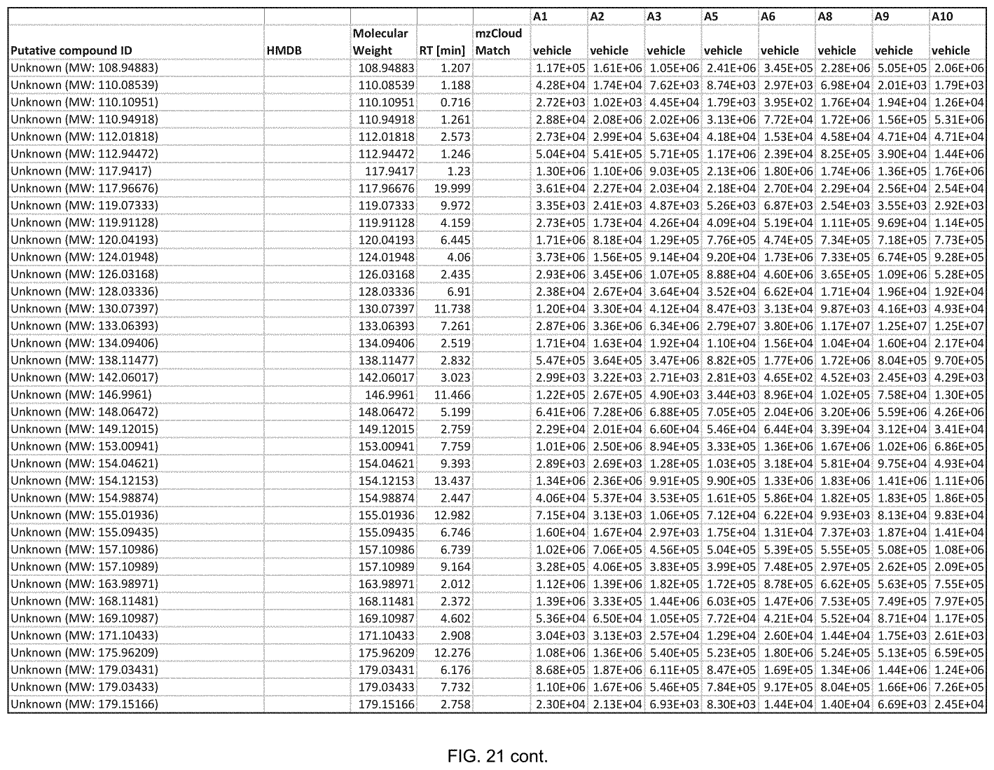

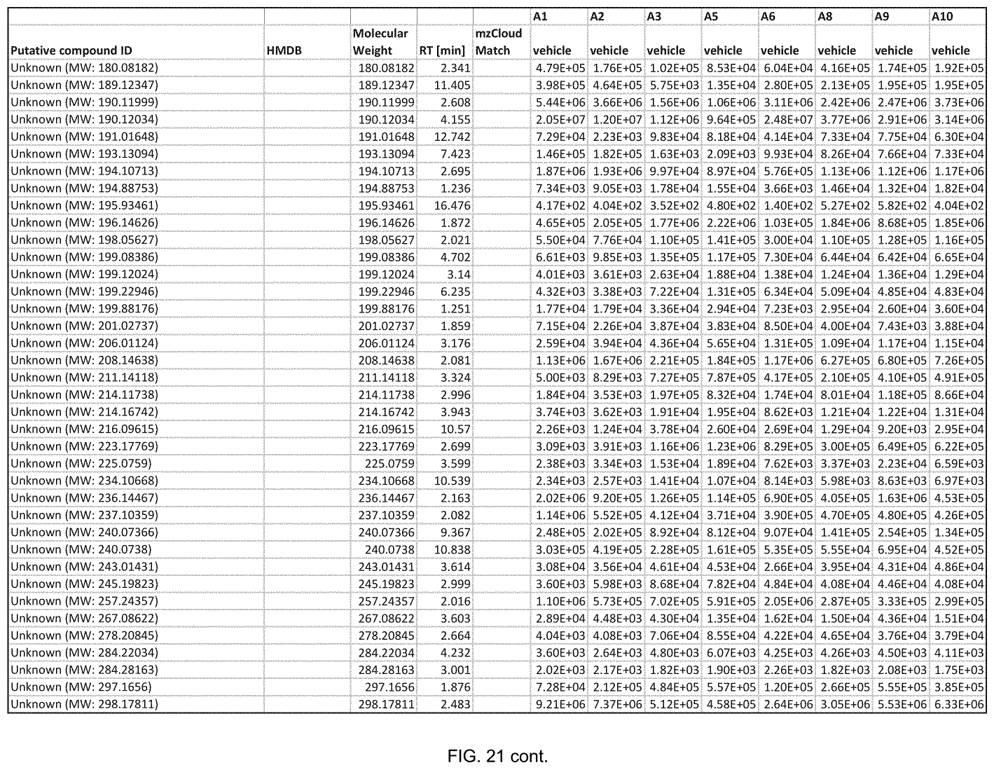

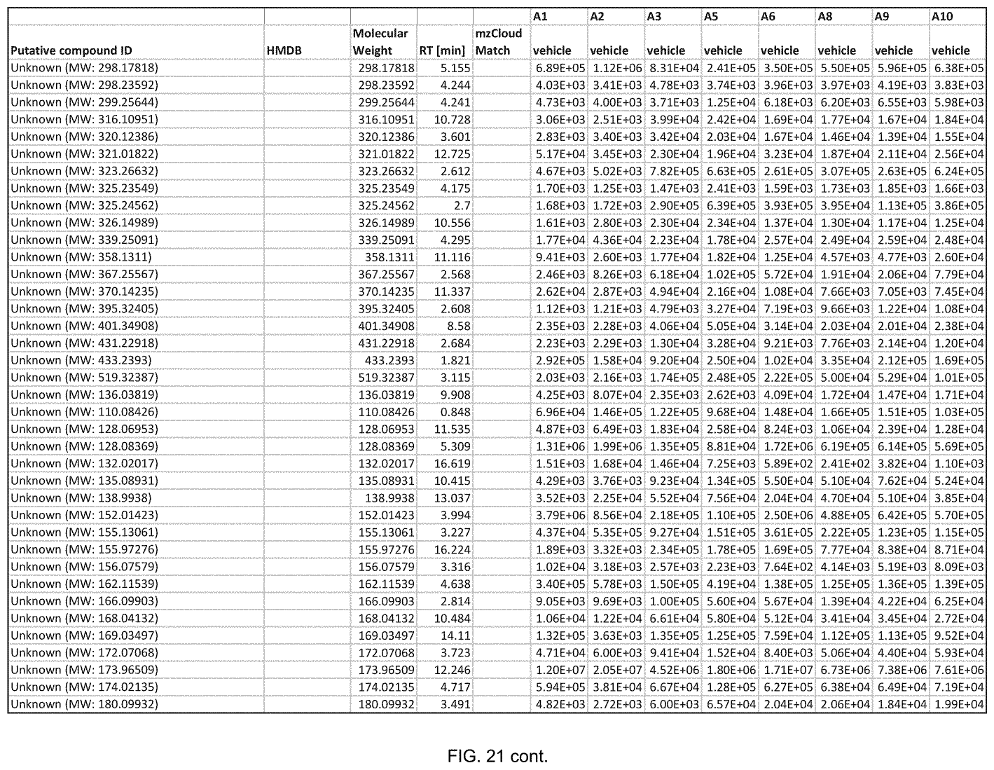

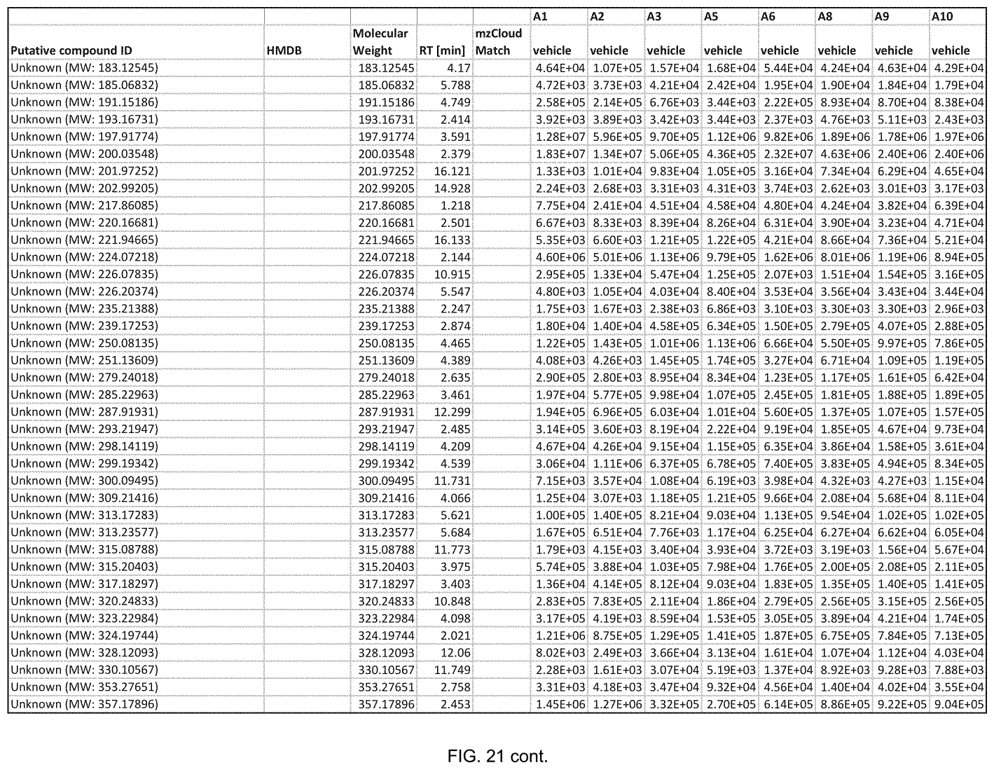

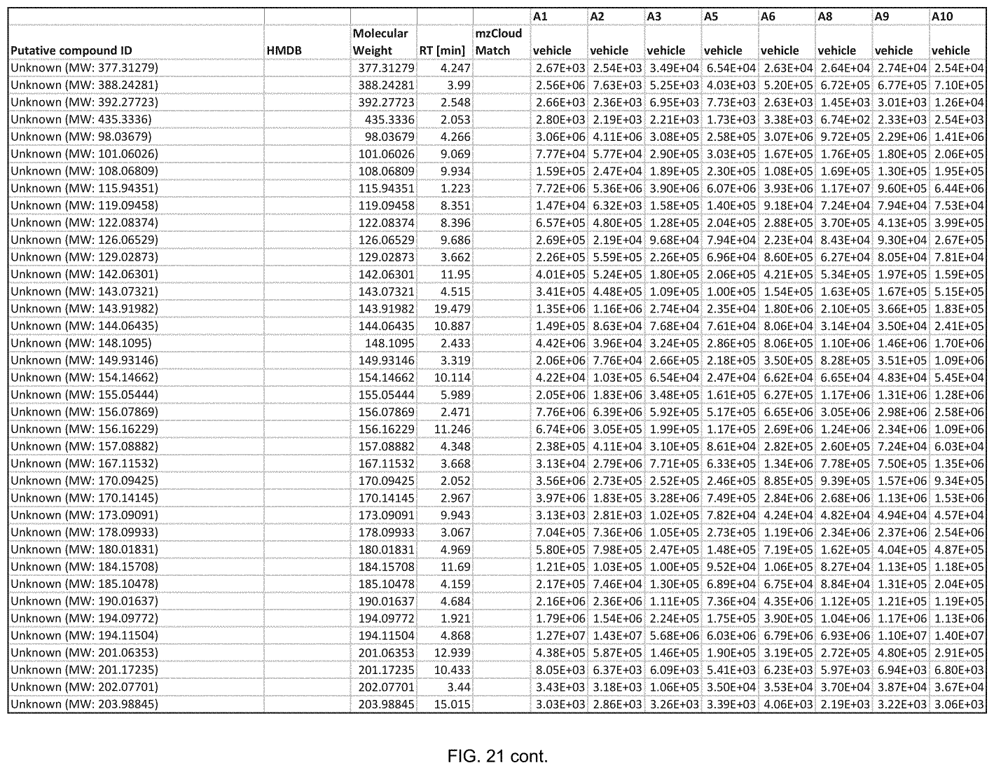

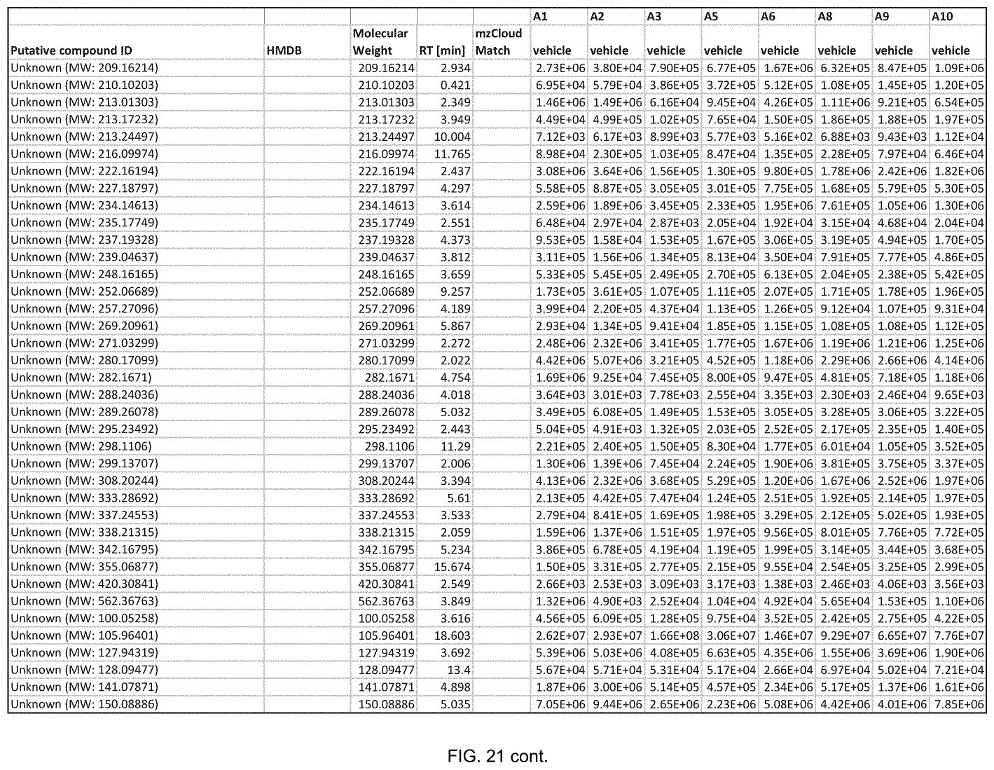

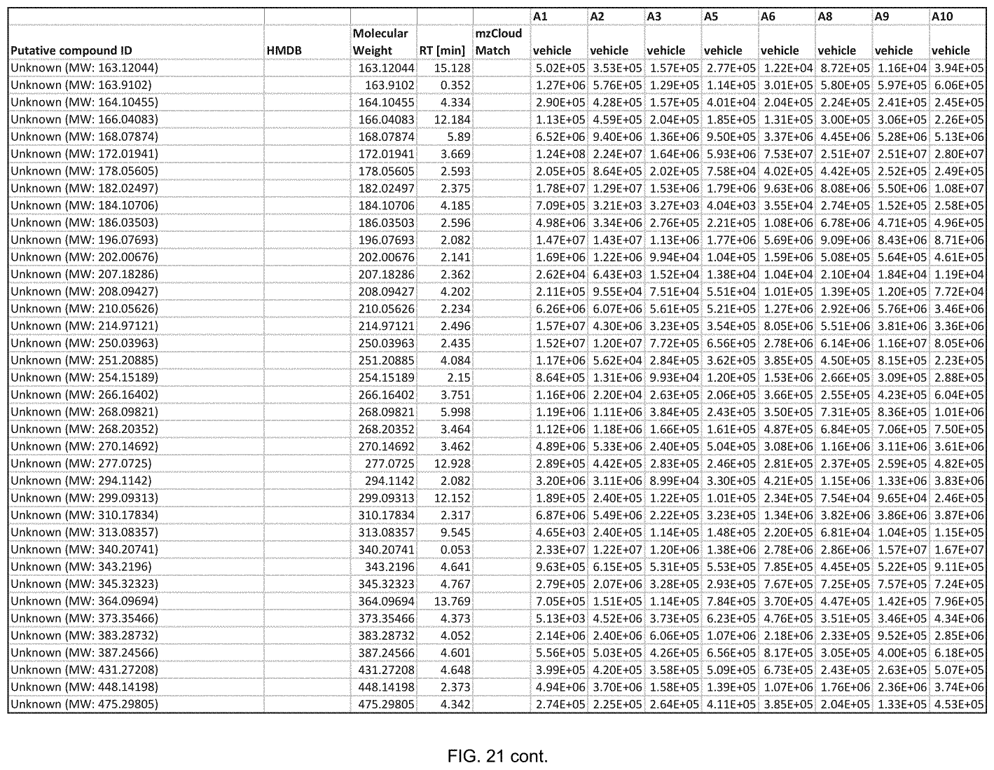

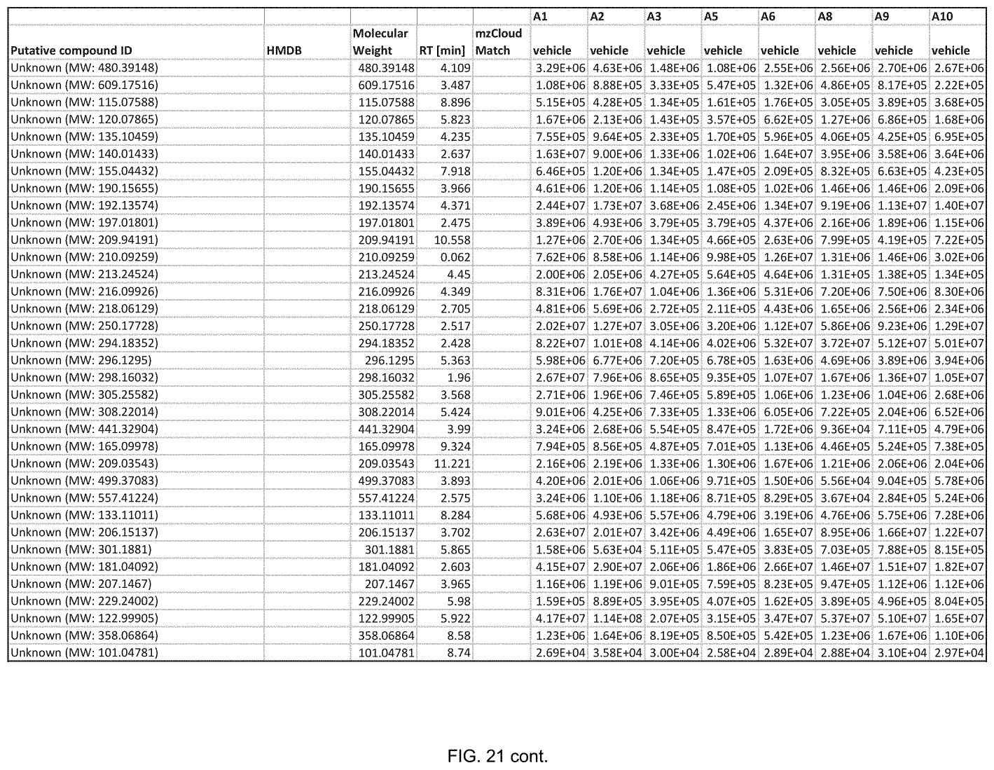

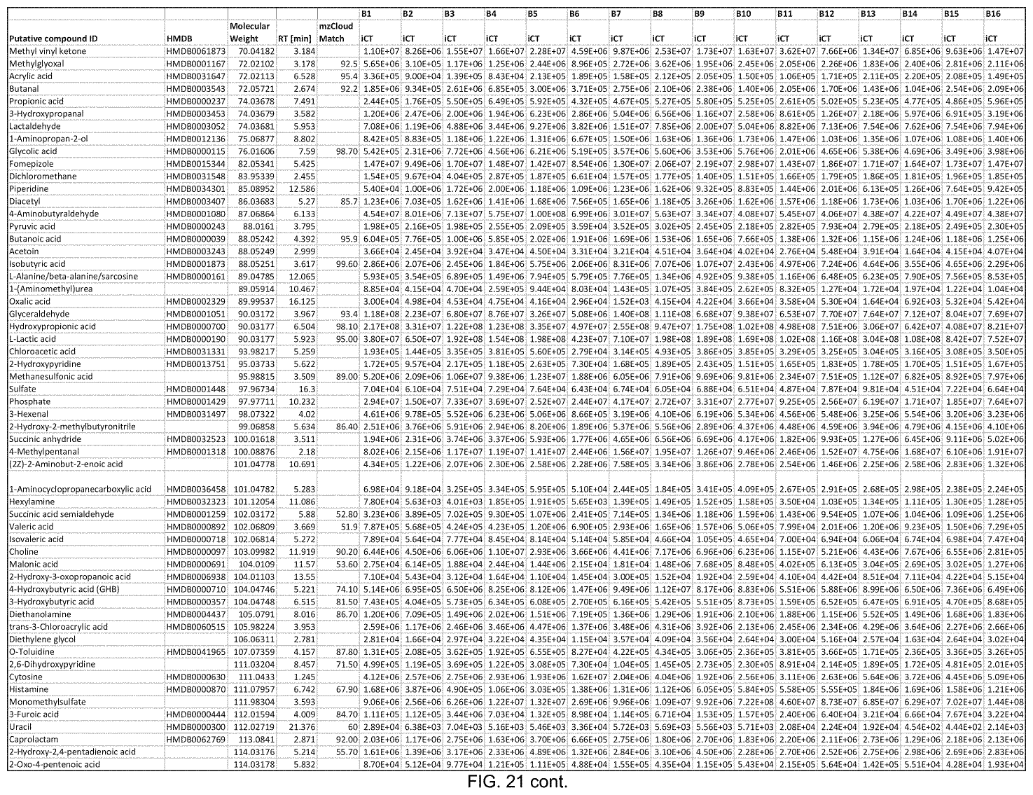

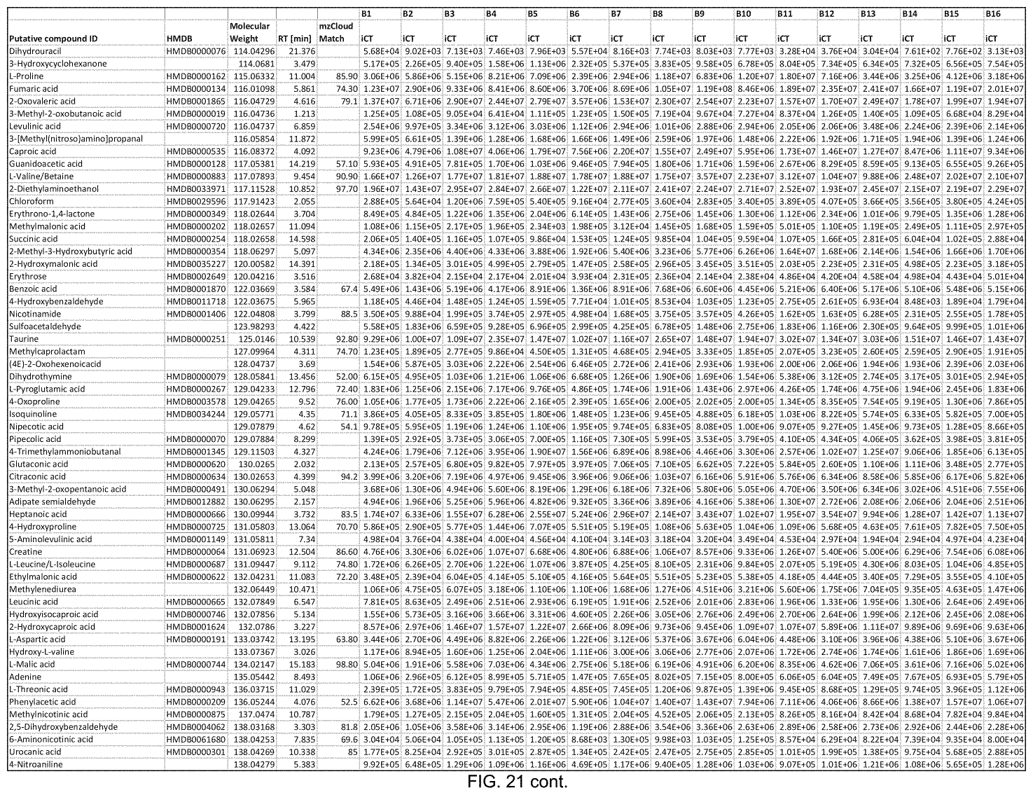

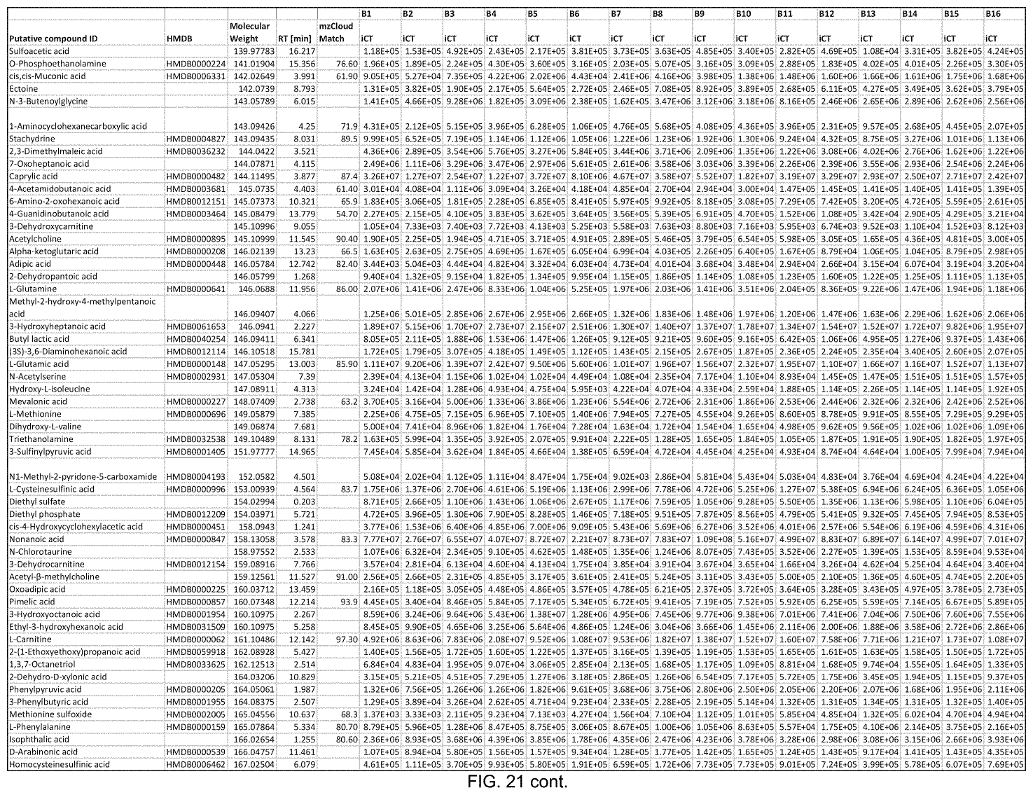

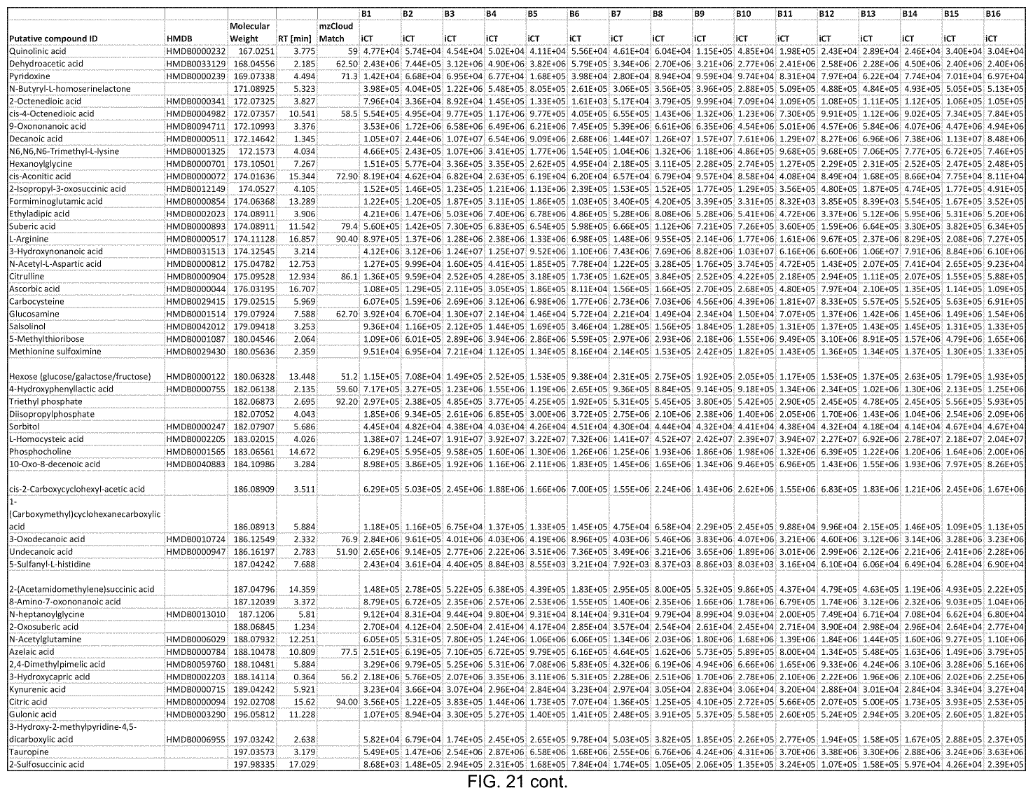

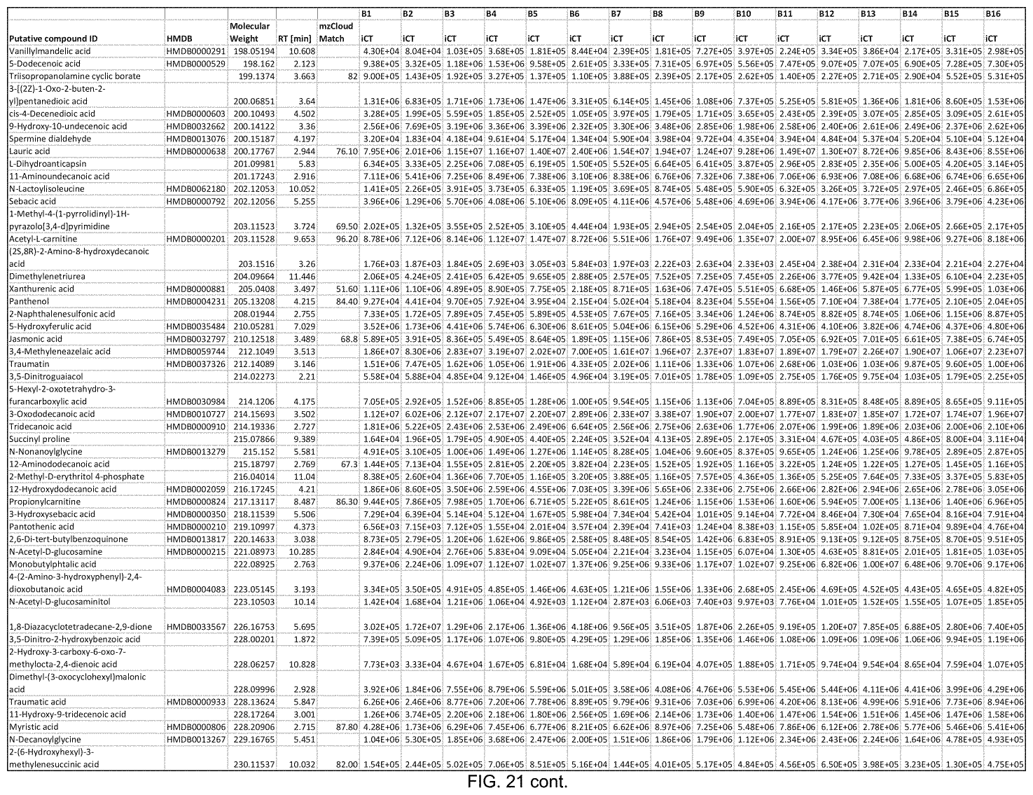

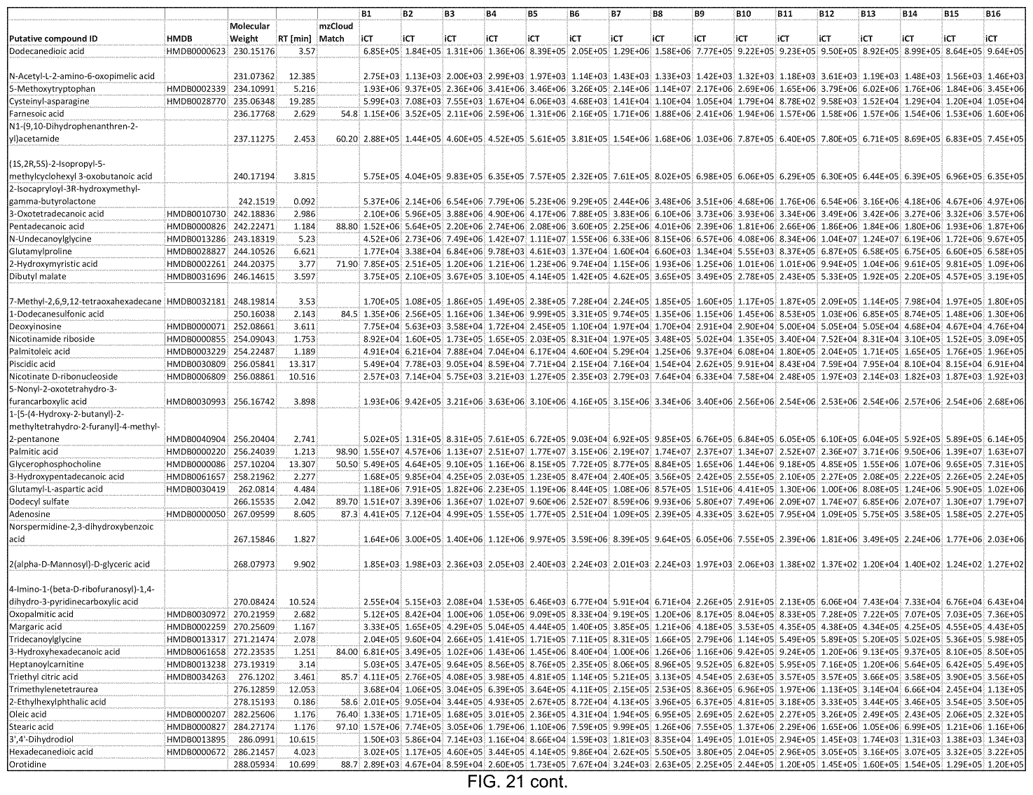

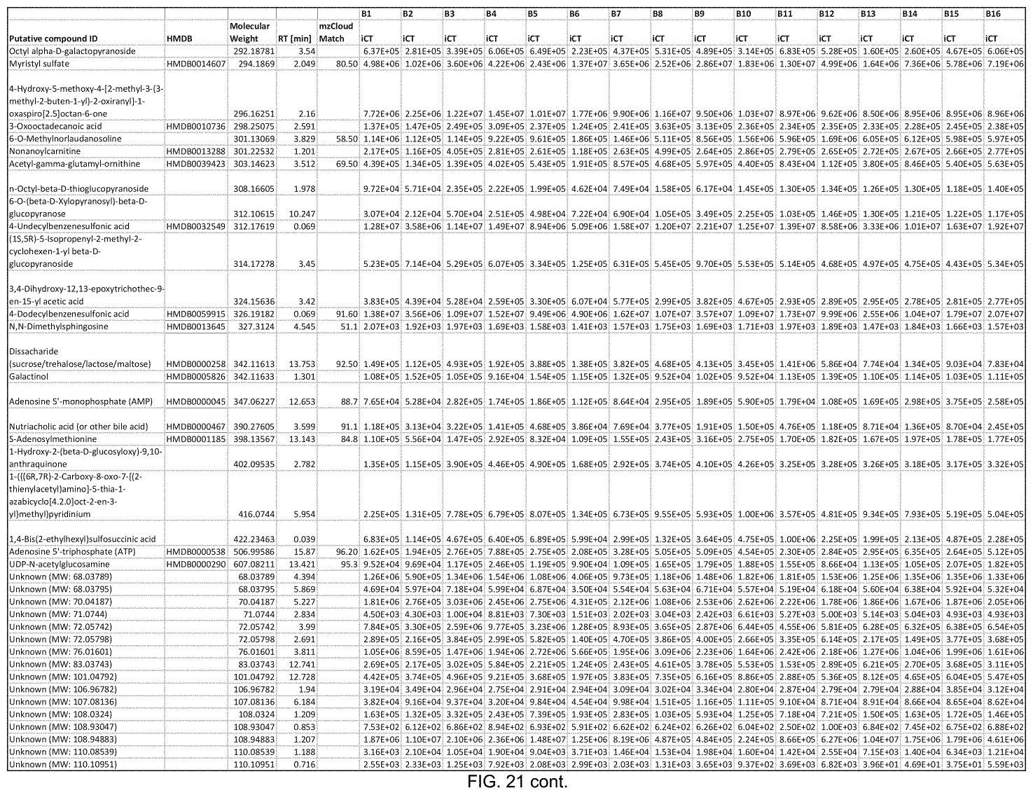

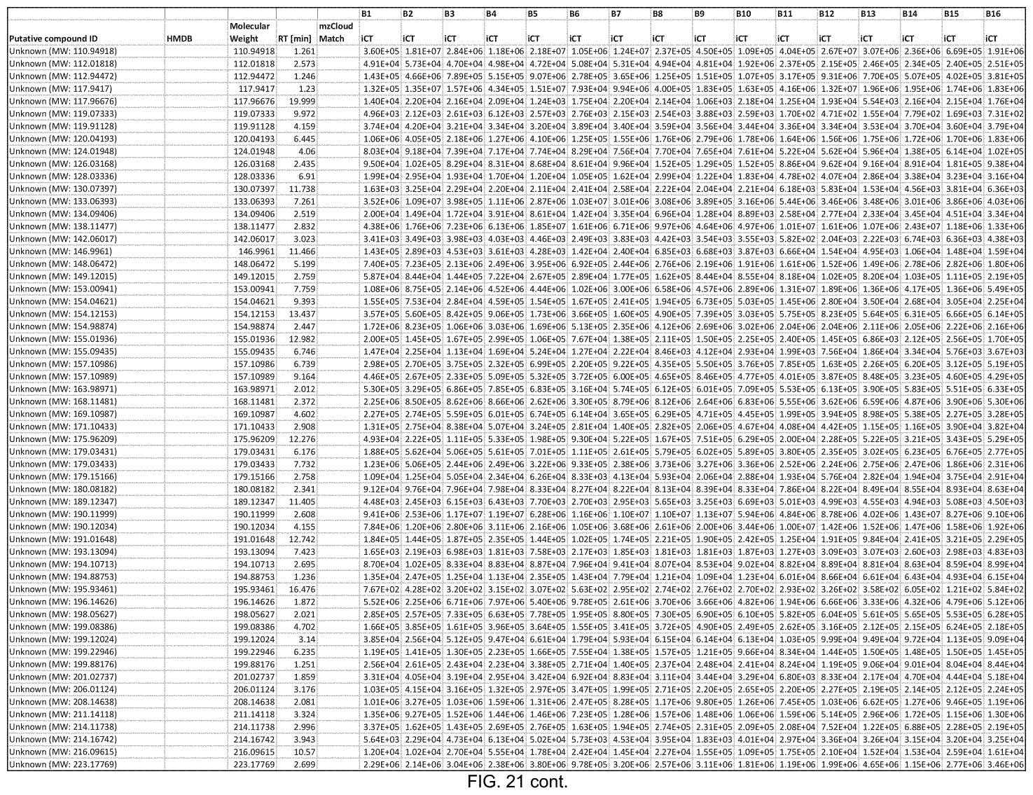

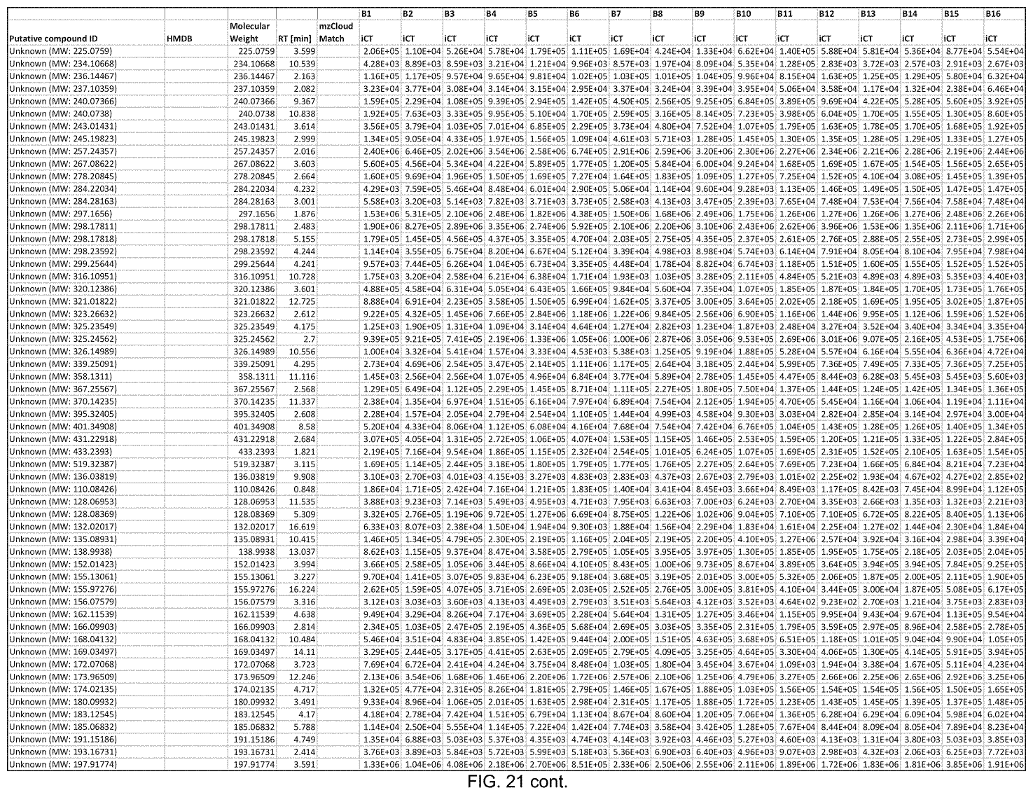

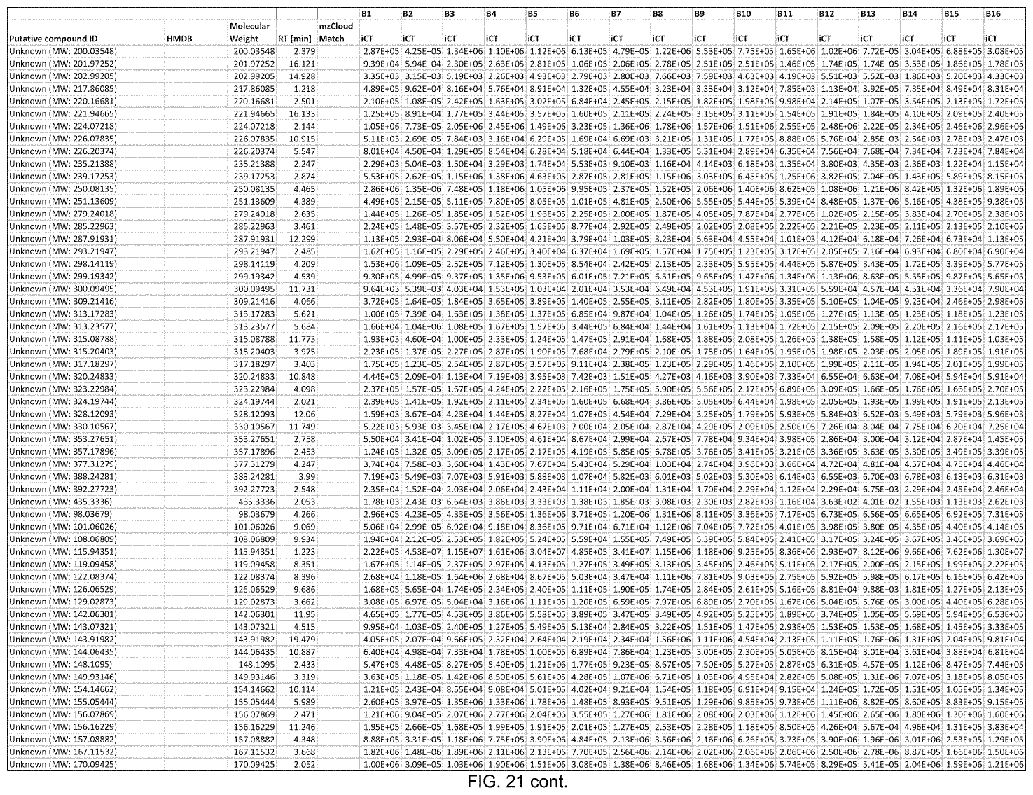

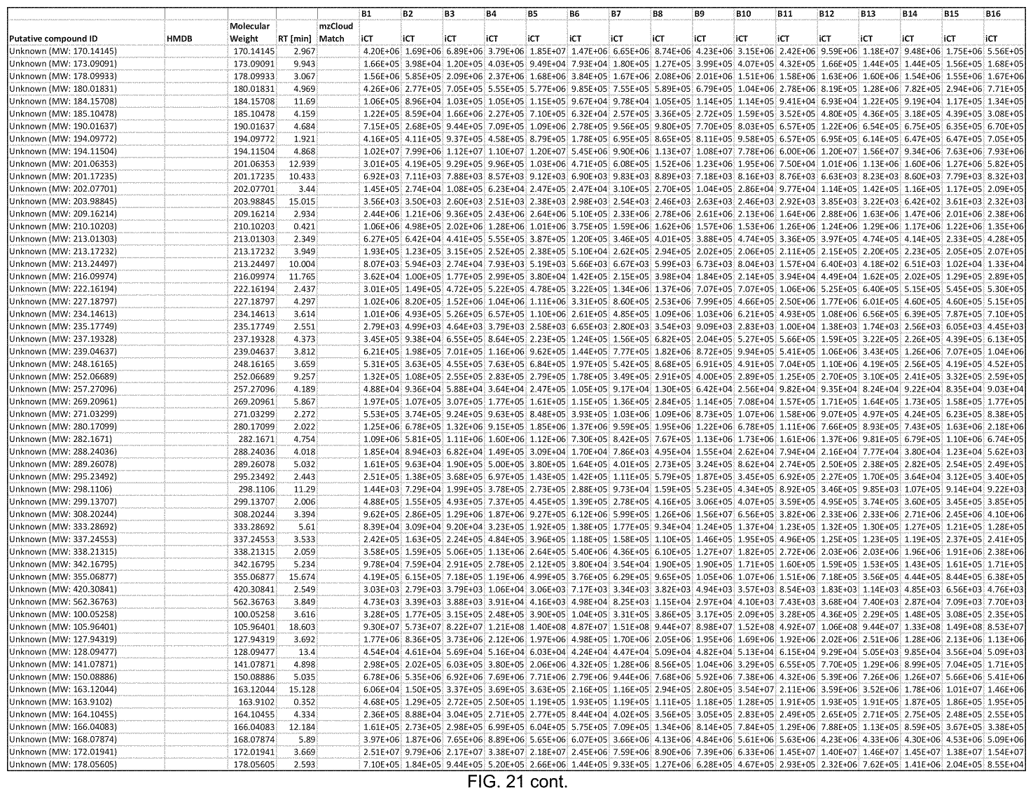

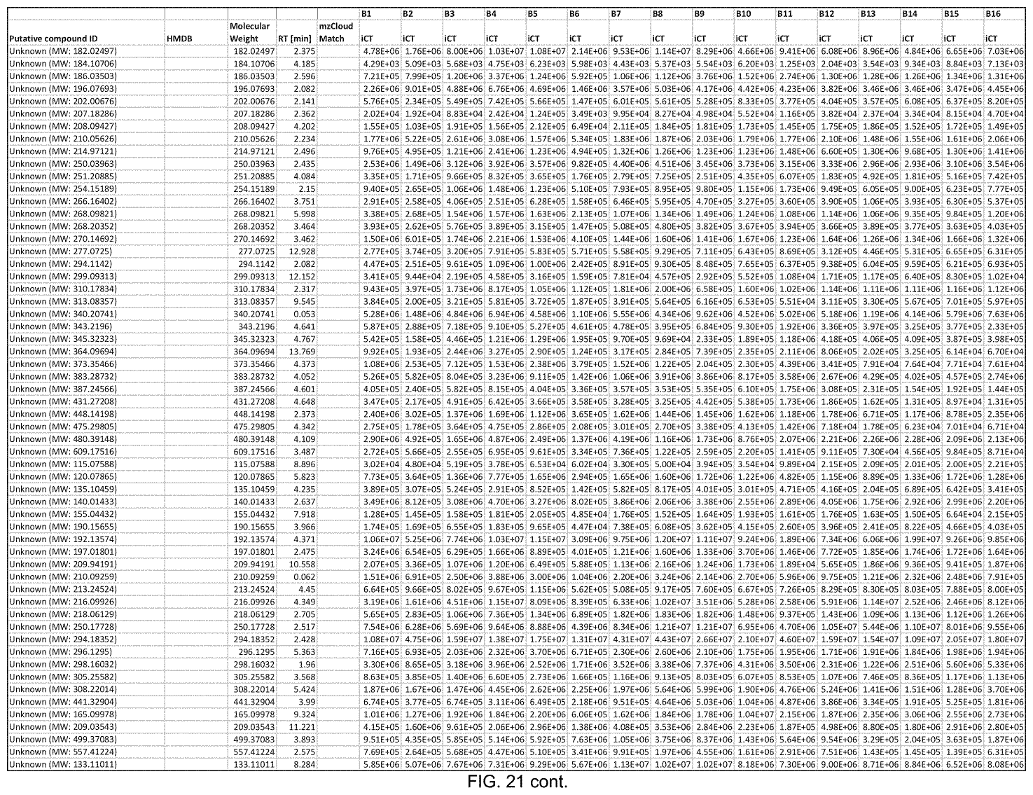

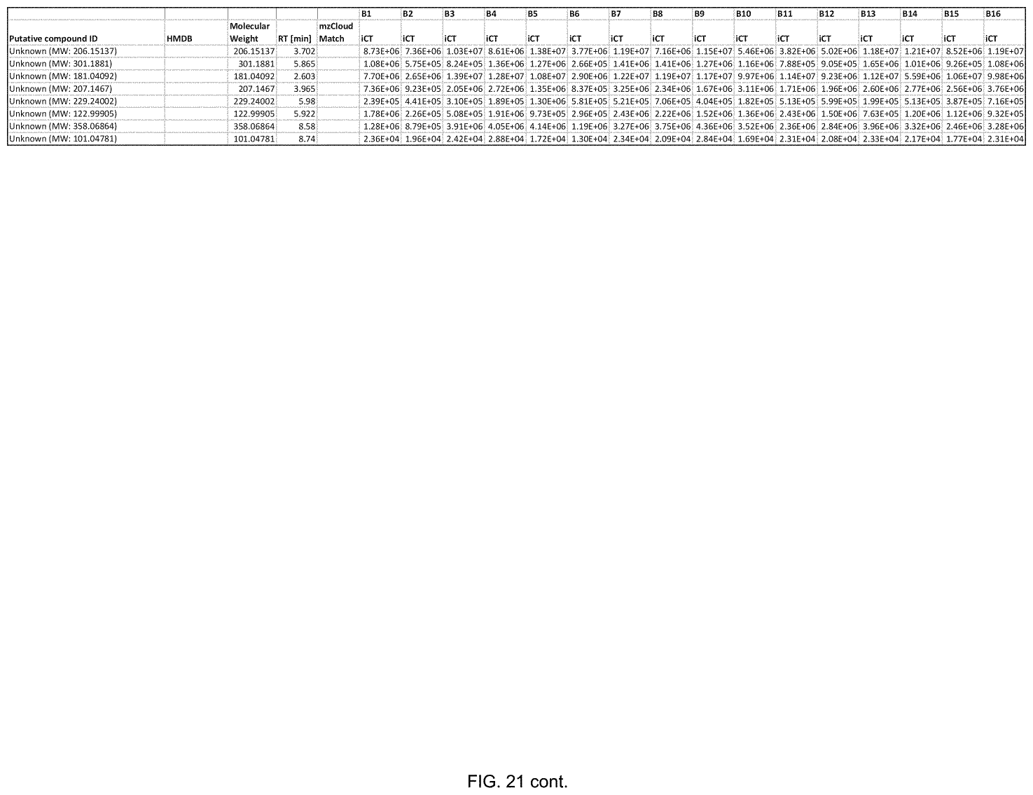

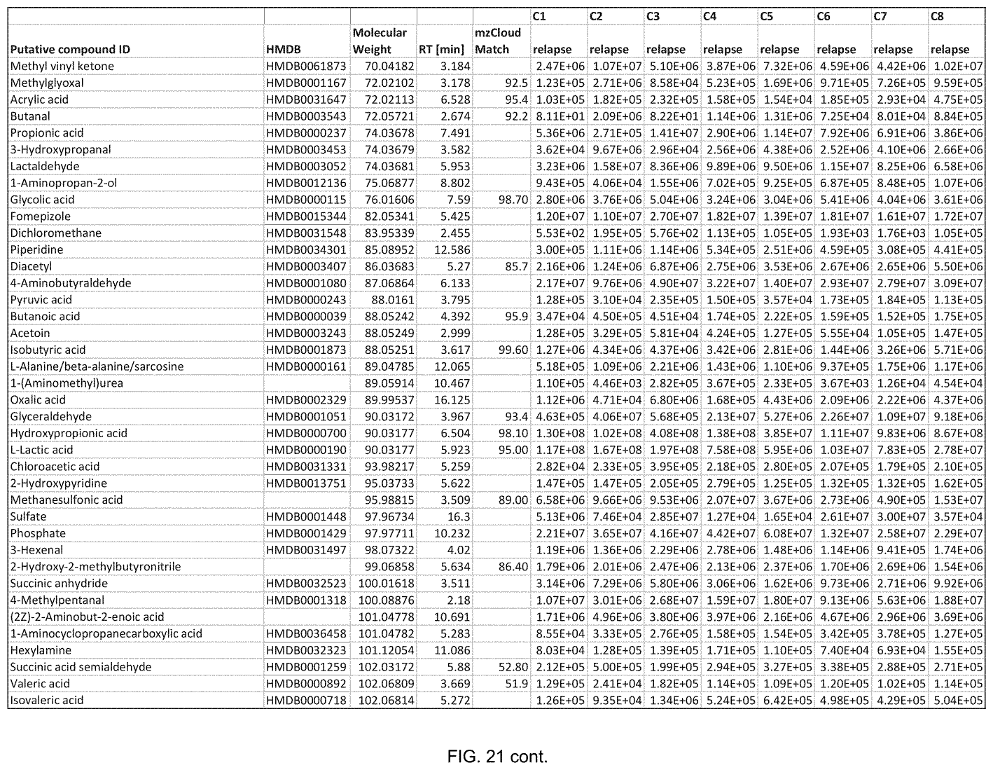

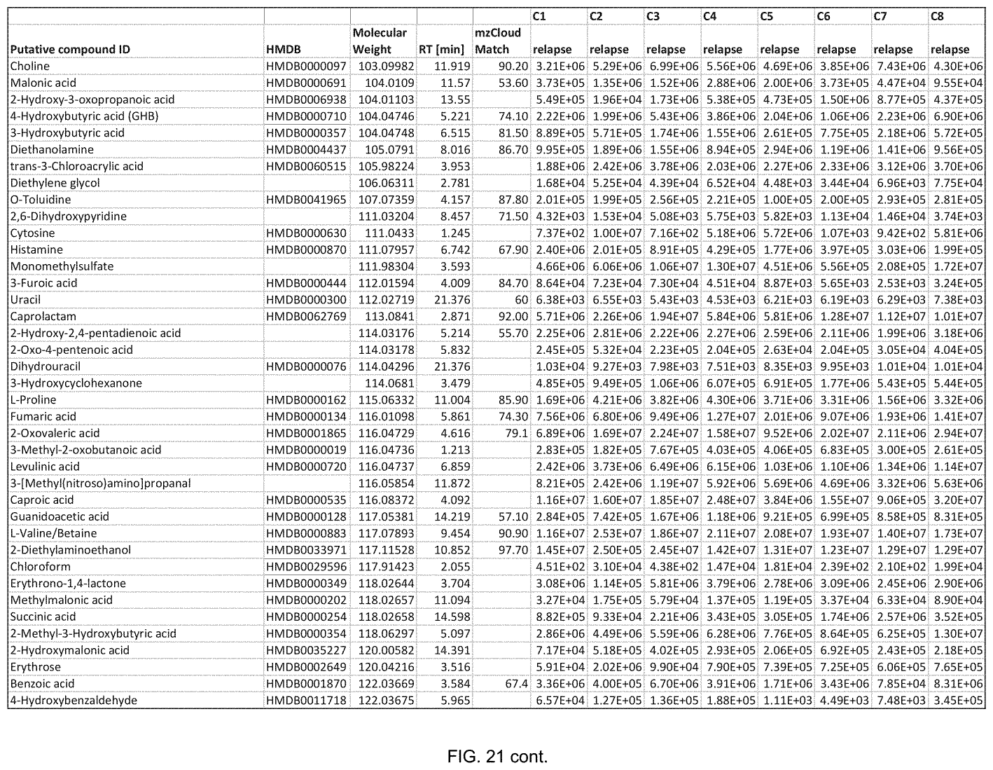

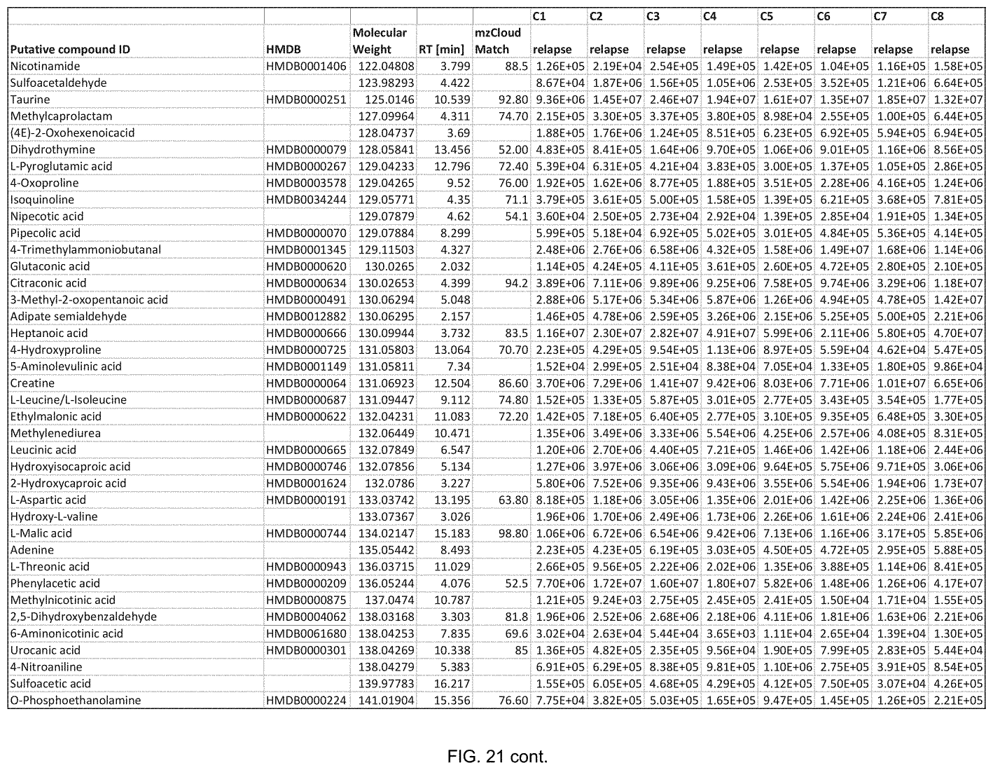

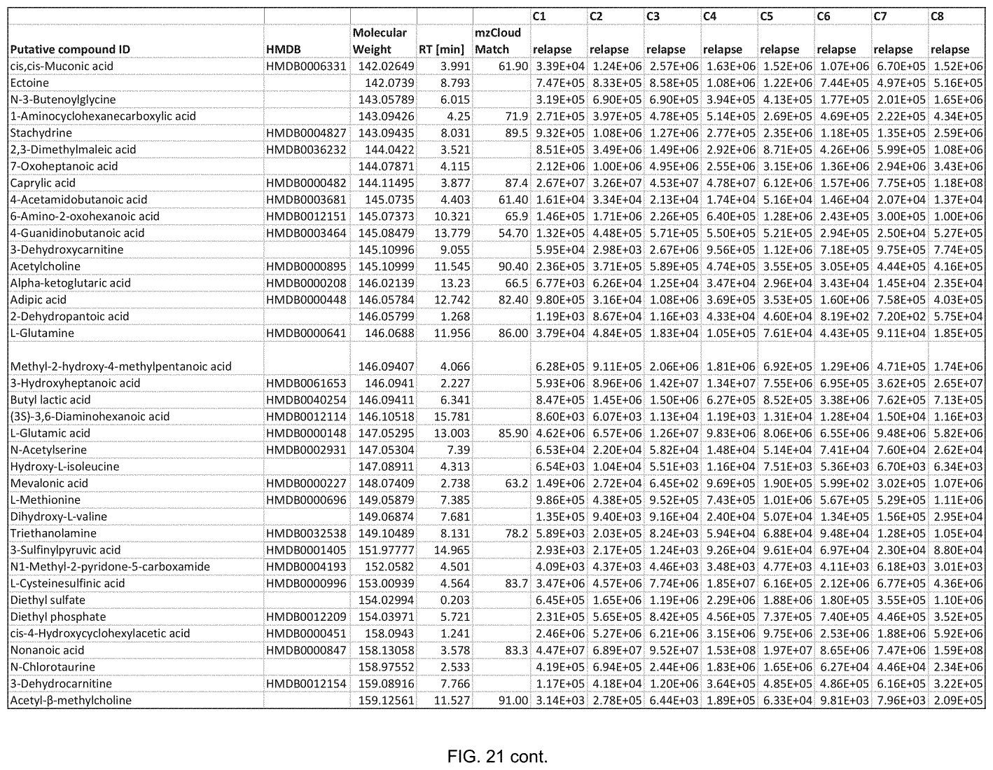

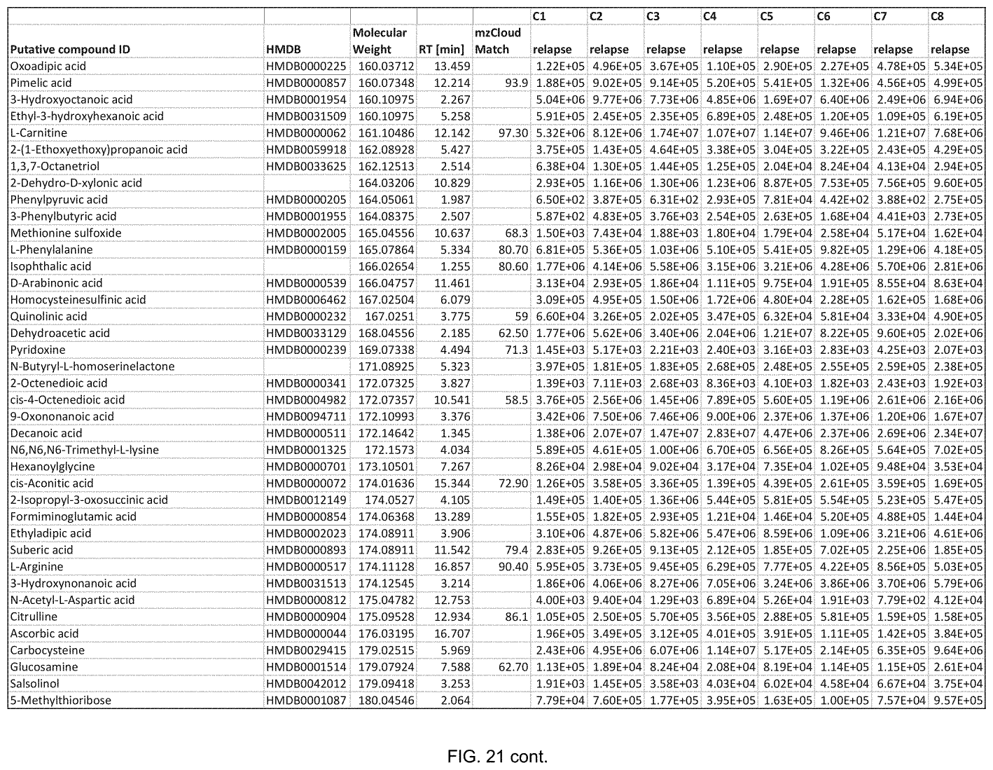

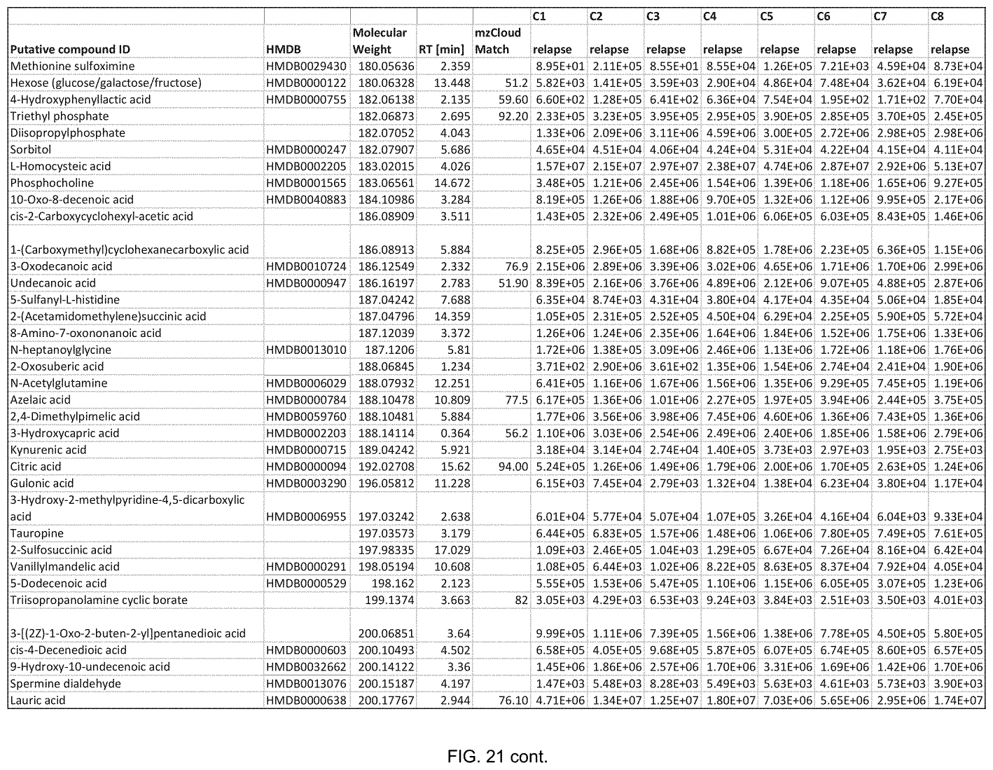

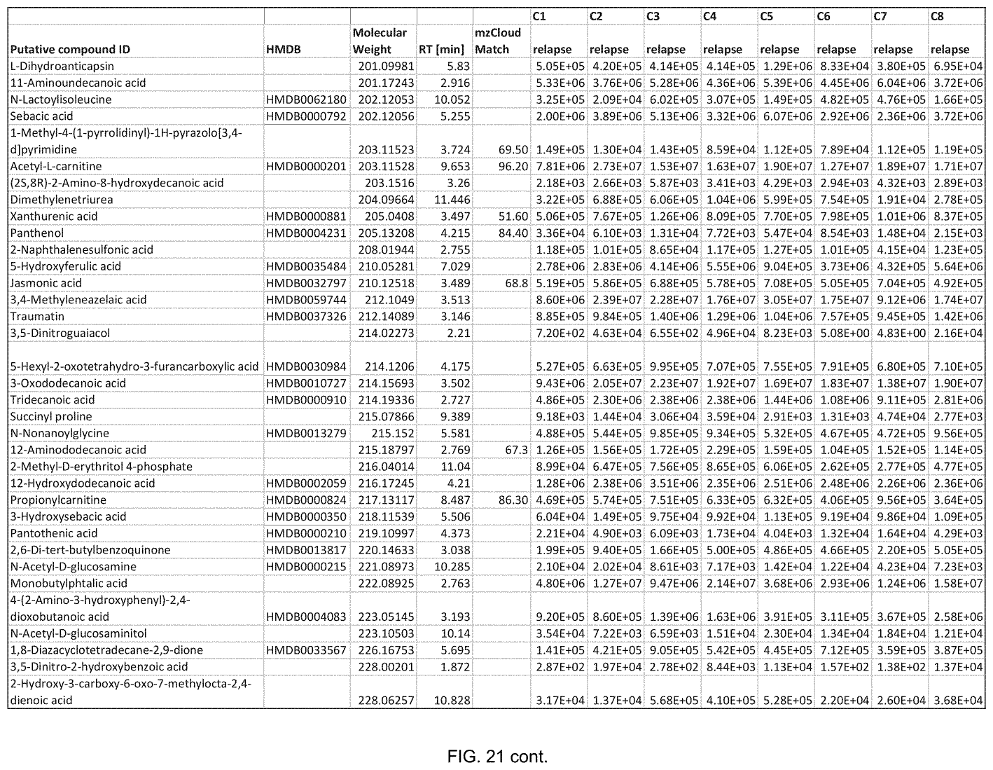

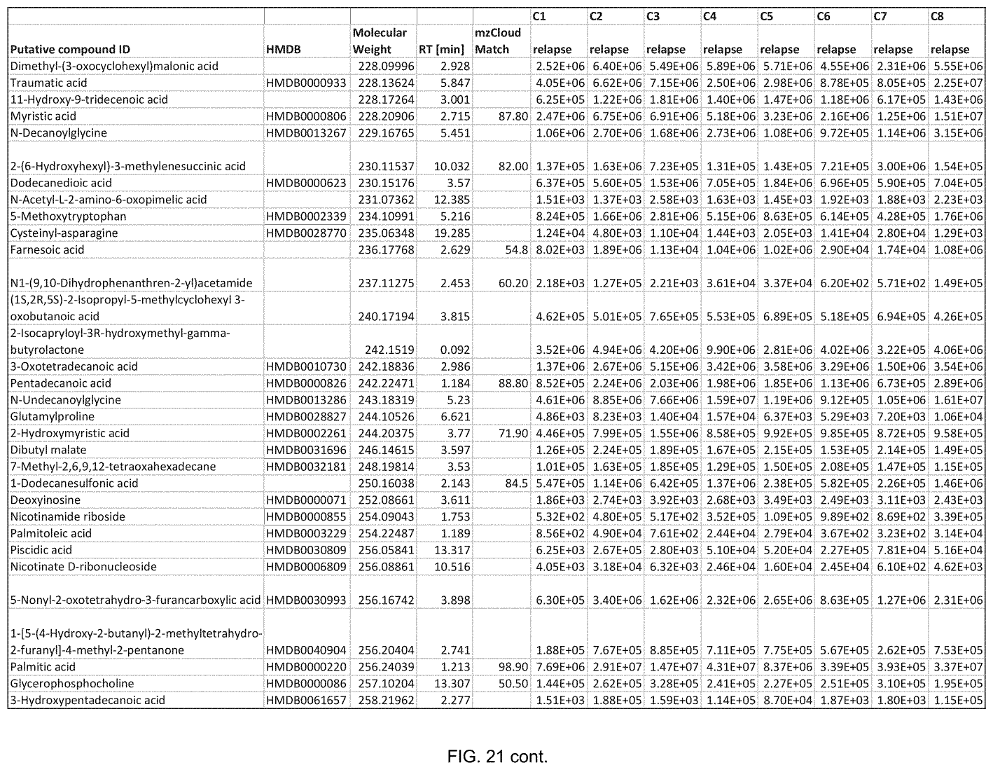

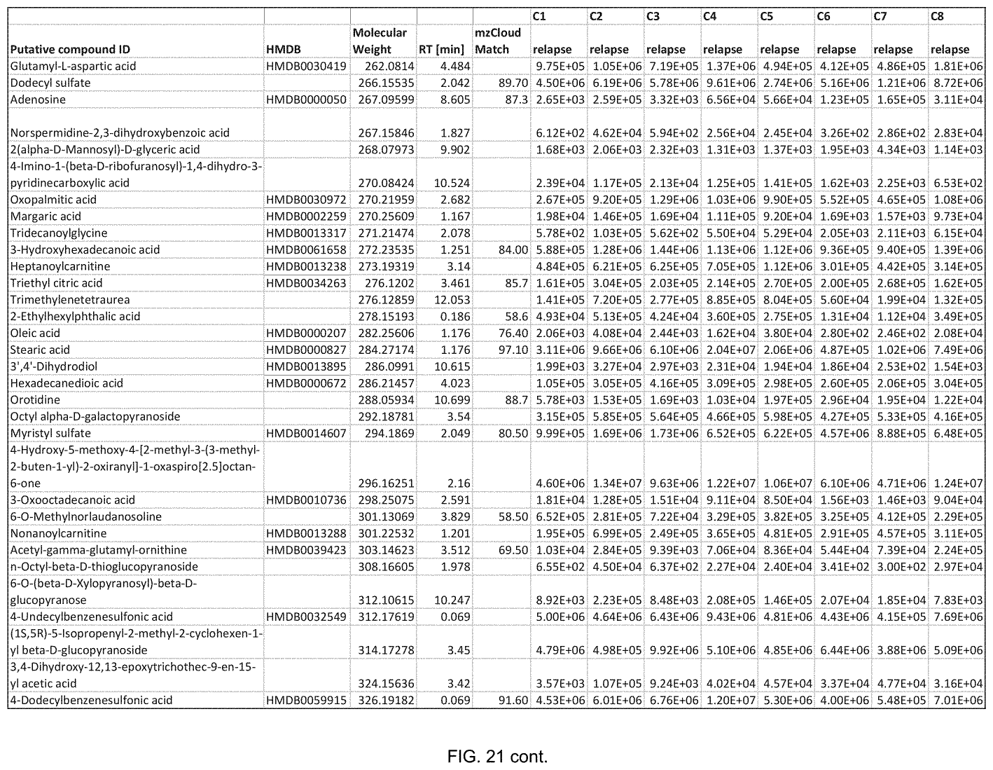

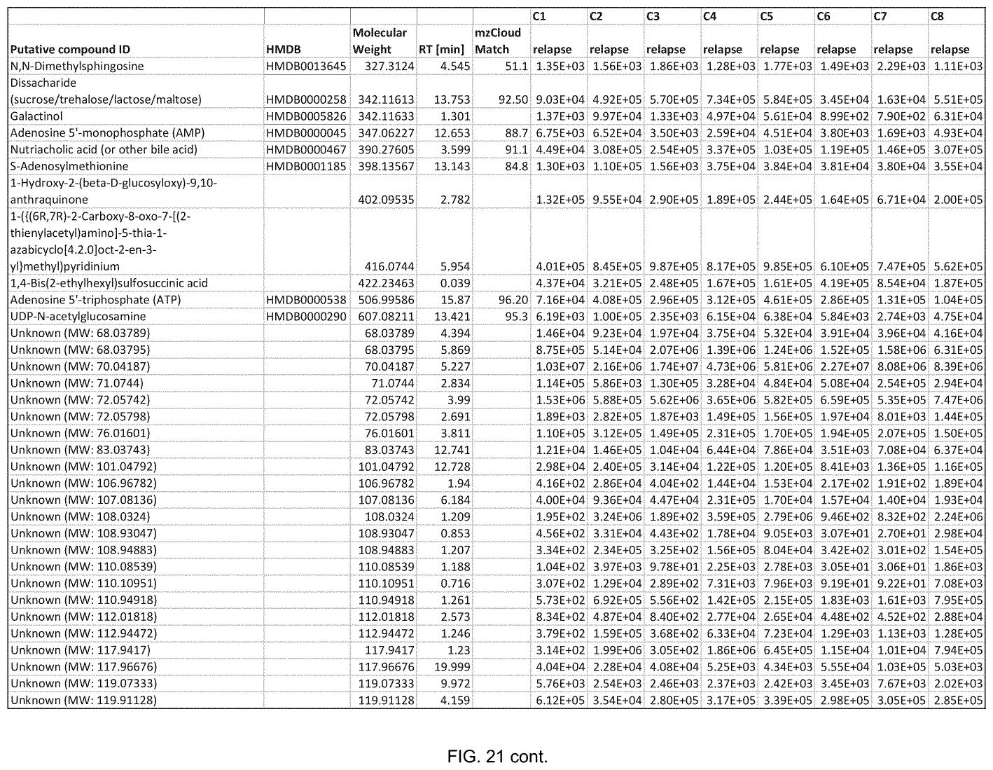

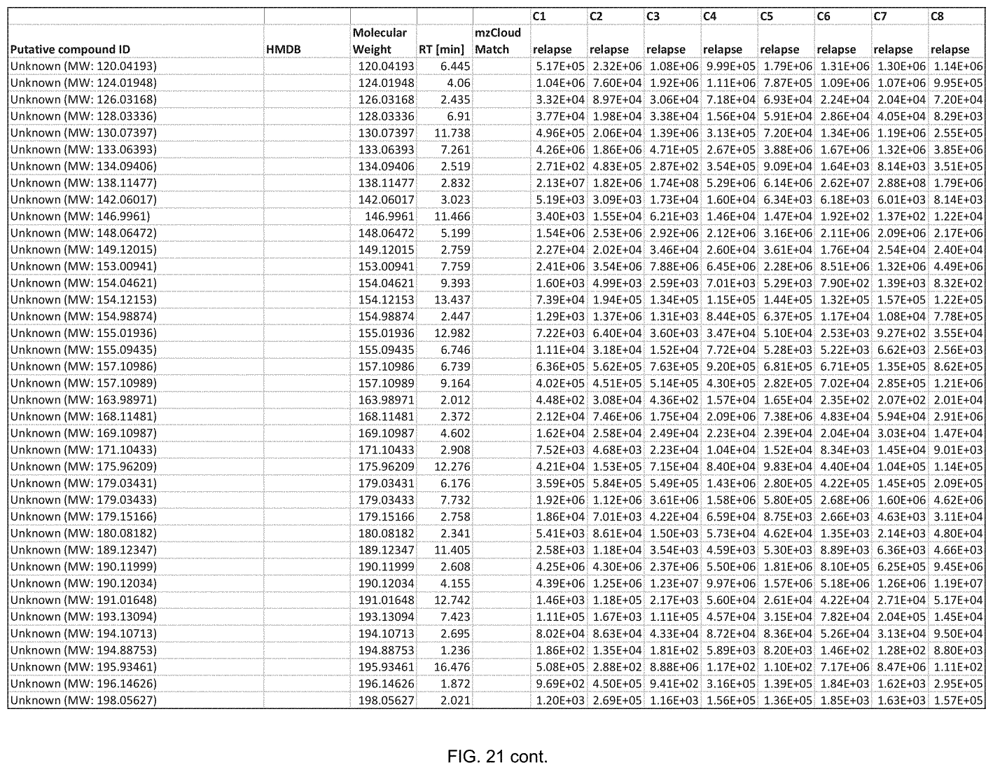

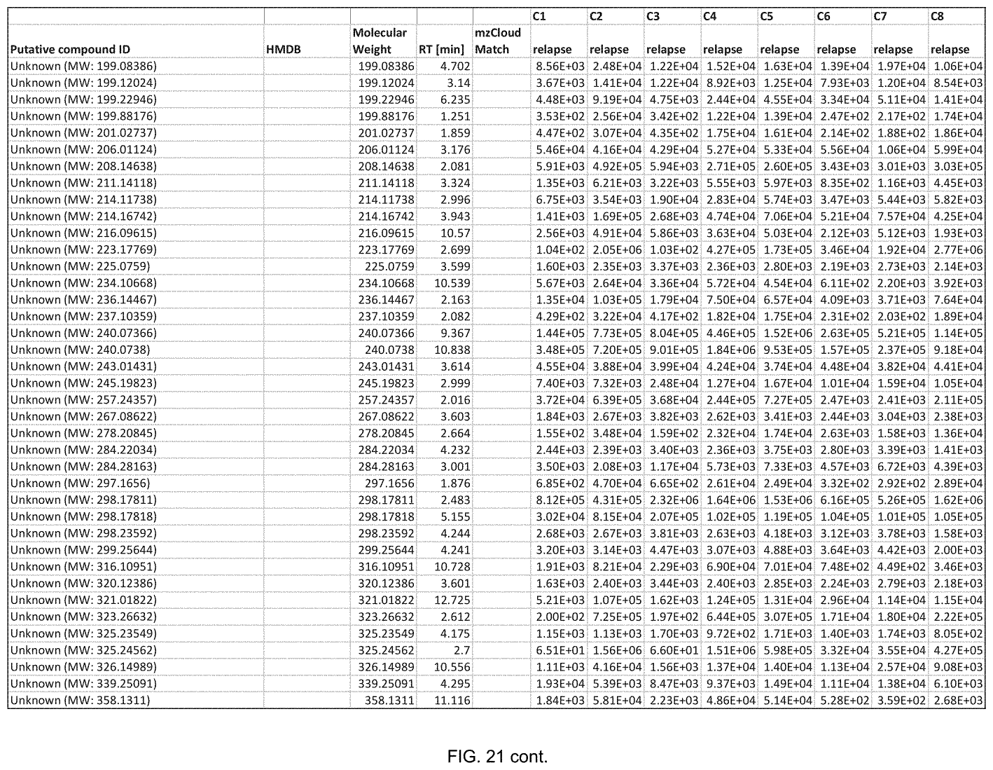

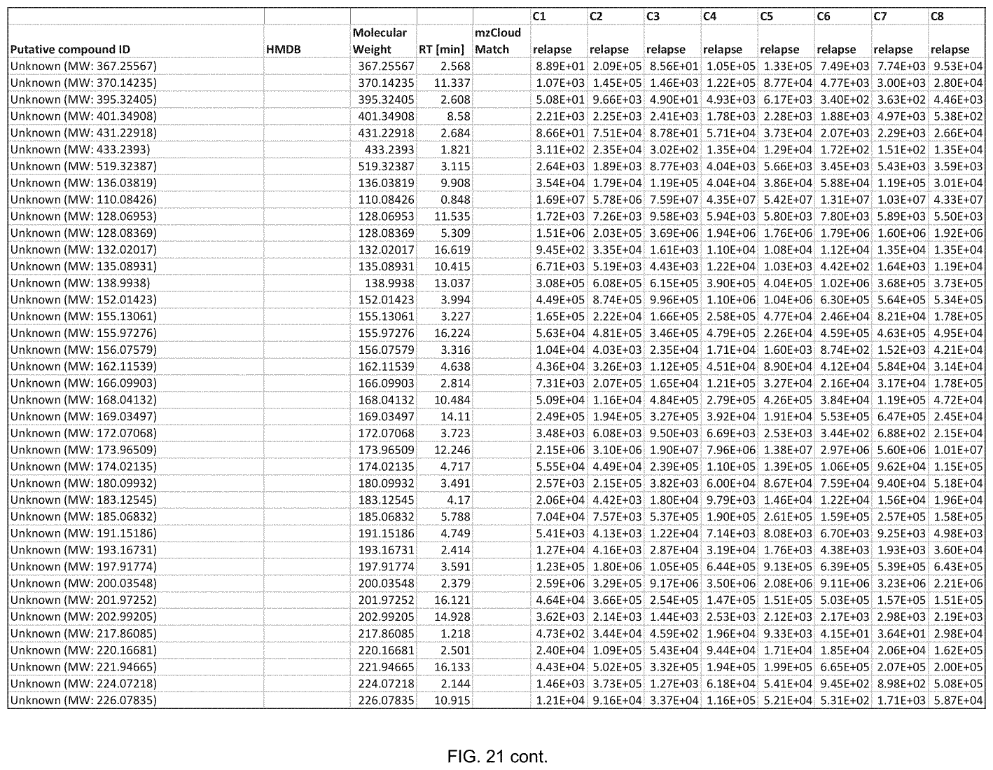

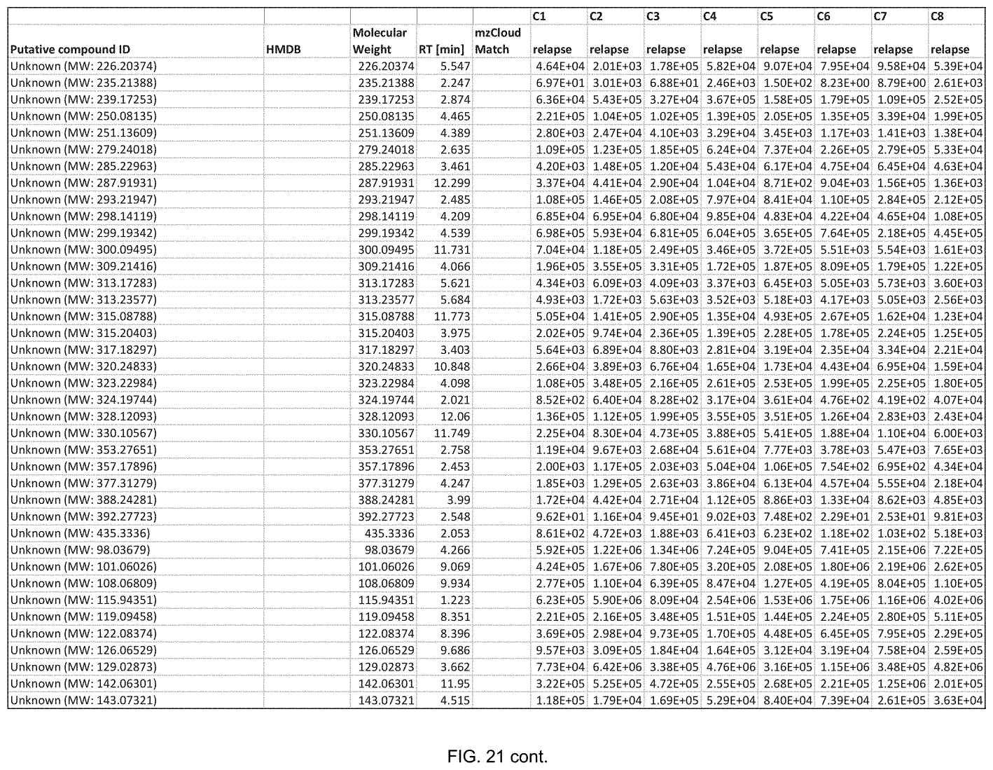

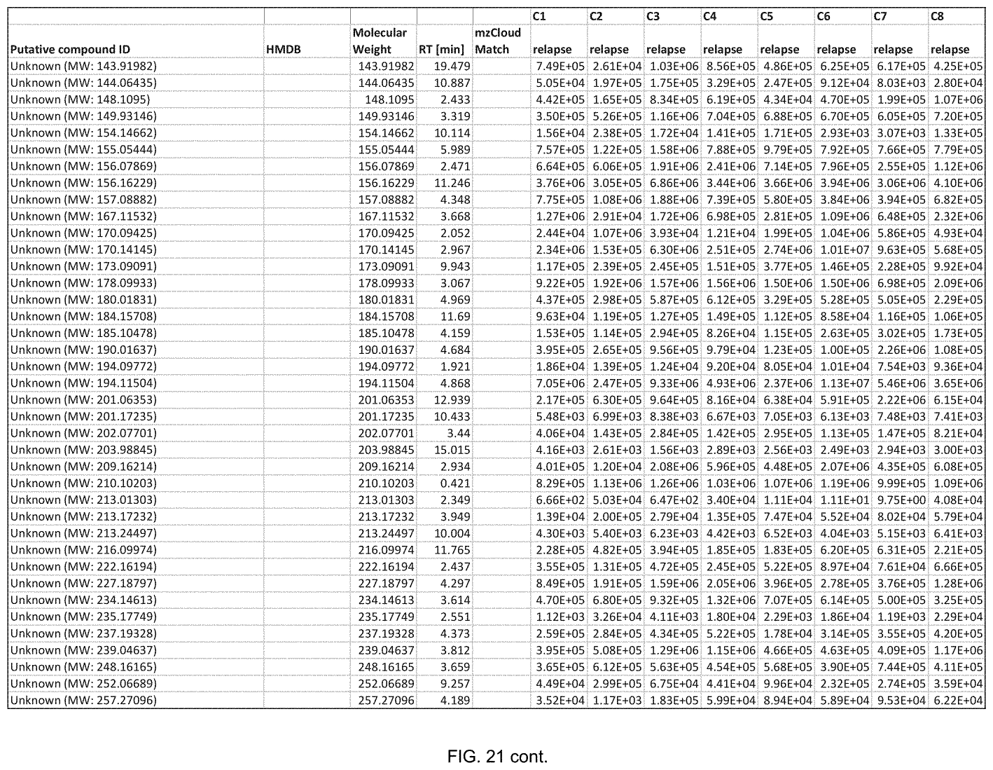

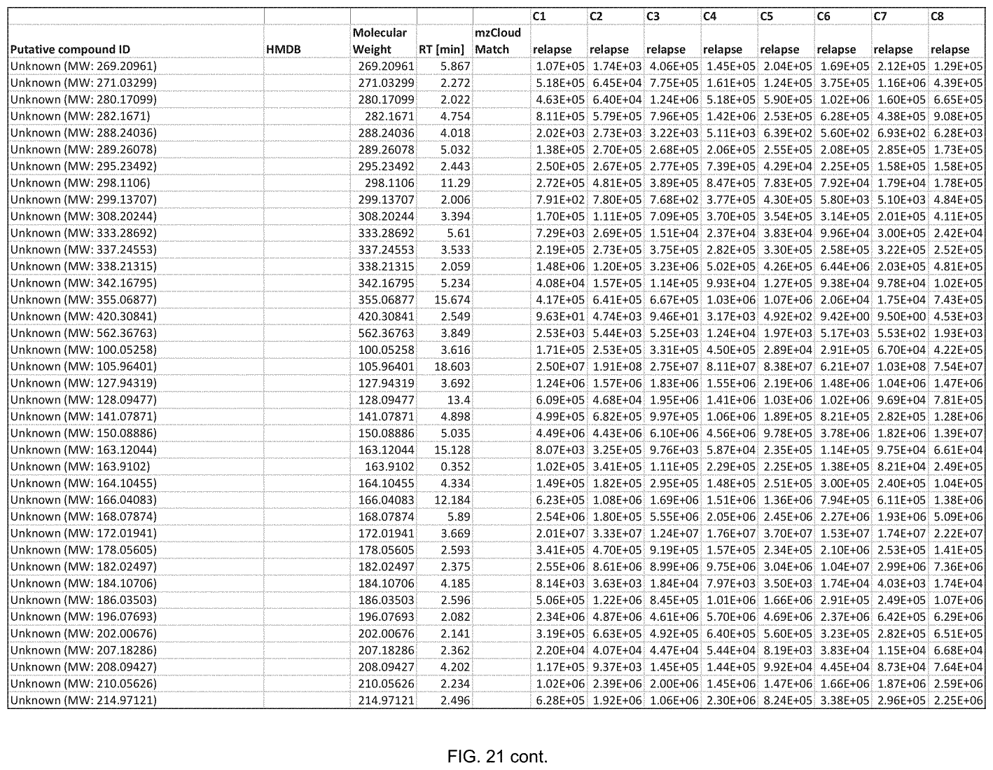

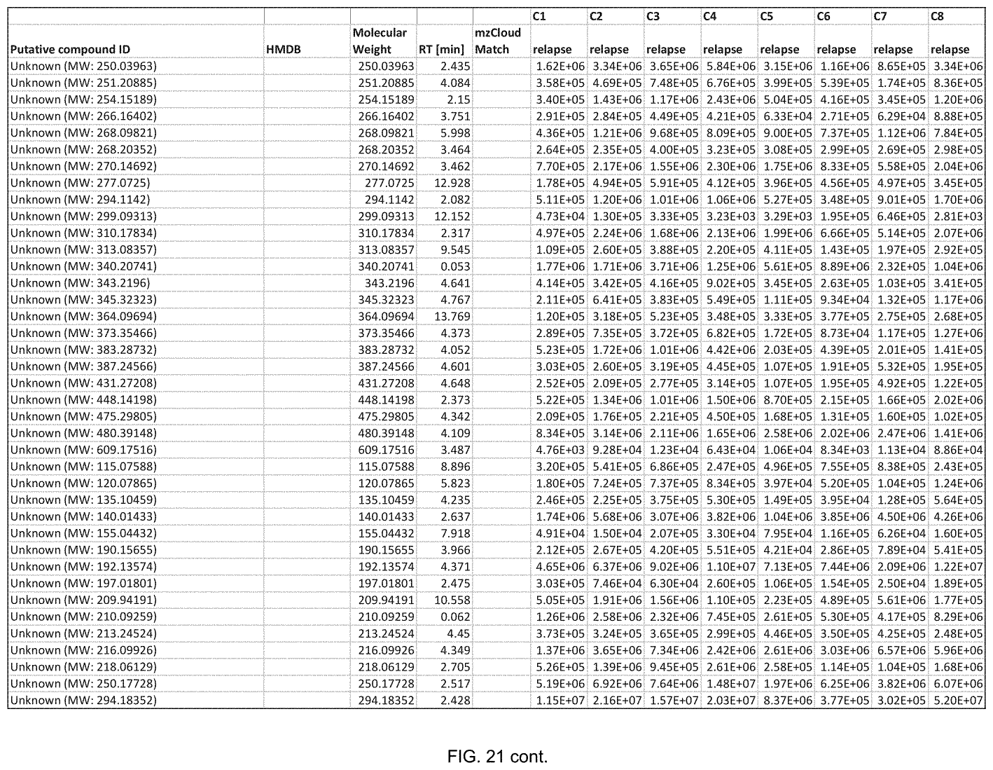

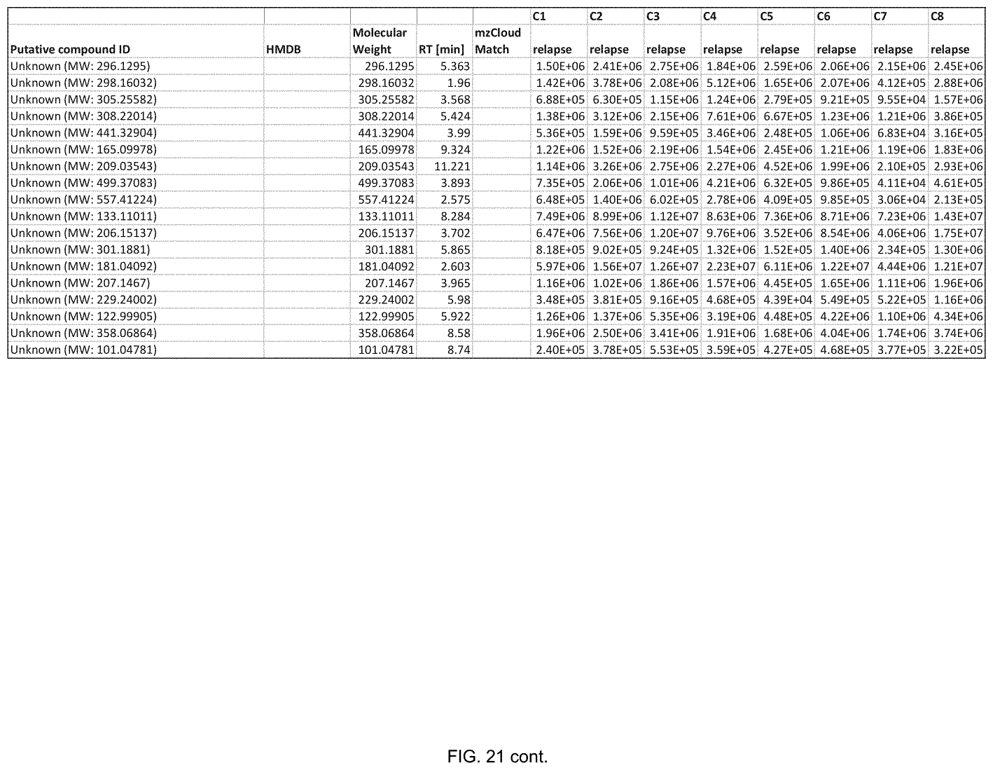

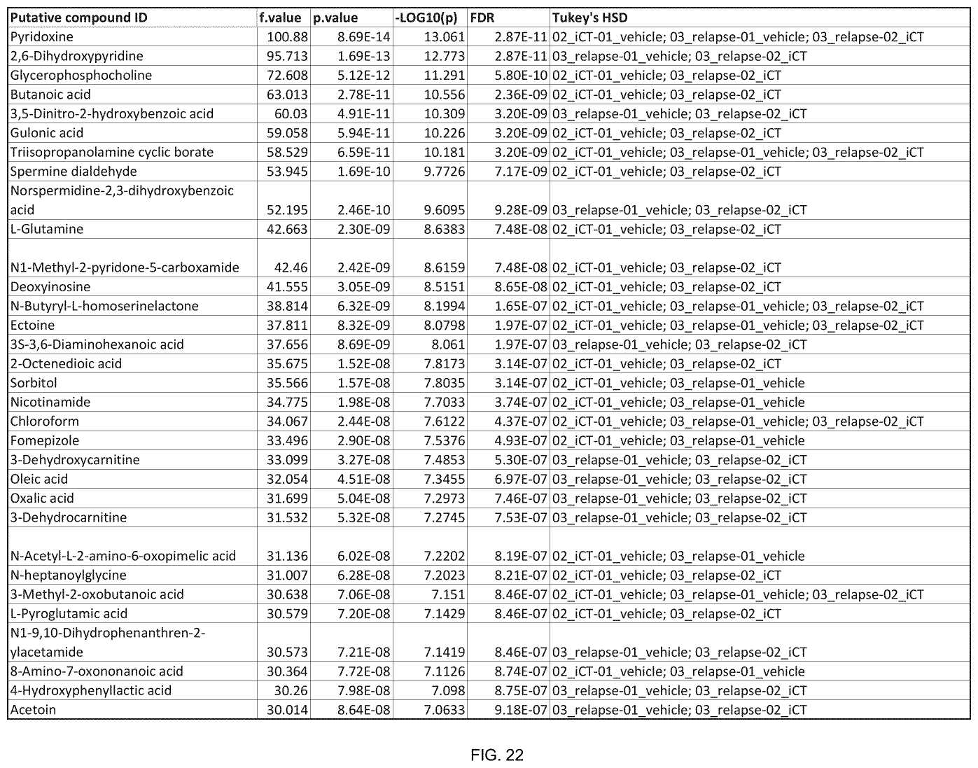

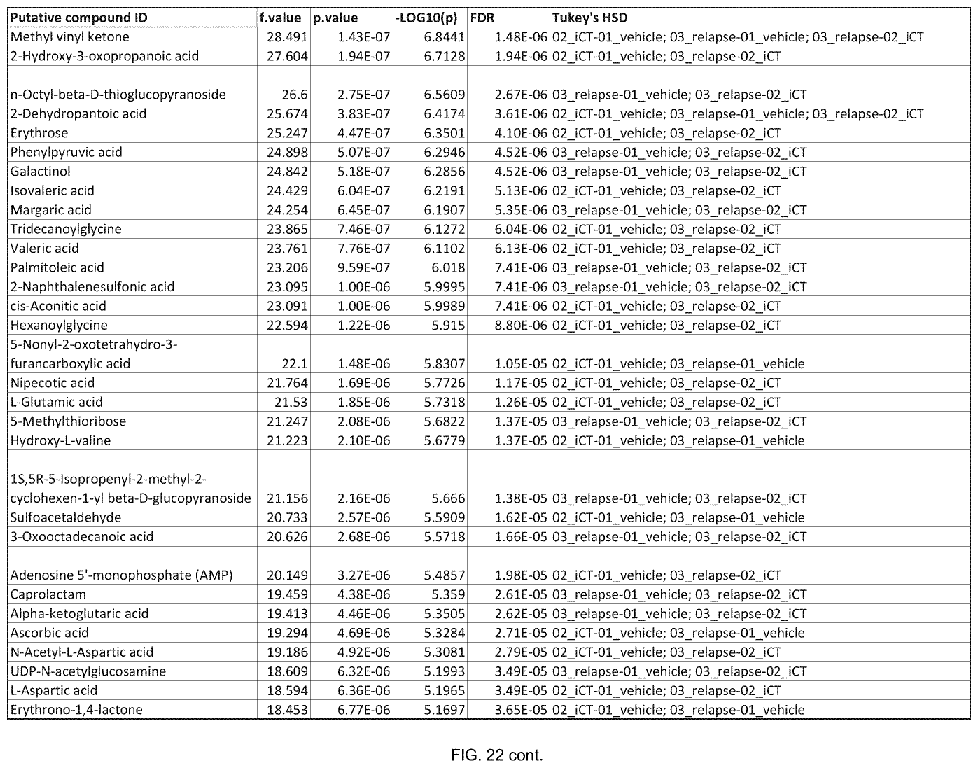

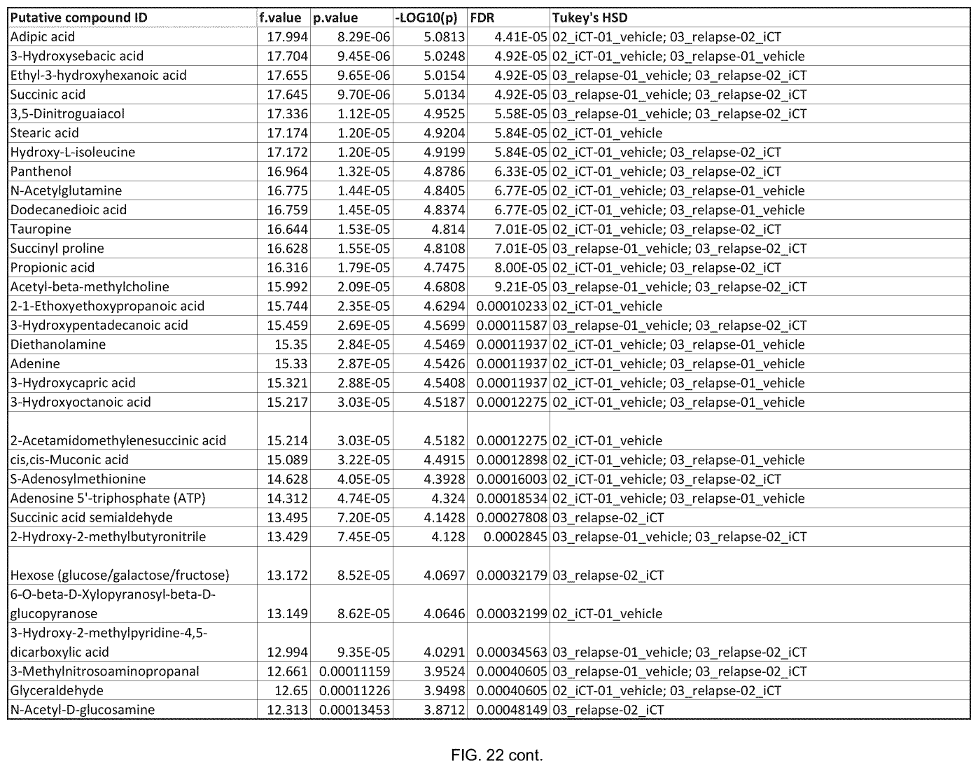

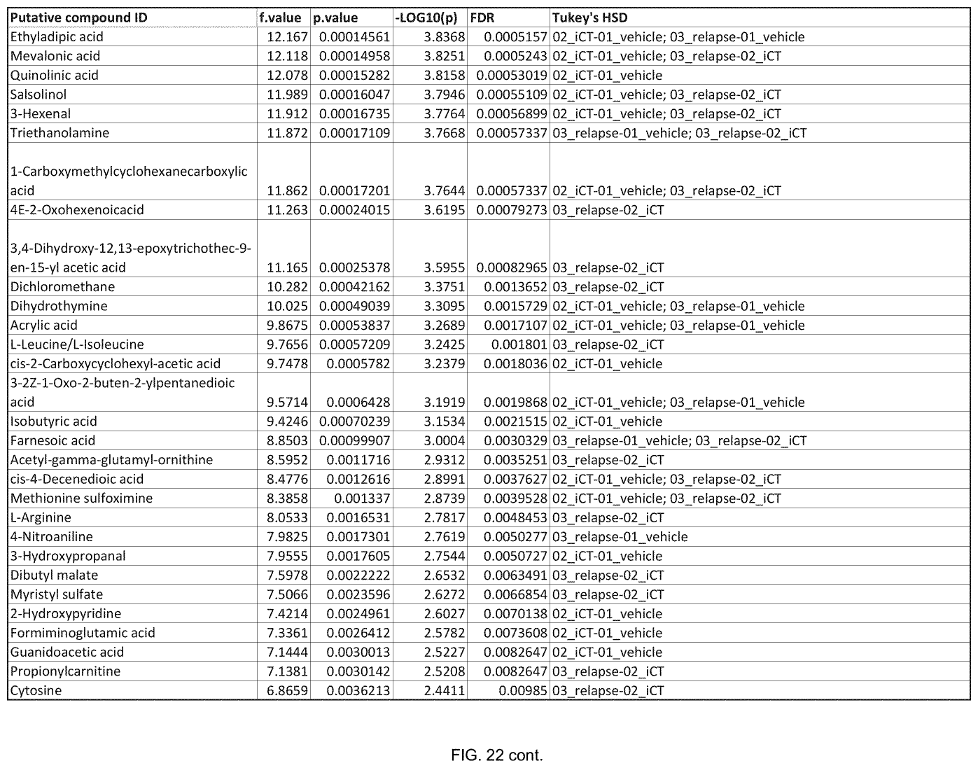

A methodology to investigate the metabolic profile of freshly isolated AML cells was then developed. Bone marrow cells of mice were isolated after being subjected to vehicle treatment (day 3 after vehicle), at the time of maximal response after iCT (day 3 after iCT), or after relapse (day 10 after iCT), GFP⁺ AML cells were sorted using fluorescence-activated cell sorting (FACS) and polar metabolites were extracted (FIG. 11A). Samples were kept at 4° C. in phosphate-buffered saline (PBS) at all times to minimize metabolic disturbance (Dietmair et al., 2010). The total time needed for cell processing and FACS sorting was 45 minutes, which was kept the same for all samples irrespective of the treatment group. Experiments were performed to confirm that subjecting the cells to FACS minimally impacts their metabolite profile (FIG. 16D). The levels of intracellular metabolites in 25,000 sorted cells were then determined by untargeted metabolomics analysis using liquid chromatography-coupled tandem mass spectrometry (LC-MS/MS). 654 metabolites were measured, of which 129 were identified based on their MS/MS spectra using the mzCloud database, and another 211 could be putatively identified based on their molecular weight by searching the Human Metabolome database and KEGG Compound database (FIG. 21). The 340 putatively identified metabolites were then further analyzed using the MetaboAnalyst software. Principal component analysis showed that while groups clustered closely together, the iCT group separated from the vehicle and relapse groups (FIG. 16E). Statistically, 125 metabolites differed significantly between the groups (ANOVA, FDR<0.01; FIG. 22), and unsupervised clustering of samples based on the 75 most significantly different metabolites revealed the specific metabolic signature of each group (FIG. 11B).

Metabolic pathway enrichment analysis using the (putatively) identified metabolites revealed the related “glutamine and glutamate metabolism” and “alanine, aspartate and glutamate metabolism” as the top pathways distinguishing iCT-treated cells from vehicle-treated and relapsed cells (FIG. 11C). Glutamine, glutamate and aspartate, central metabolites in these pathways, are low in vehicle-treated cells, high in the residual cells at the moment of maximal response to iCT, and low again after relapse (FIG. 11D), suggesting a role in the immediate stress response to chemotherapy. Tricarboxylic acid (TCA) cycle metabolites (alpha-ketoglutarate, succinate, fumarate, malate, citrate, cis-aconitate), lactate, and most other amino acids (alanine, arginine, leucine/isoleucine, methionine, phenylalanine, proline, valine) did not show similar changes (FIG. 16F). Targeted analysis of polar metabolites in the aforementioned treatment conditions corroborated the observed changes in glutamine, glutamate and aspartate (FIG. 16G). Interestingly, when AML cells from vehicle or iCT-treated mice were isolated at the moment of maximal response and cultured in vitro for 24 hours, the differences in glutamine, glutamate and aspartate reversed (FIG. 16H). A difference in glutamine levels was not found when cells were treated with iCT in culture (FIG. 16I), showing that the metabolic profile of AML cells is highly dependent on the local microenvironment.

Timed Inhibition of Glutamine Metabolism Overcomes Chemoresistance in AML

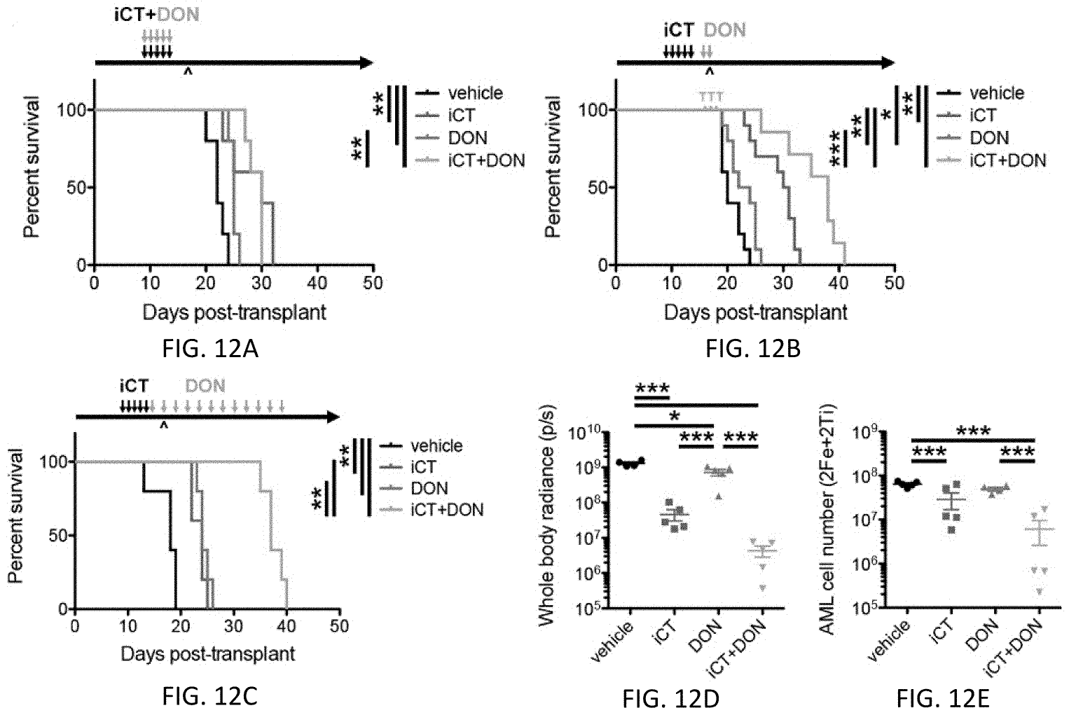

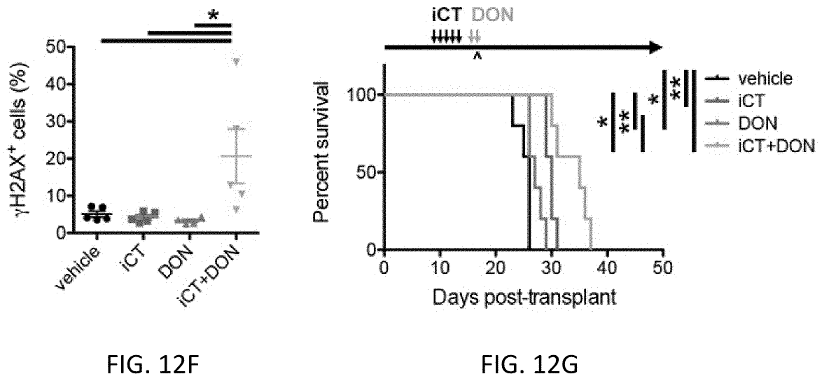

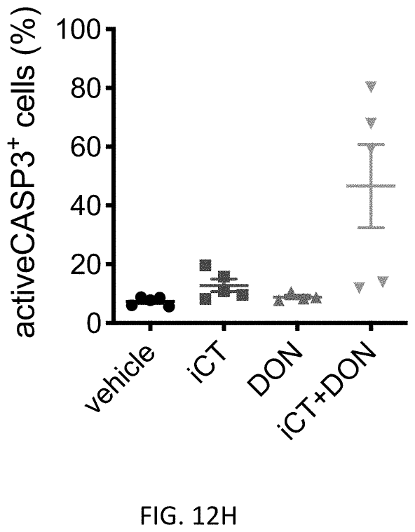

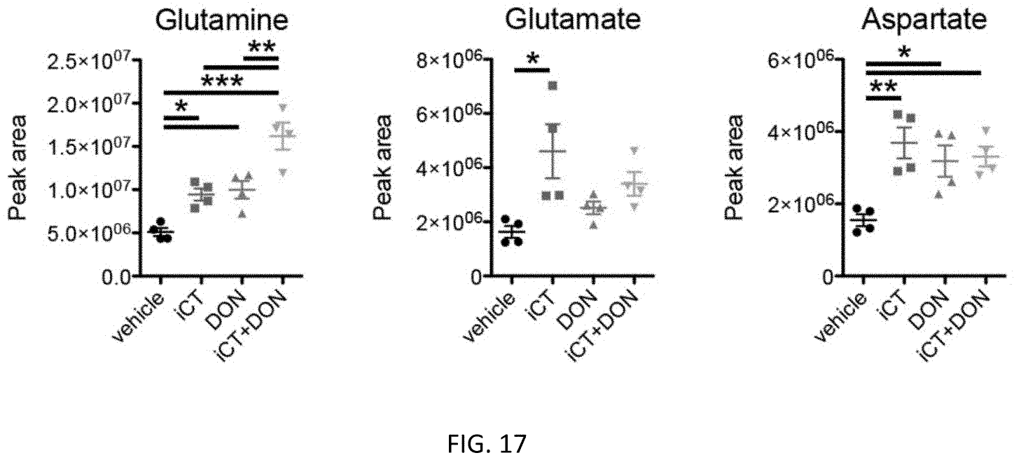

To determine whether the observed increase in glutamine metabolism-associated metabolites causes chemoresistance, it was assessed whether residual AML cells would be more susceptible to broad inhibition of glutamine metabolism using 6-diazo-5-oxo-L-norleucine (DON), a glutamine analog and antagonist that inhibits all glutamine-dependent enzymes (Lemberg et al., 2018). When DON was given for 5 days at the same time as iCT, no difference in survival was seen (FIG. 12A). However, when DON treatment was started after completion of the iCT regimen and covered the moment of maximal response, either daily for a short time (FIG. 12B) or continuously every other day (FIG. 12C), a clear survival benefit was observed. Short term treatment with DON after iCT improved elimination of AML cells (FIGS. 12D-12E), and increased double stranded DNA breaks in residual cells (FIG. 12F). Treatment of mice with DON lead to accumulation of glutamine in AML cells while partially reducing the increase in glutamate induced by iCT treatment, showing that DON effectively inhibits glutamine metabolism in AML cells (FIG. 17). Interestingly, aspartate levels did not follow a similar profile as glutamate, and remained high in mice treated with iCT followed by DON (FIG. 17). The effect of DON on chemoresistant cells was confirmed in a different mouse model of AML, driven by lentiviral HoxA9/Meis1 overexpression, which also showed improved mouse survival when treated with DON after completion of the iCT regimen (FIG. 12G). These data show that activation of glutamine metabolism protects AML cells at the moment of maximal stress after chemotherapy and reveal the potential of timed inhibition of glutamine metabolism to eliminate chemoresistant AML cells.

Chemoresistant AML Cells do not Depend on Glutaminase

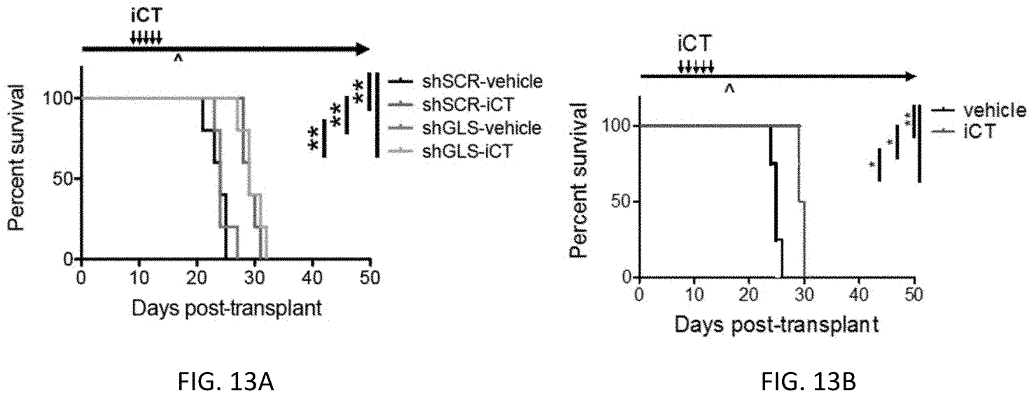

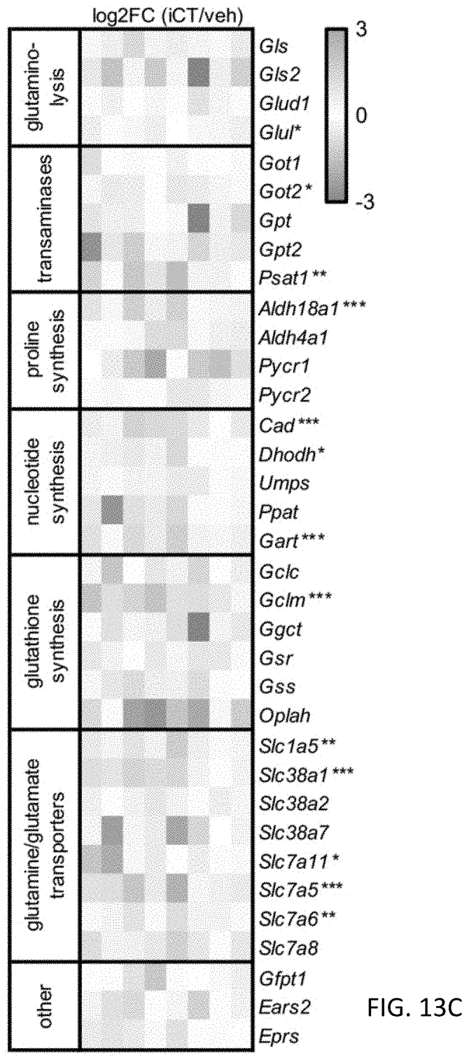

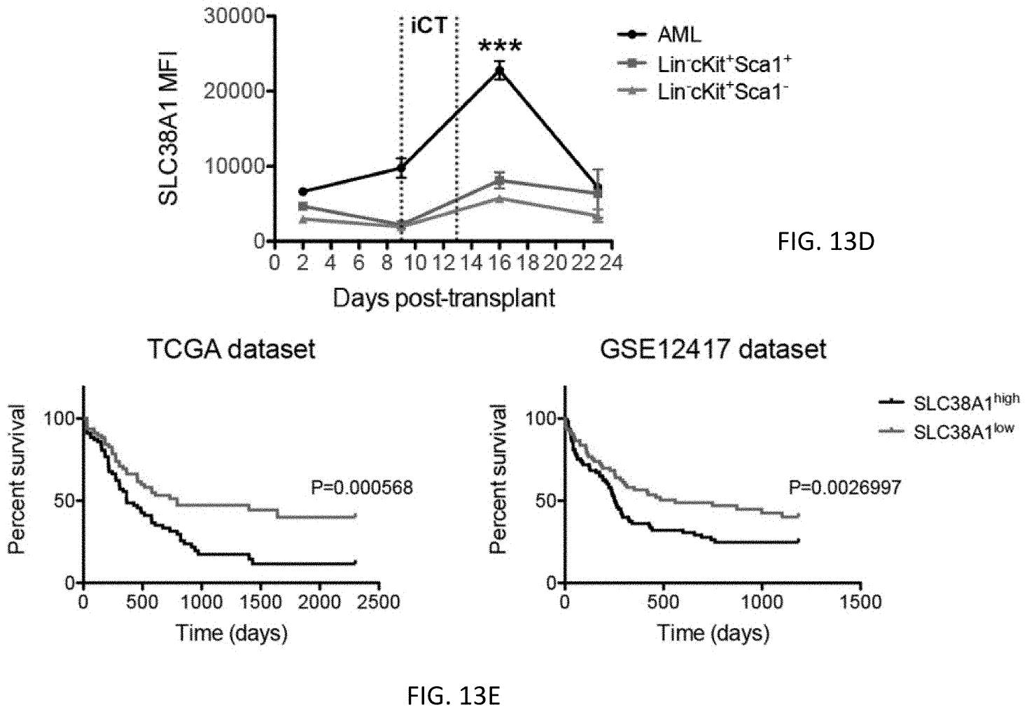

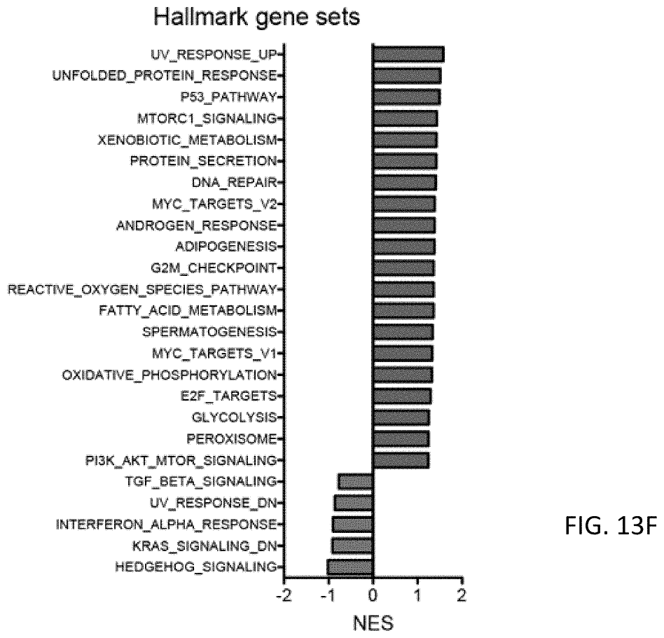

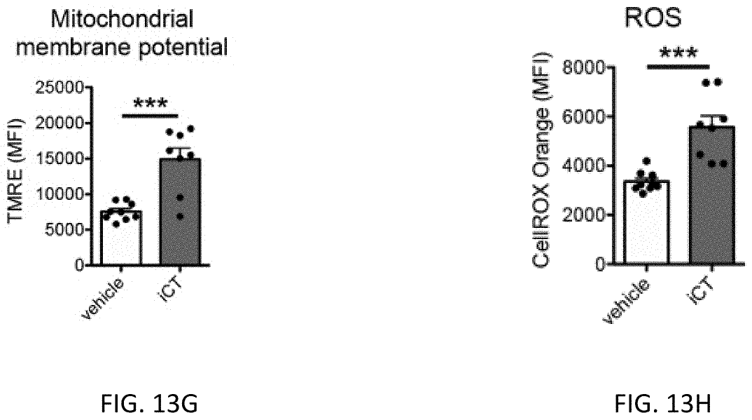

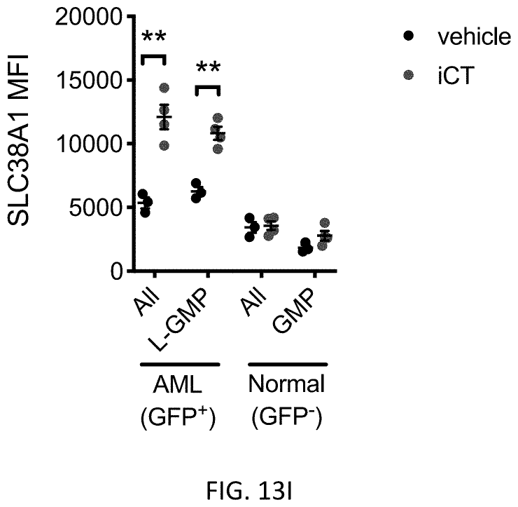

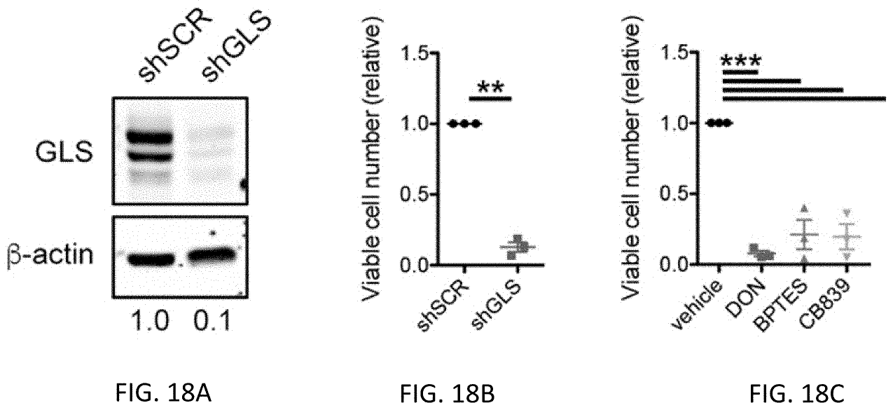

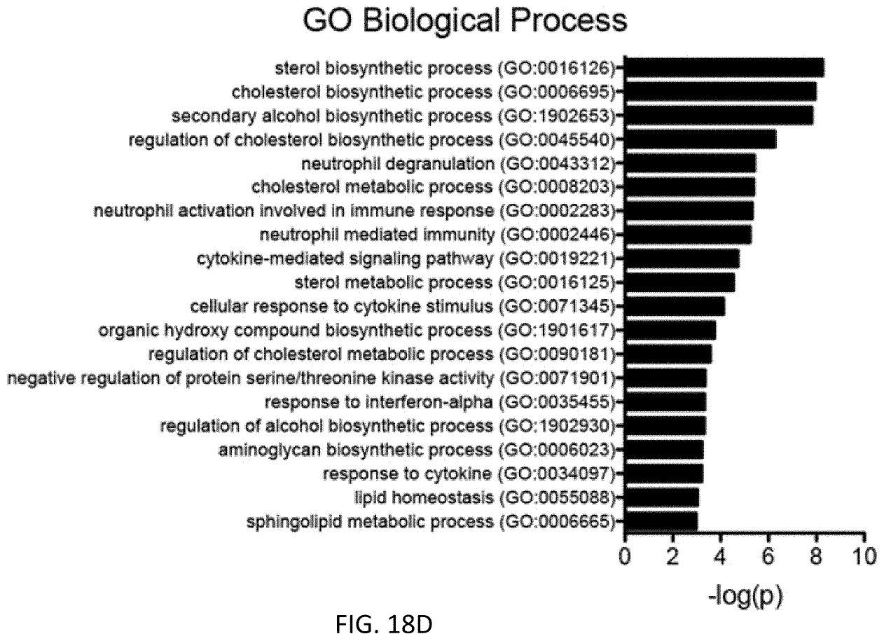

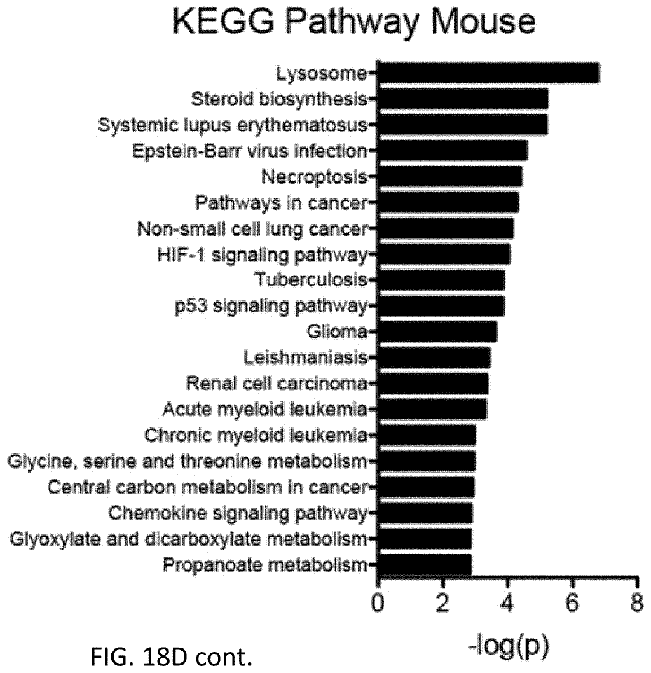

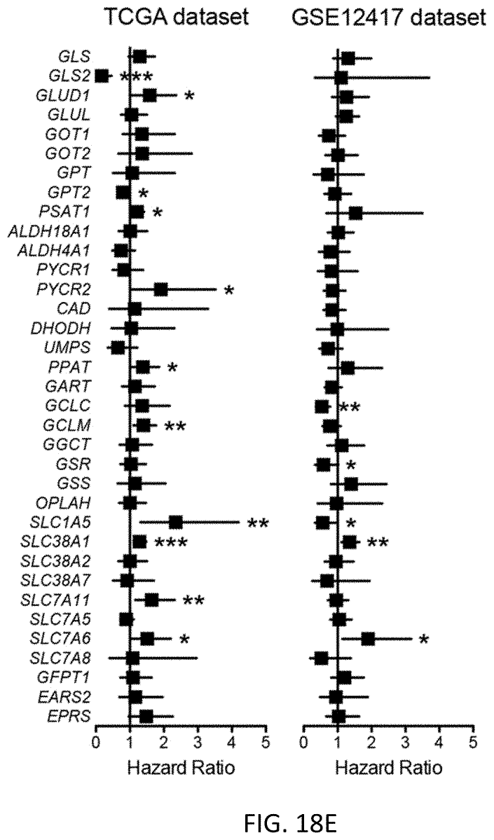

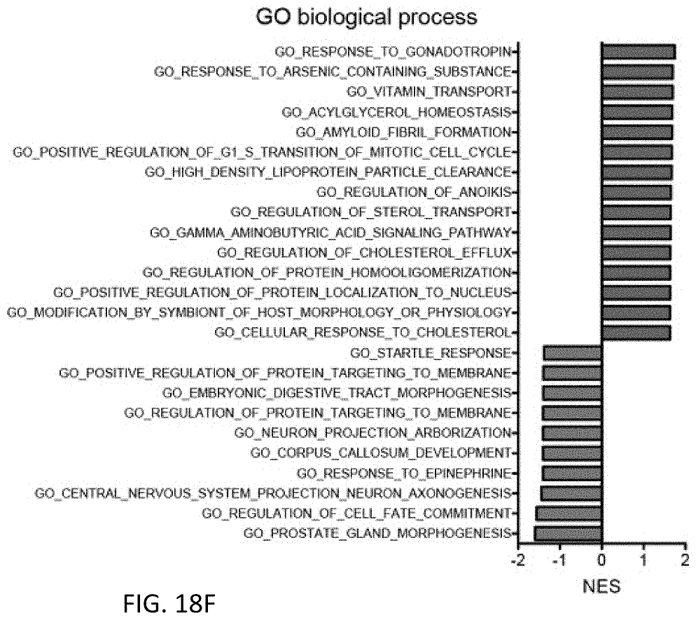

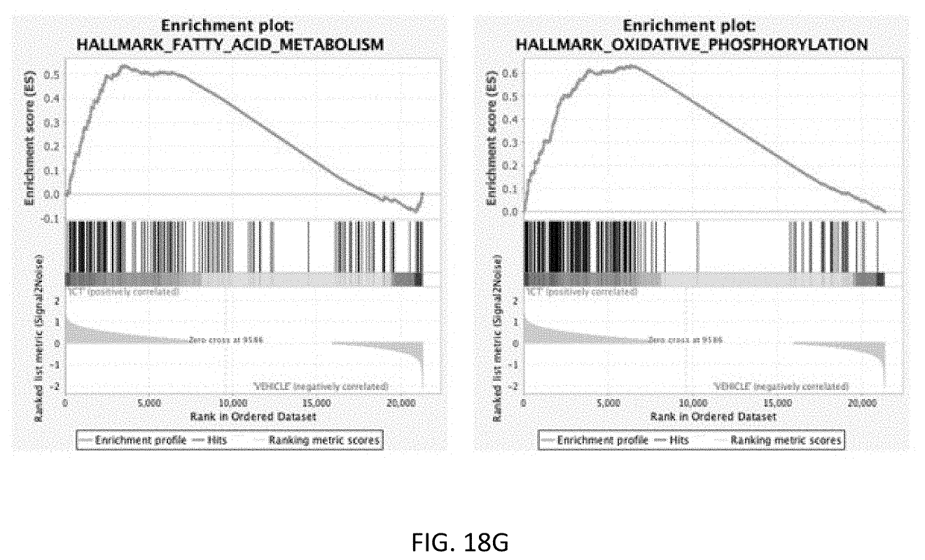

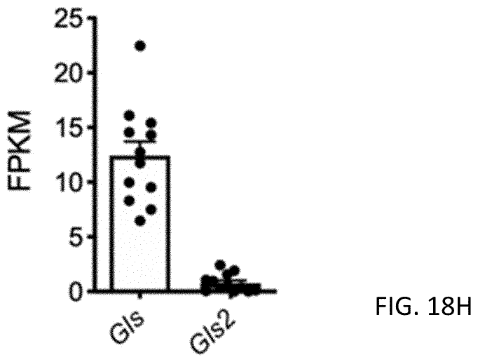

While DON has been tested in Phase I and II clinical trials for both solid tumors and leukemia in the 1980s, it was abandoned due to limited efficacy as a single agent and the occurrence of dose-dependent side effects including nausea, vomiting and mucositis (Lemberg et al., 2018). Due to these toxicity issues (FIG. 12B), alternative glutamine pathway inhibitors were sought. The enzyme glutaminase (GLS) has been shown to be a metabolic vulnerability in AML (Gregory et al., 2019, Jacque et al., 2015, Matre et al., 2016), but whether it mitigates the metabolic stress following chemotherapy is unknown. It was first confirmed that genetic or pharmacological inhibition of GLS reduced AML cell viability in vitro (FIGS. 18A-18C), in line with previous studies. Unexpectedly, GLS knockdown had no effect on disease progression or response to iCT in vivo (FIG. 13A). In addition, treatment of mice with a GLS inhibitor during a short window at the moment of maximal response after iCT did not enhance AML cell elimination or mouse survival. These results again highlight that cellular metabolism and metabolic dependencies are microenvironment dependent, and further show that AML cells persisting after chemotherapy, while critically depending on glutamine metabolism, do not require the enzyme glutaminase for their survival.

To better understand the metabolic fate of glutamine in chemopersistant AML cells, the comparative transcriptomic profiles of vehicle- and iCT-treated AML cells at the moment of maximal response were analyzed. The expression levels of most enzymes that metabolize glutamine or glutamate were unchanged in chemopersistant cells (FIG. 13C). While some individual enzymes showed increased expression post chemotherapy, no single pathway stood out as altered (FIG. 13C). In accordance, gene set enrichment analysis comparing vehicle- and iCT-treated cells did not return any glutamine-related pathways amongst the top hits (FIG. 13D, FIG. 18D). A recent study has shown that persisting human AML cells require oxidative metabolism fueled by fatty acid oxidation, and display high levels of reactive oxygen species and an increased mitochondrial activity (Farge et al. 2017). Similar observations were made in the mouse model of MLL-AF9 AML, with residual cells showing a higher mitochondrial membrane potential and creased ROS levels (FIGS. 13G-13H), thus confirming previous findings.

Closer examination of glutamine metabolism-related genes revealed increased expression of several glutamine transporters, including Slc1a5, Slc38a1, Slc7a5 and Slc7a6, was observed in chemoresistant AML cells (FIG. 13C). Flow cytometric analysis confirmed a transient increase in SLC38A1 protein levels after chemotherapy. This was not seen in normal hematopoietic stem and progenitor cell populations (FIG. 13D), suggesting that the changes in glutamine metabolism in response to chemotherapy are specific for AML cells. Two publicly available human AML survival datasets were analyzed, and it was found that high expression of SLC38A1 strongly associates with poor overall survival in both datasets (FIG. 13E), which was not found for other glutamine metabolism-related genes (FIG. 18E). Together, these data show that the transient increase in glutamine metabolism in chemoresistant AML cells seen at the metabolite level is not reflected at the mRNA level. The single exception is that of glutamine transporters.

Glutamine Metabolism Drives AML Chemoresistance by Fueling Pyrimidine Synthesis

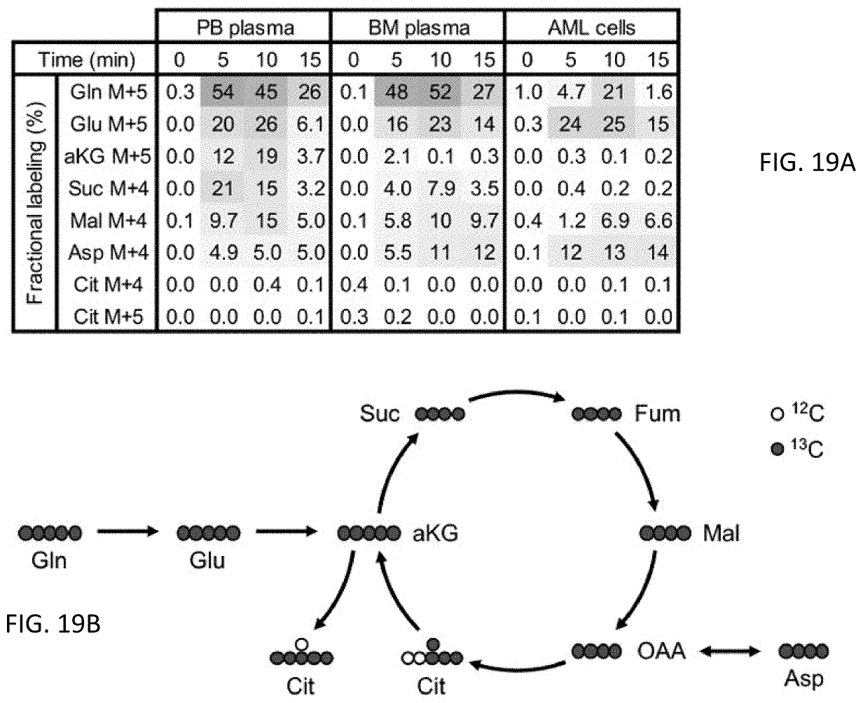

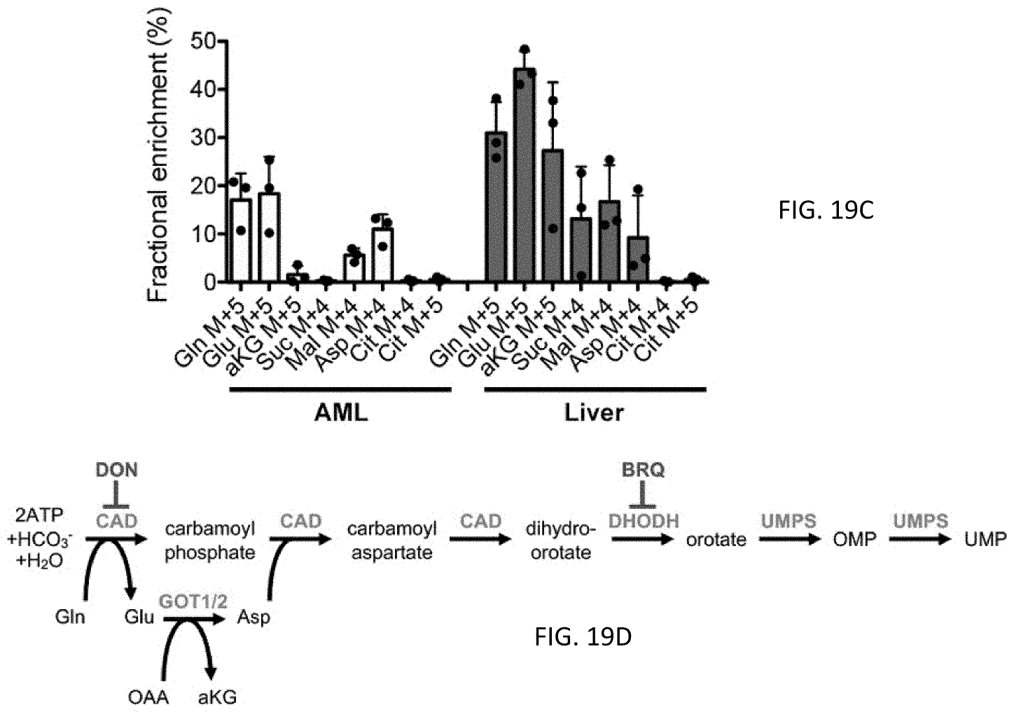

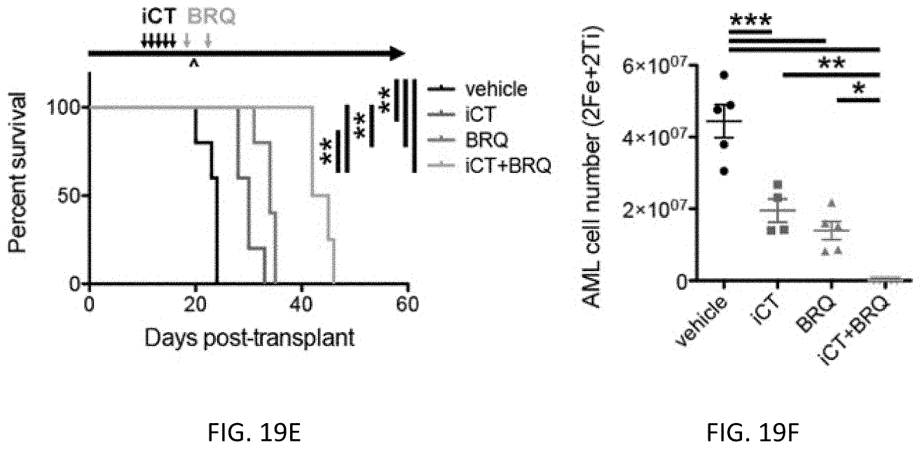

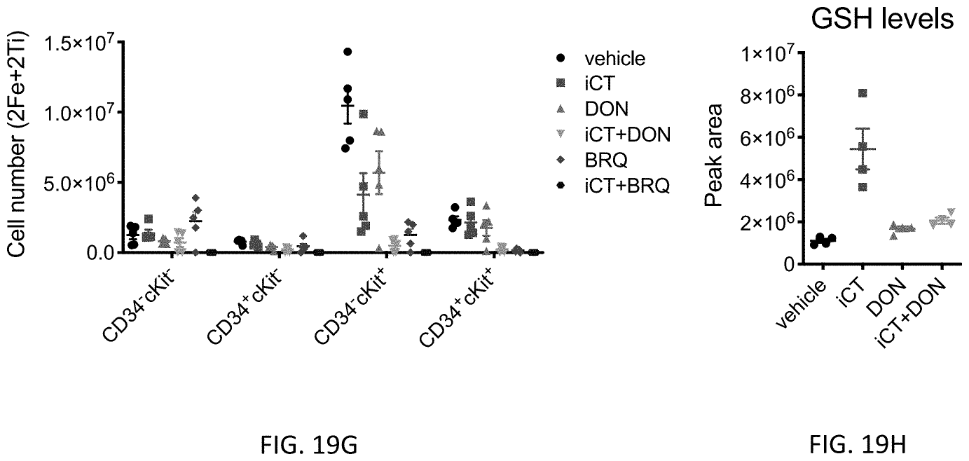

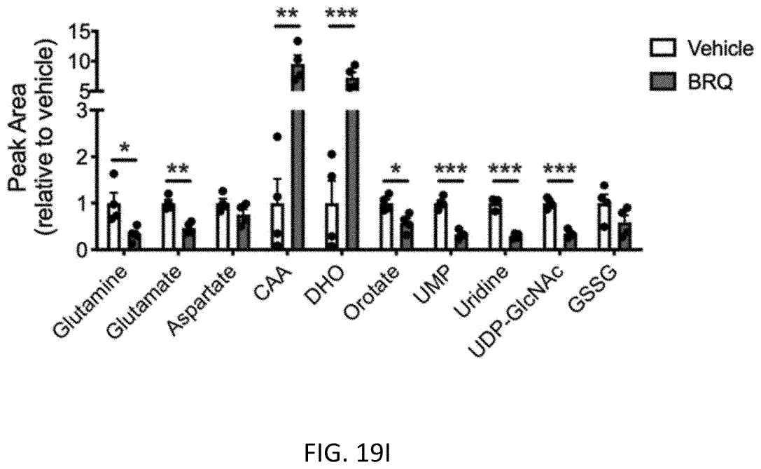

To assess the metabolic fate of glutamine in chemopersistant cells, in vivo stable isotope tracing was performed. Bolus intravascular injection of ¹³C₅-glutamine lead to rapid glutamine pool labeling in the peripheral blood and bone marrow plasma, maintained at 45-55% labeling for 10 minutes, after which it declined (FIG. 19A). Labeling of glutamine in AML cells occurred slower, with only 21% of the pool labeled at 10 minutes, and a rapid decline thereafter. Based on these results, the 10 minutes time point was chosen for analyses. Notably, bolus infusion revealed labeling of glutamine and glutamate pools in AML cells, but strikingly minimal labeling of the TCA cycle intermediates alpha-ketoglutarate and succinate (FIGS. 19A-19B). To validate that the LC-MS techniques were capable of detecting glutamine-derived carbon incorporation into the TCA cycle, metabolites in the liver of the same mice that were utilized for the AML experiments were profiled (Haussinger and Schliess, 2007). The liver showed extensive conversion of glutamine into TCA cycle metabolites including alpha-ketoglutarate and succinate, in line with the expected pattern for cells using glutamine to fuel the TCA cycle (FIG. 19C) (Haussinger and Schliess, 2007). This shows that in contrast to previous reports describing glutamine contribution to the TCA cycle in vitro in AML cells (Gregory et al., 2019, Matre et al., 2016), in vivo this does not seem to be a predominant fate of glutamine. These results mirror previous findings in mouse models of lung cancer, where similar discrepancies between in vitro and in vivo glutamine metabolism have been described (Davidson et al., 2016)

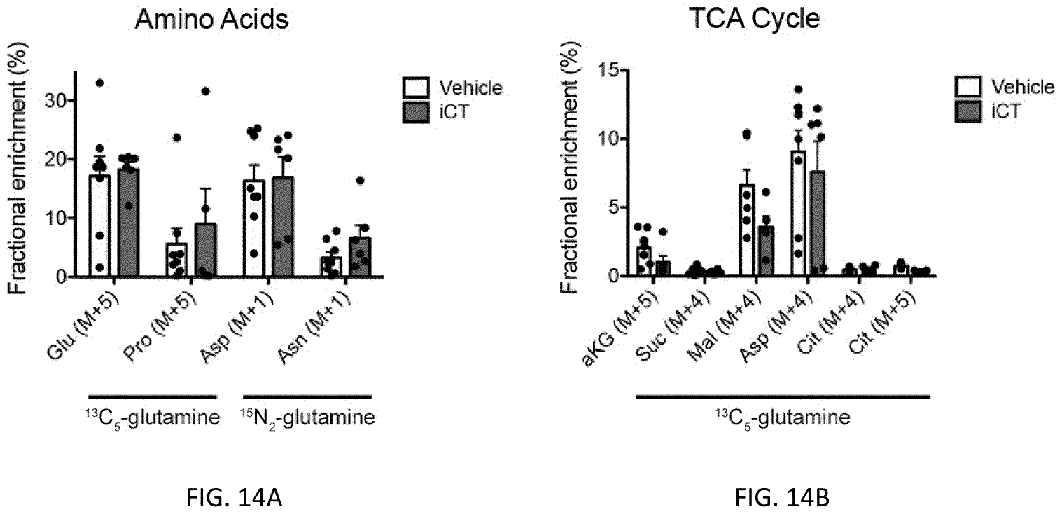

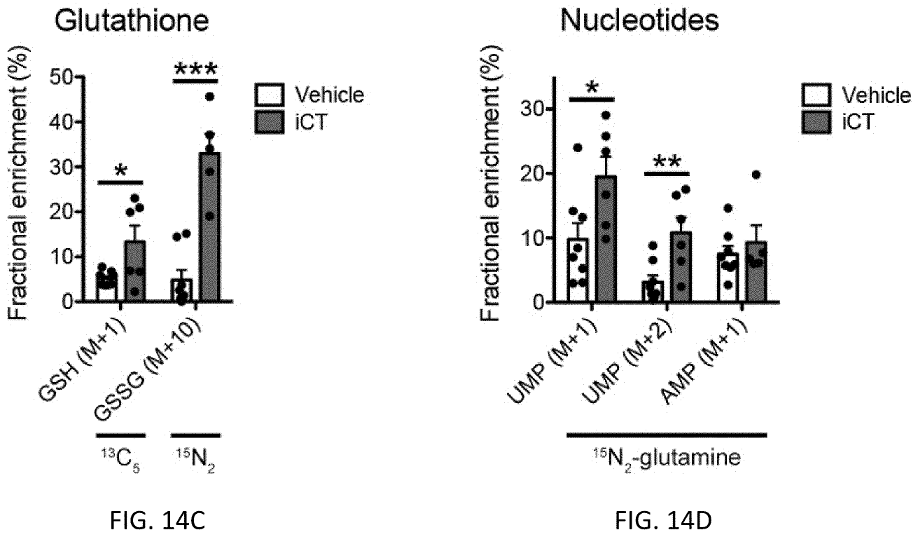

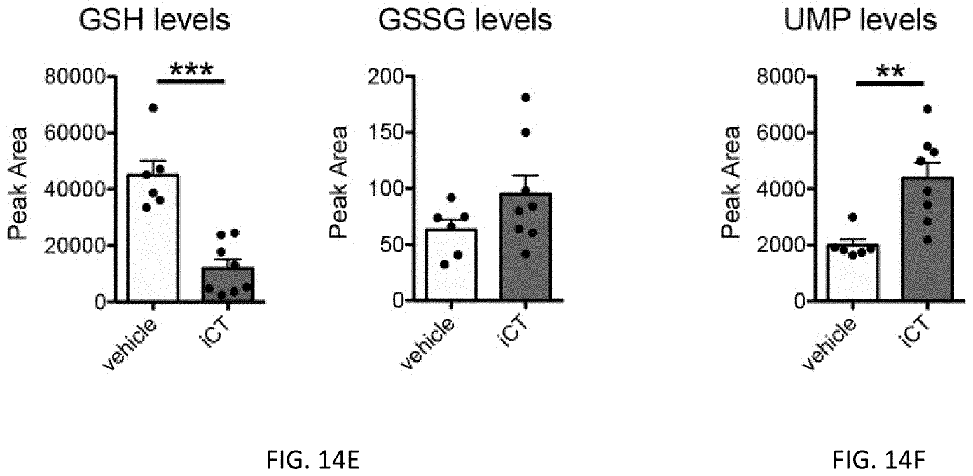

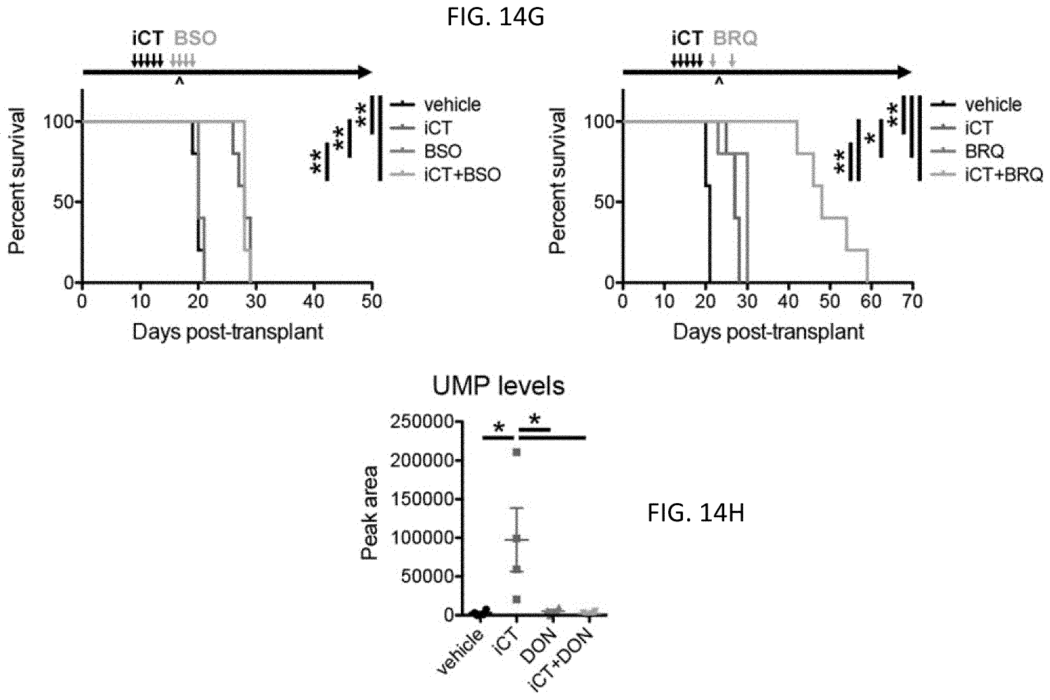

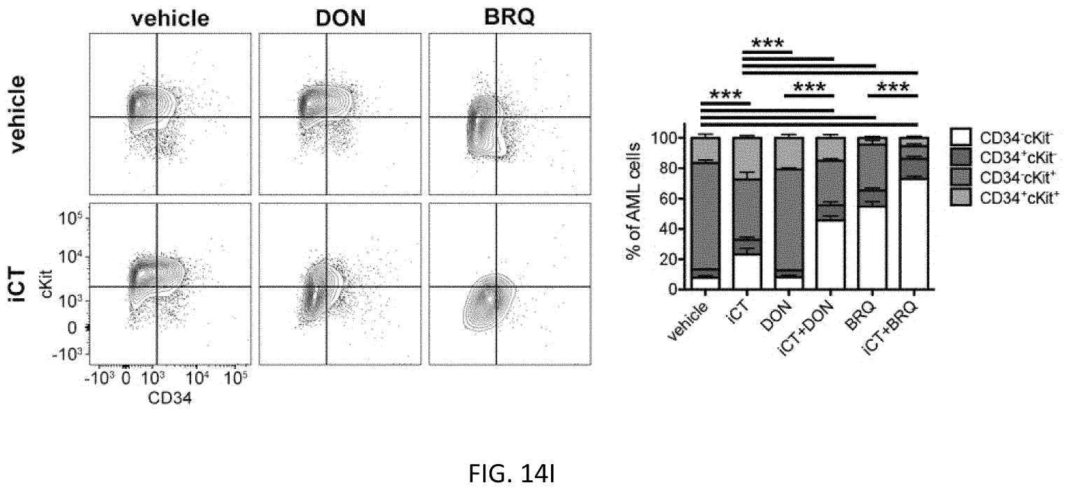

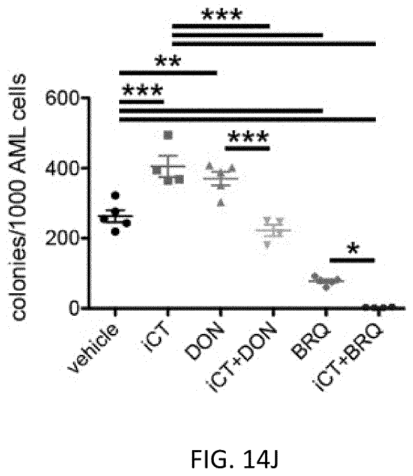

To understand how chemoresistant AML cells utilize glutamine, ¹³C₅-glutamine or ¹⁵N₂-glutamine was injected intravenously into vehicle- and iCT-treated mice at the moment of maximal response. Analysis of labeling patterns in AML cells showed no changes in the contribution of glutamine-derived carbon or nitrogen to amino acids or TCA cycle metabolites (FIGS. 14A-14B). Rather, a substantial increase of glutamine carbon and nitrogen incorporation into glutathione was observed (FIG. 14C). In addition, glutamine nitrogen incorporation into nucleotides was markedly elevated (FIG. 14D), especially into the pyrimidine uridine 5′-monophosphate (UMP). UMP gets one nitrogen from glutamine and one from aspartate, which is synthetized from glutamine-derived glutamate through transaminase activity (FIG. 19D). Increased fractions of UMP both containing one or two labeled nitrogen atoms were observed, showing that chemoresistant AML cells fuel pyrimidine synthesis with glutamine nitrogen both directly and indirectly through aspartate. Investigation of metabolite pool sizes revealed decreased levels of reduced glutathione (GSH) and unchanged levels of oxidized glutathione (GSSG) in chemoresistant cells (FIG. 14E). Together with the tracing results (FIG. 14C), this suggests that chemoresistant AML cells ramp up glutathione synthesis to replenish depleted GSH pools. Absolute levels of UMP in contrast were increased in chemoresistant cells (FIG. 14F), in line with increased pyrimidine synthesis fueled by glutamine. AML cells persisting after chemotherapy thus require glutamine-derived carbon and nitrogen to fuel glutathione synthesis and glutamine nitrogen to generate high levels of pyrimidine nucleotides.

Next, the potential of targeting glutathione synthesis and nucleotide synthesis individually to eliminate chemoresistant AML cells was investigated. To inhibit glutathione biosynthesis the compound L-buthionine-sulfoximine (BSO) was used, which inhibits glutamate-cysteine ligase (GCL). Treatment of AML-bearing mice with BSO for 4 days following iCT showed no difference in survival compared to iCT alone (FIG. 14G). Then pyrimidine synthesis was blocked using brequinar (BRQ), which inhibits the enzyme dihydroorotate dehydrogenase (DHODH) (FIG. 19D). Administration of only two doses of BRQ following iCT was sufficient to significantly extend survival of AML-bearing mice compared to iCT treatment alone (FIG. 14G, FIG. 19E). Similar to the effects of DON, short term treatment with BRQ after iCT improved elimination of AML cells from the bone marrow (FIG. 19F). These data show that chemoresistant AML cells require glutamine-dependent pyrimidine synthesis for their survival, while glutathione synthesis is not essential. In line with these findings, it was found that the pan-glutamine metabolism inhibitor DON prevented the increase of UMP levels in chemoresistant AML cells (FIG. 14H), confirming that these cells require glutamine to synthesize pyrimidines.

To test whether metabolic targeting strategies effectively eliminate leukemia-initiating cells (Krivtsov et al., 2006), expression of the immature cell markers CD34 and cKit were analyzed and the colony-forming ability of AML cells obtained from mice treated with vehicle or iCT alone or in combination with DON or BRQ was tested (FIGS. 14I-14J). Treatment with iCT alone increased the percentage of CD34⁺cKit⁺ cells as well as the number of colony-forming units (CFUs), while DON alone had no effect on marker expression but moderately increased the number of CFUs compared to vehicle-treated mice, indicating that these strategies eliminate bulk AML cells but relatively spare leukemia-initiating cells. The combination of iCT and DON normalized both the percentage of CD34⁺cKit⁺ cells as well as the number of CFUs back to vehicle levels, showing that iCT sensitizes both bulk AML cells and leukemia-initiating cells to DON treatment. BRQ in line with its pro-differentiation effect on AML cells (Sykes et al, 2016), strongly reduced the percentage of CD34⁺cKit⁺ cells as well as the number of CFUs. The combination of iCT and BRQ had similar effects on the percentage of CD34⁺cKit⁺ cells compared to BRQ alone, but further reduced the percentage of CD34⁻cKit+ cells and the number of CFUs. Together these data show that leukemia-initiating cells persisting after chemotherapy are effectively targeted by inhibitors of glutamine metabolism or pyrimidine synthesis.

Bone Marrow Stromal Cell-Derived Aspartate Supports Pyrimidine Synthesis in AML Cells

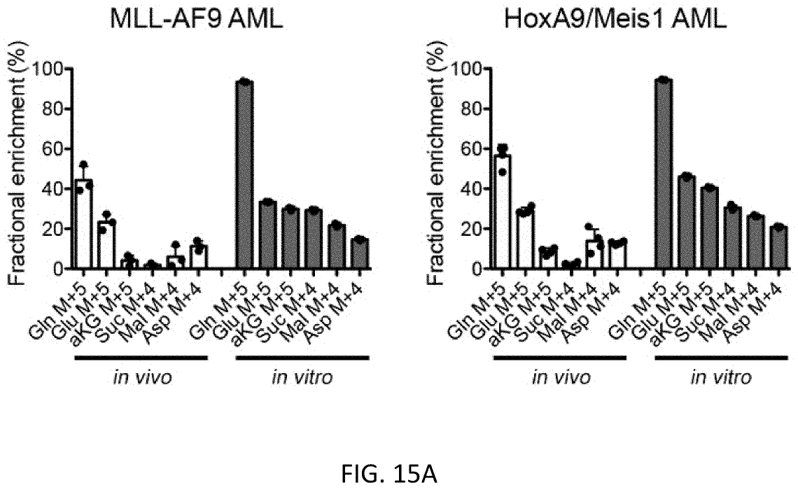

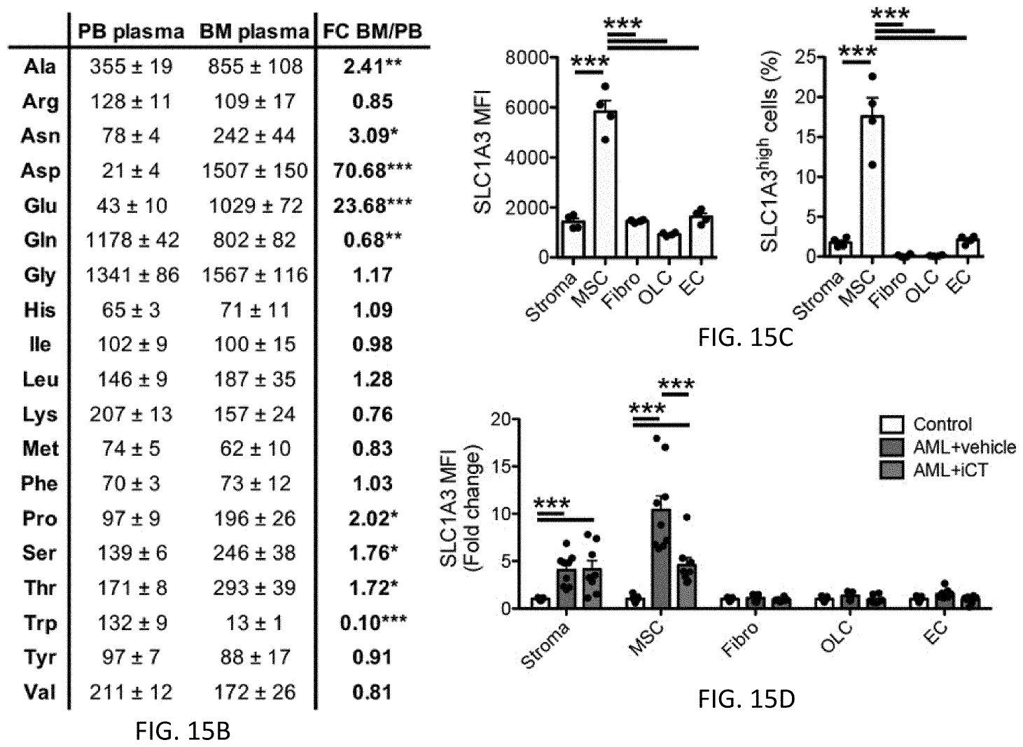

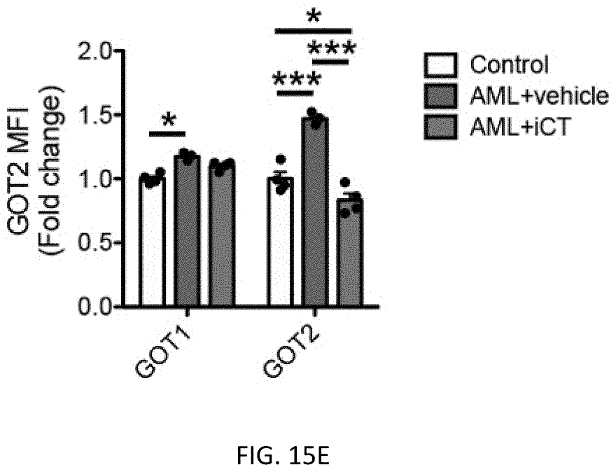

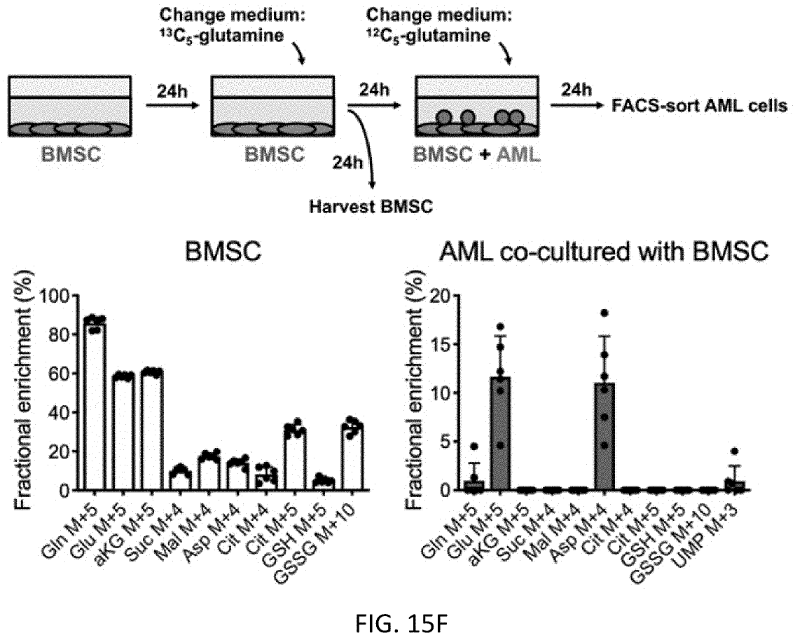

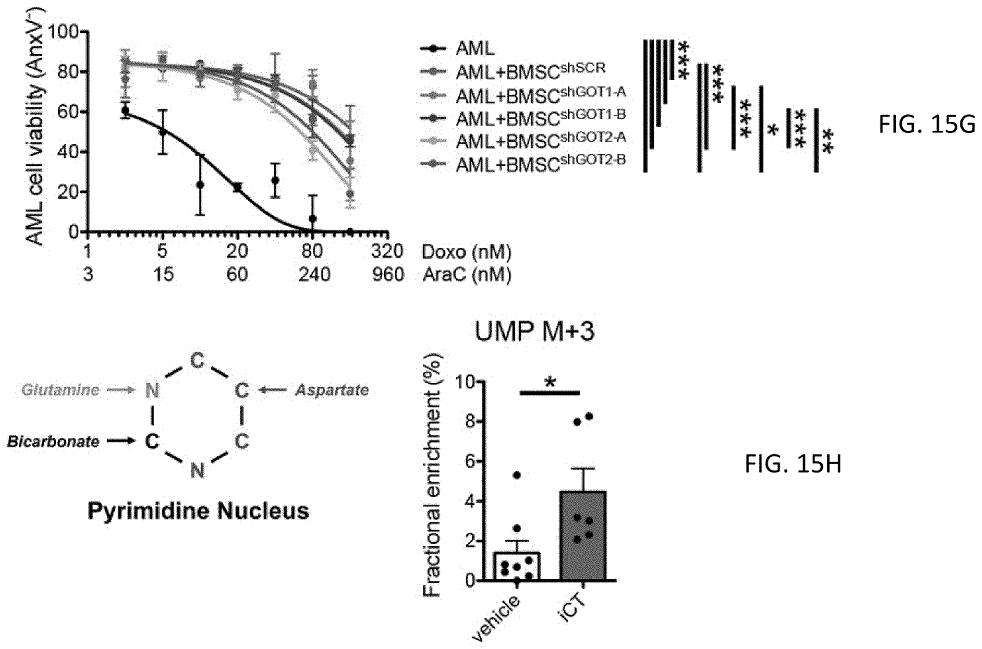

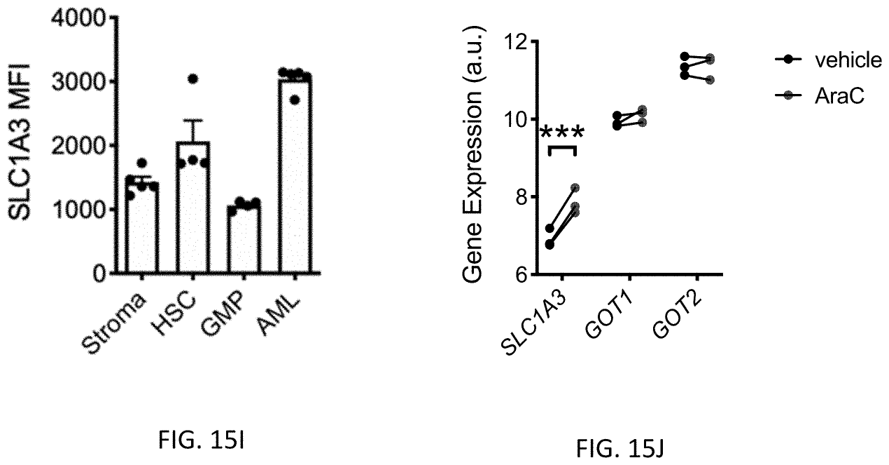

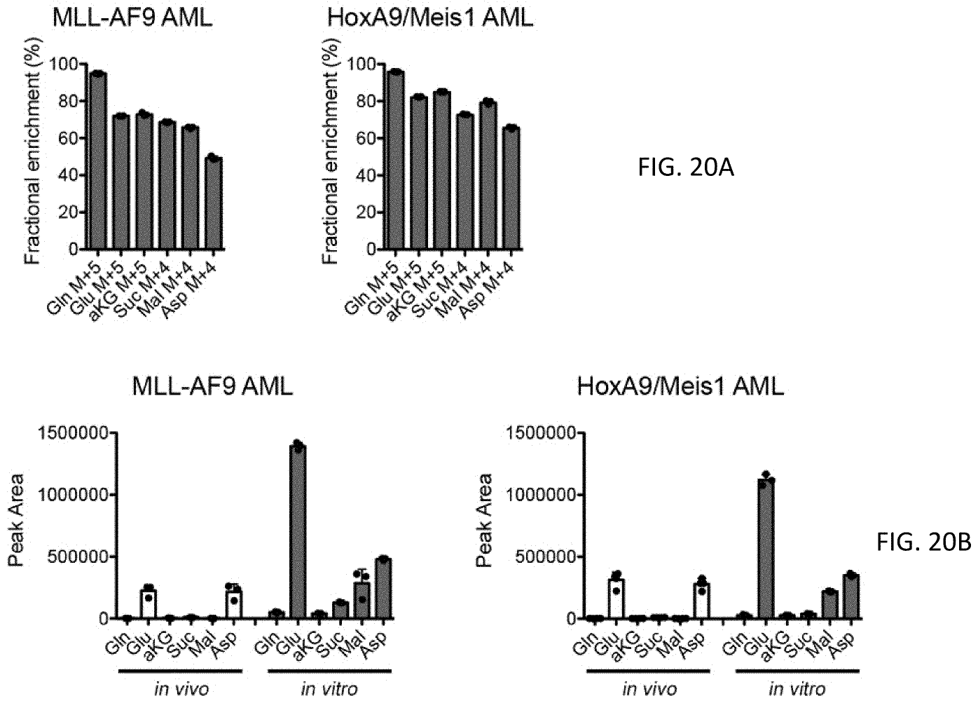

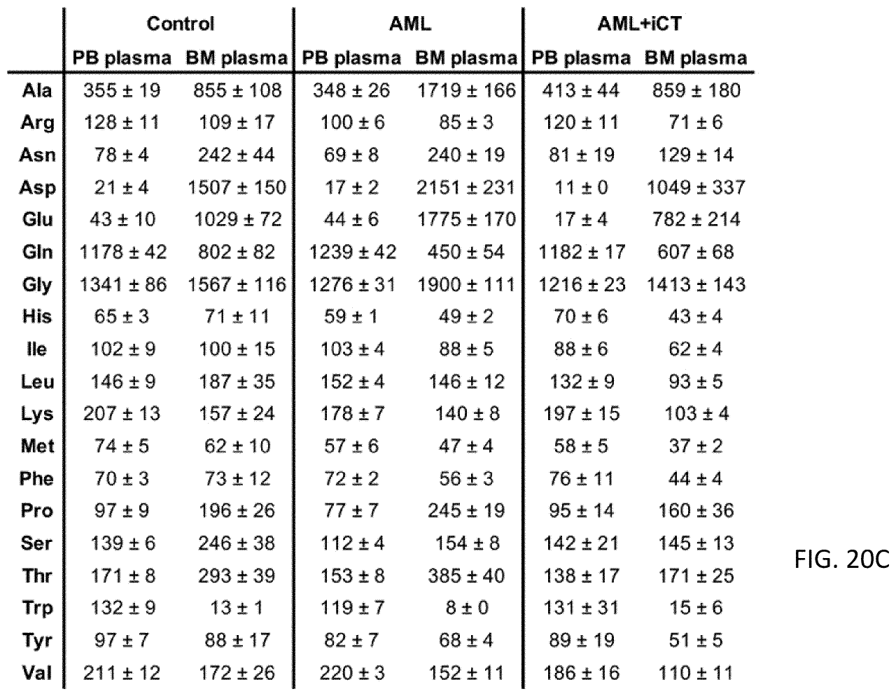

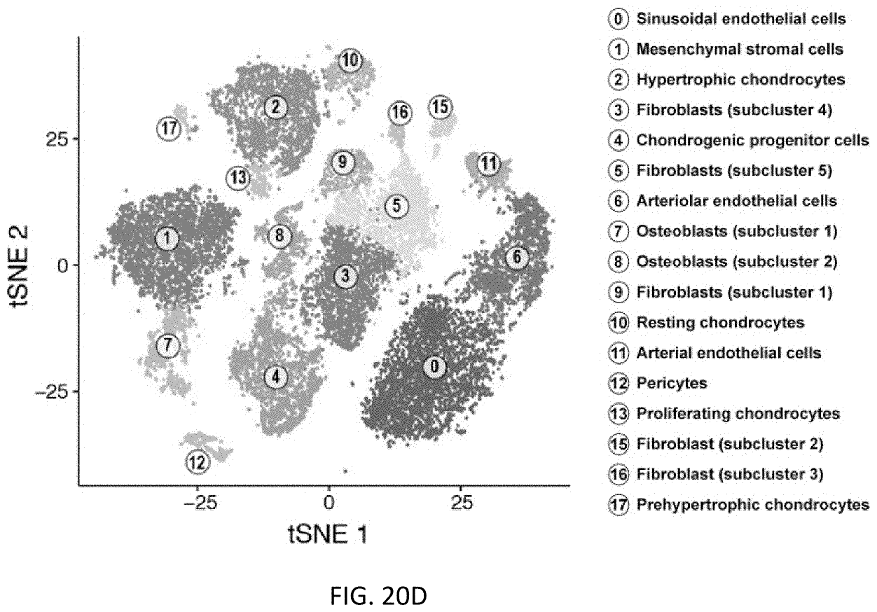

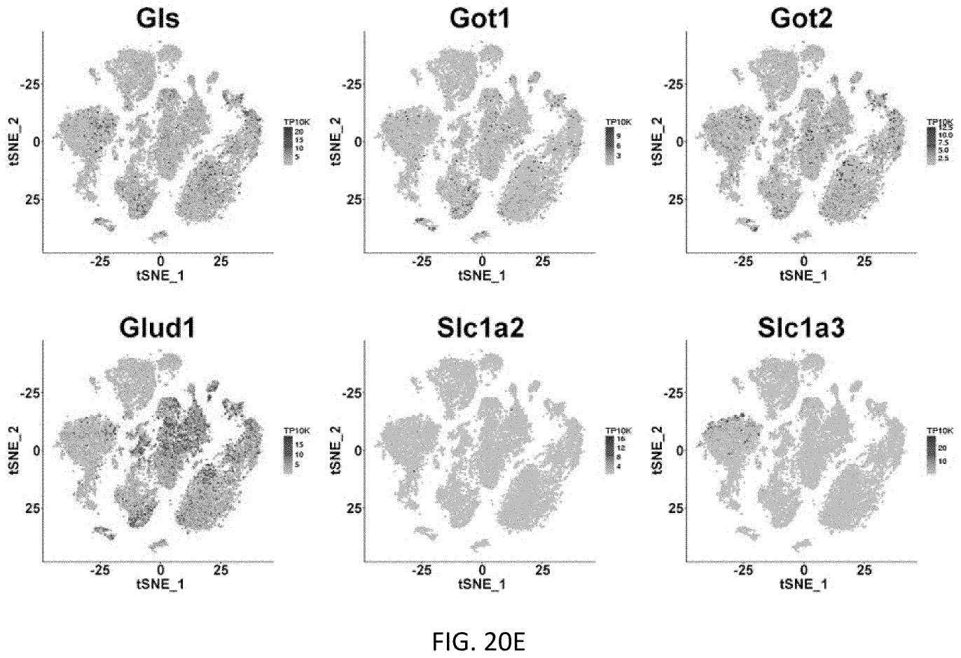

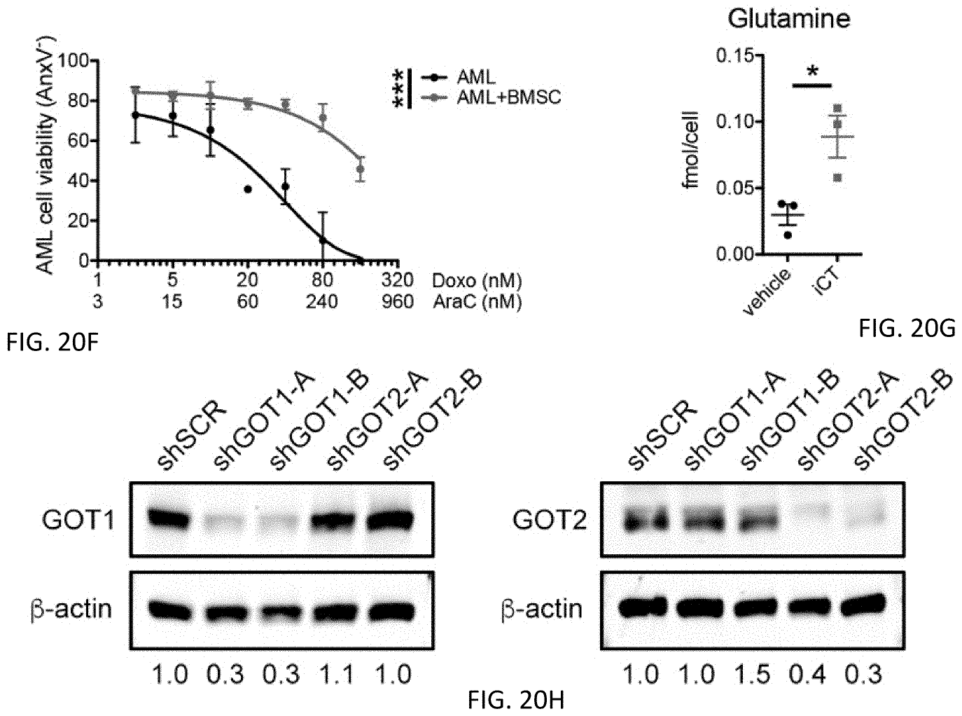

The in vivo ¹³C₅-glutamine tracing experiments showed that AML cells have substantially different labeling patterns compared to the liver (FIG. 19C). While in liver the TCA cycle is fueled by glutamine, as demonstrated by labeling of TCA cycle intermediates by ¹³C₅-glutamine, AML cells show hardly any labeling of alpha-ketoglutarate, succinate and citrate, but substantial labeling of the malate and especially aspartate pools. Since aspartate is another metabolite essential for pyrimidine synthesis, the origins of this particular labeling pattern were explored. Metabolic tracing was performed using ¹³C₅-glutamine in MLL-AF9- and HoxA9/Meis1-driven AML lines in vivo and in vitro. Interestingly, both AML models showed the unique labeling patterns only in vivo, while in vitro they exhibited the same labeling pattern as observed in the liver samples in vivo (FIG. 15A). Longer in vitro labeling increased overall tracer enrichment, but did not change the labeling patterns (FIG. 20A). These results show that the ¹³C₅-glutamine labeling pattern seen in AML cells in vivo is not due to technical issues such as labeling time or the cell model used, but reflects a unique metabolic program of AML cells in vivo.

Glutamine carbon cannot end up in aspartate without going through either alpha-ketoglutarate and succinate (oxidative glutamine catabolism) or citrate (reductive carboxylation) in the TCA cycle (FIG. 19B). One possibility is that the interconversion of glutamate to aspartate occurs so rapidly that labeling in the intermediates is not seen. However, further analysis of the in vivo ¹³C₅-glutamine tracing experiments revealed that labeling of glutamate and aspartate pools reached its peak in AML cells before labeling of glutamine pools (FIG. 19A). It was also found that AML cells in vivo have very small pools of alpha-ketoglutarate, succinate and malate compared to glutamate and aspartate (FIG. 20B), making it unlikely to not see substantial labeling of these metabolites even if the interconversion rates are high. The data thus suggests that AML cells in vivo obtain (glutamine-derived) aspartate from another source. Analysis of amino acid levels in peripheral blood revealed that aspartate is the lowest of all amino acids in the circulation (FIG. 15B), in accordance with previous reports (Cantor et al., 2017, Tardito et al., 2015). However, when amino acid levels in the bone marrow plasma were compared to those in the peripheral blood plasma it was found that while most amino acids do not differ substantially, aspartate levels are 70-fold higher, making it the second most abundant amino acid in bone marrow plasma (FIG. 15B). Glutamate, also of low abundance in peripheral blood, was 23-fold higher in bone marrow, while glutamine levels were 30 percent lower. Interestingly, in mice engrafted with AML cells, aspartate levels were further increased in the bone marrow plasma, while after iCT treatment aspartate levels were significantly lower (FIG. 20C). Glutamate levels followed a similar trend, while glutamine concentrations showed the opposite profile. These data suggest that AML cells may obtain aspartate from a local bone marrow source, which may be of particular importance after iCT given the increased levels of aspartate in chemoresistant AML cells (FIG. 11E) and the necessity of this metabolite for pyrimidine synthesis.

To identify the bone marrow cells responsible for local aspartate secretion, expression of genes encoding enzymes involved in glutamine-to-aspartate conversion was analyzed, as well as analyzing for aspartate transporters in a single cell RNA sequencing dataset of the mouse bone marrow stroma that was recently generated (FIG. 20D) (Baryawno et al., 2019). Most enzymes involved in aspartate synthesis from glutamine, including Gls, glutamate-oxaloacetate transaminase 1 (Got1), Got2 and glutamate dehydrogenase (Glud1), were expressed throughout the bone marrow stroma, including in Leptin Receptor (LepR)⁺CXCL12⁺ mesenchymal stromal cells (cluster 1), S100A4⁺ fibroblasts (clusters 3, 5, 9, 15, 16), CD31⁺ endothelial cells (clusters 0, 6, 11), osteocalcin osteolineage cells (clusters 7, 8), aggrecan⁺ chondrocytes (2, 4, 10, 13, 17) and Nestin⁺NG2⁺ pericytes (cluster 12) (FIG. 20E). In contrast, the aspartate/glutamate transporter Slc1a3 was expressed almost exclusively in a subpopulation of the LepR⁺ mesenchymal stromal cells (FIG. 20E), which was confirmed at the protein level (FIG. 15C). The presence of AML cells increased SLC1A3 levels in stromal cells and particularly in LepR⁺ cells. In contrast, iCT treatment reduced SLC1A3 levels in LepR⁺ cells, although they remained significantly higher compared to control mice without AML (FIG. 15D). The presence of AML cells also increased the levels of the aspartate transaminases, GOT1 and particularly GOT2, in LepR⁺ cells (FIG. 15E). These data are consistent with AML cells inducing BMSCs to alter their metabolism to increase aspartate production and secretion.

A possible exchange of aspartate between bone marrow stromal cells (BMSCs) and AML cells was tested by in vitro co-culture. In line with previous studies (Behrmann et al., 2018), it was found that the presence of BMSCs strikingly protected AML cells from iCT treatment (FIG. 20F). Of note, an increase in glutamine levels was observed in AML cells exposed to iCT in co-cultures (FIG. 20G). This was not seen in AML mono-cultures (FIG. 16I), but does mirror the in vivo response of AML cells to iCT (FIG. 11D, FIG. 16G). This shows that the presence of stromal cells better mimics the in vivo metabolic microenvironment of AML cells.

¹³C₅-glutamine tracing experiments were then performed where stromal cells were labeled for 24 hours before washing away the tracer and adding AML cells in medium containing unlabeled glutamine. 24 hours later, AML cells were sorted and stable isotope labeling patterns analyzed. BMSCs metabolized glutamine in the TCA cycle, and synthetized aspartate using glutamine carbons, with ˜15% of the aspartate pool fully labeled (FIG. 15F). At least part of this aspartate was then transferred to the AML cells, as evident by 11% of their aspartate pool being labeled. These data confirm the metabolic crosstalk between BMSCs and AML cells. Interestingly, AML cells also showed uptake of labeled glutamate produced by BMSCs, the only other amino acid enriched in the bone marrow plasma (FIG. 15B, FIG. 20C), highly abundant in AML cells in vivo (FIG. 20B) and increased after iCT treatment (FIG. 11D, FIG. 20G). The importance of the aspartate exchange was analyzed by knocking down GOT1 or GOT2 in BMSCs (FIG. 20H). Loss of GOT2 in BMSCs partially sensitized AML cells to iCT treatment, confirming that aspartate exchange contributes to the chemoprotective effect of BMSCs on AML cells (FIG. 15G). Together, these data show that BMSCs convert glutamine to aspartate, which is then transferred to AML cells enabling them to survive iCT exposure.

Since aspartate is an essential metabolite for pyrimidine synthesis, evidence was looked for in the in vivo tracing data that glutamine-derived aspartate is used by chemoresistant AML cells to fuel UMP synthesis. The ¹⁵N₂-glutamine tracing data showed that both single and double ¹⁵N-labeled UMP was increased in chemoresistant AML cells (FIG. 14D). Since pyrimidines get one nitrogen each from glutamine and aspartate (FIG. 15H), this suggests that the glutamine-derived aspartate provided by the BMSCs is indeed used to support increased pyrimidine synthesis in chemoresistant AML cells. To further confirm this, UMP was looked for containing three ¹³C atoms in the ¹³C₅-glutamine in vivo tracing data, since aspartate, but not glutamine, contributes three carbons to UMP. Increased pools of ¹³C₃-UMP were in fact found in chemoresistant AML cells (FIG. 15H), further supporting the concept that glutamine fuels pyrimidine synthesis in chemoresistant AML cells both directly and indirectly via stromal cell-derived aspartate.

### Discussion

Despite the clear clinical need to prevent or delay cancer relapse, it remains poorly understood how certain tumor cells manage to survive the extreme stress of chemotherapy. In the current study, the metabolic profile of AML cells as they pass through that time of intense selection pressure was defined. It was found that residual AML cells exhibit transient metabolic adaptations driving their resistance to chemotherapy, and it was shown in mouse models of AML that timed manipulation of specific metabolic pathways, such as glutamine metabolism and pyrimidine synthesis, holds therapeutic potential. These findings underscore the importance of cell metabolism as a primitive, first line stress protection mechanism that can be targeted to eliminate chemoresistant cancer cells.

To investigate the metabolic profile of AML cells at a time when the milieu of the bone marrow microenvironment is maximally hostile, a pipeline for untargeted metabolomics of freshly-isolated AML cells from mice was developed. While this approach provides a broad snapshot of cellular metabolism in an unbiased manner, it also bring several challenges. First, cell isolation, sorting and sample processing must be performed in a fast but standardized manner, limiting the time between animal euthanasia and metabolite extraction to minimize metabolic changes. It was found that performing all steps at 4° C. greatly reduces procedural artefacts. Second, as with all metabolomics analyses, batch effects occur between individual experiments, but these seem to be even stronger when using low cell numbers. Including a reference sample that is taken along in each experiment helps significantly in correcting for this. Thirdly, and most importantly, data processing and analysis for untargeted metabolomics is highly labor-intensive. Inefficient peak alignment and limited MS/MS data, both more prominent due to the use of low cell numbers, requires substantial manual data processing and brings more uncertainty in metabolite identification. The use of both a reference sample and a pooled sample helps, especially with improving automatic metabolite annotation from cloud-based spectral databases. These approaches proved to be highly valuable in revealing the metabolic program of AML cells in their native environment, which would not have been obtained through transcriptomic analysis alone. In addition, a key metabolic feature of chemoresistant cells, high glutamine levels, was lost once cells were isolated and cultured, and was also not observed when AML cells were treated with iCT in vitro. These results indicate that the dynamic metabolic adaptations of AML cells persisting after chemotherapy are regulated at the pathway activity level and are highly dependent on the local microenvironment. This notion is further underscored by the metabolic tracer analysis, which revealed striking differences in glutamine metabolism in AML cells in vitro versus in vivo. The low contribution of glutamine to the TCA cycle in AML cells in vivo may explain the poor efficacy of GLS inhibition, even though this appears to be a promising target based on in vitro assays, and the peculiar nutritional composition of the bone marrow microenvironment with high glutamate levels may even further contribute to this lack of effect. The importance of examining the metabolism of cells in their native environment is supported by other recent studies that have shown that artificial in vitro conditions can create metabolic dependencies that do not exist in vivo (Cantor et al., 2017, Davidson et al., 2016, Muir et al., 2017).

Many studies have investigated the role of the bone marrow niche in AML progression and chemoresistance (Behrmann et al., 2018). However, most of these studies have focused on the role of adhesion molecules and cytokines. Very few studies have investigated the nutritional aspects of niche support in leukemia, with one report highlighting a role for bone marrow stromal cell-derived cysteine in chronic lymphocytic leukemia (Zhang et al., 2012), and a second study showing the importance of lipids provided by adipocytes for AML stem cells residing in adipose tissue (Ye et al., 2016). It is now shown that aspartate provided by LepR⁺ stromal cells in the bone marrow can fuel nucleotide synthesis in AML cells, which becomes of particular importance in the setting of chemotherapy treatment. These findings again highlight the question of how much of chemoresistance development is determined by genetic alterations versus microenvironmental signals. Are there hotspots of aspartate production in the bone marrow that constitute a protective niche, and is there competition between AML subclones for occupancy of these aspartate-rich locations? The documentation of the SLC1A3^{high} subset of LepR⁺ stromal cells may provide the means to identify this putative chemoprotective niche in AML models that capture clonal evolution, such as transgenic mouse models with more mutational complexity (Heckl et al., 2014, Vassiliou et al., 2011), patient-derived xenografts (Potter et al., 2019, Wang et al., 2017), or even directly in patient samples. In addition, it will be of interest to examine whether this metabolic crosstalk with stromal cell subsets also plays a role in normal hematopoiesis, given that the bone marrow is unexpectedly rich in aspartate compared to peripheral blood (also true in control mice), and the known role of LepR⁺ stromal cells in supporting hematopoietic stem cells (Ding et al. 2012b).

Translating the findings to clinical application may best be accomplished by exploiting the transient dependency on pyrimidine metabolism that has been observed. It is currently not clear whether the basis for this dependency is nucleic acid polymer generation or other functions of UMP such as its role in O-linked N-acetylglycosylation (Bond and Hanover, 2015). In prior studies pyrimidine synthesis inhibition was identified as inducing differentiation of AML cells, indicating a broad range of effects in malignancy (Sykes et al., 2016). Notably, a number of pyrimidine synthesis inhibitors are now in clinical trial, including brequinar, which is shown here, can remarkably extend animal survival when used for a brief interval post-chemotherapy. Clinical testing of such inhibitors should occur in a similar clinical context, given that the transient moment of stress that drives initial leukemia regeneration has also been described in AML patients (Boyd et al., 2018). In addition, the underlying hypothesis of a unique metabolic bottleneck associated with chemotherapy should be examined in the treatment of other tumor types.

### Materials and Methods

KEY RESOURCES TABLE

| | | |
|---|---|---|
| REAGENT or RESOURCE | SOURCE | IDENTIFIER |

Antibodies

| | | |
|---|---|---|
| Pacific Blue Lineage cocktail | BioLegend | Cat#: 133310 |
| | | RRID: AB_11150779 |
| APC anti-c-Kit | BioLegend | Cat#: 135108 |
| | | RRID: AB_2028407 |
| Brilliant Violet 510 anti-Sca-1 | BioLegend | Cat#: 108129 |
| | | RRID: AB_2561593 |
| eFluor 450 anti-CD34 | eBioscience | Cat#: 48-0341-82 |
| | | RRID: AB_2043837 |
| PE-Cy7 anti-CD45 | BioLegend | Cat#: 103114 |
| | | RRID: AB_312979 |
| PE-Cy7 anti-Ter-119 | BioLegend | Cat#: 116222 |
| | | RRID: AB_2281408 |
| PerCP-Cy5.5 anti-CD31 | BioLegend | Cat#: 102420 |
| | | RRID: AB_10613644 |
| APC anti-CD90.2 | BioLegend | Cat#: 105312 |
| | | RRID: AB_313183 |
| Pacific Blue anti-CD105 | BioLegend | Cat#: 120412 |
| | | RRID: AB_2098890 |
| Biotin anti-LepR | R&D Systems | Cat#: BAF497 |
| | | RRID: AB_2296953 |
| Anti-SLC1A3 (rabbit polyclonal) | Novus Biologicals | Cat#: NB100-1869 |
| | | RRID: AB_531518 |
| Anti-SLC38A1 (rabbit polyclonal) | ThermoFisher Scientific | Cat#: PA5-42420 |
| | | RRID: AB_2605455 |
| Alexa Fluor 647 anti-H2AX (pS139) | BD Biosciences | Cat#: 560447 |
| (mouse monoclonal) | | RRID: AB_1645414 |
| Anti-GOT1 (rabbit monoclonal) | Abcam | Cat#: ab170950 |
| Anti-GOT2 (rabbit monoclonal) | Abcam | Cat#: ab171739 |
| Anti-GLS (rabbit monoclonal) | ThermoFisher Scientific | Cat#: 701965 |
| | | RRID: AB_2633041 |
| Anti-β-actin (mouse monoclonal) | Sigma-Aldrich | Cat#: A2228 |
| | | RRID: AB_476697 |
| Alexa Fluor 546 anti-rabbit IgG (H + L) | ThermoFisher Scientific | Cat#: A11035 |
| | | RRID: AB_2633041 |
| IRDye 800CW anti-rabbit IgG (H + L) | LI-COR | Cat#: 925-32211 |
| | | RRID: AB_2651127 |
| IRDye 680RD anti-mouse IgG (H + L) | LI-COR | Cat#: 925-68070 |
| | | RRID: AB_2651128 |

Chemicals, Peptides, and Recombinant Proteins

| | | |
|---|---|---|
| Recombinant Mouse SCF Protein | R&D Systems | 455-MC |
| Recombinant Mouse IL-3 Protein | R&D Systems | 403-ML |
| Recombinant Mouse IL-6 Protein | R&D Systems | 406-ML |
| D-luciferin | GoldBio | LUCK |
| Cytarabine hydrochloride | Sigma-Aldrich | C6645 |
| Doxorubicin hydrochloride | Sigma-Aldrich | D1515 |
| 6-Diazo-5-oxo-L-norleucine (DON) | Sigma-Aldrich | D2141 |
| (2-Hydroxypropyl)-β-cyclodextrin | Sigma-Aldrich | H107 |
| Solutol HS 15 | Sigma-Aldrich | 42966 |
| Poly(ethylene glycol)-400 | Sigma-Aldrich | 202398 |
| Brequinar (BRQ) | Broad Institute | N/A |
| 7-aminoactinomycin D (7-AAD) | BD Biosciences | 559925 |
| BPTES | Sigma-Aldrich | SML0601 |
| CB839 | MedKoo Biosciences | 206153 |
| APC Annexin V | BioLegend | 640941 |
| APC-eFluor 780 Streptavidin | eBioscience | 47-4317-82 |
| CellROX Orange | ThermoFisher Scientific | C10443 |
| Tetramethylrhodamine, Ethyl Ester | ThermoFisher Scientific | T669 |
| (TMRE) | | |
| RPMI 1640 Medium | Lonza | 12-167F |
| DMEM Medium | Lonza | 12-614F |
| Canonical Amino Acid Mix (¹³C-, ¹⁵N- | Cambridge Isotope | MSK-CAA-1 |
| labeled) | Laboratories | |
| L-Glutamine (13C5, 99%) | Cambridge Isotope | CLM-1822-H |
| | Laboratories | |
| L-Glutamine (15N2, 98%) | Cambridge Isotope | NLM-1328 |
| | Laboratories | |

Critical Commercial Assays

| | | |
|---|---|---|
| BD Cytofix/Cytoperm Kit | BD Biosciences | 554714 |
| RNeasy Plus Mini Kit | Qiagen | 74134 |
| MethoCult GF M3434 | StemCell Technologies | 03434 |

Deposited Data

| | | |
|---|---|---|
| RNA sequencing of MLL-AF9 leukemia | GEO | GSE139159 |
| cells sorted from mice treated with or | | |
| without iCT, at the moment of maximal | | |
| response or after relapse | | |
| Single cell RNA sequencing of mouse | GEO | GSE128423 |
| bone marrow stromal cells | | |

Experimental Models: Cell Lines

| | | |
|---|---|---|
| MLL-AF9 mouse AML | Sykes et al., 2016 | N/A |
| HoxA9/Meis1 mouse AML | Sykes et al., 2016 | N/A |

Experimental Models: Organisms/Strains

| | | |
|---|---|---|
| C57Bl/6J mice | Jackson Laboratories | 000664 |

Recombinant DNA

| | | |
|---|---|---|
| Mouse GLS shRNA (TRCN0000253167) | Sigma-Aldrich | SHCLND- |
| | | NM_001081081 |
| Mouse GOT1 shRNA | Sigma-Aldrich | SHCLND- |
| (TRCN0000119792, TRCN0000119795) | | NM_010324 |
| Mouse GOT2 shRNA | Sigma-Aldrich | SHCLND- |
| (TRCN0000326018, TRCN0000326020) | | NM_010325 |

Software and Algorithms

| | | |
|---|---|---|
| Compound Discoverer (version 3.0) | ThermoFisher Scientific | N/A |
| TraceFinder (version 4.1) | ThermoFisher Scientific | N/A |
| MassHunter (version B.08.00) | Agilent | N/A |
| MetaboAnalyst (version 4.0) | Chong et al., 2018 | N/A |
| GSEA (version 4.0.2) | Mootha et al., 2003; | N/A |
| | Subramanian et al., 2005 | |
| Prism (version 5.0) | GraphPad Software | N/A |
| FlowJo (version 10) | FlowJo | N/A |

Experimental Model and Subject Details

Mice

C57Bl/6J mice were purchased from Jackson Laboratories.

Syngeneic Leukemia Experiments

The MLL-AF9 and HoxA9/Meis1 leukemia models have been described previously (Sykes et al., 2016). Briefly, the MLL-AF9 model was established by crossing MLL-AF9 knockin mice (Corral et al., 1996) with mice expressing GFP under the control of the ubiquitin promoter as well as mice expressing luciferase under the control of the β-actin promoter. The retroviral transduction model of HoxA9/Meis1 leukemia was generated by the infection of bone marrow mononuclear cells with an MSCV-HoxA9-IRES-Meis1 construct (originally designed by Dr. Guy Sauvageau; Kroon et al., 1998). For both models, leukemic bone marrow cells from a terminally ill mouse were harvested and expanded ex vivo in RPMI1640 medium (Lonza) supplemented with 10% fetal bovine serum (FBS; Gibco), 100 I.U./ml penicillin, 100 μg/ml streptomycin, 2 mM L-glutamine (all from Corning), 20 ng/ml recombinant mouse SCF (rmSCF), 10 ng/ml recombinant mouse interleukin 3 (rmIL-3) and ng/ml rmIL-6 (all from R&D Systems).

Recipient male mice (age 8-10 weeks) were injected intravenously (retro-orbital) with 1 million leukemia cells in a volume of 100 μl of saline. Disease progression was tracked intravitally using bioluminescence imaging. Mice were anaesthetized using isoflurane, injected with luciferin (150 μl of a 15 mg/ml solution in PBS), and imaged on an IVIS 200 Spectrum In Vivo Imaging System (PerkinElmer). When indicated, mice were treated with iCT (cytarabine, 100 mg/kg, once daily for 5 days and doxorubicin, 3 mg/kg, once daily for the first 3 days) or vehicle (saline) delivered via I.P. injection. To inhibit glutamine metabolism, mice were treated with DON (0.3 mg/kg, once daily) or vehicle (saline) delivered via I.P. injection. To inhibit GLS, mice were treated with a GLS inhibitor (150 mg/kg, twice daily) or vehicle (20% HP-β-cyclodextrin+20% Solutol, 50 mM citrate buffer, pH 3) delivered by oral gavage. To inhibit DHODH, mice were treated with BRQ (50 mg/kg, once daily) or vehicle (70% PBS, 30% poly(ethylene glycol)-400, pH 7.2) delivered via I.P. injection. To inhibit GCL, mice were treated with BSO (twice daily by I.P. injection at 2.23 mg/kg+in the drinking water at 4.45 mg/ml) or vehicle (saline by I.P. injection+untreated drinking water). For survival experiments, mice were checked twice daily and euthanized by CO₂ asphyxiation when they showed signs of extensive distress or became moribund.

Method Details

Cell Isolation and Culture

The MLL-AF9 or HoxA9/Meis1 primary leukemia cells were expanded in RPMI1640 medium supplemented with 10% FBS, 100 I.U./ml penicillin, 100 μg/ml streptomycin, 2 mM L-glutamine, 20 ng/ml rmSCF, 10 ng/ml rmIL-3 and 10 ng/ml rmIL-6. Leukemia cells were cultured in a humidified incubator at 37° C. in ambient air with 5% CO₂.

For BMSC, femurs and tibias of mice were isolated and cleaned to remove muscle. Epiphyses were cut away and bone marrow was flushed out with PBS containing 2% FBS using a 25 gauge needle attached to a 20 ml syringe. Bone marrow fractions were kept on ice. Bones were cut into small pieces using scissors and digested in DMEM medium (Lonza) containing 3 mg/ml collagenase type 2 (Gibco) and 4 mg/ml dispase (Gibco) at 37° C. for 45 minutes. Bone cell suspensions were then pooled with the bone marrow fraction and passed through a 70 μm cell strainer. Cells were used for immediate analysis or cultured in DMEM medium supplemented with 20% FBS, 100 I.U./ml penicillin, 100 μg/ml streptomycin and 2 mM L-glutamine, in a humidified incubator at 37° C. in 2% 02 with 7.5% CO₂.

For co-cultures, BMSC were seeded in 96-well plates at 25,000 cells/well and AML cells were added at 5,000 cells/well. Cells were cultured in DMEM medium supplemented with 20% dialyzed FBS (Gibco), 100 I.U./ml penicillin, 100 μg/ml streptomycin, 0.4 mM L-glutamine, 20 ng/ml rmSCF, 10 ng/ml rmIL-3 and 10 ng/ml rmIL-6, in a humidified incubator at 37° C. in 2% 02 with 7.5% CO₂.

Isolation of Peripheral Blood and Bone Marrow Plasma

Mice were euthanized by CO₂ asphyxiation and peripheral blood was obtained by cardiac puncture and collected in K2-EDTA Microtainer tubes (BD Biosciences). Peripheral blood plasma was obtained by centrifuging samples for 5 minutes at 2,500×g. Femurs and tibias were dissected, cleaned and epiphyses cut off. Bone marrow plasma was obtained by centrifugation for 5 minutes at 2,500×g.

Fluorescence-Activated Cell Sorting (FACS)

Mice were euthanized by CO₂ asphyxiation and femurs and tibias were isolated and cleaned to remove muscle. Bones were then crushed using a mortar and pestle in ice-cold flow buffer (PBS containing 2% FBS), and the obtained cell suspension was passed through a 70 μm cell strainer. Red blood cells were removed by incubating cells for 5 minutes in ACK lysing buffer (Gibco) on ice, cells were washed and resuspended in ice-cold flow buffer containing the viability dye 7-aminoactinomycin D (7-AAD; BD Biosciences). Viable AML cells (GFP⁺7-AAD⁻) were sorted on a BD FACSAria II (BD Biosciences) with the sample collector cooled to 4° C. Sorted cells were immediately processed.

Flow Cytometry

Cells were suspended in flow buffer and stained for 30 minutes at 4° C. in the dark with antibodies against c-Kit, Sca-1, CD34, CD45, Ter-119, CD31, LepR, CD90, CD105, SLC1A3, SLC38A1 or a lineage cocktail (CD3, Gr-1, CD11b, B220, Ter-119). 7-AAD was included as a viability dye to help identify dead cells. When necessary, cells were stained with a secondary antibody (Alexa Fluor 546-conjugated anti-rabbit IgG) or APC-eFluor 780-conjugated streptavidin (eBioscience) for 15 minutes at 4° C. in the dark. For intracellular antibody staining cells were first stained for surface markers and then fixed and permeabilized (BD Cytofix/Cytoperm Kit, BD Biosciences). Samples were then stained with antibodies against GOT1, GOT2 or gamma H2AX in perm/wash buffer, washed and, for GOT1 and GOT2, stained with a secondary antibody (Alexa Fluor 546-conjugated anti-rabbit IgG). Cell viability was analyzed using Annexin V-APC (BioLegend) and propidium iodide (PI; ThermoFisher Scientific) with Annexin V⁻PI⁻ cells considered as viable cells. The number of viable cells was determined by taking into account the total number of cells per well as measured using a Cellometer (Nexcelom). Mitochondrial membrane potential was measured by Tetramethylrhodamine, ethyl ester (TMRE; ThermoFisher) staining and levels of ROS were measured by CellROX Orange staining. Cells were incubated with 200 nM TMRE or 2.5 μM CellROX Orange in RPMI1640 medium supplemented with 10% FBS, 100 I.U./ml penicillin, 100 μg/ml streptomycin and 2 mM L-glutamine at 37° C. for 30 minutes, washed in flow buffer and analyzed. For all flow cytometry experiments, single color controls were used to set compensations and fluorescence minus one controls were used to set gates. All flow cytometry data was collected on a BD LSR II flow cytometer and analyzed using FlowJo software.

Colony Forming Unit Assay

FACS-sorted viable AML cells (GFP⁺7-AAD⁻) were resuspended in MethoCult GF M3434 (StemCell Technologies) at 1000 cells/ml and plated in SmartDish 6-well plates (StemCell Technologies) at 1 ml/well in duplicate. Cells were cultured in a humidified incubator at 37° C. in ambient air with 5% CO₂. After 5 days colonies were counted.

Metabolomics Analysis

FACS-sorted cells or plasma samples were lysed in ice-cold methanol and polar metabolites were extracted using methanol-chloroform phase separation (methanol:water:chloroform in 2:1:2 ratio). Samples were then dried under nitrogen flow, resuspended in 70% acetonitrile in water containing 2.5 μM of an internal standard (¹³C-, ¹⁵N-labeled amino acid mix; Cambridge Isotope Laboratories) and run on a ThermoFisher Q-exactive equipped with Zic-pHILIC column (150×2.1 mm, 5 μm; Merck). A volume of 5 μl was injected and the full mass spectrum was obtained in both positive and negative mode (0 to 45 minutes, resolution 70000, AGC target 3e6, mz range 66 to 990). Mobile phase A for chromatography consisted of 20 mM ammonium carbonate, 0.1% ammonium hydroxide, in water and mobile phase B of 97% acetonitrile in water. For untargeted metabolomics, a pooled sample was created from all samples and used for MS/MS runs. For targeted analysis, a standard mix at 1 μM of each compound of interest was prepared and run after the samples to confirm retention times.

For untargeted metabolomics analysis, data were processed with Compound Discoverer 3.0 (ThermoFisher Scientific) using the mzCloud mass spectral library for compound annotation. For compounds for which an MS/MS spectrum was not available, the Kyoto Encyclopedia of Genes and Genomes (KEGG) Compound database (genome.jp/kegg; Kanehisa and Goto, 2000) and human metabolome database (HMDB; hmdb.ca; Wishart et al., 2007, Wishart et al., 2019) were used for putative compound annotation based on the monoisotopic molecular weight. For targeted metabolomics analysis, data were analyzed with Tracefinder4.1 (ThermoFisher Scientific).

In Vitro Metabolic Tracing

Cells in culture were incubated with 2 mM ¹³C₅-glutamine (Cambridge Isotope Laboratories) in glutamine-free media for the indicated times and harvested by centrifugation at 4° C. An aliquot of each well was taken at the time of harvesting to determine cell numbers. After washing with PBS, cells were lysed in ice-cold methanol and polar metabolites were extracted using methanol-chloroform phase separation (methanol:water:chloroform in 2:1:2 ratio). Samples were then dried under nitrogen flow, resuspended in 70% acetonitrile in water containing 2.5 μM of an internal standard (¹³C-, ¹⁵N-labeled amino acid mix) and run on a ThermoFisher Q-exactive equipped with Zic-pHILIC column (150×2.1 mm, 5 μm; Merck). A volume of 5 μl was injected and the full mass spectrum was obtained in both positive and negative polarity mode (0 to 45 minutes, resolution 70000, AGC target 3e6, m/z range 66 to 990). Mobile phase A for chromatography consisted of 20 mM ammonium carbonate, 0.1% ammonium hydroxide, in water and mobile phase B of 97% acetonitrile in water. A standard mix at 5 μM of each compound of interest was prepared and run after the samples to confirm retention times and to obtain the experimental value of natural isotopic distribution. Data were analyzed with Tracefinder 4.1. Each compound peak was integrated as the sum of all isotopes. The isotope ratios were then extracted from the mass spectral data.

In Vivo Metabolic Tracing

Mice were injected via the tail vein with 200 μl of a 54.3 mg/ml ¹³C₅-glutamine or ¹⁵N₂-glutamine (Cambridge Isotope Laboratories) in saline solution. At the indicated times after injection, mice were euthanized by CO₂ asphyxiation and femurs and tibias were isolated and processed for FACS. FACS-sorted cells were lysed in ice-cold 80% methanol in water. Samples were dried down using a SpeedVac Vacuum Concentrator (ThermoFisher) and re-suspended in mobile phase buffer A (97% H₂O, 3% MeOH, 10 mM Tributylamine, 15 mM Glacial Acetic Acid, pH 5.5). Metabolites were analyzed on a reverse phase ion-pairing chromatography (ZORBAX Extend-C18, 2.1×150 mm, 1.8 μm; Agilent) coupled to tandem mass spectrometry (Agilent), and analytes were eluted in buffer A and buffer B (10 mM Tributylamine, 15 mM Glacial Acetic Acid in 100% MeOH). Samples were ionized (with negative polarity) using Agilent Jet Spray ionization; nebulizer 45 psi, capillary—2000 V, nozzle voltage: 500 V, sheath gas temperature 325° C., and sheath gas flow 12 L/min. Peaks were integrated in Mass Hunter (Agilent).

RNA Sequencing

Total RNA was isolated using the RNeasy Plus Mini kit (Qiagen), with additional on-column DNase treatment to eliminate traces of genomic DNA. Nucleic acid concentration was quantified using a NanoDrop (Thermo Scientific). Libraries were prepared with an RNA library preparation kit (E7490, NEB) using 100 ng of RNA. RNA-seq libraries were sequenced with a 1×50-bp strand-specific protocol on a HiSeq 2500 (I lumina). Data were analyzed using a high-throughput next generation sequencing analysis pipeline: FASTQ files were aligned to the mouse genome (mm9) and gene expression profile for the individual samples was calculated for RPKM values using STAR and Cufflinks.

Single Cell RNA Sequencing

The single cell RNA sequencing dataset of the mouse long bone and bone marrow stroma was generated previously and detailed information on cell isolation, cell sorting, library preparation, RNA sequencing and data processing is provided in the original manuscript (Baryawno et al. 2019).

Immunoblotting

Total cell lysates were obtained by lysing equal amounts of cells in RIPA buffer (ThermoFisher Scientific) supplemented with 1× cOmplete protease inhibitor cocktail (Roche). Proteins were separated by SDS-PAGE and transferred to a nitrocellulose membrane (Bio-Rad). Membranes were blocked with 5% dry milk in Tris-buffered saline with 0.1% Tween-20 (TBS-T) for 1 hour at room temperature and incubated overnight at 4° C. with primary antibodies (rabbit-anti-GLS, 1/1,000; rabbit-anti-GOT1, 1/1000; rabbit-anti-GOT2, 1/1000; mouse-anti-β-actin, 1/10,000) diluted in 5% BSA (Cell Signaling Technology) in TBS-T. Signals were detected using an Odyssey CLx imaging system (LI-COR) after incubation with IRDye-conjugated secondary antibodies (LI-COR).

shRNAs

To silence GLS, GOT1 and/or GOT2 cells were transduced, in the presence of 7 μg/ml polybrene (Sigma-Aldrich), with a lentivirus carrying a shRNA against mouse GLS (MISSION, Sigma-Aldrich; TRCN0000253167), mouse GOT1 (MISSION, Sigma-Aldrich; TRCN0000119792, TRCN0000119795) and/or mouse GOT2 (MISSION, Sigma-Aldrich; TRCN0000326018, TRCN0000326020). A nonsense scrambled (SCR) shRNA sequence was used as a negative control. Cells were transduced by spinfection (90 minutes at 1,000×g), incubated for 3 hours, washed and plated in fresh medium.

Histology

Bones were harvested, fixed overnight in 4% paraformaldehyde in PBS and decalcified using 10% EDTA (pH 8). Bones were then embedded in gelatin-polyvinylpyrrolidone-sucrose and sectioned on a cryostat (50 μm sections) according to a previously published protocol (Kusumbe et al., 2015). Cell nuclei were visualized by Hoechst33342 (ThermoFisher Scientific) and mounted. Images were taken on a Zeiss LSM880 confocal laser scanning microscope.

Statistical Analysis

Analysis of the untargeted metabolomics dataset was performed using MetaboAnalyst (metaboanalyst.ca; Chong et al., 2018). The “Statistical Analysis” and “Pathway Analysis” nodes were used for analysis. Data was normalized by log transformation (generalized logarithm transformation) and auto-scaling (mean-centered and divided by the standard deviation of each variable). For pathway analysis, the Global Test and Relative-betweenness Centrality algorithms were used, and data were mapped to the mouse KEGG pathway library.

Gene set enrichment analysis was performed using the Gene Set Enrichment Analysis (GSEA) software (Broad Institute; Mootha et al., 2003, Subramanian et al., 2005) using the hallmark and biological process gene sets.

Correlation between AML patient survival and gene expression was performed using the PROGgeneV2 prognostics database (Goswami and Nakshatri, 2014). The GSE12417 and TCGA datasets were used for analysis.

All numerical results are reported as mean±standard error of the mean (s.e.m.). Statistical significance of the difference between experimental groups was analyzed by two-tailed unpaired Student's t-test, one-way ANOVA with Bonferroni post-hoc test or the log-rank (Mantel-Cox) test for the survival curve analyses using the GraphPad PRISM 5 software. Differences were considered statistically significant for P<0.05.

Data and Software Availability

The bulk mRNA sequencing data that support the findings of the study have been deposited in GEO with the accession number GSE139159. The single cell RNA sequencing data were generated previously (Baryawno et al. 2019) and are deposited in GEO (GSE128423). A portal for exploring the entire single cell atlas is available (portals.broadinstitute.org/single_cell/study/mouse-bone-marrow-stroma-in-homeostasis).

### REFERENCES

1. Baryawno N, Przybylski D, Kowalczyk M S, Kfoury Y, Severe N, Gustafsson K, Kokkaliaris K D, Mercier F, Tabaka M, Hofree M, Dionne D, Papazian A, Lee D, Ashenberg O, Subramanian A, Vaishnav E D, Rozenblatt-Rosen O, Regev A, Scadden D T. A cellular taxonomy of the bone marrow stroma in homeostasis and leukemia. Cell 2019; 177:1915-1932.
2. Behrmann L, Wellbrock J, Fiedler W. Acute myeloid leukemia and the bone marrow niche—take a closer look. Front Oncol 2018; 8:444.
3. Bond M R, Hanover J A. A little sugar goes a long way: the cell biology of O-GlcNAc. J Cell Biol 2015; 208:869-880.
4. Boyd A L, Aslostovar L, Reid J, Ye W, Tanasijevic B, Porras D P, Shapovalova Z, Almakadi M, Foley R, Leber B, Xenocostas A, Bhatia M. Identification of chemotherapy-induced leukemic-regenerating cells reveals a transient vulnerability of human AML recurrence. Cancer Cell 2018; 34:483-498.
5. Cantor J R, Abu-Remaileh M, Kanarek N, Freinkman E, Gao X, Louissaint A Jr, Lewis C A, Sabatini D M. Physiologic medium rewires cellular metabolism and reveals uric acid as an endogenous inhibitor of UMP synthase. Cell 2017; 169:258-272.
6. Chen E Y, Tan C M, Kou Y, Duan Q, Wang Z, Meirelles G V, Clark N R, Ma′ayan A. Enrichr: interactive and collaborative HTML5 gene list enrichment analysis tool. BMC Bioinformatics 2013; 14:128.
7. Chen W L, Wang Y Y, Zhao A, Xia L, Xie G, Su M, Zhao L, Liu J, Qu C, Wei R, Rajani C, Ni Y, Cheng Z, Chen Z, Chen S J, Jia W. Enhanced fructose utilization mediated by SLC2A5 is a unique metabolic feature of acute myeloid leukemia with therapeutic potential. Cancer Cell 2016; 30:779-791.
8. Chong J, Soufan O, Li C, Caraus I, Li S, Bourque G, Wishart D S, Xia J. MetaboAnalyst 4.0: towards more transparent and integrative metabolomics analysis. Nucleic Acids Res 2018; 46:W486-W494.
9. Corral J, Lavenir I, Impey H, Warren A J, Forster A, Larson T A, Bell S, McKenzie A N, King G, Rabbitts T H. An Mll-AF9 fusion gene made by homologous recombination causes acute leukemia in chimeric mice: a method to create fusion oncogenes. Cell 1996; 85:853-861.
10. Davidson S M, Papagiannakopoulos T, Olenchock B A, Heyman J E, Keibler M A, Luengo A, Bauer M R, Jha A K, O'Brien J P, Pierce K A, Gui D Y, Sullivan L B, Wasylenko T M, Subbaraj L, Chin C R, Stephanopolous G, Mott B T, Jacks T, Clish C B, Vander Heiden M G. Environment impacts the metabolic dependencies of Ras-driven non-small cell lung cancer. Cell Metab 2016; 23:517-528.
11. Dietmair S, Timmins N E, Gray P P, Nielsen L K, Kromer J O. Towards quantitative metabolomics of mammalian cells: development of a metabolite extraction protocol. Anal Biochem 2010; 404:155-164.
12. Ding L, Ley T J, Larson D E, Miller C A, Koboldt D C, Welch J S, Ritchey J K, Young M A, Lamprecht T, McLellan M D, McMichael J F, Wallis J W, Lu C, Shen D, Harris C C, Dooling D J, Fulton R S, Fulton L L, Chen K, Schmidt H, Kalicki-Veizer J, Magrini V J, Cook L, McGrath S D, Vickery T L, Wendl M C, Heath S, Watson M A, Link D C, Tomasson M H, Shannon W D, Payton J E, Kulkarni S, Westervelt P, Walter M J, Graubert T A, Mardis E R, Wilson R K, DiPersio J F. Clonal evolution in relapsed acute myeloid leukaemia revealed by whole-genome sequencing. Nature 2012a; 481:506-510.
13. Ding L, Saunders T L, Enikolopov G, Morrison S J. Endothelial and perivascular cells maintain haematopoietic stem cells. Nature 2012b; 481:457-62.
14. German N J, Yoon H, Yusuf R Z, Murphy J P, Finley L W, Laurent G, Haas W, Satterstrom F K, Guarnerio J, Zaganjor E, Santos D, Pandolfi P P, Beck A H, Gygi S P, Scadden D T, Kaelin W G Jr, Haigis M C. PHD3 loss in cancer enables metabolic reliance on fatty acid oxidation via deactivation of ACC2. Mol Cell 2016; 63:1006-1020.
15. Gregory M A, Nemkov T, Park H J, Zaberezhnyy V, Gehrke S, Adane B, Jordan C T, Hansen K C, D'Alessandro A, DeGregori J. Targeting Glutamine Metabolism and Redox State for Leukemia Therapy. Clin Cancer Res 2019; 25:4079-4090.
16. Häussinger D, Schliess F. Glutamine metabolism and signaling in the liver. Front Biosci 2007; 12:371-391.
17. Heckl D, Kowalczyk M S, Yudovich D, Belizaire R, Puram R V, McConkey M E, Thielke A, Aster J C, Regev A, Ebert B L. Generation of mouse models of myeloid malignancy with combinatorial genetic lesions using CRISPR-Cas9 genome editing. Nat Biotechnol 2014; 32:941-946.
18. Jacque N, Ronchetti A M, Larrue C, Meunier G, Birsen R, Willems L, Saland E, Decroocq J, Maciel T T, Lambert M, Poulain L, Hospital M A, Sujobert P, Joseph L, Chapuis N, Lacombe C, Moura I C, Demo S, Sarry J E, Recher C, Mayeux P, Tamburini J, Bouscary D. Targeting glutaminolysis has antileukemic activity in acute myeloid leukemia and synergizes with BCL-2 inhibition. Blood 2015; 126:1346-1356.
19. Kanehisa M, Goto S. KEGG: Kyoto Encyclopedia of Genes and Genomes. Nucleic Acids Res 2000; 28:27-30.
20. Keller M A, Kampjut D, Harrison S A, Ralser M. Sulfate radicals enable a non-enzymatic Krebs cycle precursor. Nat Ecol Evol 2017; 1:83.
21. Kuleshov M V, Jones M R, Rouillard A D, Fernandez N F, Duan Q, Wang Z, Koplev S, Jenkins S L, Jagodnik K M, Lachmann A, McDermott M G, Monteiro C D, Gundersen G W, Ma'ayan A. Enrichr: a comprehensive gene set enrichment analysis web server 2016 update. Nucleic Acids Research 2016; 44:W90-W97.
22. Kültz D. Molecular and evolutionary basis of the cellular stress response. Annu Rev Physiol 2005; 67:225-257.
23. Kusumbe A P, Ramasamy S K, Starsichova A, Adams R H. Sample preparation for high-resolution 3D confocal imaging of mouse skeletal tissue. Nat Protoc 2015; 10:1904-1914.
24. Lagadinou E D, Sach A, Callahan K, Rossi R M, Neering S J, Minhajuddin M, Ashton J M, Pei S, Grose V, O'Dwyer K M, Liesveld J L, Brookes P S, Becker M W, Jordan C T. BCL-2 inhibition targets oxidative phosphorylation and selectively eradicates quiescent human leukemia stem cells. Cell Stem Cell 2013; 12:329-341.
25. Lemberg K M, Vornov J J, Rais R, Slusher B S. We're Not “DON” Yet: Optimal Dosing and Prodrug Delivery of 6-Diazo-5-oxo-L-norleucine. Mol Cancer Ther 2018; 17:1824-1832.
26. Magee J A. Comment on: Genomics of primary chemoresistance and remission induction failure in paediatric and adult acute myeloid leukaemia. Br J Haematol 2017; 176:5-6.
27. Matre P, Velez J, Jacamo R, Qi Y, Su X, Cai T, Chan S M, Lodi A, Sweeney S R, Ma H, Davis R E, Baran N, Haferlach T, Su X, Flores E R, Gonzalez D, Konoplev S, Samudio I, DiNardo C, Majeti R, Schimmer A D, Li W, Wang T, Tiziani S, Konopleva M. Inhibiting glutaminase in acute myeloid leukemia: metabolic dependency of selected AML subtypes. Oncotarget 2016; 7:79722-79735.
28. Muir A, Danai L V, Gui D Y, Waingarten C Y, Lewis C A, Vander Heiden M G. Environmental cystine drives glutamine anaplerosis and sensitizes cancer cells to glutaminase inhibition. Elife 2017; 6:e27713.
29. Naviaux R K. Metabolic features of the cell danger response. Mitochondrion 2014; 16:7-17.
30. Ni F, Yu W M, Li Z, Graham D K, Jin L, Kang S, Rossi M R, Li S, Broxmeyer H E, Qu C K. Critical role of ASCT2-mediated amino acid metabolism in promoting leukaemia development and progression. Nat Metab 2019; 1:390-403.
31. Nowell, P C. The clonal evolution of tumor cell populations. Science 1976; 194:23-28.
32. Pei S, Minhajuddin M, Callahan K P, Balys M, Ashton J M, Neering S J, Lagadinou E D, Corbett C, Ye H, Liesveld J L, O'Dwyer K M, Li Z, Shi L, Greninger P, Settleman J, Benes C, Hagen F K, Munger J, Crooks P A, Becker M W, Jordan C T. Targeting aberrant glutathione metabolism to eradicate human acute myelogenous leukemia cells. J Biol Chem 2013; 288:33542-33558.
33. Potter N, Miraki-Moud F, Ermini L, Titley I, Vijayaraghavan G, Papaemmanuil E, Campbell P, Gribben J, Taussig D, Greaves M. Single cell analysis of clonal architecture in acute myeloid leukaemia. Leukemia 2019; 33, 1113-1123.
34. Sykes D B, Kfoury Y S, Mercier F E, Wawer M J, Law J M, Haynes M K, Lewis T A, Schajnovitz A, Jain E, Lee D, Meyer H, Pierce K A, Tolliday N J, Waller A, Ferrara S J, Eheim A L, Stoeckigt D, Maxcy K L, Cobert J M, Bachand J, Szekely B A, Mukherjee S, Sklar L A, Kotz J D, Clish C B, Sadreyev R I, Clemons P A, Janzer A, Schreiber S L, Scadden D T. Inhibition of dihydroorotate dehydrogenase overcomes differentiation blockade in acute myeloid leukemia. Cell 2016; 167:171-186.
35. Tardito S, Oudin A, Ahmed S U, Fack F, Keunen O, Zheng L, Miletic H, Sakariassen P Ø, Weinstock A, Wagner A, Lindsay S L, Hock A K, Barnett S C, Ruppin E, Mørkve S H, Lund-Johansen M, Chalmers A J, Bjerkvig R, Niclou S P, Gottlieb E. Glutamine synthetase activity fuels nucleotide biosynthesis and supports growth of glutamine-restricted glioblastoma. Nat Cell Biol 2015; 17:1556-1568.
36. Vassiliou G S, Cooper J L, Rad R, Li J, Rice S, Uren A, Rad L, Ellis P, Andrews R, Banerjee R, Grove C, Wang W, Liu P, Wright P, Arends M, Bradley A. Mutant nucleophosmin and cooperating pathways drive leukemia initiation and progression in mice. Nat Genet 2011; 43:470-475.
37. Wang K, Sanchez-Martin M, Wang X, Knapp K M, Koche R, Vu L, Nahas M K, He J, Hadler M, Stein E M, Tallman M S, Donahue A L, Frampton G M, Lipson D, Roels S, Stephens P J, Sanford E M, Brennan T, Otto G A, Yelensky R, Miller V A, Kharas M G, Levine R L, Ferrando A, Armstrong S A, Krivtsov A V. Patient-derived xenotransplants can recapitulate the genetic driver landscape of acute leukemias. Leukemia 2017; 31:151-158.
38. Wang Y H, Israelsen W J, Lee D, Yu V W C, Jeanson N T, Clish C B, Cantley L C, Vander Heiden M G, Scadden D T. Cell-state-specific metabolic dependency in hematopoiesis and leukemogenesis. Cell 2014; 158:1309-1323.
39. Weissman I L. Stem cells are units of natural selection for tissue formation, for germline development, and in cancer development. Proc Natl Acad Sci USA
40. Wishart D S, Tzur D, Knox C, Eisner R, Guo A C, Young N, Cheng D, Jewell K, Arndt D, Sawhney S, Fung C, Nikolai L, Lewis M, Coutouly M A, Forsythe I, Tang P, Shrivastava S, Jeroncic K, Stothard P, Amegbey G, Block D, Hau D D, Wagner J, Miniaci J, Clements M, Gebremedhin M, Guo N, Zhang Y, Duggan G E, Macinnis G D, Weljie A M, Dowlatabadi R, Bamforth F, Clive D, Greiner R, Li L, Marrie T, Sykes B D, Vogel H J, Querengesser L. HMDB: the Human Metabolome Database. Nucleic Acids Res 2007; 35:D521-D526.
41. Wishart D S, Feunang Y D, Marcu A, Guo A C, Liang K, Vazquez-Fresno R, Sajed T, Johnson D, Li C, Karu N, Sayeeda Z, Lo E, Assempour N, Berjanskii M, Singhal S, Arndt D, Liang Y, Badran H, Grant J, Serra-Cayuela A, Liu Y, Mandal R, Neveu V, Pon A, Knox C, Wilson M, Manach C, Scalbert A. HMDB 4.0: the human metabolome database for 2018. Nucleic Acids Res 2018; 46:D608-D617.
42. Ye H, Adane B, Khan N, Sullivan T, Minhajuddin M, Gasparetto M, Stevens B, Pei S, Balys M, Ashton J M, Klemm D J, Woolthuis C M, Stranahan A W, Park C Y, Jordan C T. Leukemic stem cells evade chemotherapy by metabolic adaptation to an adipose tissue niche. Cell Stem Cell 2016; 19:23-37.
43. Zhang W, Trachootham D, Liu J, Chen G, Pelicano H, Garcia-Prieto C, Lu W, Burger J A, Croce C M, Plunkett W, Keating M J, Huang P. Stromal control of cystine metabolism promotes cancer cell survival in chronic lymphocytic leukaemia. Nat Cell Biol 2012; 14:276-286.

1. A method of treating acute myeloid leukemia in a subject in need thereof, the method comprising administering to the subject an effective amount of at least one pyrimidine synthesis inhibitor and a chemotherapy treatment regimen, thereby treating acute myeloid leukemia in the subject,
wherein the chemotherapy treatment regimen is administered for a pre-designated period of time, and
wherein the pyrimidine synthesis inhibitor is administered in a single dose following completion of the chemotherapy treatment regimen.

2. The method of claim 1, further comprising administering a second dose of at least one pyrimidine synthesis inhibitor following completion of the chemotherapy treatment regimen.

3. (canceled)

4. (canceled)

5. The method of claim 1, wherein the pyrimidine synthesis inhibitor comprises a dihydroorotate dehydrogenase inhibitor or an orotate-phosphoribosyltransferase (OPRT) inhibitor.

6. The method of claim 1, wherein the pyrimidine synthesis inhibitor is selected from the group consisting of brequinar (BRQ), teriflunomide, leflunomide, ML390, pyrazofurin (PE), or an analog thereof.

7.-11. (canceled)

12. The method of claim 1, wherein the subject suffers from refractory or relapsed acute myeloid leukemia.

13. (canceled)

14. The method of claim 1, wherein the subject is a subject who relapses from complete remission of acute myeloid leukemia after induction chemotherapy.

15. (canceled)

16. (canceled)

17. The method of claim 1, further comprising administering an aspartate transporter inhibitor.

18. (canceled)

19. The method of claim 17, wherein the aspartate transporter inhibitor is selected from the group consisting of DL-threo-beta-benzyloxyaspartate (TBOA), aminooxyacetic acid (AOA), hydrazinosuccinic acid, beta-methylene-DL-aspartate, and combinations thereof

20. The method of claim 1, further comprising administering a glutamate-oxaloacetate transaminase 2 (GOT2) inhibitor.

21. The method of claim 20, wherein the GOT2 inhibitor is selected from the group consisting of DL-threo-beta-benzyloxyaspartate (TBOA), L-trans-Pyrrolidine-2,4-dicarboxylic acid (L-trans-2,4-PDC), 2-Amino-5,6,7,8-tetrahydro-4-(4-methoxyphenyl) (naphthalen-1-yl)-5-oxo-4H-chromene-3-carbonitrile (UCPH 101), an shRNA, and combinations thereof.

22. The method of claim 1, wherein the chemotherapy treatment regimen comprises administering cytarabine and doxorubicin to the subject for a period of 3-5 days, followed by administering cytarabine alone to the subject for a period of 2-4 days, and
wherein the pyrimidine synthesis inhibitor is administered for a period of time beginning 2-4 days after completing the chemotherapy treatment regimen.

23. (canceled)

24. The method of claim 22, wherein a second dose of pyrimidine synthesis inhibitor is administered nine to eleven days after completing the chemotherapy treatment regimen.

25.-44. (canceled)

45. A pharmaceutical composition comprising an effective amount of a pyrimidine synthesis inhibitor, an effective amount of at least one chemotherapeutic agent, and a pharmaceutically acceptable carrier, diluent, or excipient.

46. The pharmaceutical composition of claim 45, wherein the at least one chemotherapeutic agent comprises an antimetabolite agent and an anthracycline agent.

47. (canceled)

48. The pharmaceutical composition of claim 45, wherein the pyrimidine synthesis inhibitor comprises brequinar (BRQ) or an analog thereof.

49. The pharmaceutical composition of claim 45, further comprising an aspartate transporter inhibitor and/or a GOT2 inhibitor.

50. The pharmaceutical composition of claim 49, wherein the aspartate transporter inhibitor is selected from the group consisting of DL-threo-beta-benzyloxyaspartate (TBOA), aminooxyacetic acid (AOA), hydrazinosuccinic acid, beta-methylene-DL-aspartate, and combinations thereof.

51. (canceled)

52. The pharmaceutical composition of claim 49, wherein the GOT2 inhibitor is selected from the group consisting of DL-threo-beta-benzyloxyaspartate (TBOA), L-trans-Pyrrolidine-2,4-dicarboxylic acid (L-trans-2,4-PDC), 2-Amino-5,6,7,8-tetrahydro-4-(4-methoxyphenyl)-7-(naphthalen-1-yl)-5-oxo-4H-chromene-3-carbonitrile (UCPH 101), an shRNA, and combinations thereof.

53-59. (canceled)

60. A method of depleting chemoresistant acute myeloid leukemia cells in a subject in need thereof, the method comprising administering to the subject an effective amount of an aspartate transporter inhibitor or a GOT2 inhibitor and an induction chemotherapy treatment regimen, thereby depleting the chemoresistant acute myeloid leukemia cells in the subject.

61. The method of claim 60, wherein the aspartate transporter is SLC1A3.

62.-67. (canceled)
